(19) United States
(12) Patent Application Publication
(10) Pub. No.: US 2025/0072283 A1
(43) Pub. Date: Feb. 27, 2025

(54) ORGANIC COMPOUND, OPTOELECTRONIC DEVICE INCLUDING THE SAME, AND ELECTRONIC APPARATUS AND ELECTRONIC EQUIPMENT INCLUDING THE OPTOELECTRONIC DEVICE

(71) Applicant: Samsung Display Co., Ltd., Yongin-Si (KR)

(72) Inventors: Dongsun Yoo, Yongin-si, Gyeonggi-do (KR); Seran Kim, Yongin-si, Gyeonggi-do (KR); Hoilim Kim, Yongin-si, Gyeonggi-do (KR); Hwasook Ryu, Yongin-si, Gyeonggi-do (KR); Saerom Park, Yongin-si, Gyeonggi-do (KR); Hyejin Jung, Yongin-si, Gyeonggi-do (KR)

(21) Appl. No.: 18/809,352

(22) Filed: Aug. 20, 2024

(30) Foreign Application Priority Data
Aug. 21, 2023 (KR) 10-2023-0109124

(51) Int. Cl. H10K 85/60

(52) U.S. Cl. CPC H10K 85/6576; H10K 85/623; H10K 85/653; H10K 85/654; H10K 85/656; H10K 85/6572; H10K 85/6574

(57) ABSTRACT

An optoelectronic device includes a first electrode, a second electrode on the first electrode, a photoactive layer between the first electrode and the second electrode, and a first compound represented by the following chemical structure:

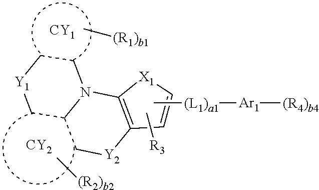

wherein constituents of the chemical structure are defined in this disclosure.

### CROSS-REFERENCE TO RELATED APPLICATION

This application is based on and claims priority under 35 U.S.C. § 119 to Korean Patent Application No. 10-2023-0109124 filed on Aug. 21, 2023 in the Korean Intellectual Property Office, the disclosure of which is incorporated by reference herein in its entirety.

### BACKGROUND

### 1. Field

Some embodiments relate to an organic compound, an optoelectronic device including the same, and an electronic apparatus and electronic equipment which include the optoelectronic device.

### 2. Description of the Related Art

Optoelectronic devices are devices that convert optical energy or optical signals into electrical energy or electrical signals. Examples of an optoelectronic device may include an optical or solar cell, which converts optical energy into electrical energy, an optical detector or sensor, which detects and converts optical energy into electrical signals, and the like.

Electronic apparatuses including optoelectronic devices and light-emitting devices are being developed. Light emitted from a light-emitting device may be reflected from an object (e.g., a finger of a user) in contact with an electronic apparatus, and the reflected light may be incident on an optoelectronic device. The contact input is recognized by the optoelectronic device's detection of incident light energy and its conversion to electrical signals. The optoelectronic device may be used as a fingerprint recognition sensor or the like.

### SUMMARY

Some embodiments include an organic compound with excellent deposition stability and heat resistance, an optoelectronic device having excellent external quantum efficiency, and a high-quality electronic apparatus and electronic equipment which employ the optoelectronic device.

Additional aspects will be set forth in part in the description which follows and, in part, will be apparent from the description, or may be learned by practice of the presented embodiments of the disclosure.

According to some embodiments, an optoelectronic device includes
a first electrode,
a second electrode on the first electrode,
a photoactive layer between the first electrode and the second electrode, and
a first compound represented by Formula 1:

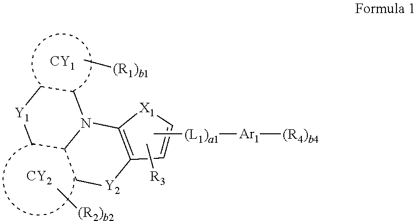

wherein, in Formula 1,
X₁ may be selected from oxygen (O), sulfur (S), selenium (Se), and tellurium (Te),
Y₁ and Y₂ may each independently be selected from a single bond, *—O—*′, *—S—*′, *—Se—*′, *—C(R₅)(R₆)—*′, *—Si(R₅)(R₆)—*′, *—Ge(R₅)(R₆)—*′, *—N(R₅)—*′, *—P(R₅)—*′, *—C(═O)—*′, *—C(═S)—*′, *—S(═O)—*′, and *—S(═O)₂—*′,
* and *′ each indicate a binding site to a neighboring atom,
L₁ may be a C₃-C₆₀carbocyclic group unsubstituted or substituted with at least one R₁₀ₐ or a C₁-C₆₀heterocyclic group unsubstituted or substituted with at least one R₁₀ₐ,
a1 may be an integer from 1 to 5,
Ar₁ may be a C₄-C₆₀ polycyclic group in which two or more cyclic groups are condensed with each other, and the two or more cyclic groups may each independently be a C₃-C₃₀ carbocyclic group or a C₁-C₃₀ heterocyclic group,
rings CY₁ and CY₂ may each independently be a C₅-C₆₀ carbocyclic group or a C₂-C₆₀ heterocyclic group,
b1, b2, and b4 may each independently be an integer from 0 to 10,
R₁ to R₆ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
two or more of R₁ to R₆ may optionally be linked to each other to form a C₅-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ may be
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof,
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof, or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, cyano group, a C₁-C₆₀ alkyl group, a C₁-C₆₀ alkoxy group, a phenyl group, a biphenyl group, or any combination thereof.

According to some embodiments, an electronic apparatus includes the optoelectronic device,
a light-emitting device not overlapping the optoelectronic device, and
a color filter, a color conversion layer, a touch screen layer, a polarizing layer, or any combination thereof.

According to some embodiments, electronic equipment includes the optoelectronic device,
wherein the electronic equipment is one of a flat panel display, a curved display, a computer monitor, a medical monitor, a television, an advertisement board, an indoor or outdoor lighting and/or signaling light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, a signboard, an automotive sensor, a home sensor, and a solar cell.

According to some embodiments, provided is the organic compound represented by Formula 1.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic view of an optoelectronic device according to an embodiment;

FIG. 2 is a schematic view of a light-emitting device included in an electronic apparatus according to an embodiment;

FIG. 3 is a schematic view of an optoelectronic device according to another embodiment;

FIG. 4 is a schematic view of an optoelectronic device according to another embodiment;

FIG. 5 is a schematic view of an electronic apparatus according to an embodiment;

FIG. 6 is a schematic view of an electronic apparatus according to another embodiment;

FIG. 7 is a schematic perspective view of electronic equipment including an optoelectronic device according to an embodiment;

FIG. 8 is a schematic view illustrating the exterior of a vehicle as electronic equipment including an optoelectronic device according to an embodiment; and

FIGS. 9A to 9C are schematic views illustrating the interior of the vehicle of FIG. 8.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are described below by referring to the figures, to explain aspects of the present description. As used herein, the term “and/or” includes any and all combinations of one or more of the associated listed items. Throughout the disclosure, the expression “at least one of a, b or c” indicates only a, only b, only c, both a and b, both a and c, both b and c, all of a, b, and c, or variations thereof.

According to some embodiments, an optoelectronic device includes:
a first electrode;
a second electrode on the first electrode;
a photoactive layer between the first electrode and the second electrode; and
a first compound represented by Formula 1:

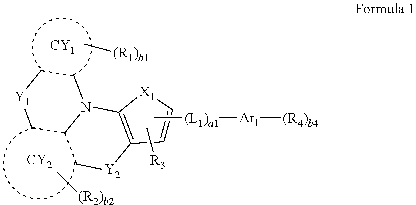

wherein, in Formula 1,
X₁ may be selected from oxygen (O), sulfur (S), selenium (Se), and tellurium (Te),
Y₁ and Y₂ may each independently be selected from a single bond, *—O—*′, *—S—*′, *—Se—*′, *—C(R₅)(R₆)—*′, *—Si(R₅)(R₆)—*′, *—Ge(R₅)(R₆)—*′, *—N(R₅)—*′, *—P(R₅)—*′, *—C(═O)—*′, *—C(═S)—*′, *—S(═O)—*′, and *—S(═O)₂—*′,
* and *′ each indicate a binding site to a neighboring atom,
L₁ may be a C₃-C₆₀ carbocyclic group unsubstituted or substituted with at least one R₁₀ₐ or a C₁-C₆₀ heterocyclic group unsubstituted or substituted with at least one R₁₀ₐ,
a1 may be an integer from 1 to 5,
Ar₁ may be a C₄-C₆₀ polycyclic group in which two or more cyclic groups are condensed with each other, and the two or more cyclic groups may each independently be a C₃-C₃₀ carbocyclic group or a C₁-C₃₀ heterocyclic group,
rings CY₁ and CY₂ may each independently be a C₅-C₆₀ carbocyclic group or a C₂-C₆₀ heterocyclic group,
b1, b2, and b4 may each independently be an integer from 0 to 10,
R₁ to R₆ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
two or more of R₁ to R₆ may optionally be linked to each other to form a C₅-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, cyano group, a C₁-C₆₀ alkyl group, a C₁-C₆₀ alkoxy group, a phenyl group, a biphenyl group, or any combination thereof.

In an embodiment, the optoelectronic device may further include a hole transport region between the first electrode and the photoactive layer and an electron transport region between the photoactive layer and the second electrode. The hole transport region may include a hole injection layer, a hole transport layer, an electron blocking layer, or any combination thereof. The electron transport region may include a buffer layer, a hole blocking layer, an electron control layer, an electron transport layer, an electron injection layer, or any combination thereof.

In an embodiment, the optoelectronic device may further include a second compound.

In an embodiment, the second compound may include fullerene, a fullerene derivative, subphthalocyanine, a subphthalocyanine derivative, thiophene, a thiophene derivative, a compound represented by one of Formulae 2-1 to 2-6, or a combination thereof.

In an embodiment, the second compound may be represented by one of Formulae 2-1 to 2-6:

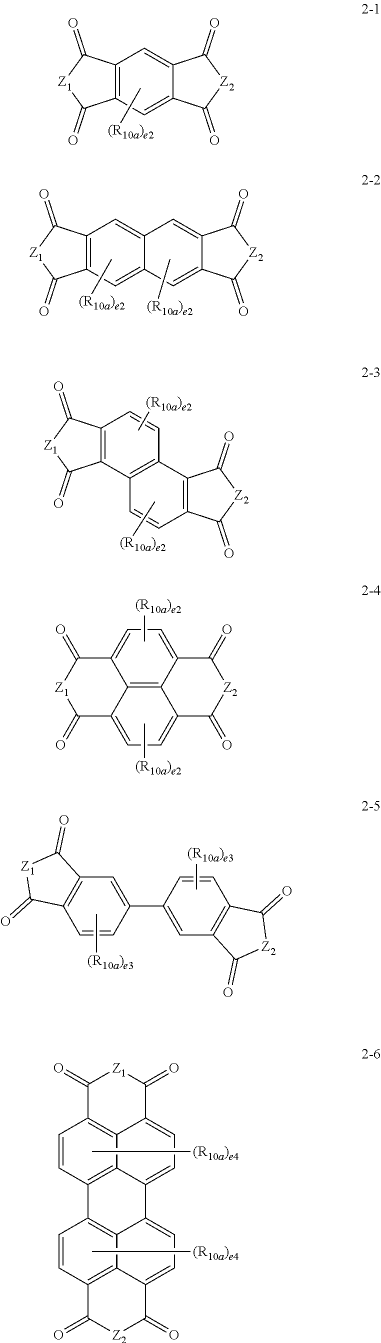

wherein in Formula 2-1 to 2-6
Z₁ an Z₂ may each independently be seleceted from O, S, N(R₂₄), C(R₂₄)(R₂₅), C(═O), C(═S), and C═C(R₂₄)(R₂₅),
R₂₄ and R₂₅ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
Q₁ to Q₃ and R₁₀ₐ may be the same as described herein,
e2 may be an integer from 0 to 2,
e3 may be an integer from 0 to 3, and
e4 may be an integer from 0 to 4.

In an embodiment, R₂₄ and R₂₅ may each independently be a C₁-C₆₀ alkyl group, a C₃-C₁₀ cycloalkyl group, a C₆-C₆₀ aryl group, or a C₁-C₆₀ heteroaryl group, each unsubstituted or substituted with deuterium, —F, —Cl, —Br, —I, a cyano group, a C₁-C₆₀ alkyl group, a C₁-C₆₀ alkoxy group, or any combination thereof.

In an embodiment, the second compound may be one of Compounds N1 to N43:

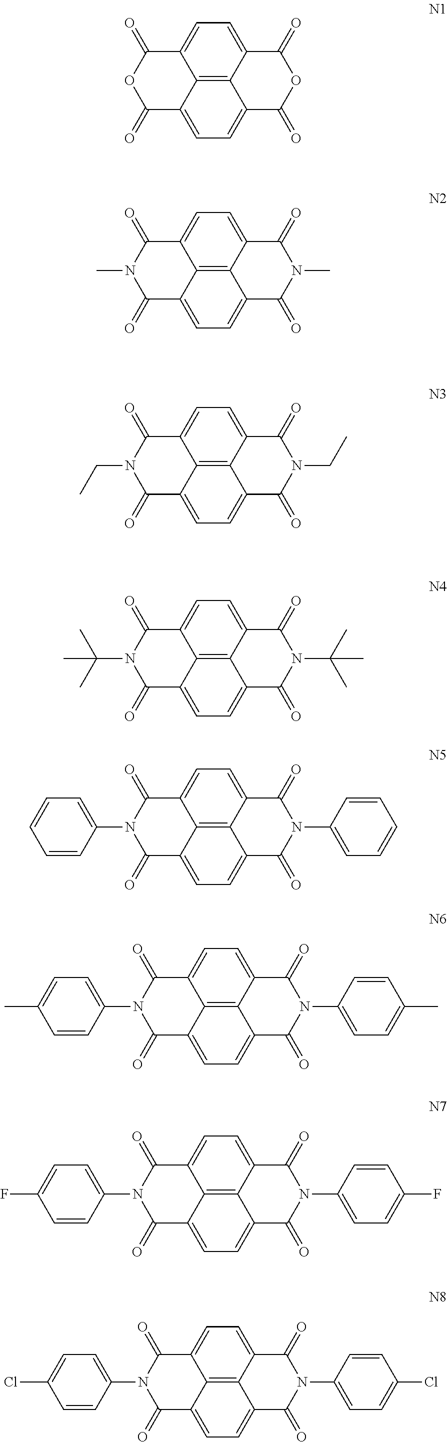

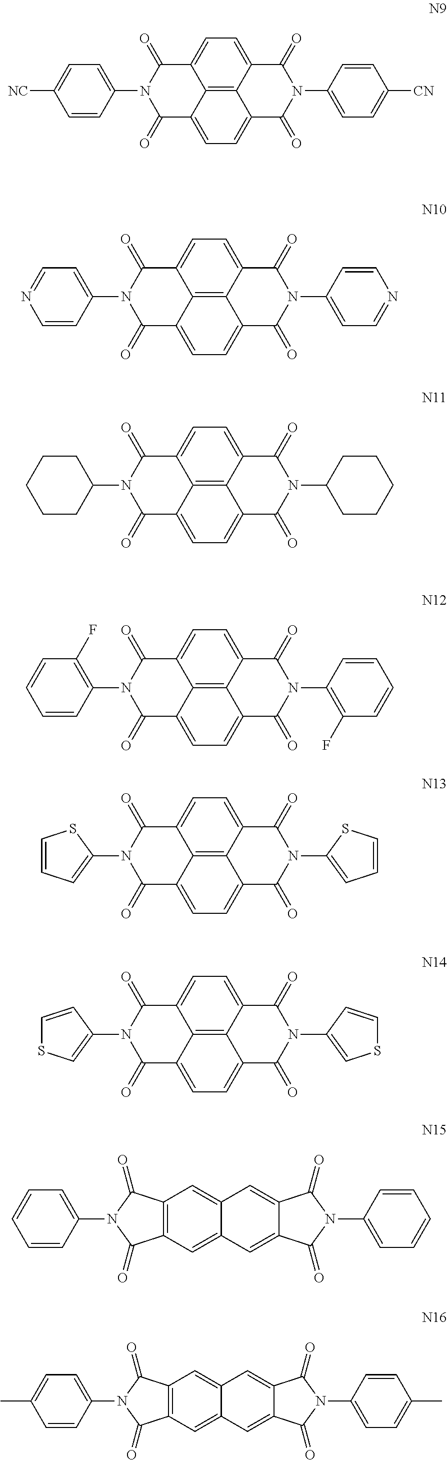

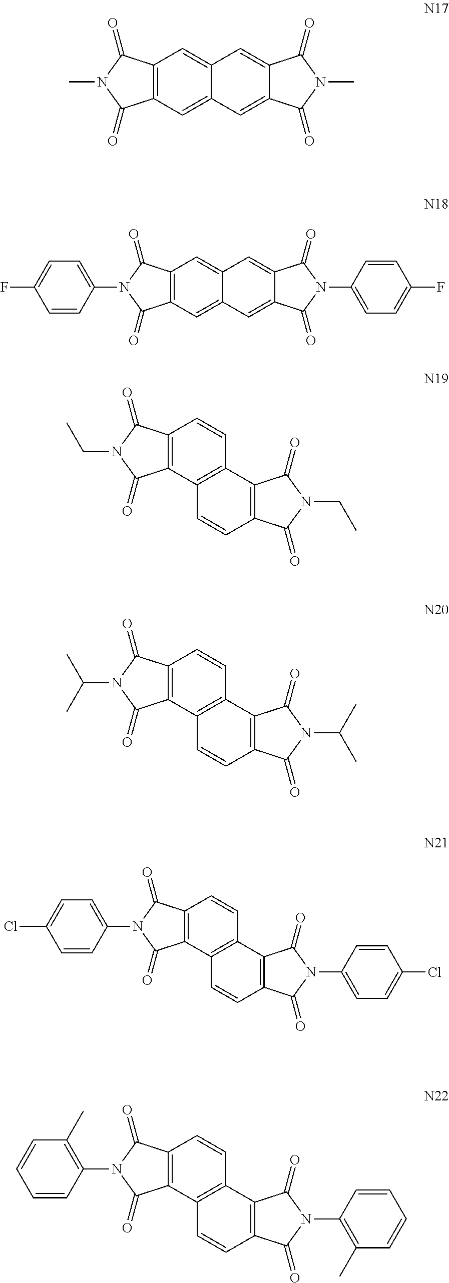

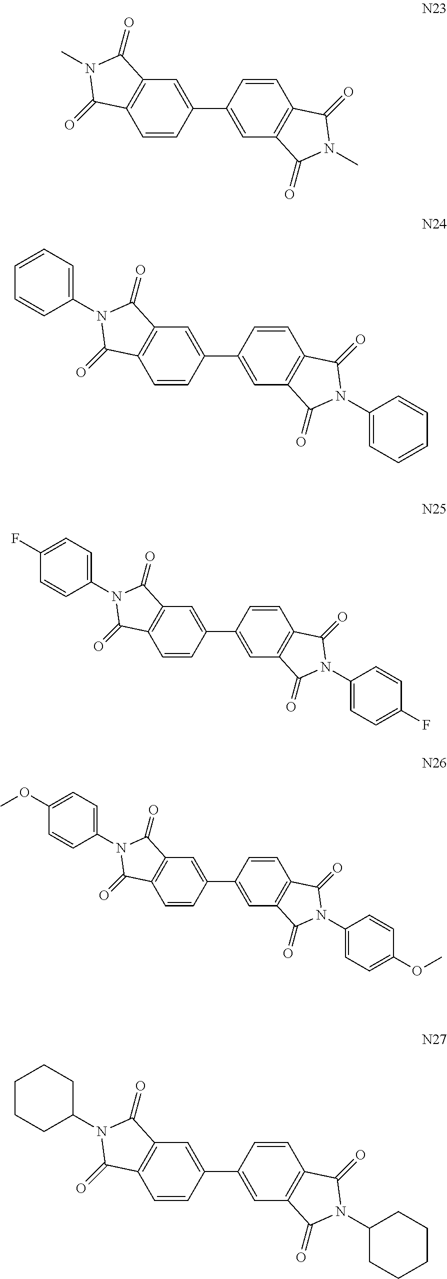

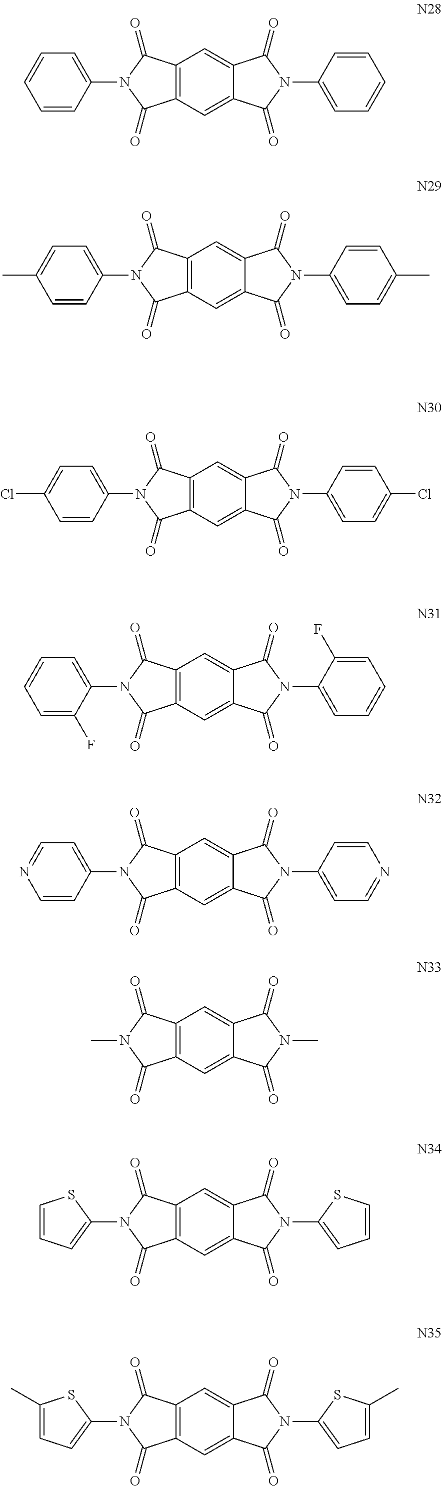

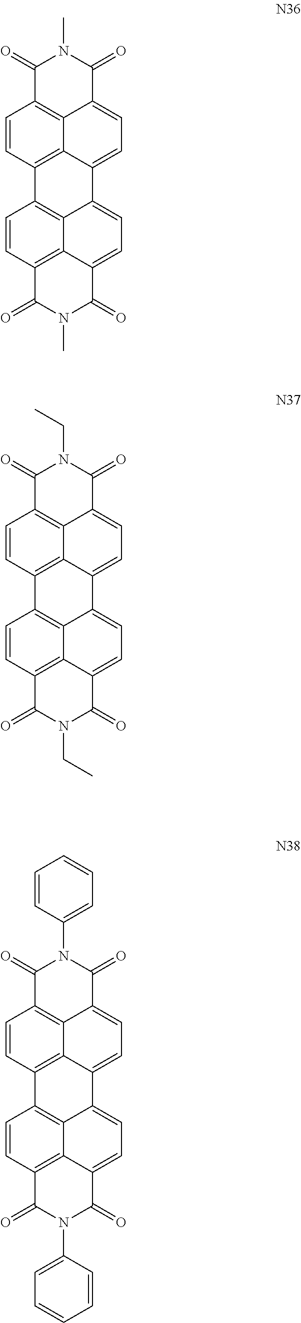 [SMILES: CN1C(=O)c2ccc3c4ccc5c6c(ccc(c7ccc(c2c37)C1=O)c64)C(=O)N(C)C5=O ; CCN1C(=O)c2ccc3c4ccc5c6c(ccc(c7ccc(c2c37)C1=O)c64)C(=O)N(CC)C5=O ; O=C1c2ccc3c4ccc5c6c(ccc(c7ccc(c2c37)C(=O)N1c1ccccc1)c64)C(=O)N(c1ccccc1)C5=O]

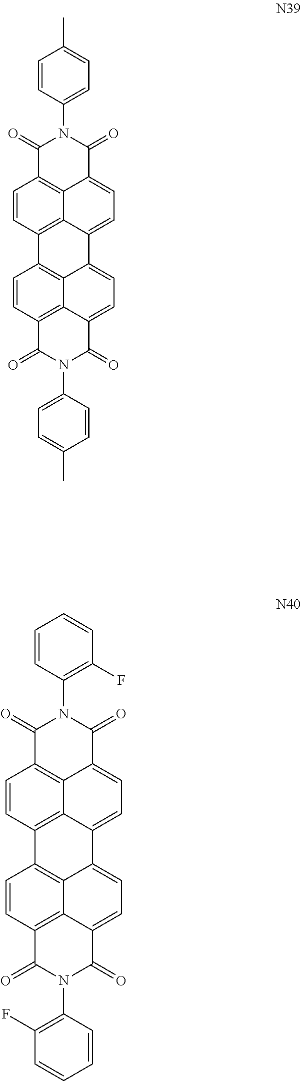 [SMILES: Cc1ccc(N2C(=O)c3ccc4c5ccc6c7c(ccc(c8ccc(c3c48)C2=O)c75)C(=O)N(c2ccc(C)cc2)C6=O)cc1 ; O=C1c2ccc3c4ccc5c6c(ccc(c7ccc(c2c37)C(=O)N1c1ccccc1F)c64)C(=O)N(c1ccccc1F)C5=O]

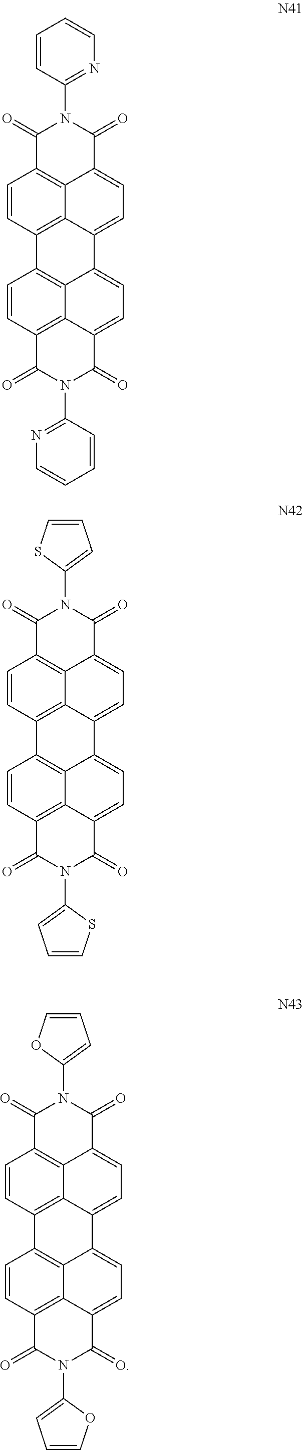

In an embodiment, the second compound may have a molecular weight of about 200 g/mol to about 1,500 g/mol.

In an embodiment, the photoactive layer may include the first compound and the second compound. The photoactive layer may include a mixture of the first compound and the second compound. The photoactive layer may be a single layer. In some embodiments, the first compound and the second compound may exist in the photoactive layer in a mixed state.

In other embodiments, the photoactive layer may include a first layer adjacent to the first electrode and a second layer adjacent to the second electrode. For example, the first layer may be disposed between the first electrode and the second layer. The first layer may be disposed between the hole transport region and the second layer. The second layer may be disposed between the first layer and the second electrode. The second layer may be disposed between the first layer and the electron transport region.

In an embodiment, the first layer may include the first compound. The first layer may not include the second compound.

In an embodiment, the second layer may include the second compound. The second layer may not include the first compound.

In some embodiments, the photoactive layer may have a double-layered structure that includes the first layer including the first compound and the second layer including the second compound. In some embodiments, the first compound and the second compound may exist in the photoactive layer in an unmixed state.

In other embodiments, the photoactive layer may further include a third layer between the first layer and the second layer. The third layer may include the first compound and the second compound. The third layer may include a mixture of the first compound and the second compound. In some embodiments, the first compound and the second compound may exist in the third layer in a mixed state. For example, the photoactive layer may have a triple-layered structure of i) the first layer including the first compound and not including the second compound, ii) the third layer including both the first compound and the second compound, and iii) the second layer including the second compound and not including the first compound.

In an embodiment, the photoactive layer may absorb light having a wavelength of about 400 nm to about 750 nm. In some embodiments, the photoactive layer may absorb red light, green light, blue light, near-infrared light, and/or any combination thereof. For example, the first compound in the photoactive layer may absorb light having a wavelength of about 400 nm to about 750 nm.

In an embodiment, the maximum absorption wavelength of the photoactive layer and/or the first compound may be in a range of about 500 nm to about 650 nm. For example, the maximum absorption wavelength of the photoactive layer and/or the first compound may be in a range of about 500 nm to about 600 nm, about 510 nm to about 565 nm, about 515 nm to about 550 nm, or about 520 nm to about 540 nm. In some embodiments, the photoactive layer and/or the first compound may absorb green light.

According to some embodiments, an electronic apparatus includes: the optoelectronic device;
a light-emitting device that does not overlap the optoelectronic device; and
a color filter, a color conversion layer, a touch screen layer, a polarizing layer, or any combination thereof.

In an embodiment, the light-emitting device may include an emission layer.

According to some embodiments, electronic equipment includes the optoelectronic device,
wherein the electronic equipment is one of a flat panel display, a curved display, a computer monitor, a medical monitor, a television, an advertisement board, an indoor or outdoor lighting and/or signaling light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, a signboard, an automotive sensor, a home sensor, and a solar cell.

According to some embodiments, provided is an organic compound (the first compound) represented by Formula 1:

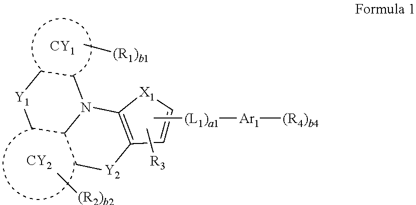

wherein, in Formula 1,
X₁ may be selected from oxygen (O), sulfur (S), selenium (Se), and tellurium (Te),
Y₁ and Y₂ may each independently be selected from a single bond, *—O—*′, *—S—*′, *—Se—*′, *—C(R₅)(R₆)—*′, *—Si(R₅)(R₆)—*′, *—Ge(R₅)(R₆)—*′, *—N(R₅)—*′, *—P(R₅)—*′, *—C(═O)—*′, *—C(═S)—*′, *—S(═O)—*′, and *—S(═O)₂—*′,
* and *′ each indicate a binding site to a neighboring atom,
L₁ may be a C₃-C₆₀ carbocyclic group unsubstituted or substituted with at least one R₁₀ₐ or a C₁-C₆₀heterocyclic group unsubstituted or substituted with at least one R₁₀ₐ,
a1 may be an integer from 1 to 5,
Ar₁ may be a C₄-C₆₀ polycyclic group in which two or more cyclic groups are condensed with each other, and the two or more cyclic groups may each independently be a C₃-C₃₀ carbocyclic group or a C₁-C₃₀ heterocyclic group,
rings CY₁ and CY₂ may each independently be a C₅-C₆₀ carbocyclic group or a C₂-C₆₀ heterocyclic group,
b1, b2, and b4 may each independently be an integer from 0 to 10,
R₁ to R₆ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
two or more of R₁ to R₆ may optionally be linked to each other to form a C₅-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, cyano group, a C₁-C₆₀ alkyl group, a C₁-C₆₀ alkoxy group, a phenyl group, a biphenyl group, or any combination thereof.

In an embodiment, the organic compound may be represented by one of Formulae 1-1 and 1-2:

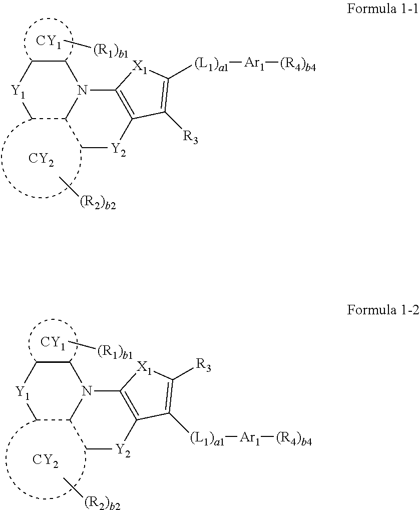

wherein, in Formulae 1-1 and 1-2,
X₁, Y₁, Y₂, L₁, a1, Ar₁, rings CY₁ and CY₂, b1, b2, b4, and R₁ to R₄ may be as defined in Formula 1.

In an embodiment, X₁ in Formulae 1, 1-1, and 1-2 may be selected from O, S, and Se.

In an embodiment, Y₁ and Y₂ in Formulae 1, 1-1, and 1-2 may each independently be a single bond, *—O—*′, *—S—*′, *—Se—*′, *—C(R₅)(R₆)—*′, *—Si(R₅)(R₆)—*′, and *—N(R₅)—*′.

In an embodiment, at least one of Y₁ and Y₂ in Formulae 1, 1-1, and 1-2 may be a single bond.

In an embodiment, L₁ in Formulae 1, 1-1, and 1-2 may be a C₆-C₆₀ arylene group unsubstituted or substituted with at least one R₁₀ₐ, a C₂-C₆₀ heteroarylene group unsubstituted or substituted with at least one R₁₀ₐ, a divalent non-aromatic condensed polycyclic group unsubstituted or substituted with at least one R₁₀ₐ, or a divalent non-aromatic condensed heteropolycyclic group unsubstituted or substituted with at least one R₁₀ₐ.

In an embodiment, L₁ in Formulae 1, 1-1, and 1-2 may be a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a selenophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluorene-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group, each unsubstituted or substituted with at least one R₁₀ₐ.

In an embodiment, L₁ in Formulae 1, 1-1, and 1-2 may be a group represented by one of Formulae 3-1 to 3-27:

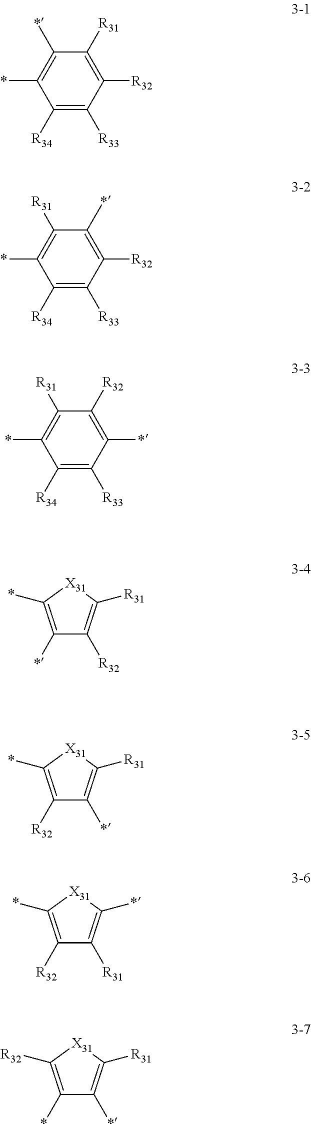

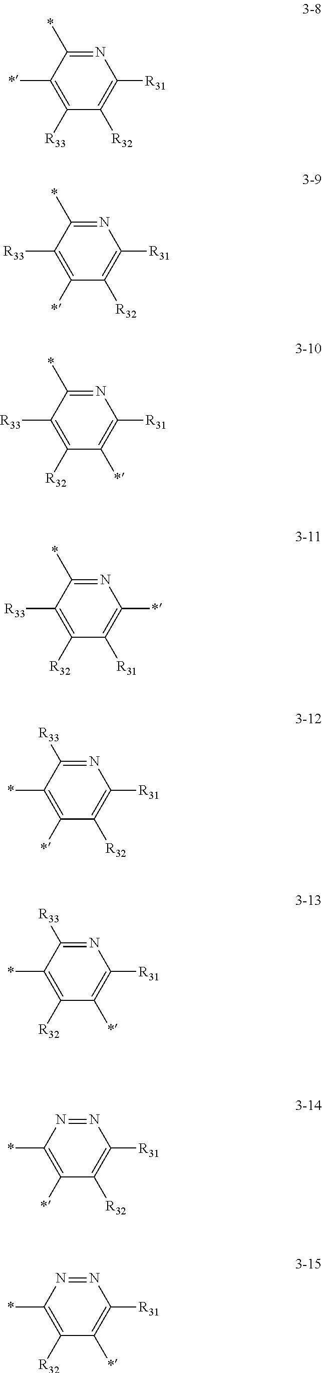

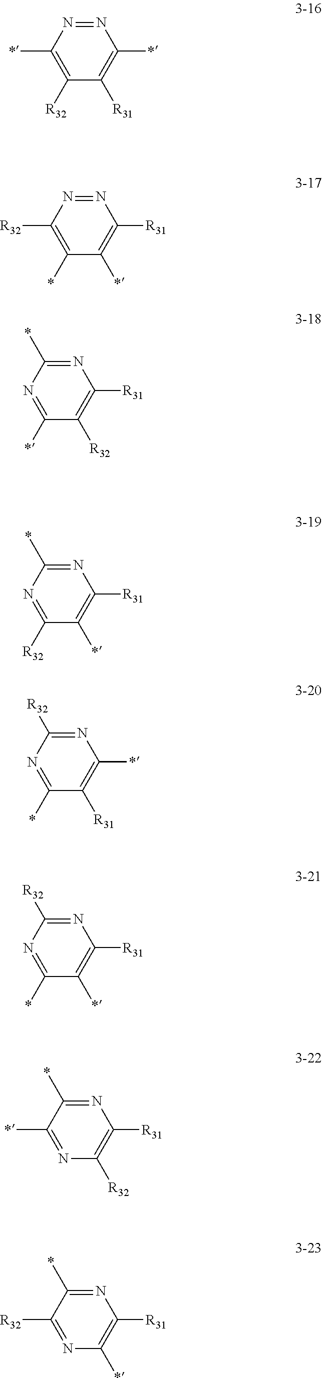

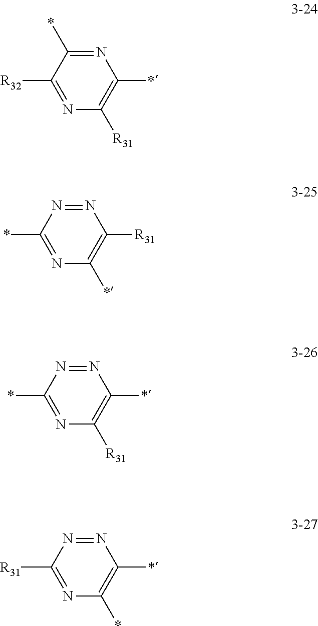

wherein, in Formulae 3-1 to 3-27,
X₃₁ may each independently be O, S, Se, and Te,
R₃₁ to R₃₄ may be each independently be the same as described herein in connection with R₁₀ₐ, and
* and *′ indicates a binding site to a neighboring atom.

In an embodiment, a1 in Formulae 1, 1-1, and 1-2 may be 1.

In an embodiment, the group represented by *—Ar₁—(R₄)_{b4} in Formulae 1, 1-1, and 1-2 may be a group represented by one of Formulae 4-1 to 4-6:

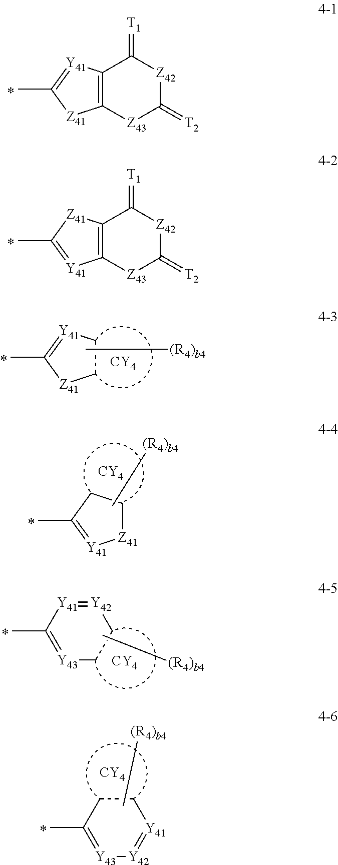

wherein, in Formulae 4-1 to 4-6,
T₁ and T₂ may each independently be O, S, Se, and Te,
Z₄₁ to Z₄₃ may each independently be selected from O, S, Se, N(R₄₁), C(R₄₁)(R₄₂), C(═O), C(═S), and C═C(R₄₁)(R₄₂),
Y₄₁ to Y₄₃ may each independently be N or C(R₄₃),
ring CY₄ may be a C₃-C₃₀ carbocyclic group or a C₁-C₃₀ heterocyclic group,
R₄₁ to R₄₃ may each independently be the same as described herein in connection with R₄,
R₄ and b4 may be as defined in Formula 1, and
* indicates a binding site to a neighboring atom.

In an embodiment, the group represented by*—Ar₁—(R₄)_{b4} in Formulae 1, 1-1, and 1-2 may be a group represented by any one of Formulae 5-1 to 5-15:

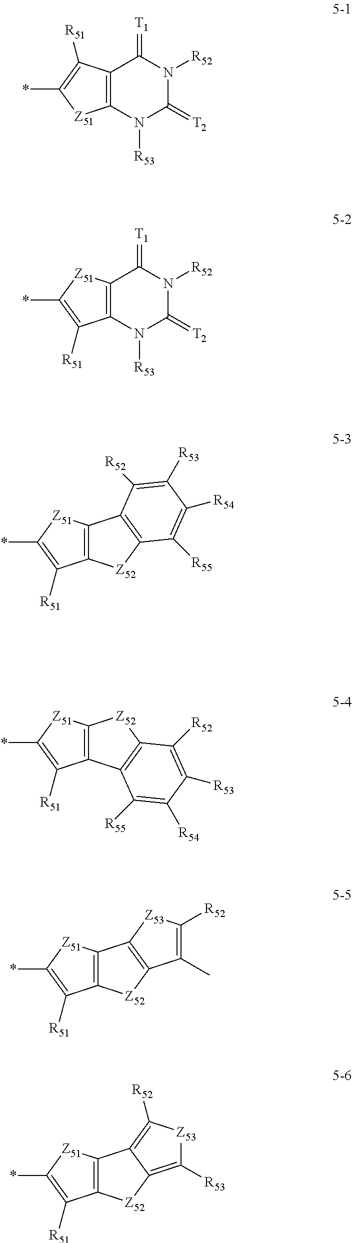

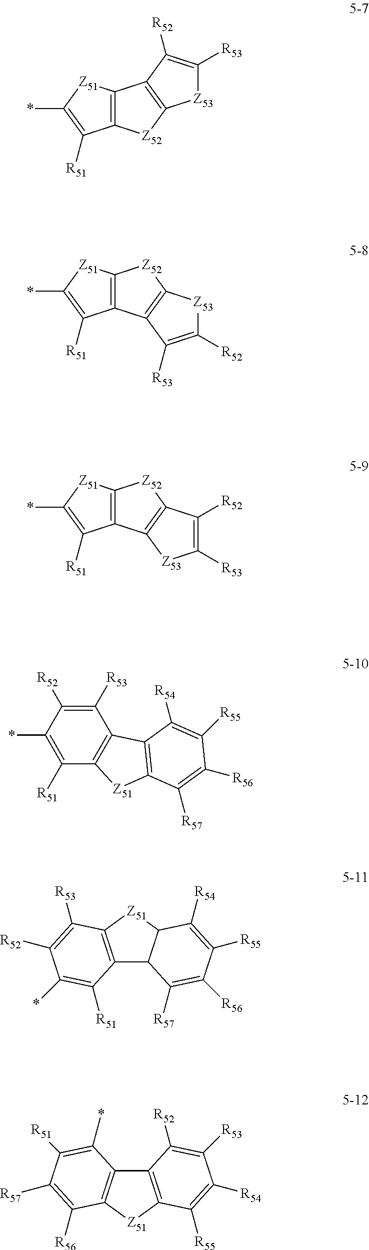

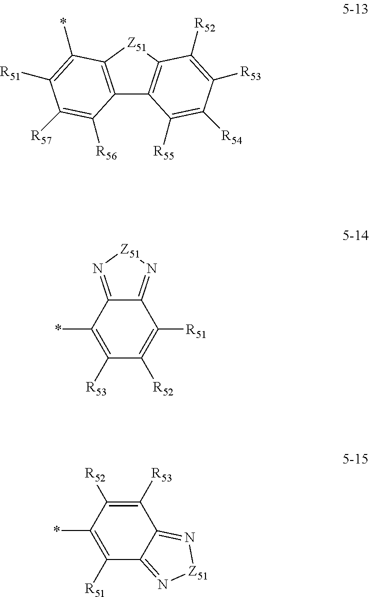

wherein, in Formulae 5-1 to 5-15,
T₁ and T₂ may each independently be O, S, Se, and Te,
Z₅₁ to Z₅₃ may each independently be selected from O, S, Se, N(R₄₁), C(R₄₁)(R₄₂), C(═O), C(═S), and C═C(R₄₁)(R₄₂),
R₄₁, R₄₂ and R₅₁ to R₅₇ may each independently be the same as described herein in connection with R₄,
two or more of R₅₁ to R₅₇ may optionally be linked to each other to form a C₅-C₃₀ carbocyclic group unsubstituted or substituted with at least one R₁₀ₐ or a C₁-C₃₀ heterocyclic group unsubstituted or substituted with at least one R₁₀ₐ, and
* indicates a binding site to a neighboring atom.

In an embodiment, the group represented by *—Ar₁—(R₄)_{b4} in Formulae 1, 1-1, and 1-2 may include an electron acceptor group.

In an embodiment, rings CY₁ and CY₂ in Formulae 1, 1-1, and 1-2 may each independently be a C₆-C₆₀ aryl group unsubstituted or substituted with at least one R₁₀ₐ or a C₂-C₆₀ heteroaryl group unsubstituted or substituted with at least one Ria.

In an embodiment, rings CY₁ and CY₂ in Formulae 1, 1-1, and 1-2 may each independently a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, 9H-fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluorene-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an iso-oxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group. For example, rings CY₁ and CY₂ may each independently be a benzene group or a naphthalene group.

In an embodiment, R₁ to R₆ in Formulae 1, 1-1, and 1-2 may each independently be hydrogen, deuterium, —F, —Cl, —Br, —I, a cyano group, a C₁-C₆₀ alkyl group unsubstituted or substituted with at least one R₁₀ₐ, a C₂-C₆₀ alkenyl group unsubstituted or substituted with at least one R₁₀ₐ, a C₁-C₆₀ alkoxy group unsubstituted or substituted with at least one R₁₀ₐ, or a C₃-C₆₀ aryl group unsubstituted or substituted with at least one Ria.

In an embodiment, the organic compound may have a highest occupied molecular orbital (HOMO) energy level of about −5.9 eV to about −5.2 eV, and the organic compound may have a lowest unoccupied molecular orbital (LUMO) energy level of about −3.5 eV to about −2.2 eV.

In an embodiment, the organic compound may be one of Compounds 1 to 72:

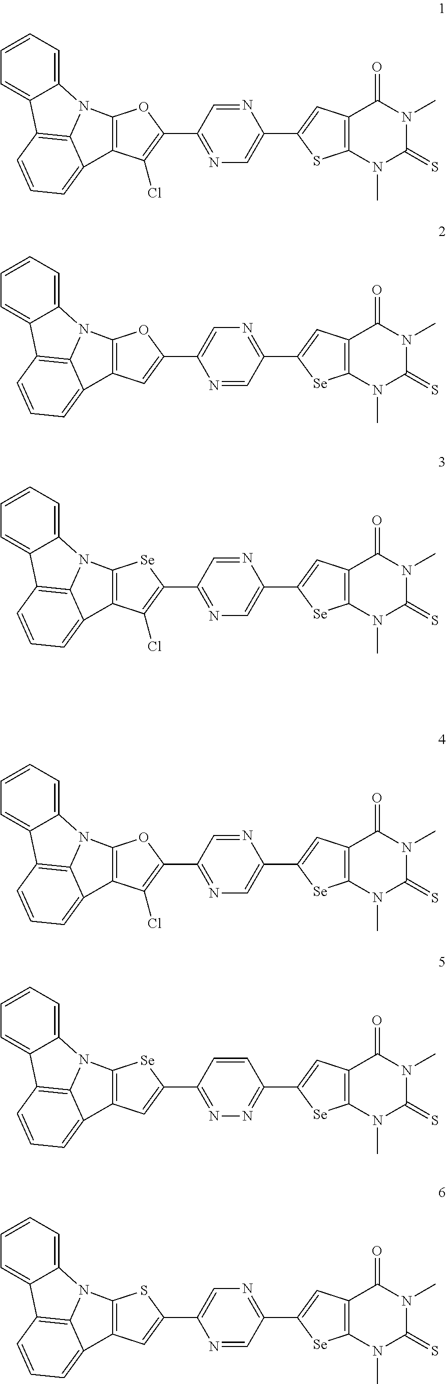 [SMILES: Cn1c(=O)c2cc(-c3cnc(-c4oc5c(c4Cl)c4cccc6c7ccccc7n5c64)cn3)sc2n(C)c1=S ; Cn1c(=O)c2cc(-c3cnc(-c4cc5c6cccc7c8ccccc8n(c5o4)c76)cn3)[se]c2n(C)c1=S ; Cn1c(=O)c2cc(-c3cnc(-c4[se]c5c(c4Cl)c4cccc6c7ccccc7n5c64)cn3)[se]c2n(C)c1=S ; Cn1c(=O)c2cc(-c3cnc(-c4oc5c(c4Cl)c4cccc6c7ccccc7n5c64)cn3)[se]c2n(C)c1=S ; Cn1c(=O)c2cc(-c3ccc(-c4cc5c6cccc7c8ccccc8n(c5[se]4)c76)nn3)[se]c2n(C)c1=S ; Cn1c(=O)c2cc(-c3cnc(-c4cc5c6cccc7c8ccccc8n(c5s4)c76)cn3)[se]c2n(C)c1=S]

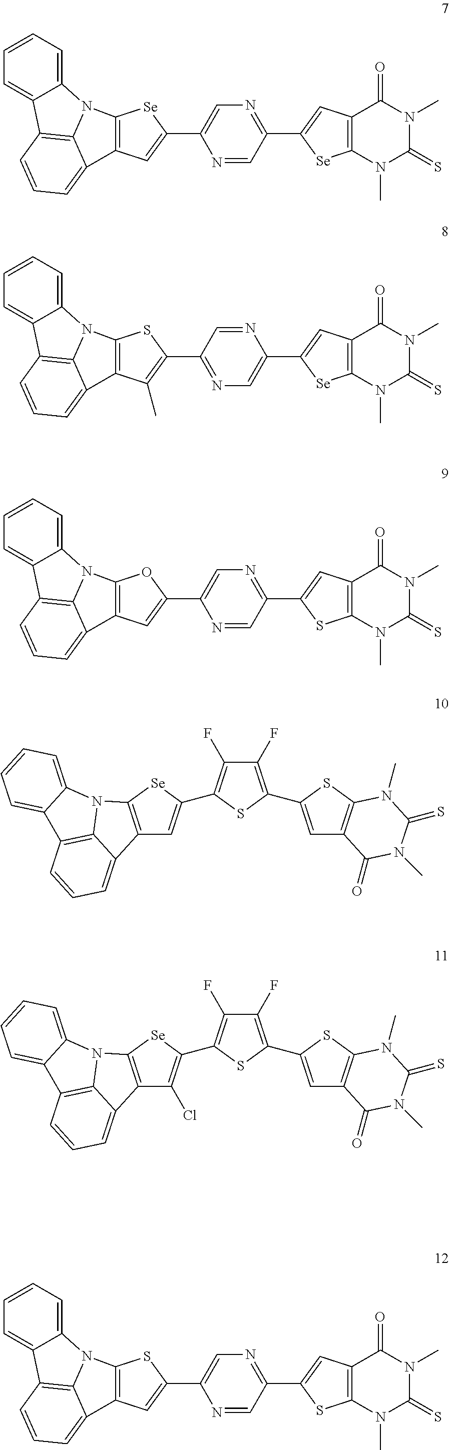 [SMILES: Cn1c(=O)c2cc(-c3cnc(-c4cc5c6cccc7c8ccccc8n(c5[se]4)c76)cn3)[se]c2n(C)c1=S ; Cc1c(-c2cnc(-c3cc4c(=O)n(C)c(=S)n(C)c4[se]3)cn2)sc2c1c1cccc3c4ccccc4n2c31 ; Cn1c(=O)c2cc(-c3cnc(-c4cc5c6cccc7c8ccccc8n(c5o4)c76)cn3)sc2n(C)c1=S ; Cn1c(=O)c2cc(-c3sc(-c4cc5c6cccc7c8ccccc8n(c5[se]4)c76)c(F)c3F)sc2n(C)c1=S ; Cn1c(=O)c2cc(-c3sc(-c4[se]c5c(c4Cl)c4cccc6c7ccccc7n5c64)c(F)c3F)sc2n(C)c1=S ; Cn1c(=O)c2cc(-c3cnc(-c4cc5c6cccc7c8ccccc8n(c5s4)c76)cn3)sc2n(C)c1=S]

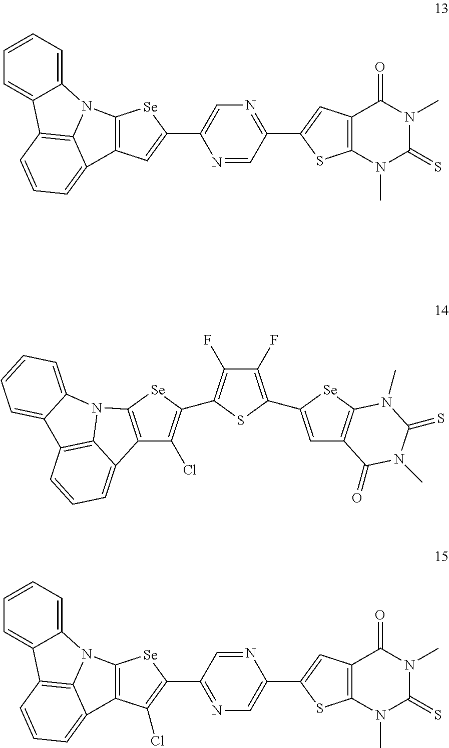 [SMILES: Cn1c(=O)c2cc(-c3cnc(-c4cc5c6cccc7c8ccccc8n(c5[se]4)c76)cn3)sc2n(C)c1=S ; Cn1c(=O)c2cc(-c3sc(-c4[se]c5c(c4Cl)c4cccc6c7ccccc7n5c64)c(F)c3F)[se]c2n(C)c1=S ; Cn1c(=O)c2cc(-c3cnc(-c4[se]c5c(c4Cl)c4cccc6c7ccccc7n5c64)cn3)sc2n(C)c1=S]

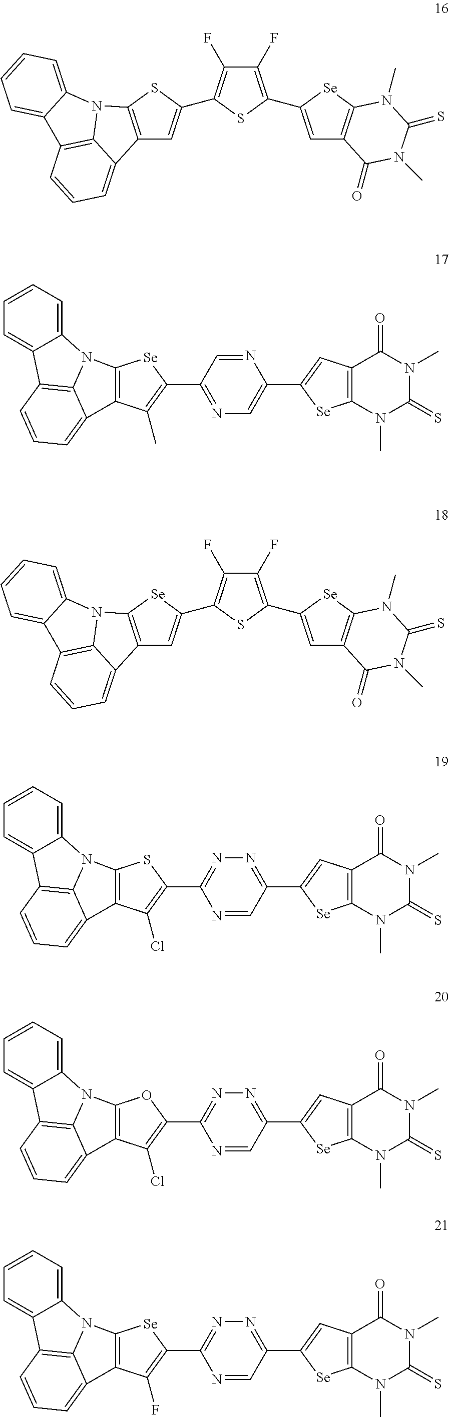 [SMILES: Cn1c(=O)c2cc(-c3sc(-c4cc5c6cccc7c8ccccc8n(c5s4)c76)c(F)c3F)[se]c2n(C)c1=S ; Cc1c(-c2cnc(-c3cc4c(=O)n(C)c(=S)n(C)c4[se]3)cn2)[se]c2c1c1cccc3c4ccccc4n2c31 ; Cn1c(=O)c2cc(-c3sc(-c4cc5c6cccc7c8ccccc8n(c5[se]4)c76)c(F)c3F)[se]c2n(C)c1=S ; Cn1c(=O)c2cc(-c3cnc(-c4sc5c(c4Cl)c4cccc6c7ccccc7n5c64)nn3)[se]c2n(C)c1=S ; Cn1c(=O)c2cc(-c3cnc(-c4oc5c(c4Cl)c4cccc6c7ccccc7n5c64)nn3)[se]c2n(C)c1=S ; Cn1c(=O)c2cc(-c3cnc(-c4[se]c5c(c4F)c4cccc6c7ccccc7n5c64)nn3)[se]c2n(C)c1=S]

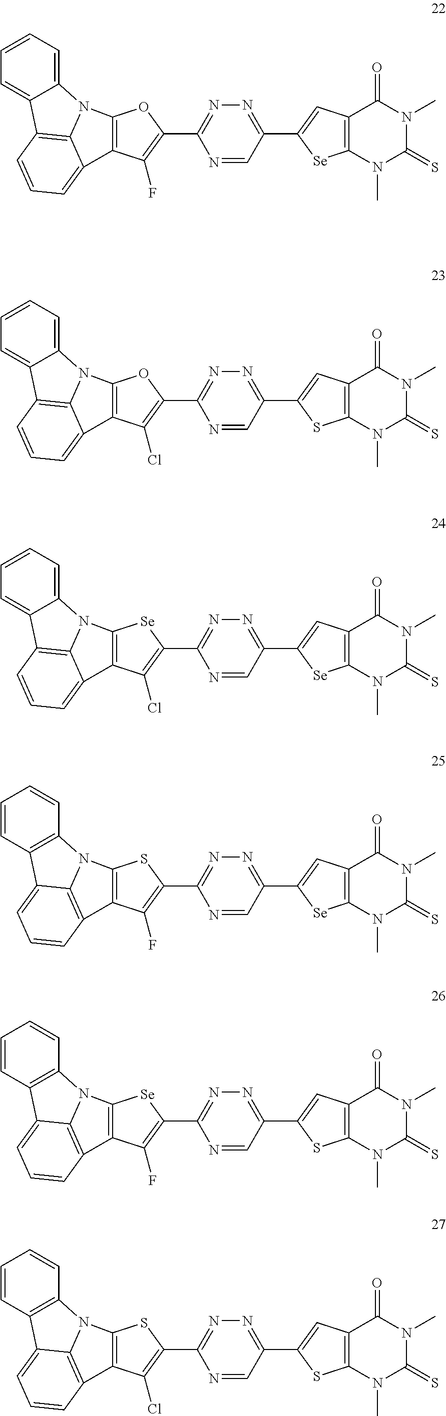 [SMILES: Cn1c(=O)c2cc(-c3cnc(-c4oc5c(c4F)c4cccc6c7ccccc7n5c64)nn3)[se]c2n(C)c1=S ; Cn1c(=O)c2cc(-c3cnc(-c4oc5c(c4Cl)c4cccc6c7ccccc7n5c64)nn3)sc2n(C)c1=S ; Cn1c(=O)c2cc(-c3cnc(-c4[se]c5c(c4Cl)c4cccc6c7ccccc7n5c64)nn3)[se]c2n(C)c1=S ; Cn1c(=O)c2cc(-c3cnc(-c4sc5c(c4F)c4cccc6c7ccccc7n5c64)nn3)[se]c2n(C)c1=S ; Cn1c(=O)c2cc(-c3cnc(-c4[se]c5c(c4F)c4cccc6c7ccccc7n5c64)nn3)sc2n(C)c1=S ; Cn1c(=O)c2cc(-c3cnc(-c4sc5c(c4Cl)c4cccc6c7ccccc7n5c64)nn3)sc2n(C)c1=S]

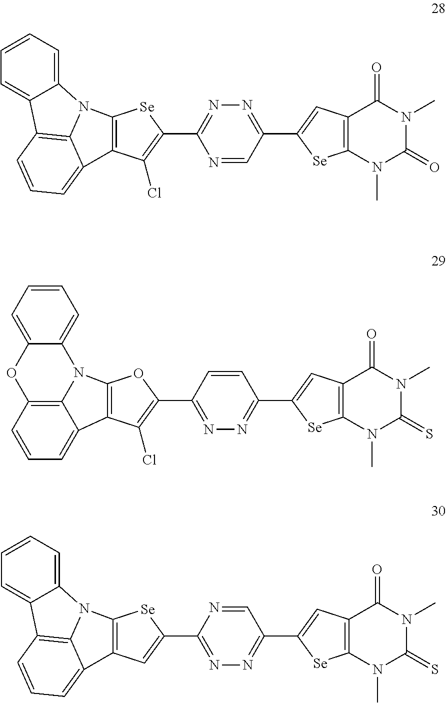 [SMILES: Cn1c(=O)c2cc(-c3cnc(-c4[se]c5c(c4Cl)c4cccc6c7ccccc7n5c64)nn3)[se]c2n(C)c1=O ; Cn1c(=O)c2cc(-c3ccc(-c4oc5c(c4Cl)c4cccc6c4n5-c4ccccc4O6)nn3)[se]c2n(C)c1=S ; Cn1c(=O)c2cc(-c3cnc(-c4cc5c6cccc7c8ccccc8n(c5[se]4)c76)nn3)[se]c2n(C)c1=S]

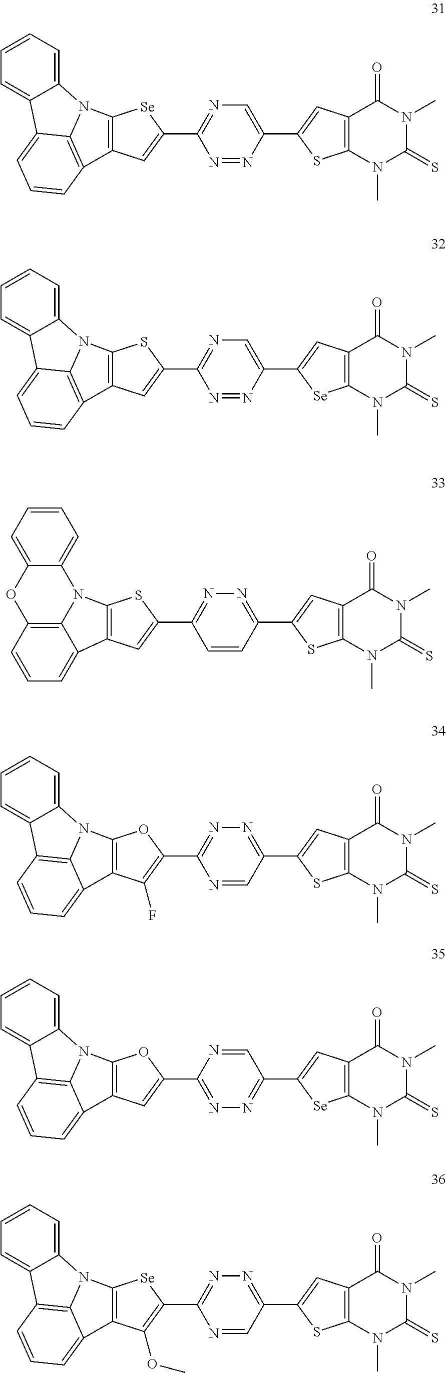 [SMILES: Cn1c(=O)c2cc(-c3cnc(-c4cc5c6cccc7c8ccccc8n(c5[se]4)c76)nn3)sc2n(C)c1=S ; Cn1c(=O)c2cc(-c3cnc(-c4cc5c6cccc7c8ccccc8n(c5s4)c76)nn3)[se]c2n(C)c1=S ; Cn1c(=O)c2cc(-c3ccc(-c4cc5c6cccc7c6n(c5s4)-c4ccccc4O7)nn3)sc2n(C)c1=S ; Cn1c(=O)c2cc(-c3cnc(-c4oc5c(c4F)c4cccc6c7ccccc7n5c64)nn3)sc2n(C)c1=S ; Cn1c(=O)c2cc(-c3cnc(-c4cc5c6cccc7c8ccccc8n(c5o4)c76)nn3)[se]c2n(C)c1=S ; COc1c(-c2ncc(-c3cc4c(=O)n(C)c(=S)n(C)c4s3)nn2)[se]c2c1c1cccc3c4ccccc4n2c31]

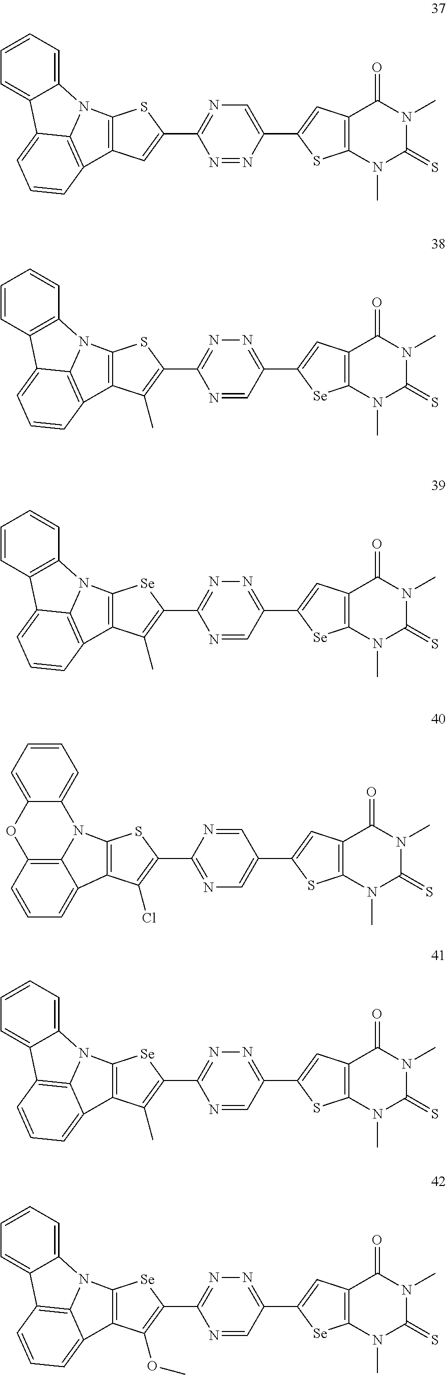 [SMILES: Cn1c(=O)c2cc(-c3cnc(-c4cc5c6cccc7c8ccccc8n(c5s4)c76)nn3)sc2n(C)c1=S ; Cc1c(-c2ncc(-c3cc4c(=O)n(C)c(=S)n(C)c4[se]3)nn2)sc2c1c1cccc3c4ccccc4n2c31 ; Cc1c(-c2ncc(-c3cc4c(=O)n(C)c(=S)n(C)c4[se]3)nn2)[se]c2c1c1cccc3c4ccccc4n2c31 ; Cn1c(=O)c2cc(-c3cnc(-c4sc5c(c4Cl)c4cccc6c4n5-c4ccccc4O6)nc3)sc2n(C)c1=S ; Cc1c(-c2ncc(-c3cc4c(=O)n(C)c(=S)n(C)c4s3)nn2)[se]c2c1c1cccc3c4ccccc4n2c31 ; COc1c(-c2ncc(-c3cc4c(=O)n(C)c(=S)n(C)c4[se]3)nn2)[se]c2c1c1cccc3c4ccccc4n2c31]

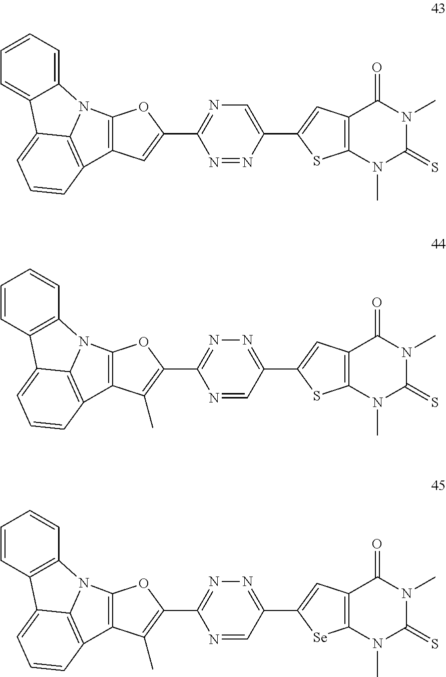 [SMILES: Cn1c(=O)c2cc(-c3cnc(-c4cc5c6cccc7c8ccccc8n(c5o4)c76)nn3)sc2n(C)c1=S ; Cc1c(-c2ncc(-c3cc4c(=O)n(C)c(=S)n(C)c4s3)nn2)oc2c1c1cccc3c4ccccc4n2c31 ; Cc1c(-c2ncc(-c3cc4c(=O)n(C)c(=S)n(C)c4[se]3)nn2)oc2c1c1cccc3c4ccccc4n2c31]

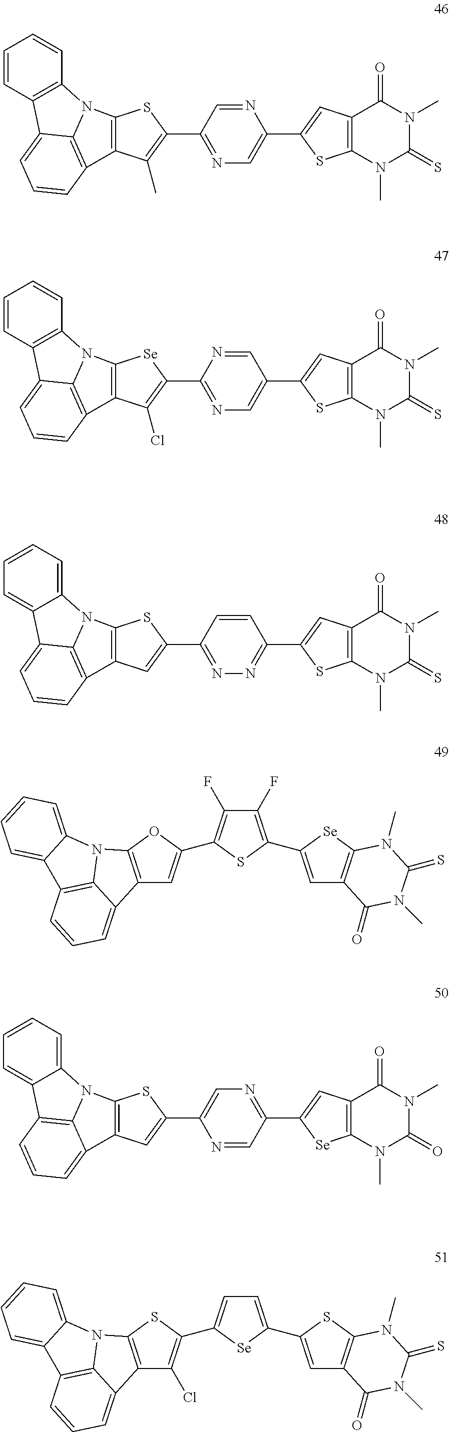 [SMILES: Cc1c(-c2cnc(-c3cc4c(=O)n(C)c(=S)n(C)c4s3)cn2)sc2c1c1cccc3c4ccccc4n2c31 ; Cn1c(=O)c2cc(-c3cnc(-c4[se]c5c(c4Cl)c4cccc6c7ccccc7n5c64)nc3)sc2n(C)c1=S ; Cn1c(=O)c2cc(-c3ccc(-c4cc5c6cccc7c8ccccc8n(c5s4)c76)nn3)sc2n(C)c1=S ; Cn1c(=O)c2cc(-c3sc(-c4cc5c6cccc7c8ccccc8n(c5o4)c76)c(F)c3F)[se]c2n(C)c1=S ; Cn1c(=O)c2cc(-c3cnc(-c4cc5c6cccc7c8ccccc8n(c5s4)c76)cn3)[se]c2n(C)c1=O ; Cn1c(=O)c2cc(-c3ccc(-c4sc5c(c4Cl)c4cccc6c7ccccc7n5c64)[se]3)sc2n(C)c1=S]

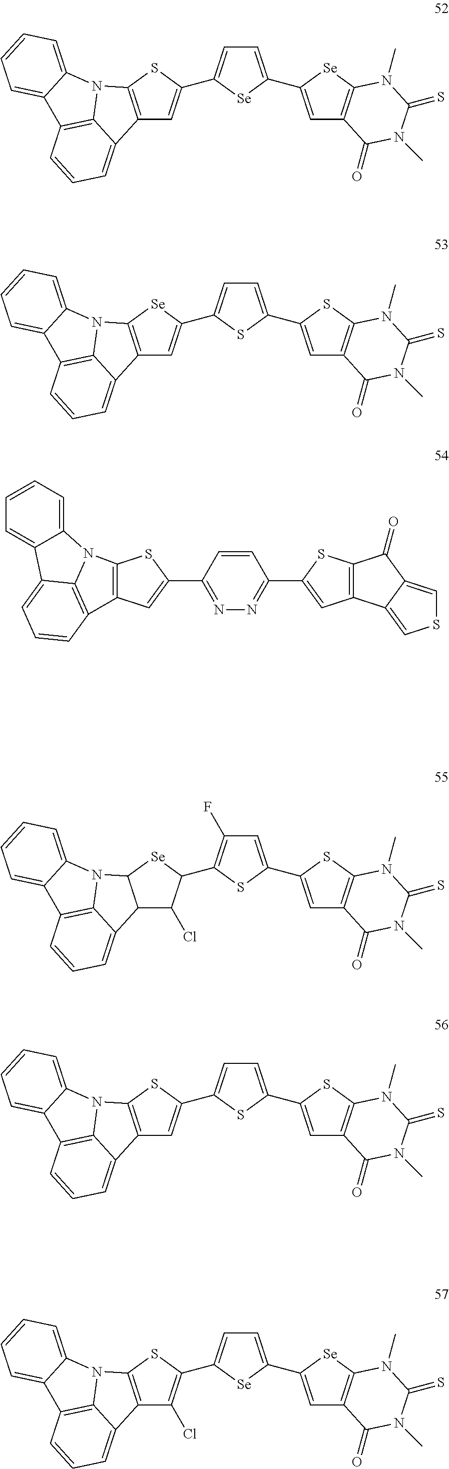 [SMILES: Cn1c(=O)c2cc(-c3ccc(-c4cc5c6cccc7c8ccccc8n(c5s4)c76)[se]3)[se]c2n(C)c1=S ; Cn1c(=O)c2cc(-c3ccc(-c4cc5c6cccc7c8ccccc8n(c5[se]4)c76)s3)sc2n(C)c1=S ; O=C1c2cscc2-c2cc(-c3ccc(-c4cc5c6cccc7c8ccccc8n(c5s4)c76)nn3)sc21 ; Cn1c(=O)c2cc(-c3cc(F)c(C4[Se]C5C(c6cccc7c8ccccc8n5c67)C4Cl)s3)sc2n(C)c1=S ; Cn1c(=O)c2cc(-c3ccc(-c4cc5c6cccc7c8ccccc8n(c5s4)c76)s3)sc2n(C)c1=S ; Cn1c(=O)c2cc(-c3ccc(-c4sc5c(c4Cl)c4cccc6c7ccccc7n5c64)[se]3)[se]c2n(C)c1=S]

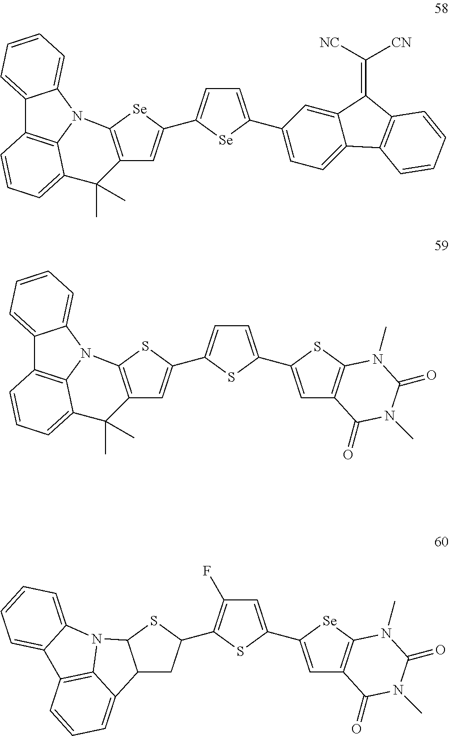 [SMILES: CC1(C)c2cc(-c3ccc(-c4ccc5c(c4)C(=C(C#N)C#N)c4ccccc4-5)[se]3)[se]c2-n2c3ccccc3c3cccc1c32 ; Cn1c(=O)c2cc(-c3ccc(-c4cc5c(s4)-n4c6ccccc6c6cccc(c64)C5(C)C)s3)sc2n(C)c1=O ; Cn1c(=O)c2cc(-c3cc(F)c(C4CC5c6cccc7c8ccccc8n(c67)C5S4)s3)[se]c2n(C)c1=O]

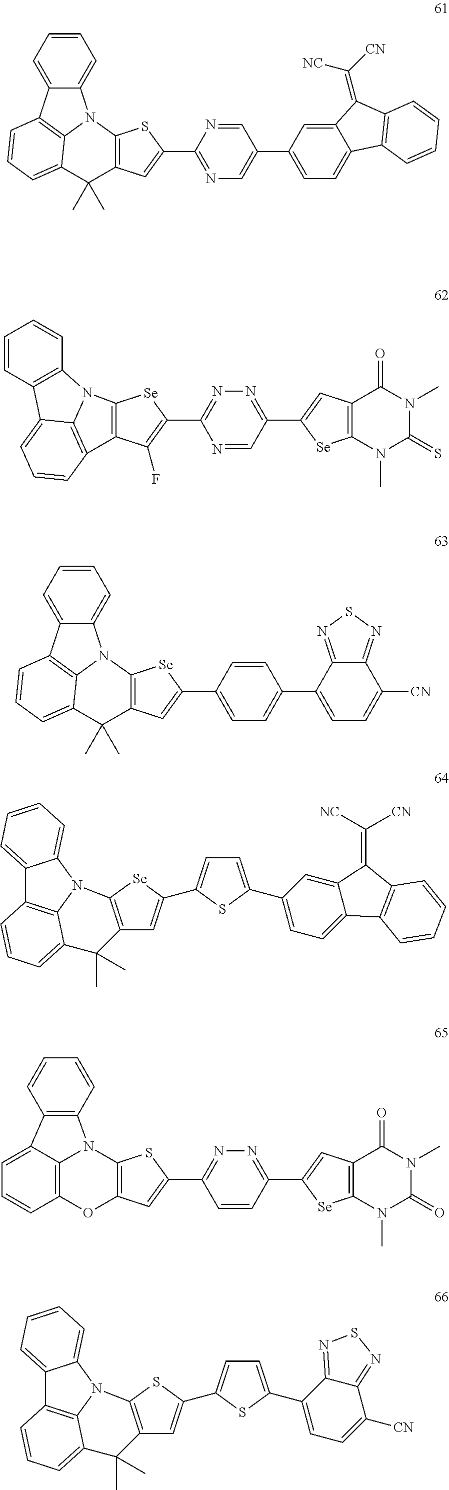 [SMILES: CC1(C)c2cc(-c3ncc(-c4ccc5c(c4)C(=C(C#N)C#N)c4ccccc4-5)cn3)sc2-n2c3ccccc3c3cccc1c32 ; Cn1c(=O)c2cc(-c3cnc(-c4[se]c5c(c4F)c4cccc6c7ccccc7n5c64)nn3)[se]c2n(C)c1=S ; CC1(C)c2cc(-c3ccc(-c4ccc(C#N)c5nsnc45)cc3)[se]c2-n2c3ccccc3c3cccc1c32 ; CC1(C)c2cc(-c3ccc(-c4ccc5c(c4)C(=C(C#N)C#N)c4ccccc4-5)s3)[se]c2-n2c3ccccc3c3cccc1c32 ; Cn1c(=O)c2cc(-c3ccc(-c4cc5c(s4)-n4c6ccccc6c6cccc(c64)O5)nn3)[se]c2n(C)c1=O ; CC1(C)c2cc(-c3ccc(-c4ccc(C#N)c5nsnc45)s3)sc2-n2c3ccccc3c3cccc1c32]

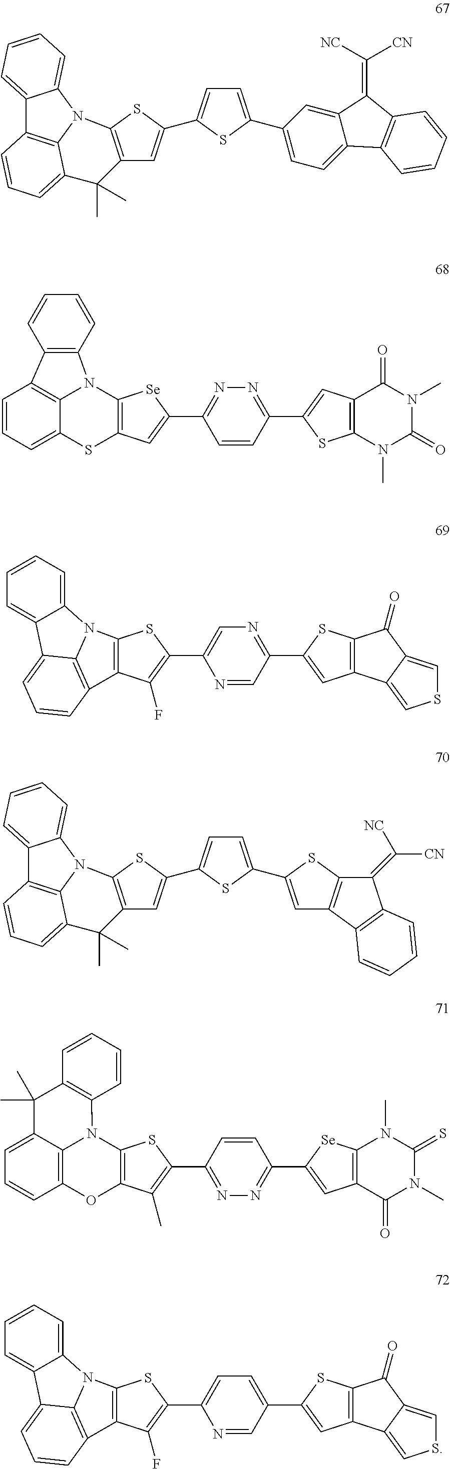 [SMILES: CC1(C)c2cc(-c3ccc(-c4ccc5c(c4)C(=C(C#N)C#N)c4ccccc4-5)s3)sc2-n2c3ccccc3c3cccc1c32 ; Cn1c(=O)c2cc(-c3ccc(-c4cc5c([se]4)-n4c6ccccc6c6cccc(c64)S5)nn3)sc2n(C)c1=O ; O=C1c2cscc2-c2cc(-c3cnc(-c4sc5c(c4F)c4cccc6c7ccccc7n5c64)cn3)sc21 ; CC1(C)c2cc(-c3ccc(-c4cc5c(s4)C(=C(C#N)C#N)c4ccccc4-5)s3)sc2-n2c3ccccc3c3cccc1c32 ; Cc1c(-c2ccc(-c3cc4c(=O)n(C)c(=S)n(C)c4[se]3)nn2)sc2c1Oc1cccc3c1N2c1ccccc1C3(C)C ; O=C1c2cscc2-c2cc(-c3ccc(-c4sc5c(c4F)c4cccc6c7ccccc7n5c64)nc3)sc21]

Generally, donor-acceptor-type materials containing both an electron donor moiety and an electron acceptor moiety are suitable in terms of light absorption and charge transfer characteristics. However, the electron donor moiety and the electron acceptor moiety are linked via a double bond that is easy to react with a nucleophile, and includes an amine structure that is easy to thermally decompose, and thus may have low heat resistance and be easily decomposed thermally in a deposition process.

However, the organic compound represented by Formula 1 has a structure in which the electron donor moiety and the electron acceptor moiety are linked via a single bond, and aryl groups linked to the amine are fused, and thus may have increased thermal stability by suppressing a deterioration reaction.

Therefore, an optoelectronic device employing the organic compound represented by Formula 1 may maintain high external quantum efficiency even under high-temperature processing conditions, and thus may simultaneously satisfy light absorption characteristics, charge transfer characteristics, and thermal characteristics.

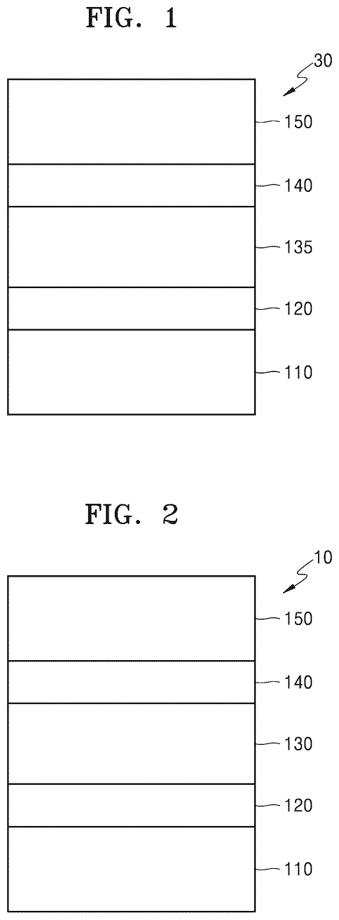

### [Description of FIGS. 1 and 2]

FIG. 1 is a schematic view of an optoelectronic device 30 according to an embodiment. The optoelectronic device 30 may include a first electrode 110, a hole transport region 120, a photoactive layer 135, an electron transport region 140, and a second electrode 150.

FIG. 2 is a schematic view of a light-emitting device 10. The light-emitting device 10 may include the first electrode 110, the hole transport region 120, the emission layer 130, the electron transport region 140, and the second electrode 150.

In an embodiment, the first electrode 110, the hole transport region 120, the electron transport region 140, and the second electrode 150 of the optoelectronic device 30 may be substantially integrated with the first electrode 110, the hole transport region 120, the electron transport region 140, and the second electrode 150 of the light-emitting device 10, respectively. In other embodiments, the first electrode 110, the hole transport region 120, the electron transport region 140, and the second electrode 150 of the optoelectronic device 30 may be spaced apart from the first electrode 110, the hole transport region 120, the electron transport region 140, and the second electrode 150 of the light-emitting device 10, respectively. Even in the latter case where the layers of the optoelectronic device 30 and the layers of the light-emitting device 10 are separated, the respective corresponding elements may substantially include the same material and be formed simultaneously.

Hereinafter, the structures of the optoelectronic device 30 and the light-emitting device 10 according to embodiments and a method of manufacturing the same will be described with reference to FIGS. 1 and 2.

### [First Electrode 110]

In FIG. 1, a substrate may be further arranged under the first electrode 110 or on the second electrode 150. As the substrate, a glass substrate or a plastic substrate may be used. The substrate may be a flexible substrate. For example, the substrate may include plastics with excellent heat resistance and durability, such as polyimide, polyethylene terephthalate (PET), polycarbonate, polyethylene naphtalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates hole injection.

The first electrode 110 may be a reflective electrode, a semi-transmissive electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In some embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may be a single-layered structure of a single layer or a multi-layered structure including a plurality of layers. For example, the first electrode 110 may have a triple-layered structure of ITO/Ag/ITO.

### [Hole Transport Region 120]

The hole transport region 120 may have i) a single-layered structure of a single layer containing a single material, ii) a single-layered structure of a single layer containing a plurality of different materials, or iii) a multi-layered structure having a plurality of layers including a plurality of different materials.

The hole transport region may include a hole injection layer, a hole transport layer, an emission auxiliary layer, an electron blocking layer, or any combination thereof.

For example, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, in which constituent layers of each structure are stacked sequentially from the first electrode 110.

The hole transport region may include a compound represented by Formula 201, a compound represented by Formula 202, or any combination thereof:

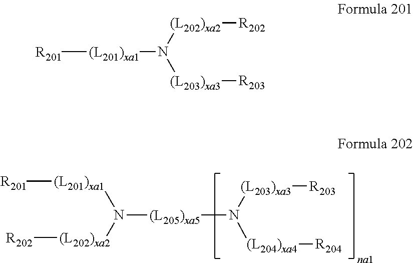

wherein, in Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S—*′, *—N(Q₂₀₁)—*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (for example, a carbazole group or the like) unsubstituted or substituted with at least one R₁₀ₐ (for example, Compound HT16 or the like),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and
na1 may be an integer from 1 to 4.

For example, each of Formulae 201 and 202 may include at least one of groups represented by Formulae CY201 to CY217:

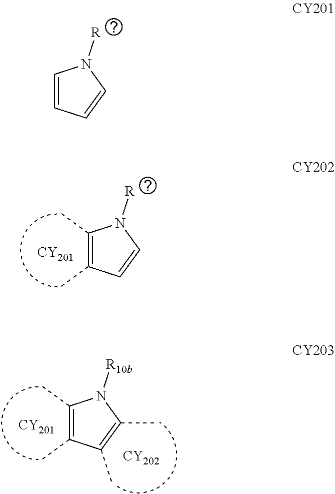

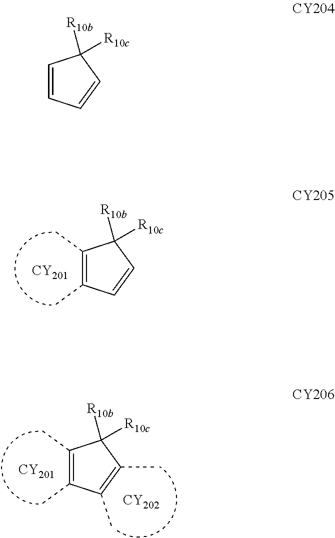

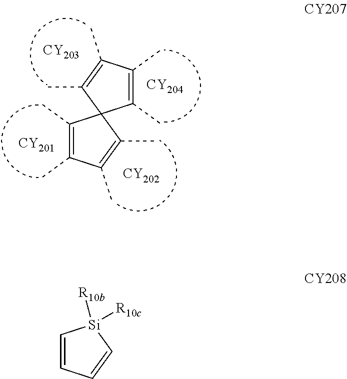

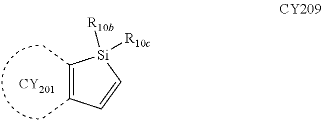

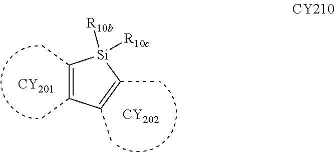

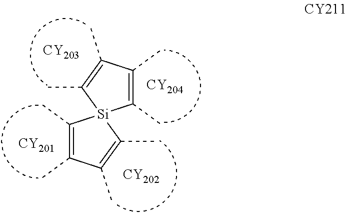

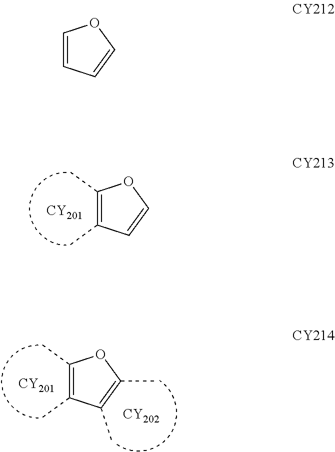

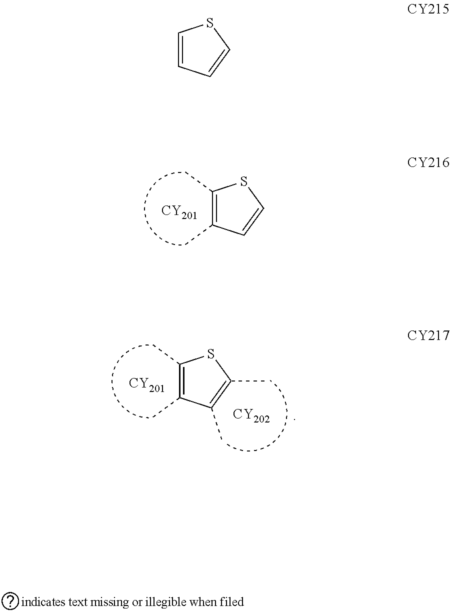

R_{10b} and R_{10c} in Formulae CY201 to CY217 may each be as described herein in connection with R₁₀ₐ, rings CY₂₀₁ to CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In an embodiment, rings CY₂₀₁ to CY₂₀₄ in Formulae CY201 to CY217 may each independently be a benzene group, a naphthalene group, a phenanthrene group, or an anthracene group.

In other embodiments, each of Formulae 201 and 202 may include at least one of the groups represented by Formulae CY201 to CY203.

In some embodiments, Formula 201 may include at least one of the groups represented by Formulae CY201 to CY203 and at least one of the groups represented by Formulae CY204 to CY217.

In some embodiments, in Formula 201, xa1 may be 1, R₂₀₁ may be a group represented by one of Formulae CY201 to CY203, xa2 may be 0, and R₂₀₂ may be a group represented by one of Formulae CY204 to CY207.

In some embodiments, each of Formulae 201 and 202 may not include the groups represented by Formulae CY201 to CY203.

In some embodiments, each of Formulae 201 and 202 may not include the groups represented by Formulae CY201 to CY203, and may include at least one of the groups represented by Formulae CY204 to CY217.

In some embodiments, each of Formulae 201 and 202 may not include the groups represented by Formulae CY201 to CY217.

In an embodiment, the hole transport region may include one of Compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

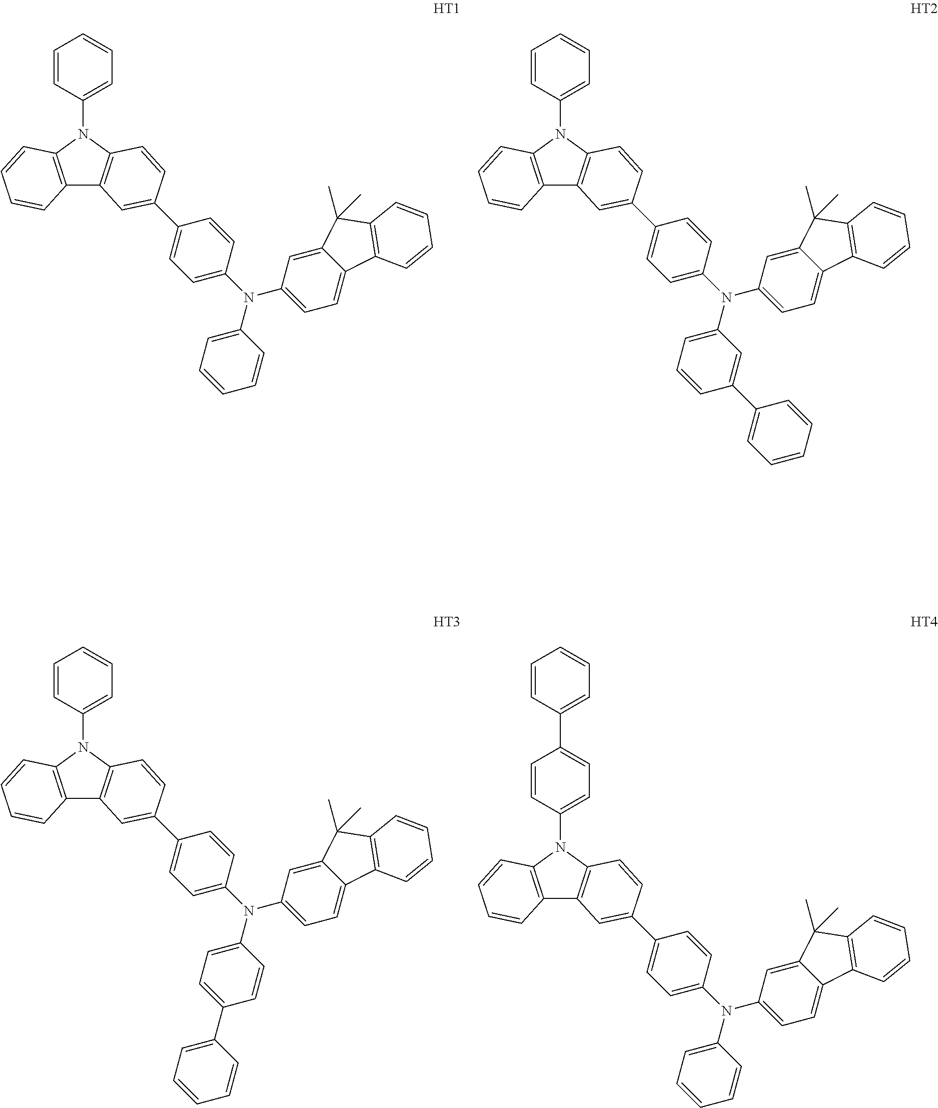 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

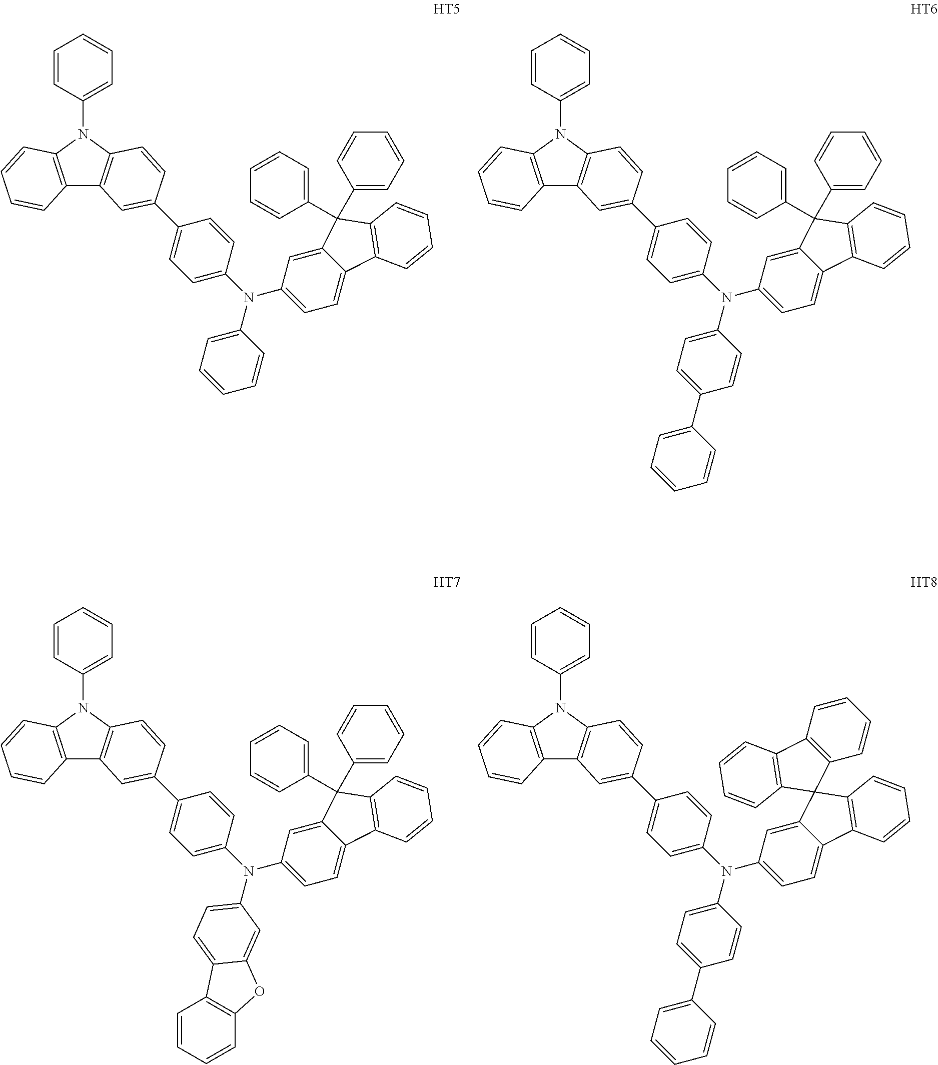 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

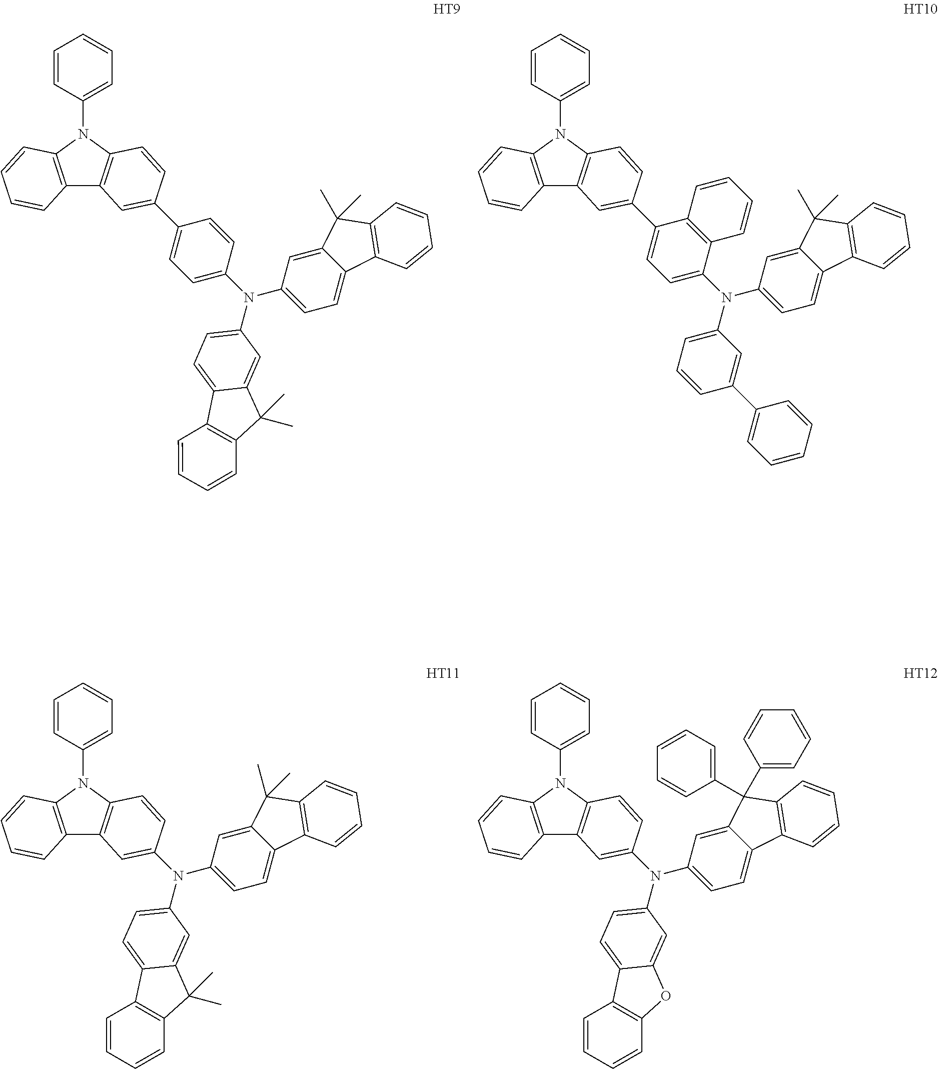 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

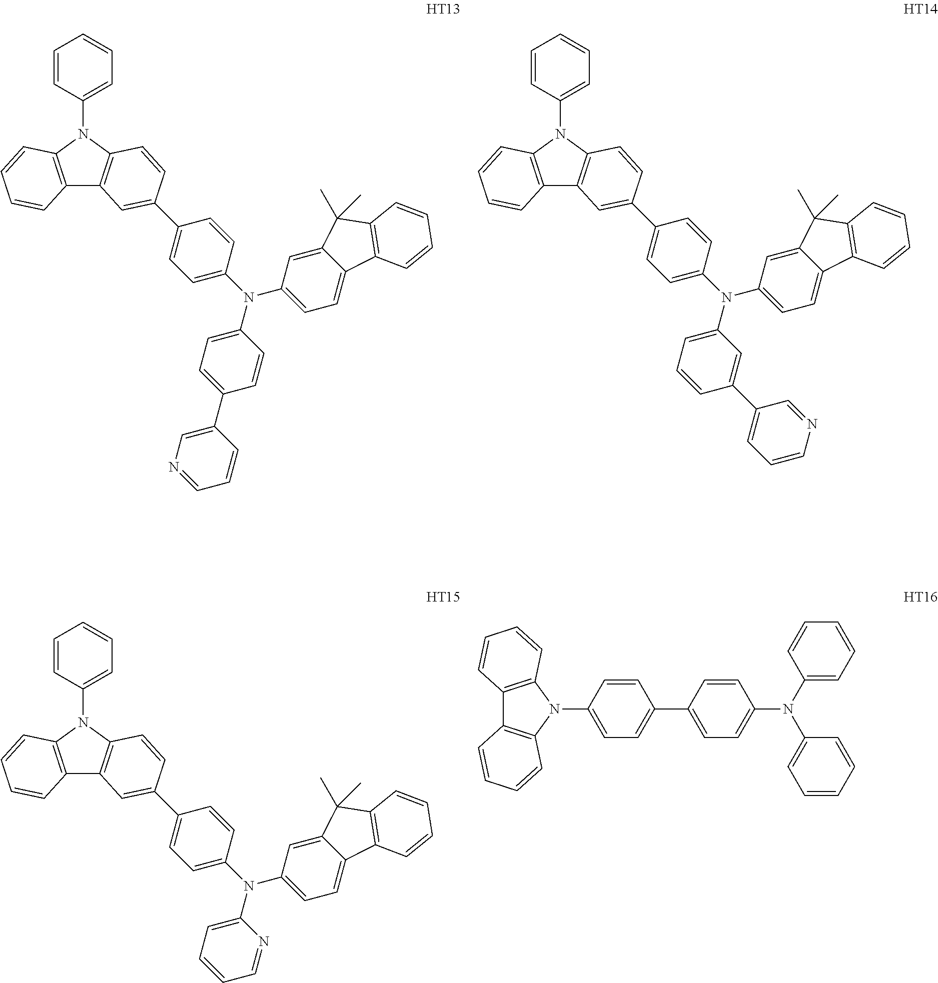 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

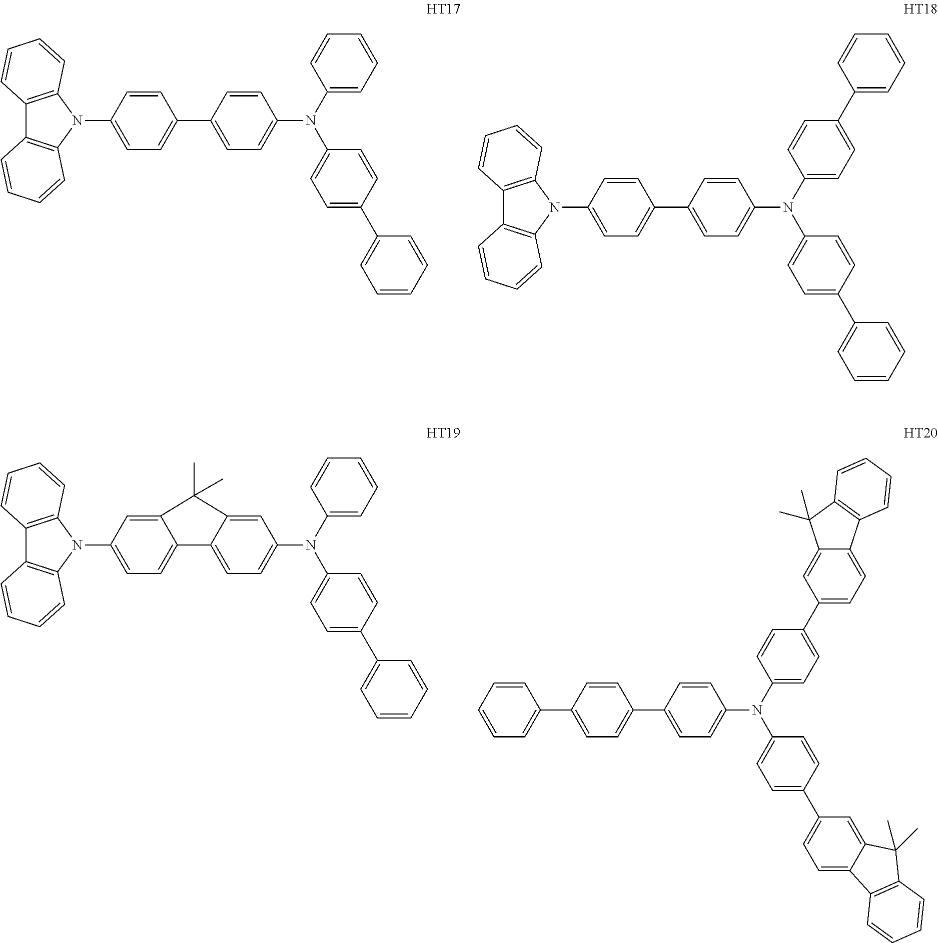 [SMILES: c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

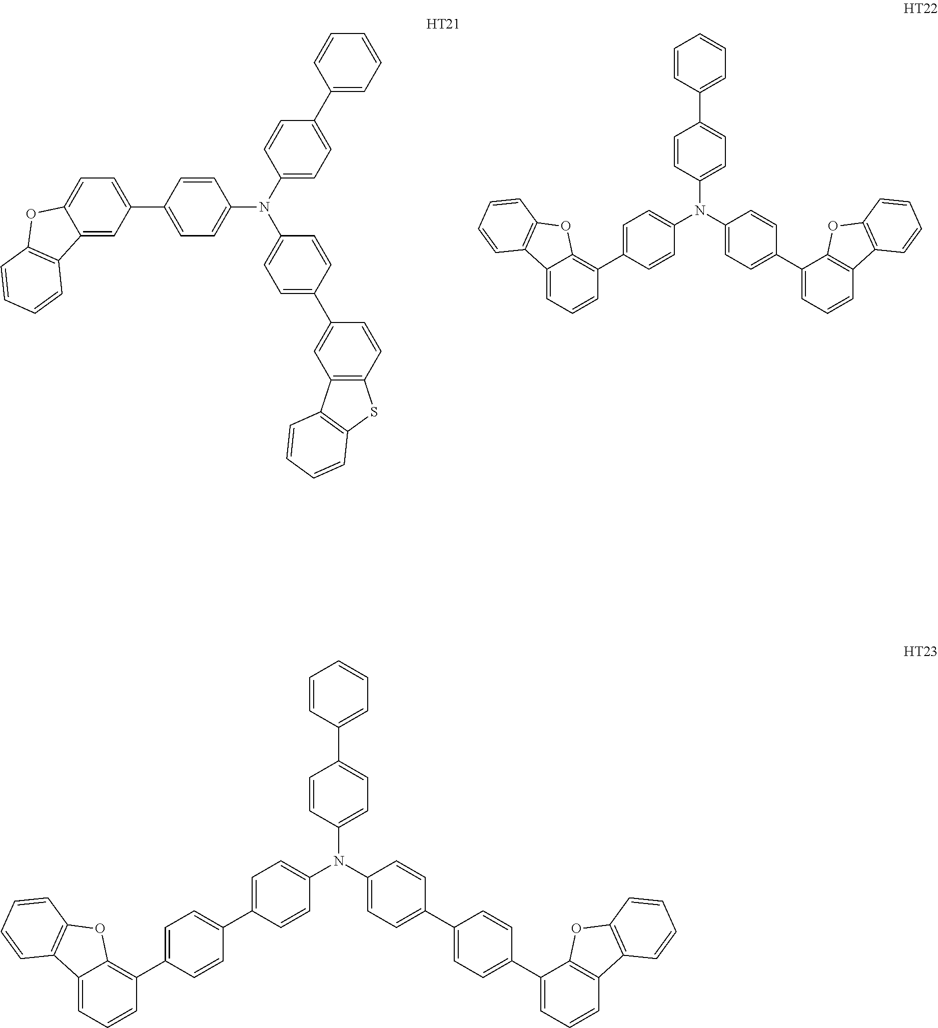 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

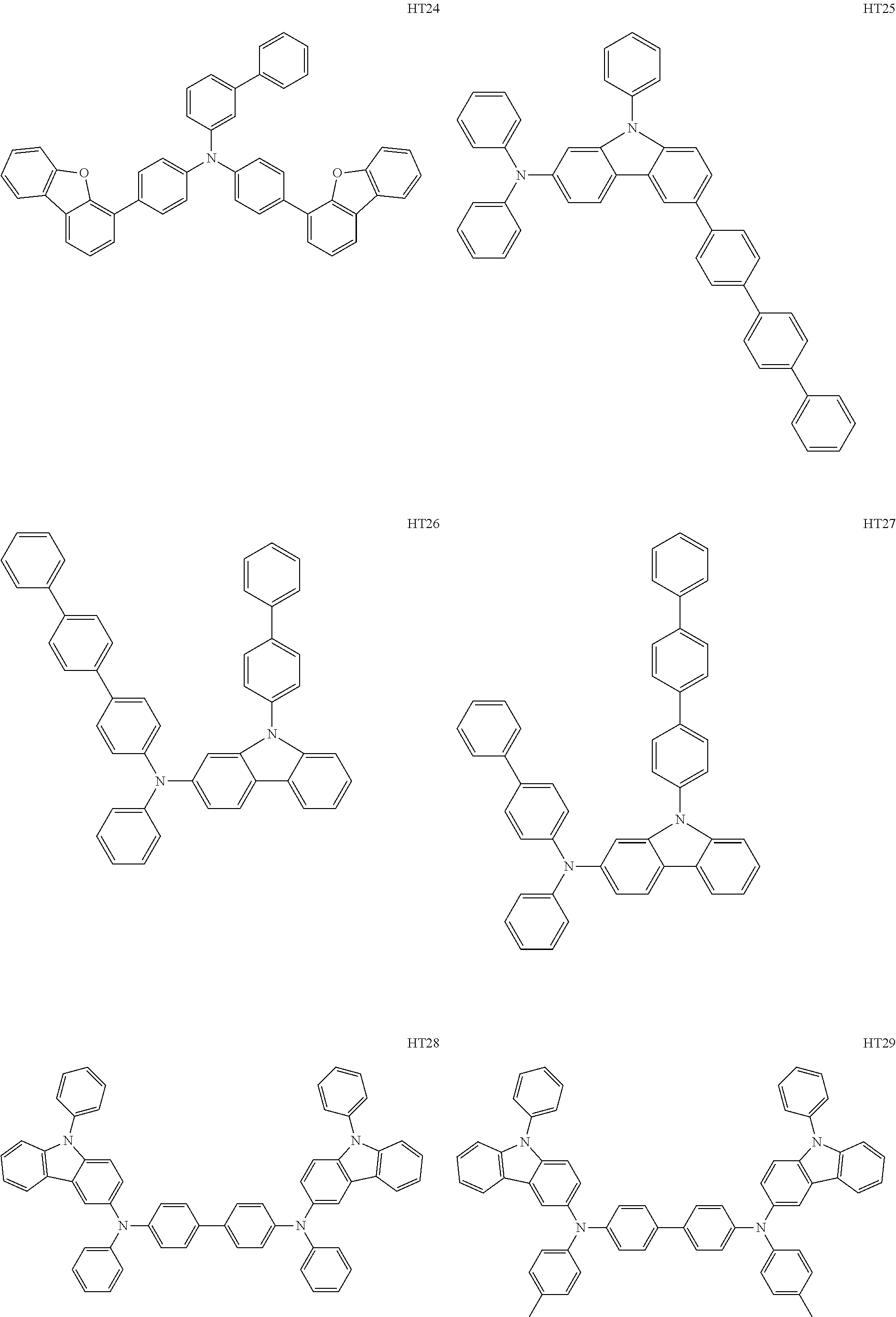

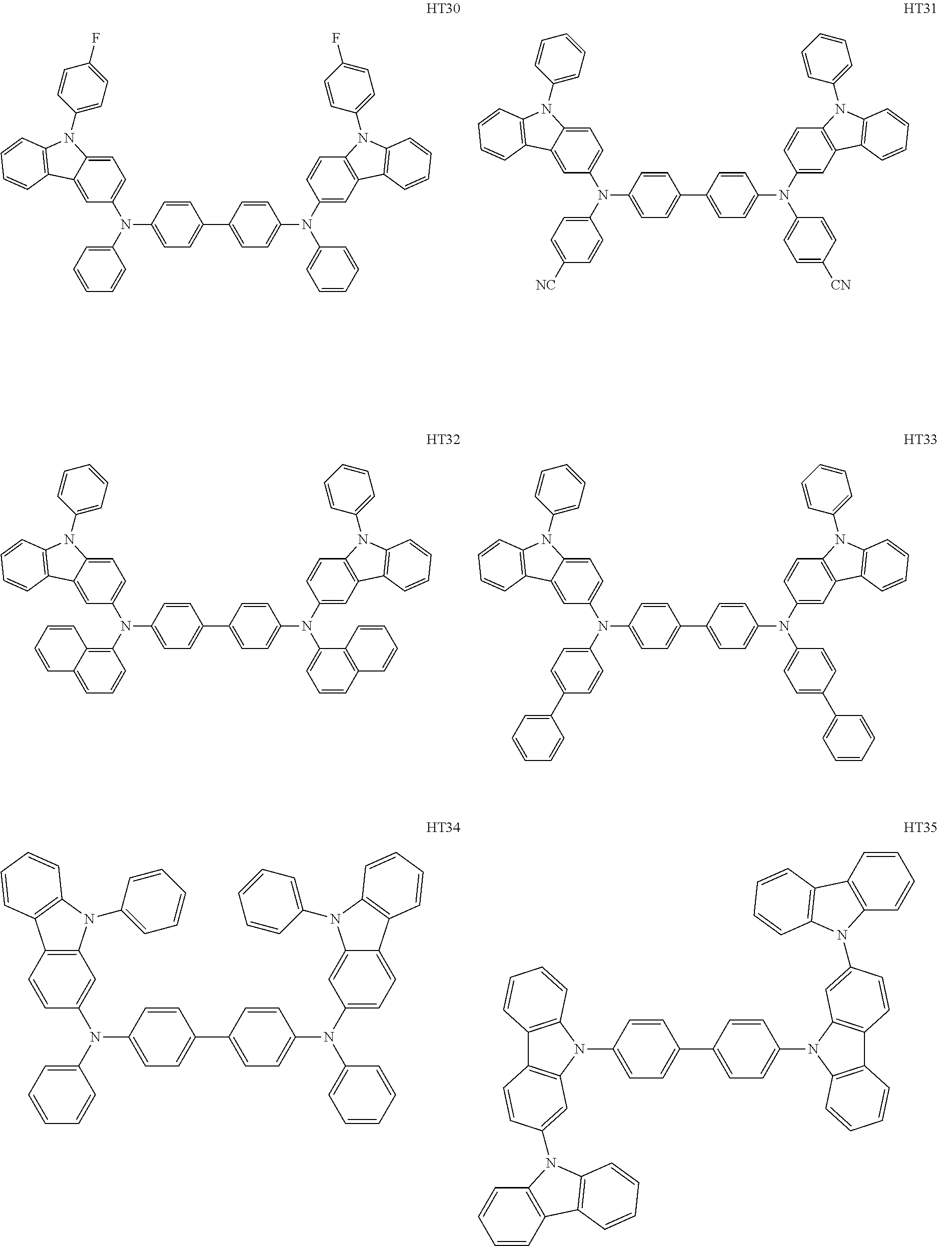

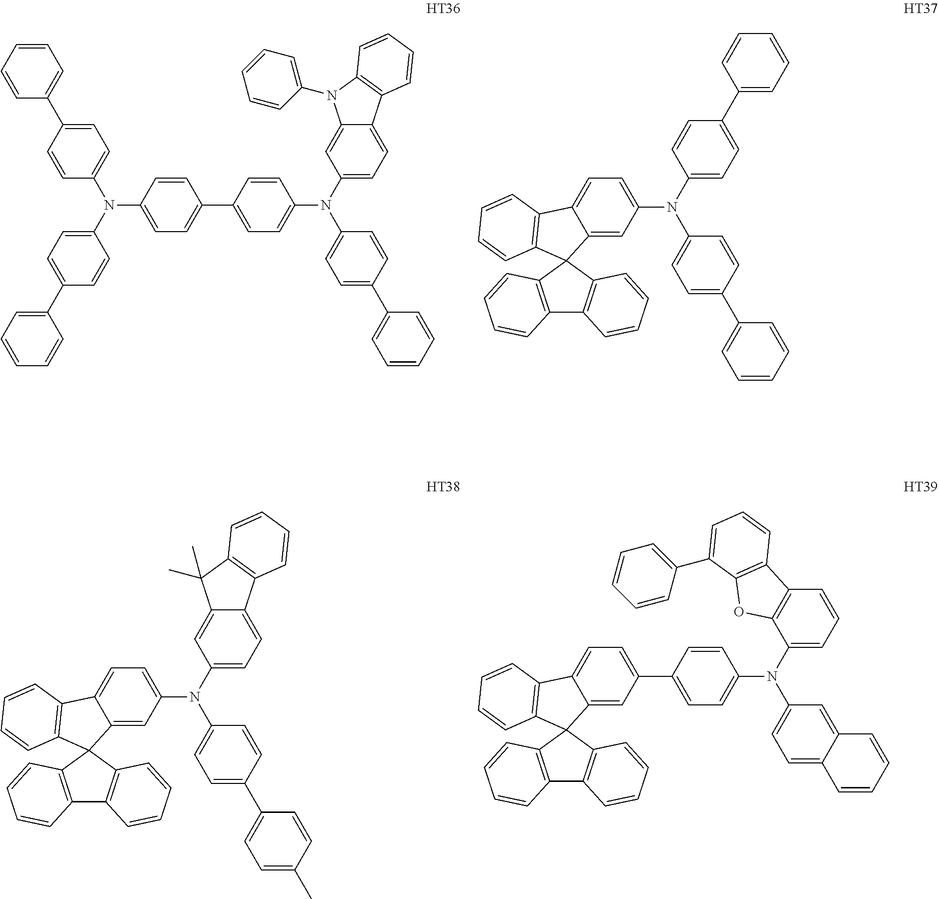 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

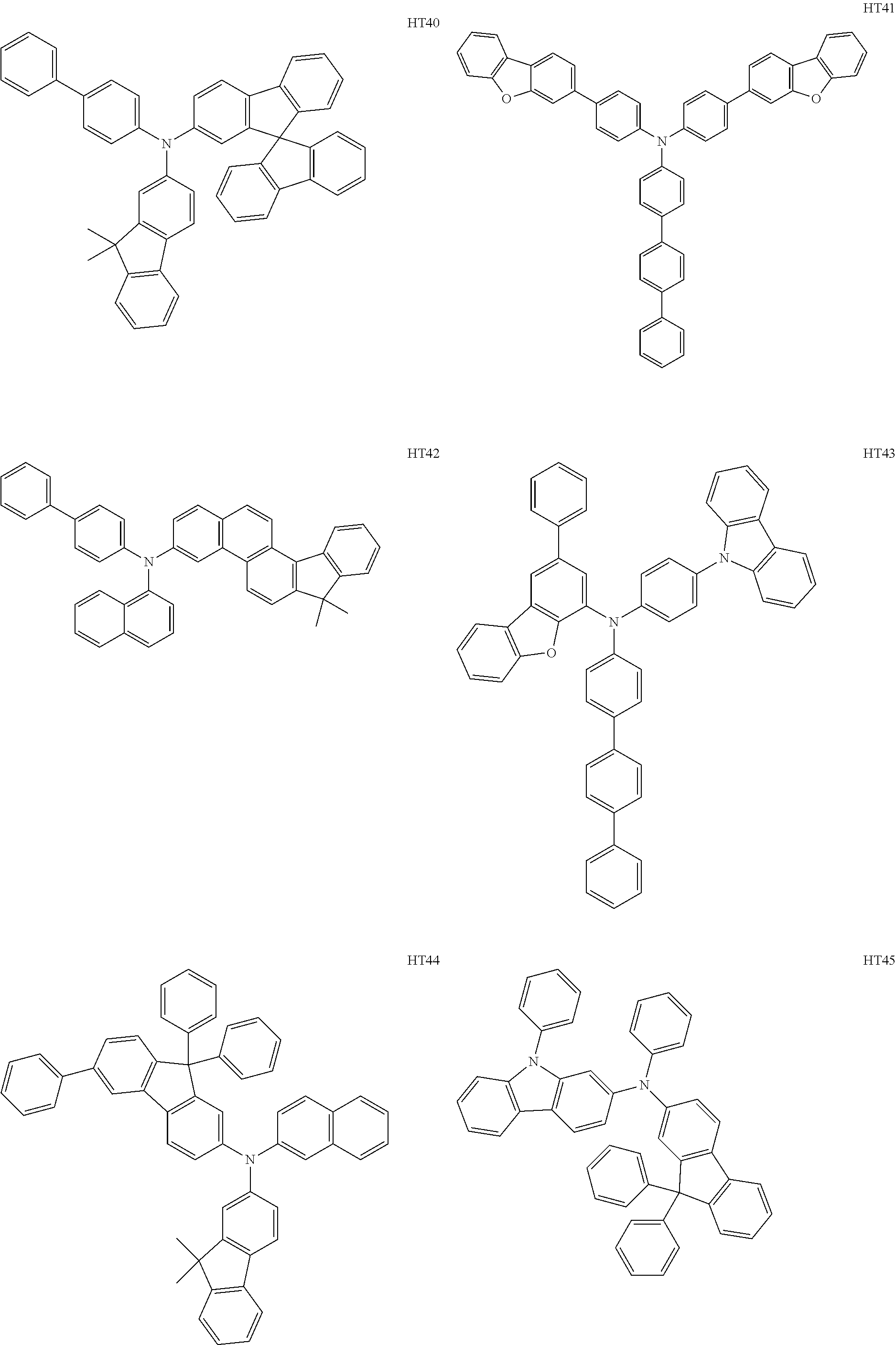

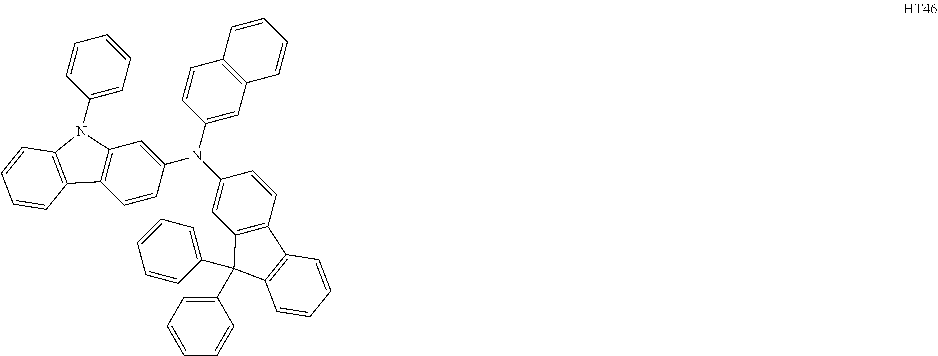 [SMILES: c1ccc(-n2c3ccccc3c3ccc(N(c4ccc5c(c4)C(c4ccccc4)(c4ccccc4)c4ccccc4-5)c4ccc5ccccc5c4)cc32)cc1]

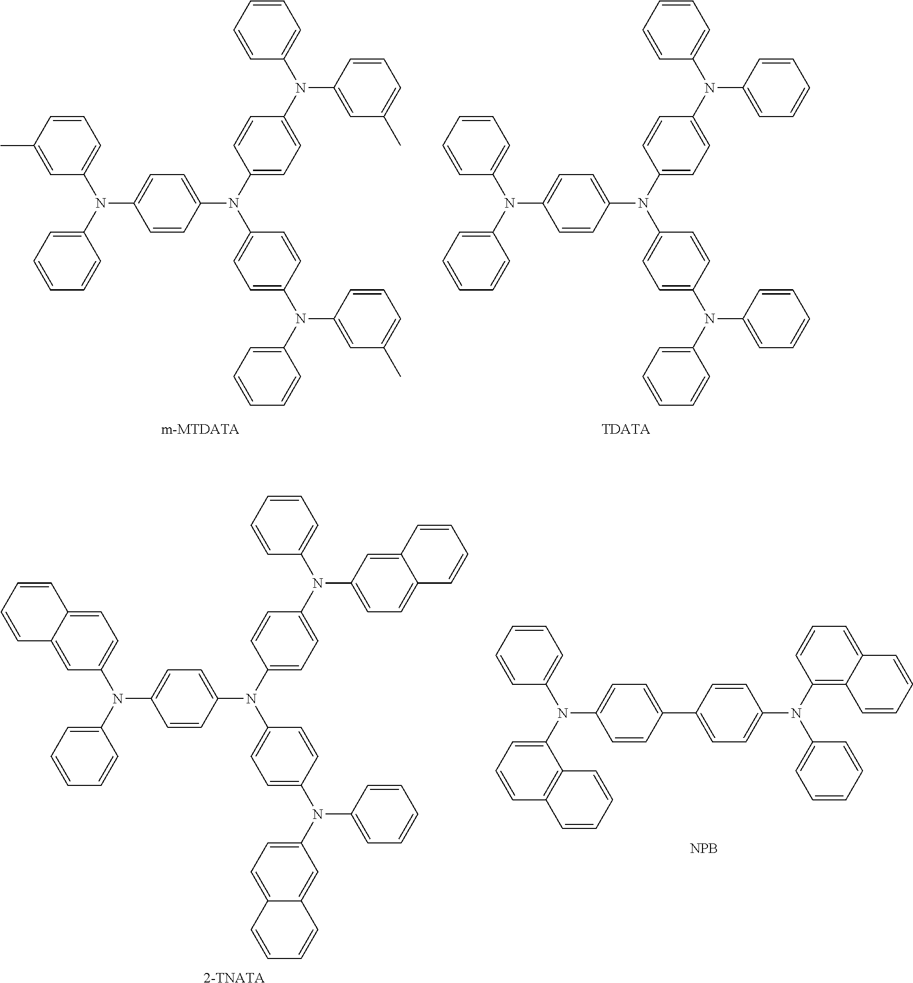

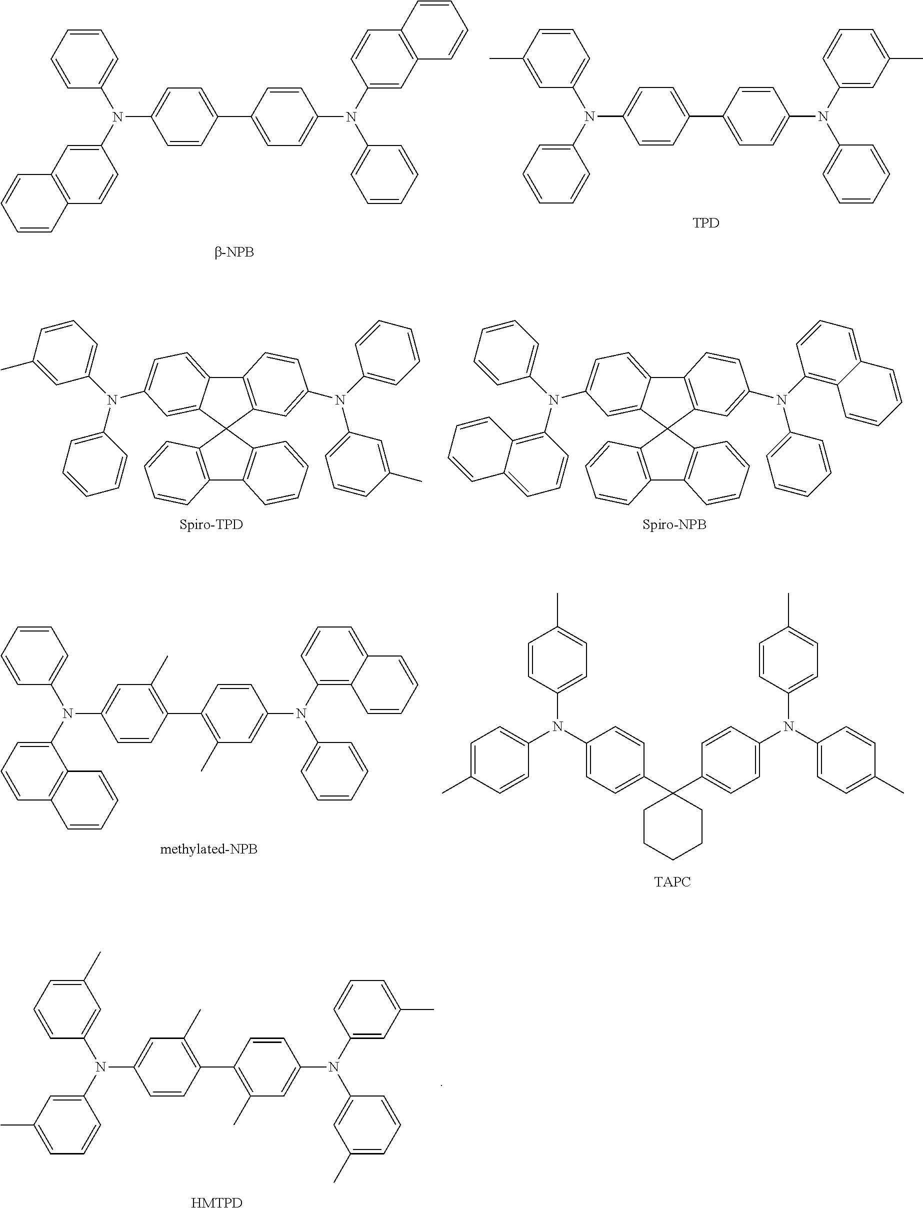

The hole transport region may have a thickness of about 50 Å to about 10,000 Å, for example, about 100 Å to about 4,000 Å. When the hole transport region includes a hole injection layer, a hole transport layer, or any combination thereof, the hole injection layer may have a thickness of about 100 Å to about 9,000 Å, for example, about 100 Å to about 1,000 Å, and the hole transport layer may have a thickness of about 50 Å to about 2,000 Å, for example, about 100 Å to about 1,500 Å. When the thicknesses of the hole transport region, the hole injection layer, and the hole transport layer are within these ranges, satisfactory hole transporting characteristics may be obtained without a substantial increase in driving voltage.

The emission auxiliary layer serves to increase light emission efficiency by compensating for an optical resonance distance according to the wavelength of light emitted by the emission layer, and the electron blocking layer serves to prevent electron leakage from the emission layer to the hole transport region. Materials that may be included in the hole transport region may be included in the emission auxiliary layer and the electron blocking layer.

[p-Dopant]

The hole transport region may further include, in addition to the materials as described above, a charge generation material to improve conductivity. The charge generation material may be uniformly or non-uniformly dispersed in the hole transport region (which may be, for example, in the form of a single layer including a charge generation material).

The charge generation material may be, for example, a p-dopant.

For example, the p-dopant may have a LUMO energy level of −3.5 eV or less.

In an embodiment, the p-dopant may include a quinone derivative, a cyano group-containing compound, a compound containing element EL1 and element EL2, or any combination thereof.

Examples of the quinone derivative may include TCNQ, F4-TCNQ, and the like.

Examples of the cyano group-containing compound may include HAT-CN, a compound represented by Formula 221, and the like:

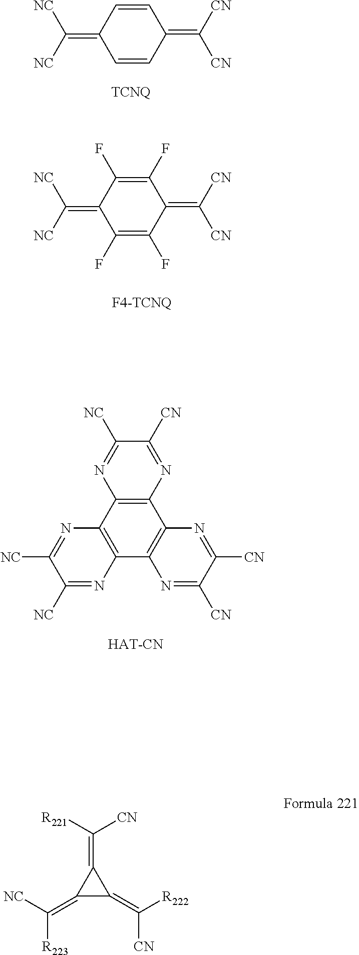

wherein, in Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one of R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound containing element EL1 and element EL2, element EL1 may be metal, a metalloid, or any combination thereof, and element EL2 may be a non-metal, a metalloid, or any combination thereof.

Examples of the metal may include: an alkali metal (for example, lithium (Li), sodium (Na), potassium (K), rubidium (Rb), cesium (Cs), or the like); an alkaline earth metal (for example, beryllium (Be), magnesium (Mg), calcium (Ca), strontium (Sr), barium (Ba), or the like); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), or the like); a post-transition metal (for example, zinc (Zn), indium (In), tin (Sn), or the like); a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), or the like); and the like.

Examples of the metalloid may include silicon (Si), antimony (Sb), tellurium (Te), and the like.

Examples of the non-metal may include oxygen (O), halogen (for example, F, Cl, Br, I, or the like), and the like.

Examples of the compound containing element ELI and element EL2 may include a metal oxide, a metal halide (for example, metal fluoride, metal chloride, metal bromide, metal iodide, or the like), a metalloid halide (for example, metalloid fluoride, metalloid chloride, metalloid bromide, metalloid iodide, or the like), a metal telluride, or any combination thereof.

Examples of the metal oxide may include a tungsten oxide (for example, WO, W₂O₃, WO₂, WO₃, W₂O₅, or the like), a vanadium oxide (for example, VO, V₂O₃, VO₂, V₂O₅, or the like), a molybdenum oxide (for example, MoO, Mo₂O₃, MoO₂, MoO₃, Mo₂O₅, or the like), a rhenium oxide (for example, ReO₃ or the like), and the like.

Examples of the metal halide may include alkali metal halide, alkaline earth metal halide, transition metal halide, post-transition metal halide, lanthanide metal halide, and the like.

Examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, CsI, and the like.

Examples of the alkaline earth metal halide may include BeF₂, MgF₂, CaF₂, SrF₂, BaF₂, BeCl₂, MgCl₂, CaCl₂, SrCl₂, BaCl₂, BeBr₂, MgBr₂, CaBr₂, SrBr₂, BaBr₂, BeI₂, MgI₂, CaI₂, SrI₂, BaI₂, and the like.

Examples of the transition metal halide may include a titanium halide (for example, TiF₄, TiCl₄, TiBr₄, TiI₄, or the like), a zirconium halide (for example, ZrF₄, ZrCl₄, ZrBr₄, ZrI₄, or the like), a hafnium halide (for example, HfF₄, HfCl₄, HfBr₄, HfI₄, or the like), a vanadium halide (for example, VF₃, VCl₃, VBr₃, VI₃, or the like), a niobium halide (for example, NbF₃, NbCl₃, NbBr₃, NbI₃, or the like), a tantalum halide (for example, TaF₃, TaCl₃, TaBr₃, TaI₃, or the like), a chromium halide (for example, CrF₃, CrCl₃, CrBr₃, CrI₃, or the like), a molybdenum halide (for example, MoF₃, MoCl₃, MoBr₃, MoI₃, or the like), a tungsten halide (for example, WF₃, WCl₃, WBr₃, WI₃, or the like), a manganese halide (for example, MnF₂, MnCl₂, MnBr₂, MnI₂, or the like), a technetium halide (for example, TcF₂, TcCl₂, TcBr₂, TcI₂, or the like), a rhenium halide (for example, ReF₂, ReCl₂, ReBr₂, ReI₂, or the like), an iron halide (for example, FeF₂, FeCl₂, FeBr₂, FeI₂, or the like), a ruthenium halide (for example, RuF₂, RuCl₂, RuBr₂, RuI₂, or the like), an osmium halide (for example, OsF₂, OsCl₂, OsBr₂, OsI₂, or the like), a cobalt halide (for example, CoF₂, CoCl₂, CoBr₂, CoI₂, or the like), a rhodium halide (for example, RhF₂, RhCl₂, RhBr₂, RhI₂, or the like), an iridium halide (for example, IrF₂, IrCl₂, IrBr₂, WI₂, or the like), a nickel halide (for example, NiF₂, NiCl₂, NiBr₂, NiI₂, or the like), a palladium halide (for example, PdF₂, PdCl₂, PdBr₂, PdI₂, or the like), a platinum halide (for example, PtF₂, PtCl₂, PtBr₂, PtI₂, or the like), a copper halide (for example, CuF, CuCl, CuBr, CuI, or the like), a silver halide (for example, AgF, AgCl, AgBr, AgI, or the like), a gold halide (for example, AuF, AuCl, AuBr, AuI, or the like), and the like.

Examples of the post-transition metal halide may include a zinc halide (for example, ZnF₂, ZnCl₂, ZnBr₂, ZnI₂, or the like), an indium halide (for example, InI₃ or the like), a tin halide (for example, SnI₂ or the like), and the like.

Examples of the lanthanide metal halide may include YbF, YbF₂, YbF₃, SmF₃, YbCl, YbCl₂, YbCl₃ SmCl₃, YbBr, YbBr₂, YbBr₃, SmBr₃, YbI, YbI₂, YbI₃, SmI₃, and the like.

Examples of the metalloid halide may include an antimony halide (for example, SbCl₅ or the like) and the like.

Examples of the metal telluride may include an alkali metal telluride (e.g., Li₂Te, Na₂Te, K₂Te, Rb₂Te, Cs₂Te, or the like), an alkaline earth metal telluride (e.g., BeTe, MgTe, CaTe, SrTe, BaTe, or the like), a transition metal telluride (e.g., TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, or the like), a post-transition metal telluride (e.g., ZnTe or the like), a lanthanide metal telluride (e.g., LaTe, CeTe, PrTe, NdTe, PmTe, EuTe, GdTe, TbTe, DyTe, HoTe, ErTe, TmTe, YbTe, LuTe, or the like), and the like.

### [Emission Layer 130]

The light-emitting device 10 may include an emission layer 130 on the hole transport region 120.

In an embodiment, the emission layer 130 may further include, in addition to various organic materials, a metal-containing compound such as an organometallic compound, an inorganic material such as a quantum dot, and the like.

In some embodiments, the emission layer 130 may include i) two or more emitting units sequentially stacked between the first electrode 110 and the second electrode 150, and ii) a charge generation layer disposed between the two or more emitting units. When the emission layer 130 includes the emitting units and the charge generation layer as described above, the light-emitting device 10 may be a tandem light-emitting device.

When the light-emitting device 10 is a full-color light-emitting device, the emission layer may be patterned into a red emission layer, a green emission layer, and/or a blue emission layer, according to a sub-pixel. In some embodiments, the emission layer may have a stacked structure of two or more layers of a red emission layer, a green emission layer, and a blue emission layer, in which the two or more layers contact each other or are separated from each other to emit white light. In some embodiments, the emission layer may include two or more materials out of a red light-emitting material, a green light-emitting material, and a blue light-emitting material that are mixed in a single layer to emit white light.

The emission layer may include a host and a dopant. The dopant may include a phosphorescent dopant, a fluorescent dopant, or any combination thereof.

The amount of the dopant in the emission layer may be in a range of about 0.01 parts by weight to about 15 parts by weight with respect to 100 parts by weight of the host.

In some embodiments, the emission layer may include a quantum dot.

In some embodiments, the emission layer may include a delayed fluorescence material. The delayed fluorescence material may act as a host or a dopant in the emission layer.

The thickness of the emission layer may be in a range of about 100 Å to about 1,000 Å, for example, about 200 Å to about 600 Å. When the thickness of the emission layer is within these ranges, excellent luminescence characteristics may be obtained without a substantial increase in driving voltage.

### [Host]

The host may include a compound represented by Formula 301:

[Ar₃₀₁]_{xb11}-[(L₃₀₁)_{xb1}-R₃₀₁]_{xb21} Formula 301
wherein, in Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ may each be as described herein in connection with Q₁.

For example, when xb11 in Formula 301 is 2 or more, two or more of Ar₃₀₁ may be linked to each other via a single bond.

In some embodiments, the host may include a compound represented by Formula 301-1, a compound represented by Formula 301-2, or any combination thereof:

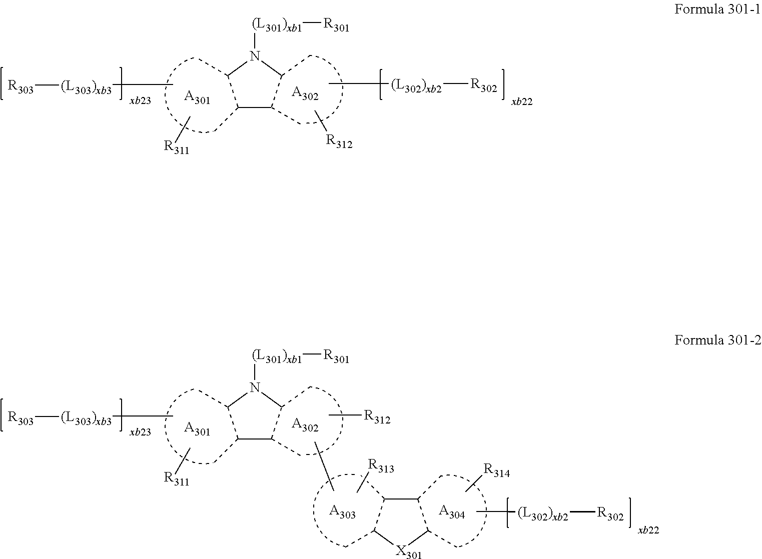

wherein, in Formula 301-1 and 301-2,
rings A₃₀₁ to A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N[(L₃₀₄)xb4-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may each be as described herein,
L₃₀₂ to L₃₀₄ may each independently be as described above in connection with L₃₀₁,
xb2 to xb4 may each independently be as described above in connection with xb1, and
R₃₀₂ to R₃₀₅ and R₃₁₁ to R₃₁₄ may each be as described above in connection with R₃₀₁.

In some embodiments, the host may include an alkali earth metal complex, a post-transition metal complex, or any combination thereof. In some embodiments, the host may include a Be complex (for example, Compound H55), an Mg complex, a Zn complex, or any combination thereof.

In some embodiments, the host may include: one of Compounds H1 to H128; 9,10-di(2-naphthyl)anthracene (ADN); 2-methyl-9,10-bis(naphthalen-2-yl)anthracene (MADN); 9,10-di-(2-naphthyl)-2-t-butyl-anthracene (TBADN); 4,4′-bis(N-carbazolyl)-1,1′-biphenyl (CBP); 1,3-di(carbazol-9-yl)benzene (mCP); 1,3,5-tri(carbazol-9-yl)benzene (TCP); or any combination thereof:

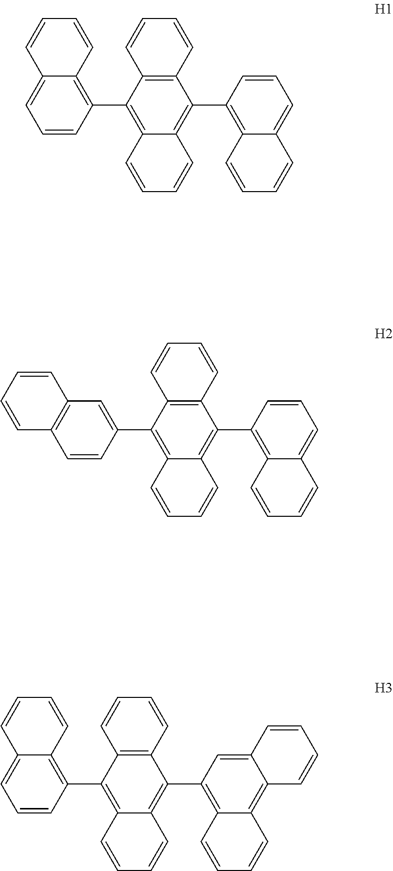

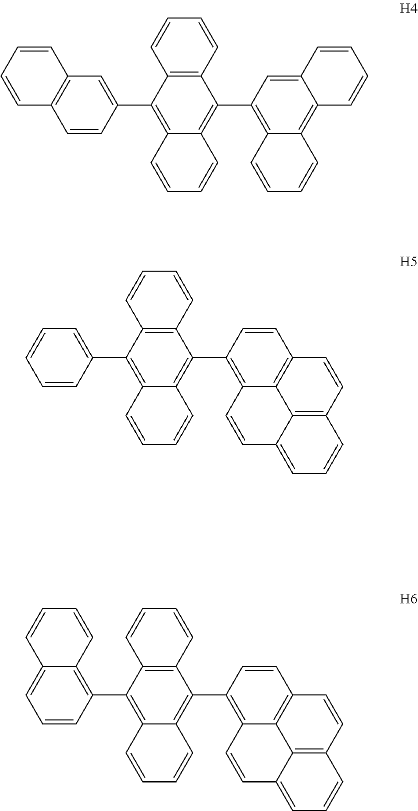

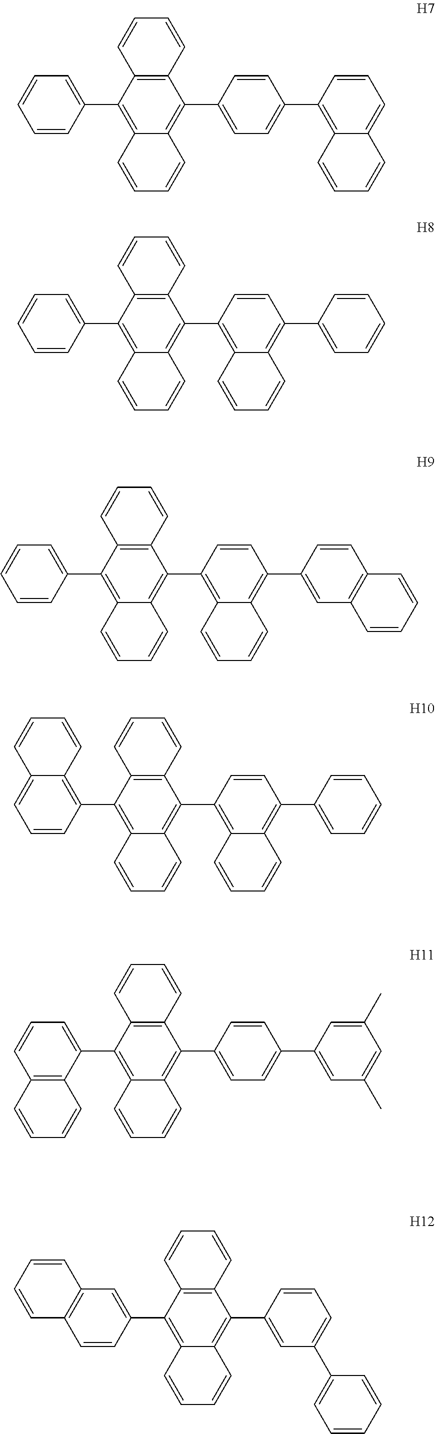

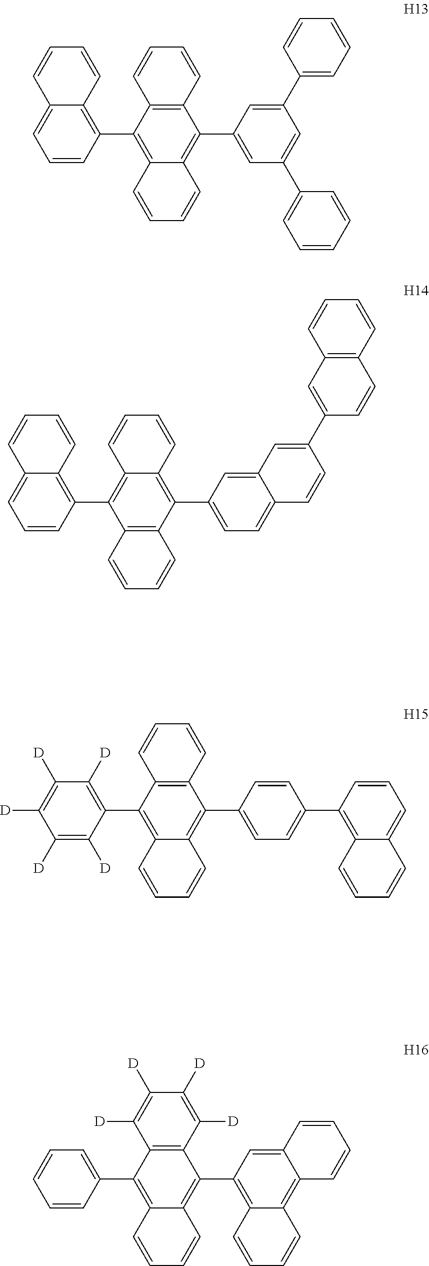

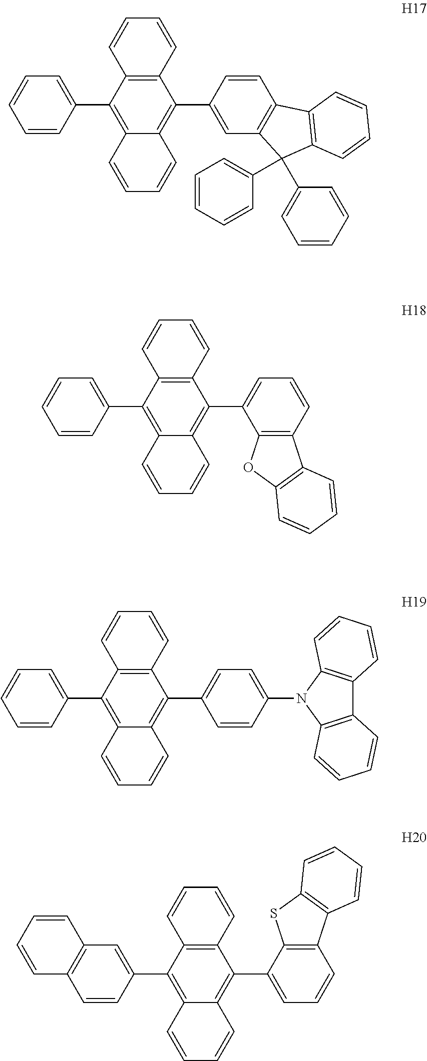

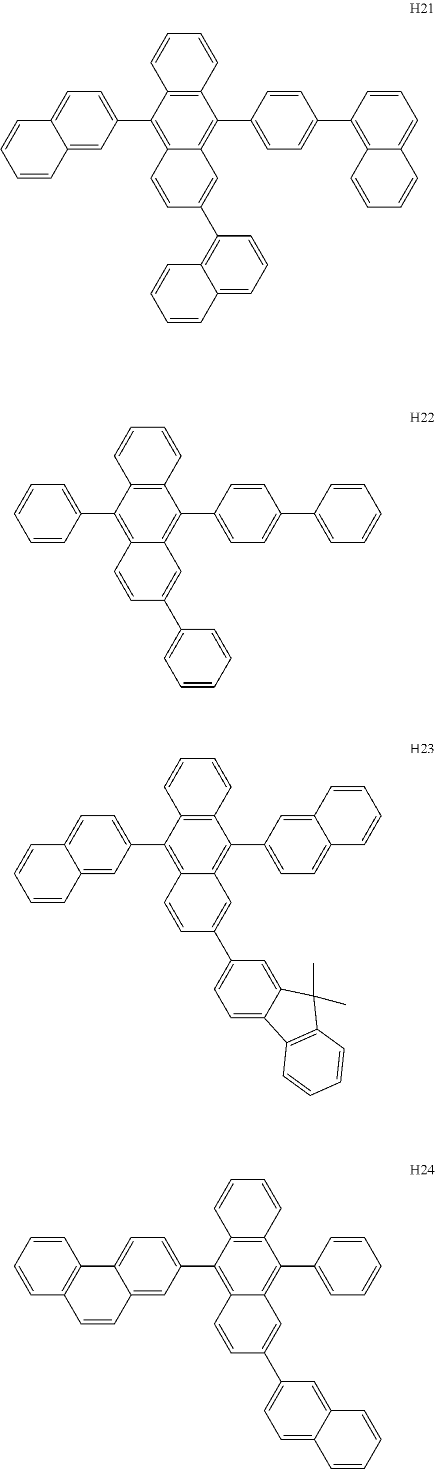

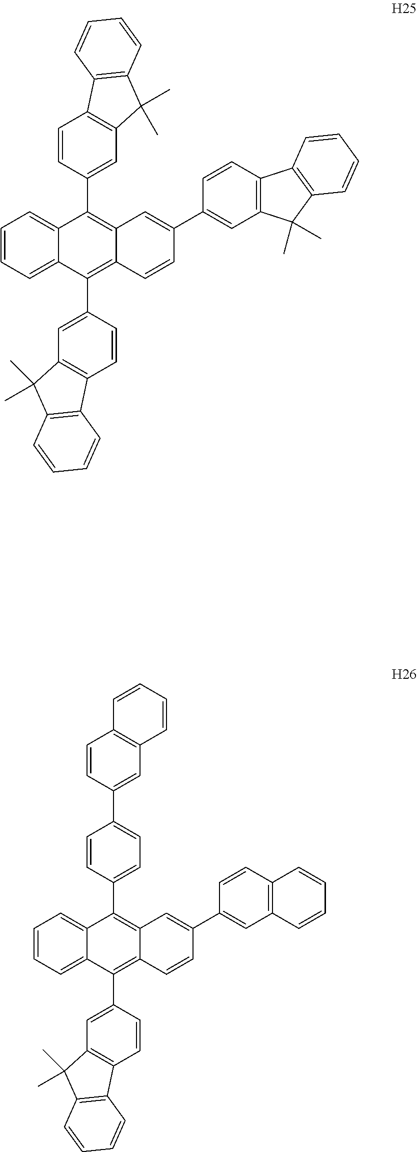

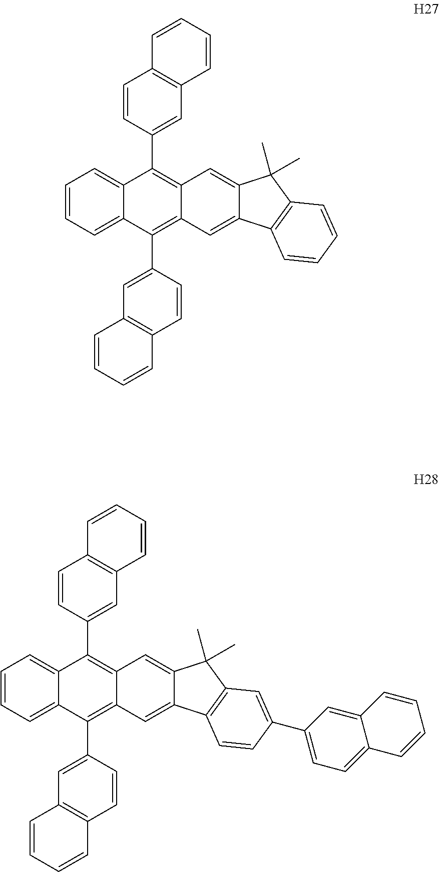

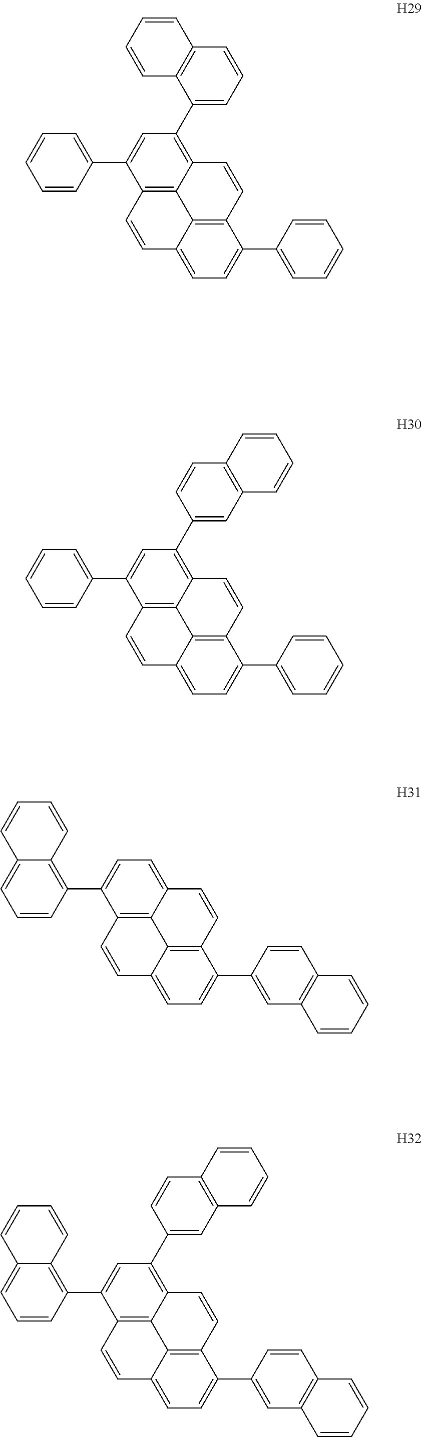

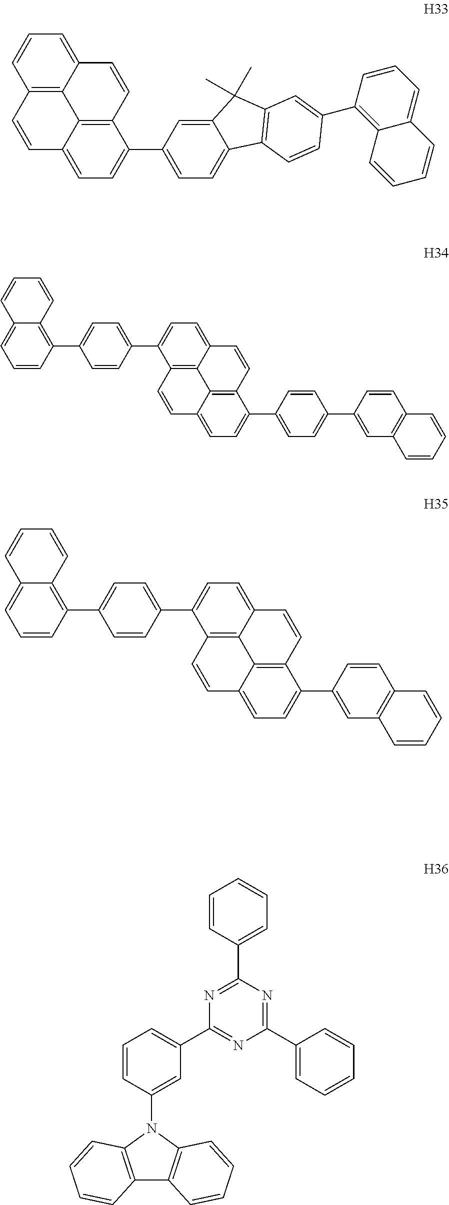

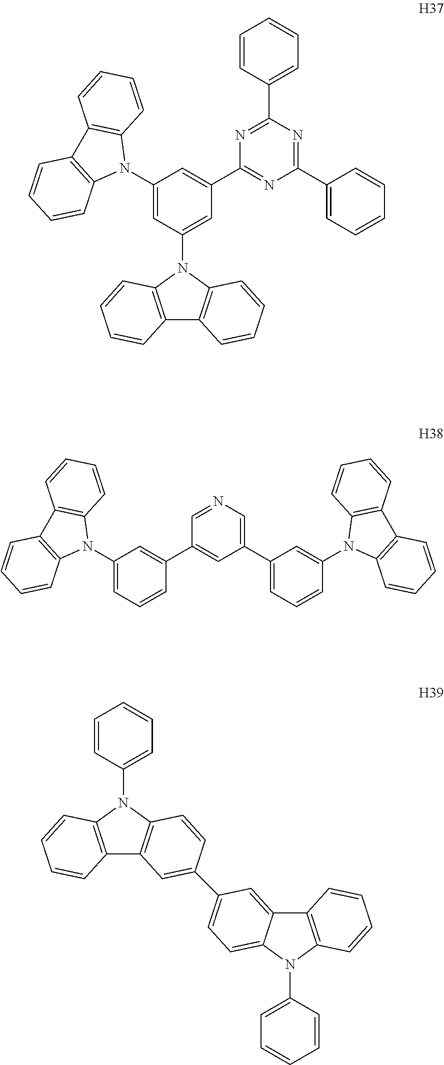 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1]

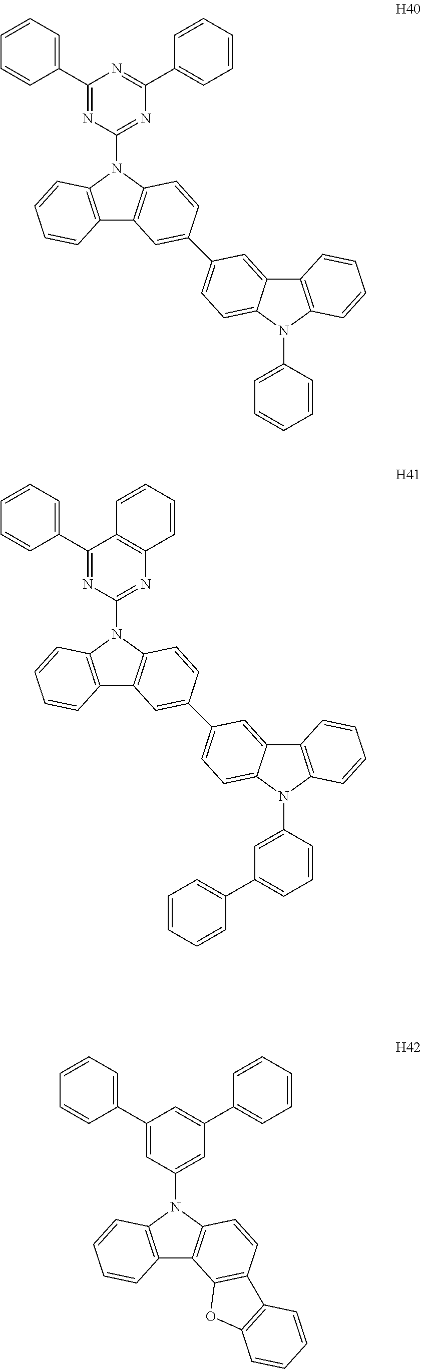 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

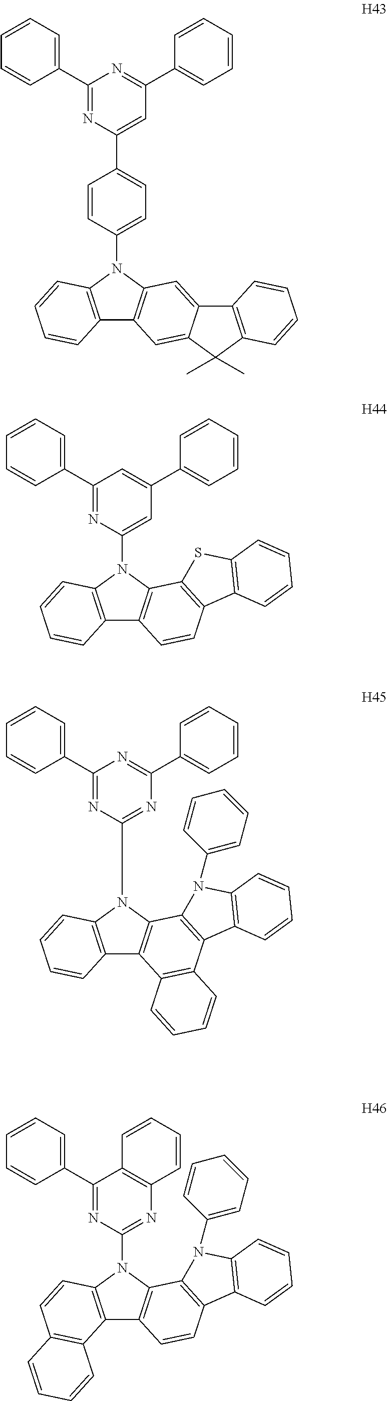 [SMILES: CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1 ; c1ccc(-c2nc(-n3c4ccc5ccccc5c4c4ccc5c6ccccc6n(-c6ccccc6)c5c43)nc3ccccc23)cc1]

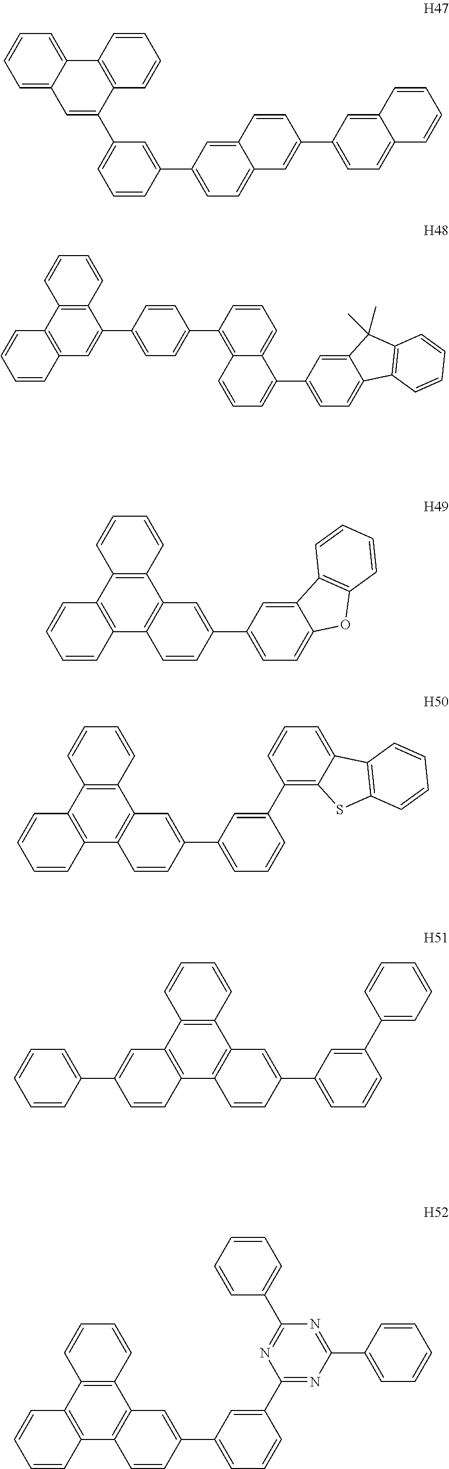

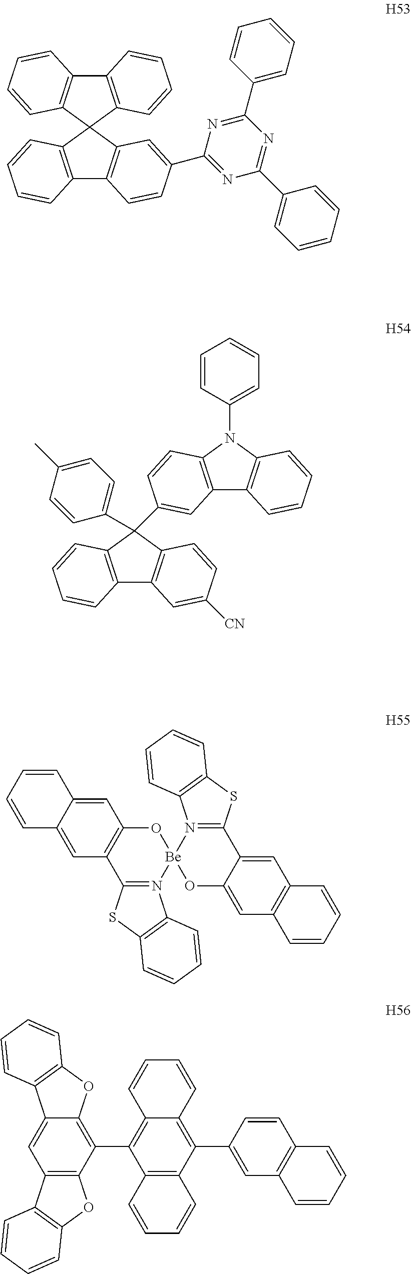

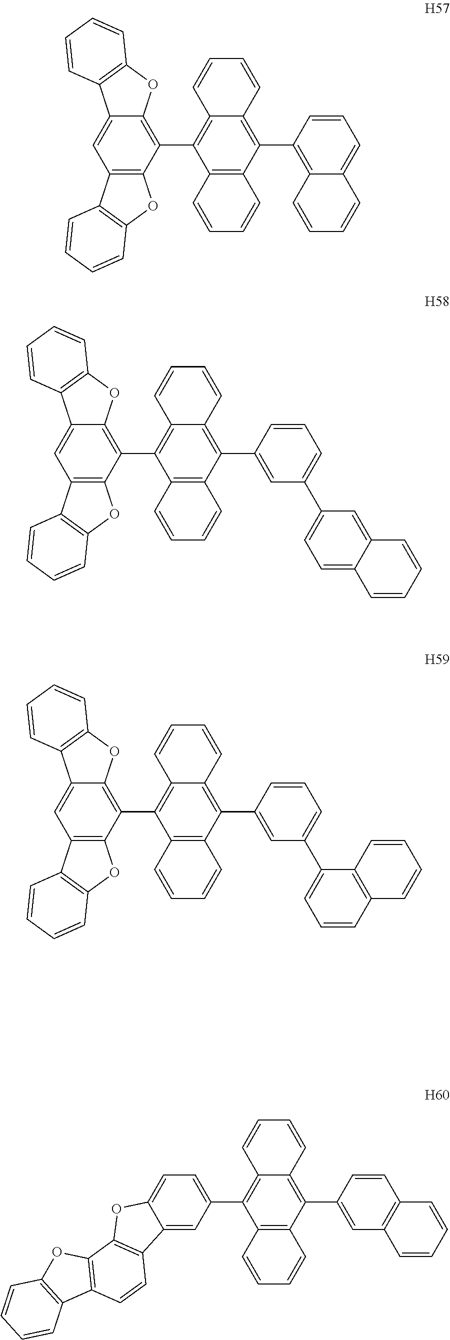 [SMILES: c1ccc2c(-c3c4ccccc4c(-c4c5oc6ccccc6c5cc5c4oc4ccccc45)c4ccccc34)cccc2c1 ; c1cc(-c2ccc3ccccc3c2)cc(-c2c3ccccc3c(-c3c4oc5ccccc5c4cc4c3oc3ccccc34)c3ccccc23)c1 ; c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1]

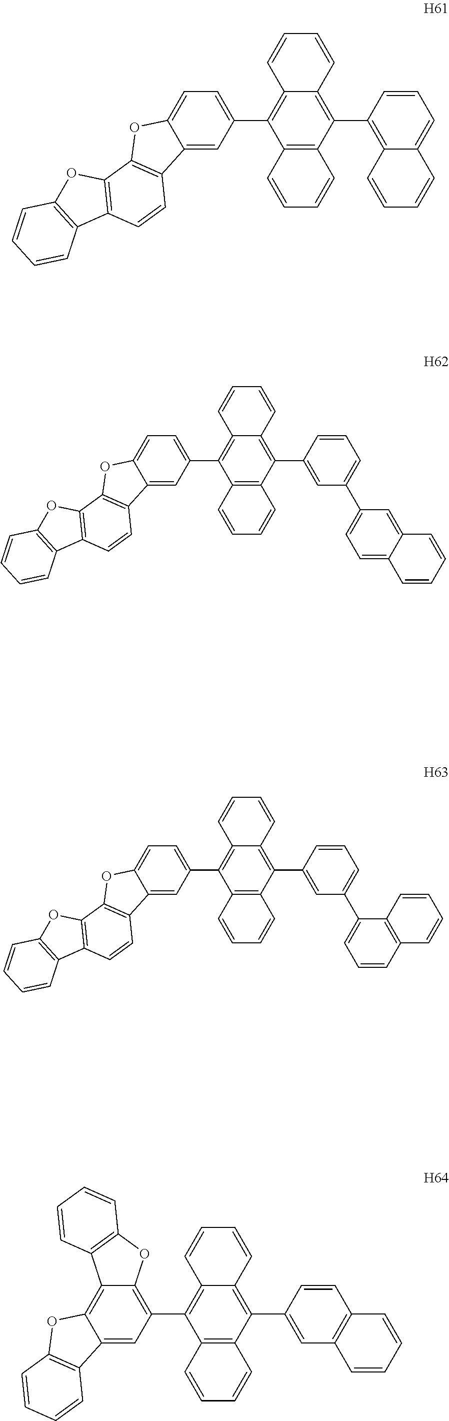 [SMILES: c1ccc2c(-c3c4ccccc4c(-c4ccc5oc6c(ccc7c8ccccc8oc76)c5c4)c4ccccc34)cccc2c1 ; c1cc(-c2ccc3ccccc3c2)cc(-c2c3ccccc3c(-c3ccc4oc5c(ccc6c7ccccc7oc65)c4c3)c3ccccc23)c1 ; c1cc(-c2cccc3ccccc23)cc(-c2c3ccccc3c(-c3ccc4oc5c(ccc6c7ccccc7oc65)c4c3)c3ccccc23)c1 ; c1ccc2cc(-c3c4ccccc4c(-c4cc5c6ccccc6oc5c5c4oc4ccccc45)c4ccccc34)ccc2c1]

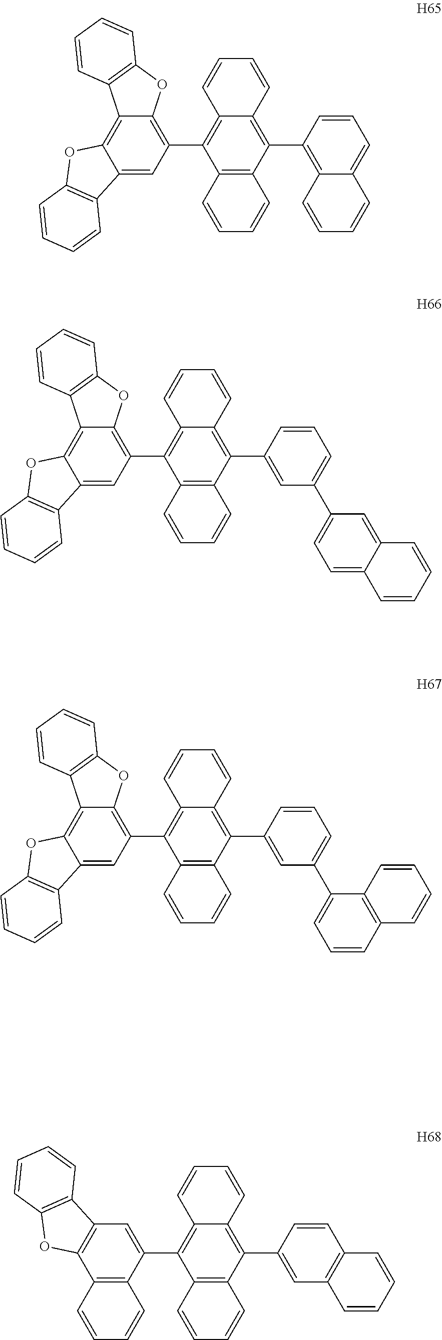 [SMILES: c1ccc2c(-c3c4ccccc4c(-c4cc5c6ccccc6oc5c5c4oc4ccccc45)c4ccccc34)cccc2c1 ; c1cc(-c2ccc3ccccc3c2)cc(-c2c3ccccc3c(-c3cc4c5ccccc5oc4c4c3oc3ccccc34)c3ccccc23)c1 ; c1cc(-c2cccc3ccccc23)cc(-c2c3ccccc3c(-c3cc4c5ccccc5oc4c4c3oc3ccccc34)c3ccccc23)c1 ; c1ccc2cc(-c3c4ccccc4c(-c4cc5c6ccccc6oc5c5ccccc45)c4ccccc34)ccc2c1]

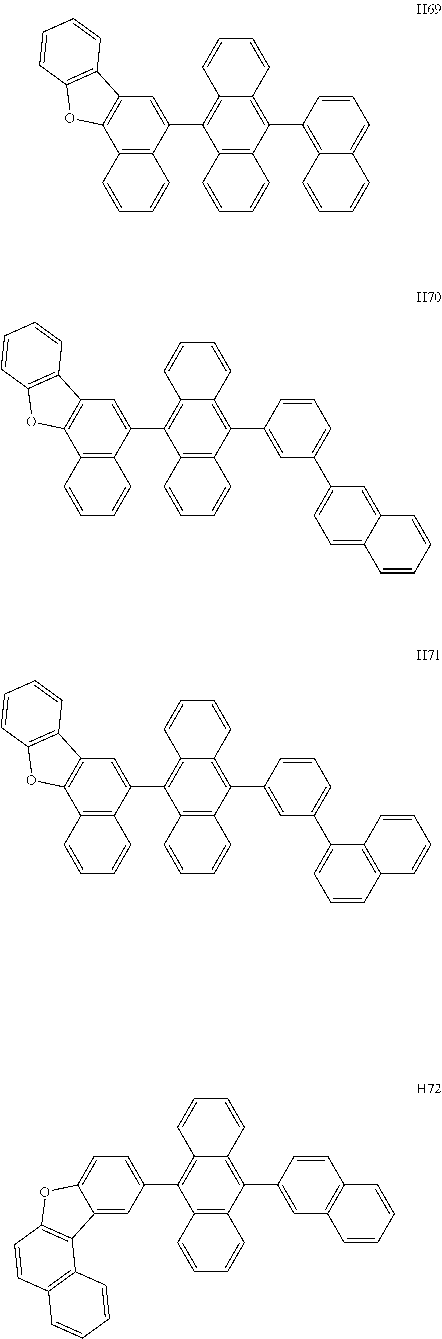 [SMILES: c1ccc2c(-c3c4ccccc4c(-c4cc5c6ccccc6oc5c5ccccc45)c4ccccc34)cccc2c1 ; c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1 ; c1ccc2cc(-c3c4ccccc4c(-c4ccc5oc6ccc7ccccc7c6c5c4)c4ccccc34)ccc2c1]

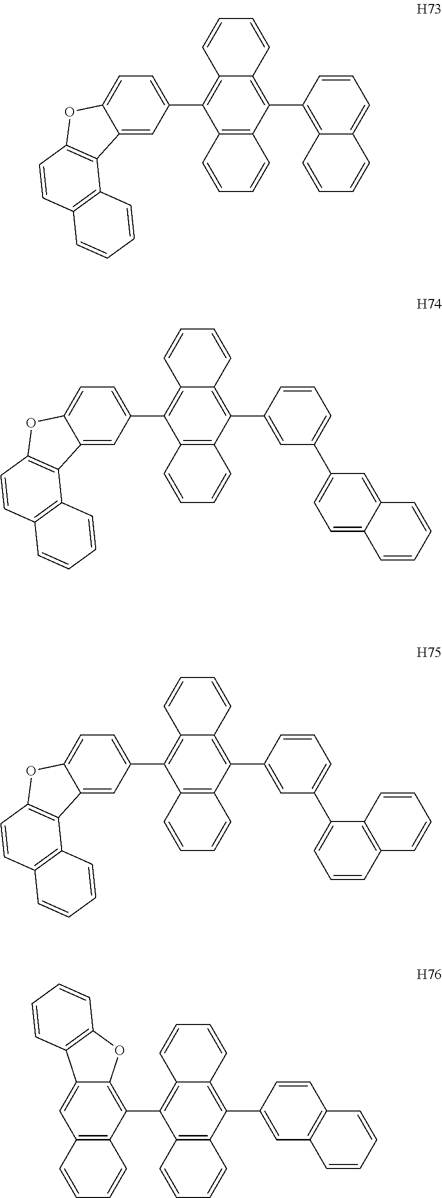 [SMILES: c1ccc2c(-c3c4ccccc4c(-c4ccc5oc6ccc7ccccc7c6c5c4)c4ccccc34)cccc2c1 ; c1cc(-c2ccc3ccccc3c2)cc(-c2c3ccccc3c(-c3ccc4oc5ccc6ccccc6c5c4c3)c3ccccc23)c1 ; c1cc(-c2cccc3ccccc23)cc(-c2c3ccccc3c(-c3ccc4oc5ccc6ccccc6c5c4c3)c3ccccc23)c1 ; c1ccc2cc(-c3c4ccccc4c(-c4c5ccccc5cc5c4oc4ccccc45)c4ccccc34)ccc2c1]

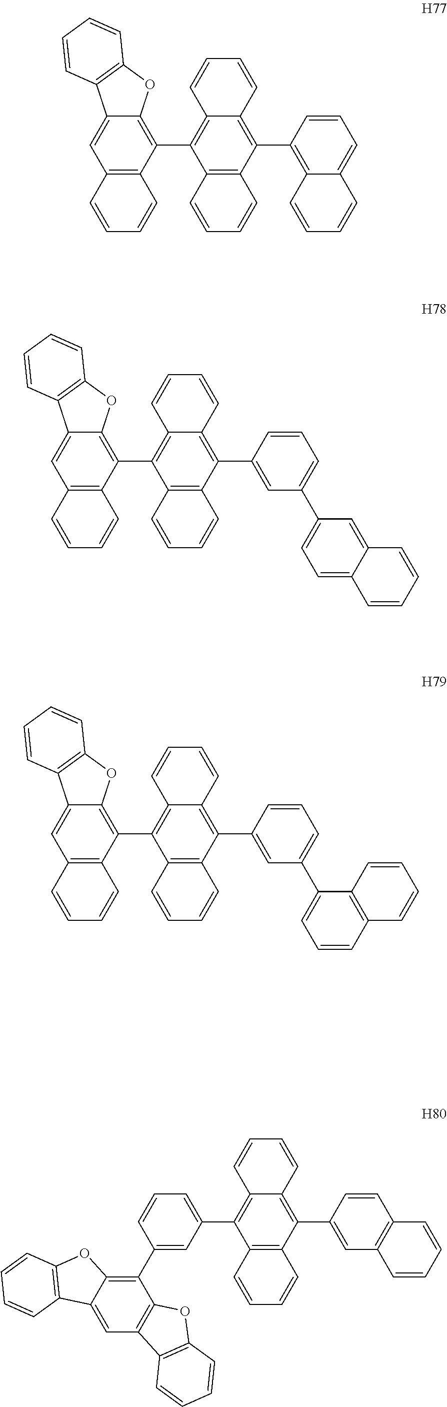 [SMILES: c1ccc2c(-c3c4ccccc4c(-c4c5ccccc5cc5c4oc4ccccc45)c4ccccc34)cccc2c1 ; c1cc(-c2ccc3ccccc3c2)cc(-c2c3ccccc3c(-c3c4ccccc4cc4c3oc3ccccc34)c3ccccc23)c1 ; c1cc(-c2cccc3ccccc23)cc(-c2c3ccccc3c(-c3c4ccccc4cc4c3oc3ccccc34)c3ccccc23)c1 ; c1cc(-c2c3ccccc3c(-c3ccc4ccccc4c3)c3ccccc23)cc(-c2c3oc4ccccc4c3cc3c2oc2ccccc23)c1]

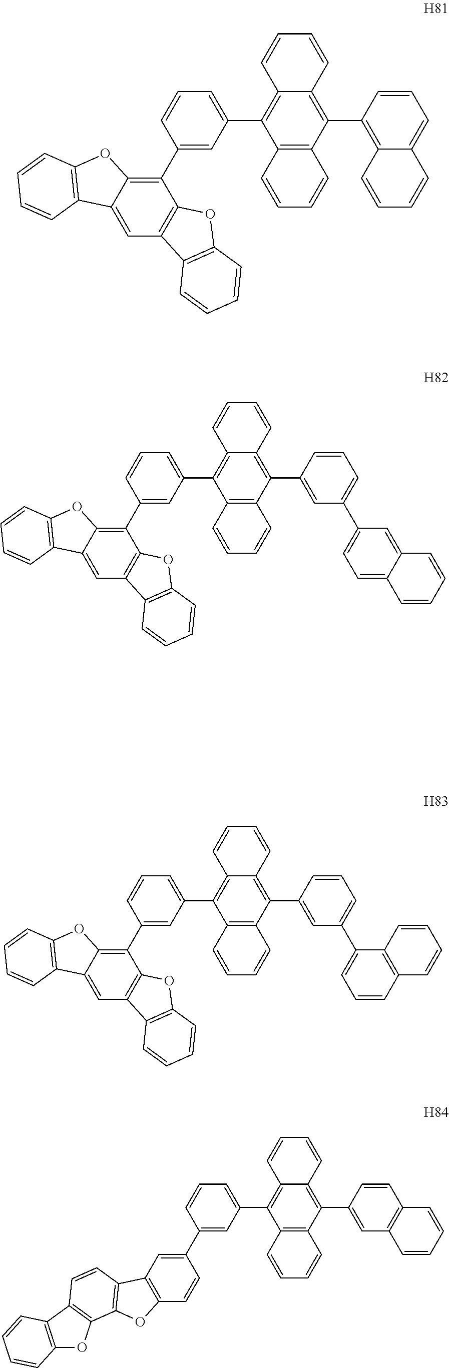 [SMILES: c1cc(-c2c3ccccc3c(-c3cccc4ccccc34)c3ccccc23)cc(-c2c3oc4ccccc4c3cc3c2oc2ccccc23)c1 ; c1cc(-c2ccc3ccccc3c2)cc(-c2c3ccccc3c(-c3cccc(-c4c5oc6ccccc6c5cc5c4oc4ccccc45)c3)c3ccccc23)c1 ; c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1]

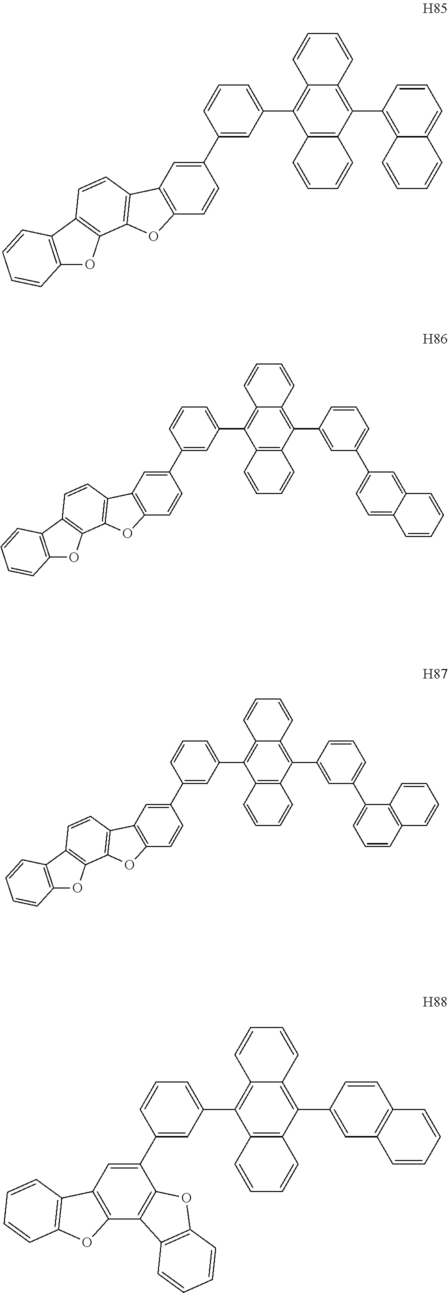 [SMILES: c1cc(-c2ccc3oc4c(ccc5c6ccccc6oc54)c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1 ; c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3c2oc2ccccc23)c1]

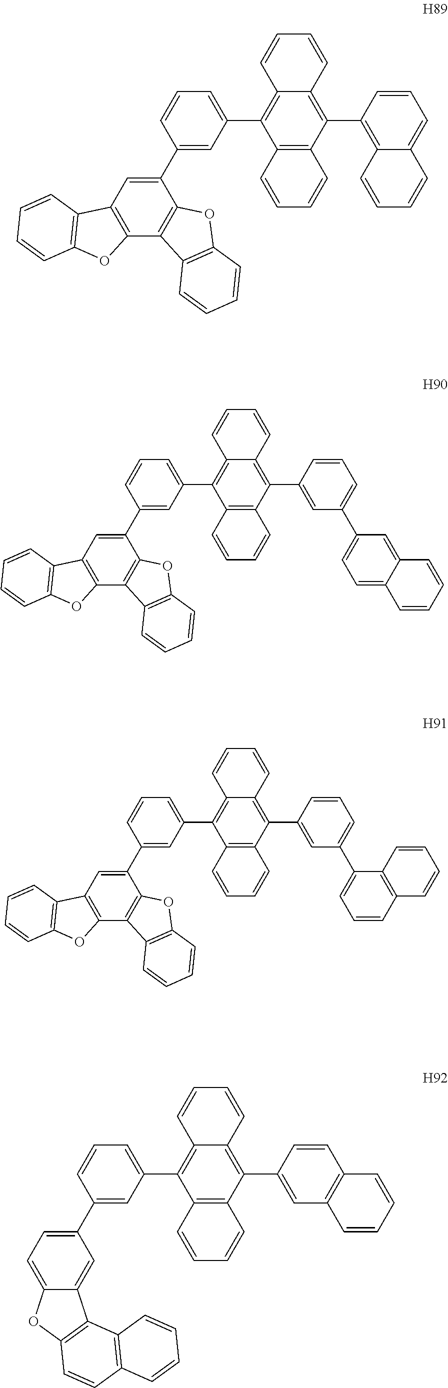 [SMILES: c1cc(-c2c3ccccc3c(-c3cccc4ccccc34)c3ccccc23)cc(-c2cc3c4ccccc4oc3c3c2oc2ccccc23)c1 ; c1cc(-c2ccc3ccccc3c2)cc(-c2c3ccccc3c(-c3cccc(-c4cc5c6ccccc6oc5c5c4oc4ccccc45)c3)c3ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1 ; c1cc(-c2ccc3oc4ccc5ccccc5c4c3c2)cc(-c2c3ccccc3c(-c3ccc4ccccc4c3)c3ccccc23)c1]

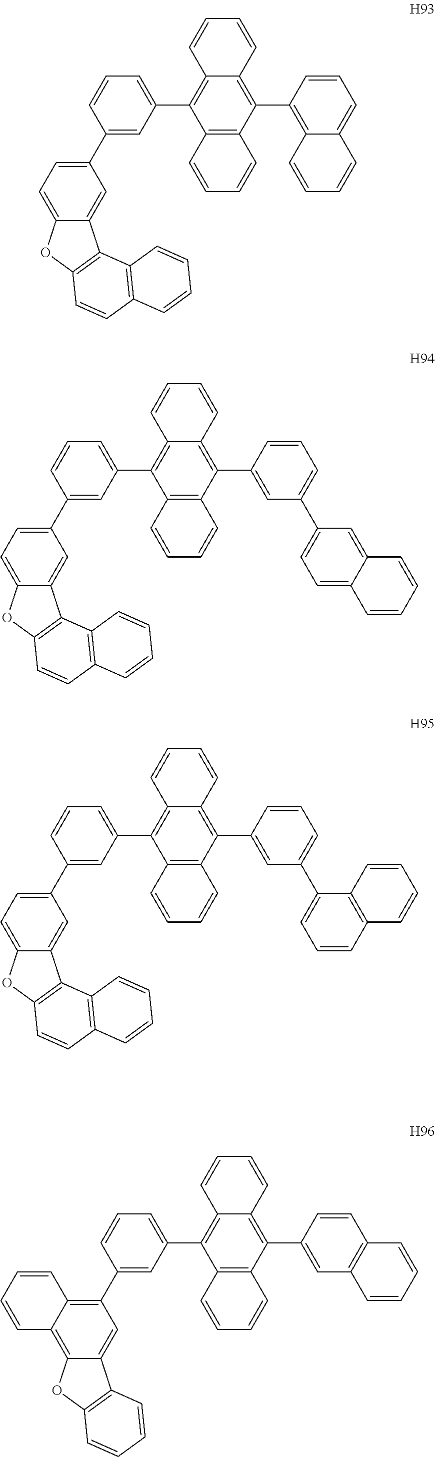 [SMILES: c1cc(-c2ccc3oc4ccc5ccccc5c4c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3ccccc23)c1]

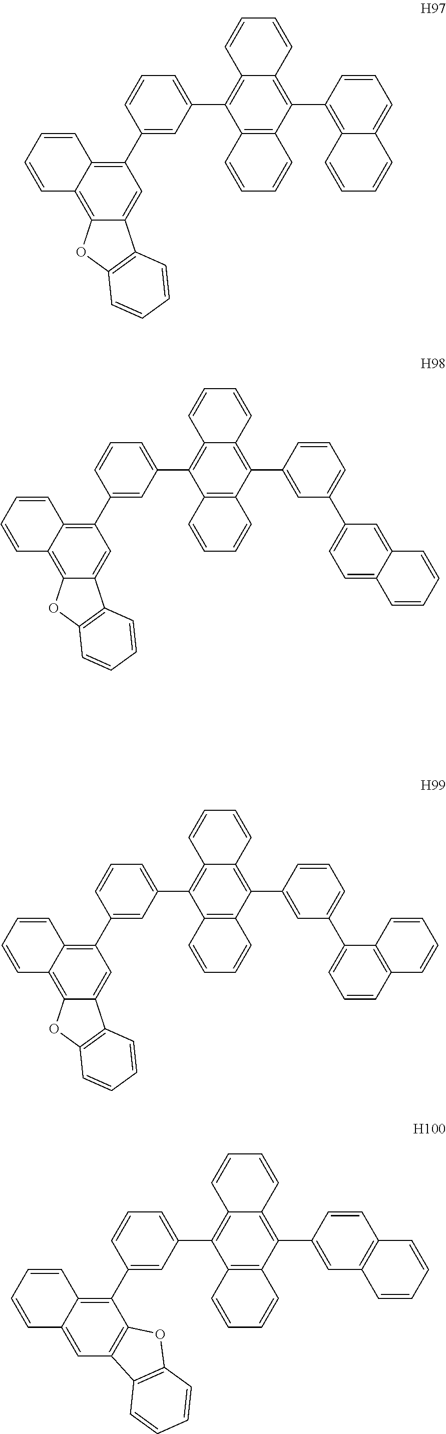 [SMILES: c1cc(-c2c3ccccc3c(-c3cccc4ccccc34)c3ccccc23)cc(-c2cc3c4ccccc4oc3c3ccccc23)c1 ; c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1 ; c1cc(-c2c3ccccc3c(-c3ccc4ccccc4c3)c3ccccc23)cc(-c2c3ccccc3cc3c2oc2ccccc23)c1]

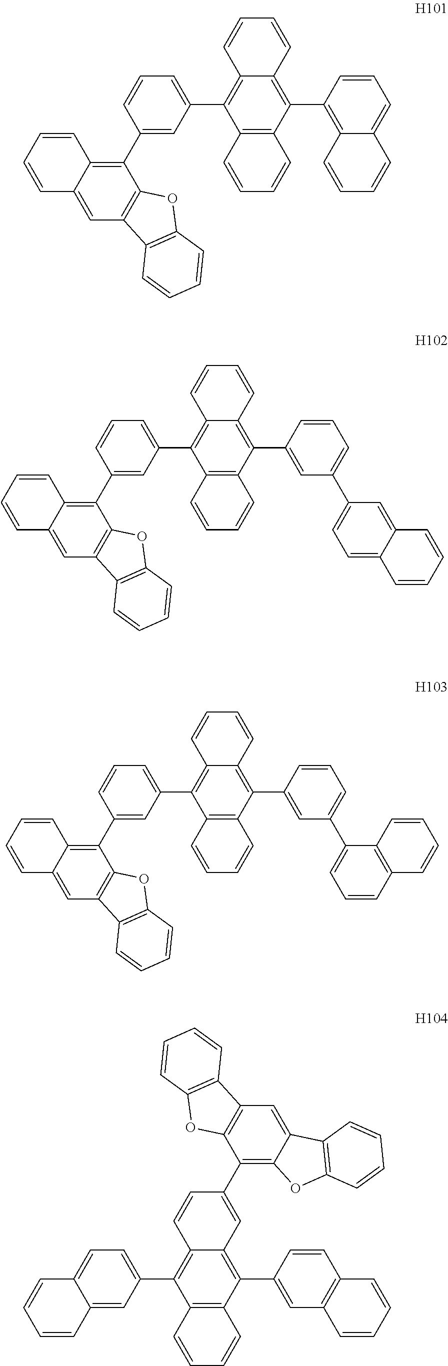 [SMILES: c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1]

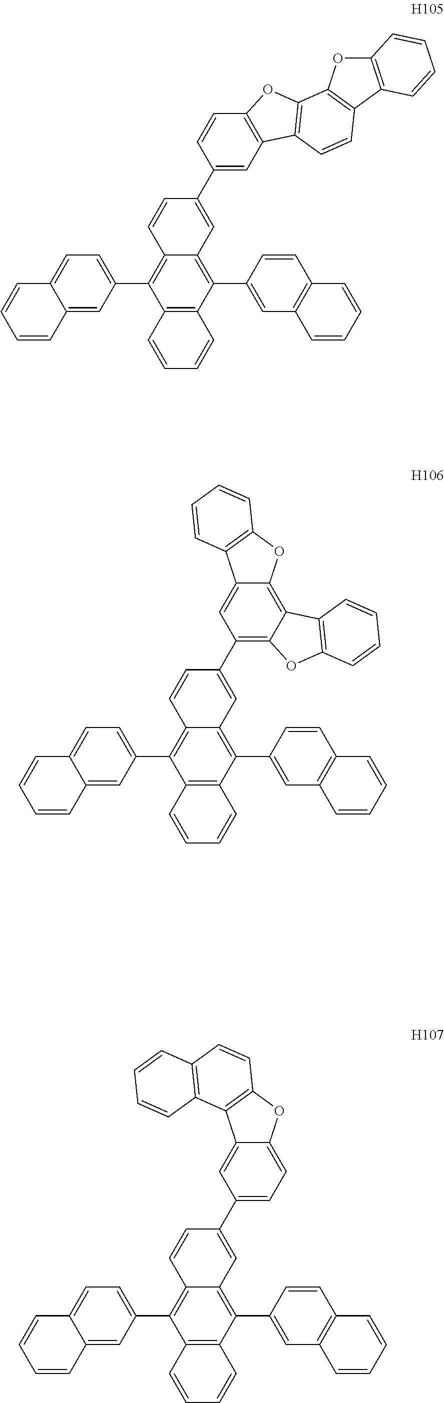 [SMILES: c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1]

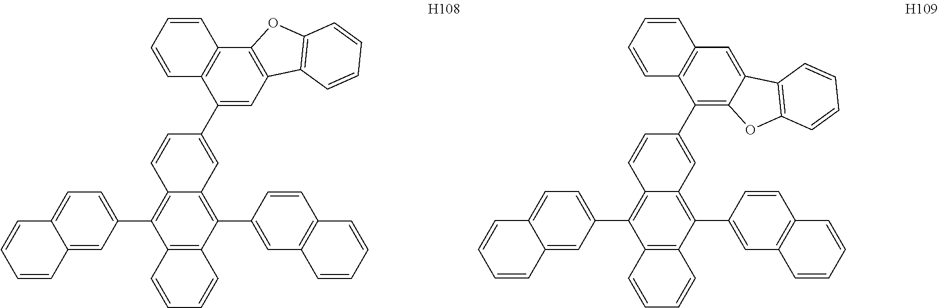 [SMILES: c1ccc2cc(-c3c4ccccc4c(-c4ccc5ccccc5c4)c4cc(-c5cc6c7ccccc7oc6c6ccccc56)ccc34)ccc2c1 ; c1ccc2cc(-c3c4ccccc4c(-c4ccc5ccccc5c4)c4cc(-c5c6ccccc6cc6c5oc5ccccc56)ccc34)ccc2c1]

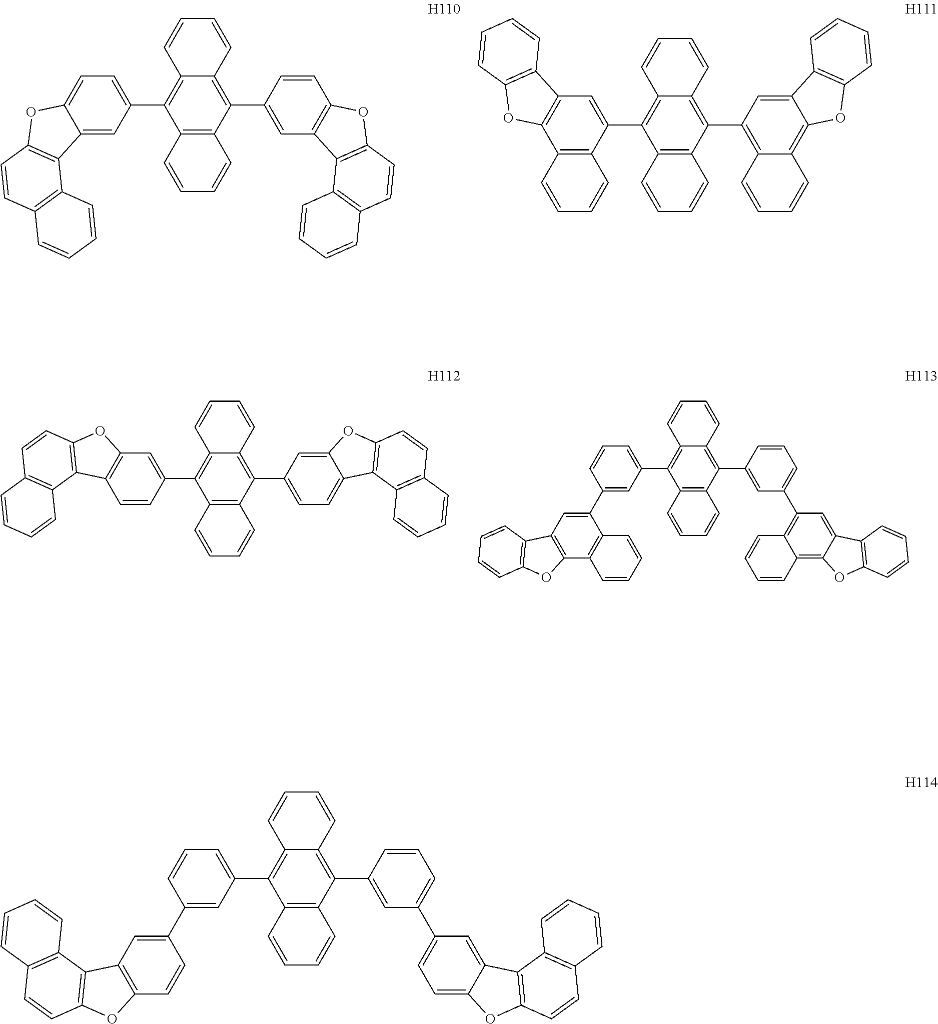

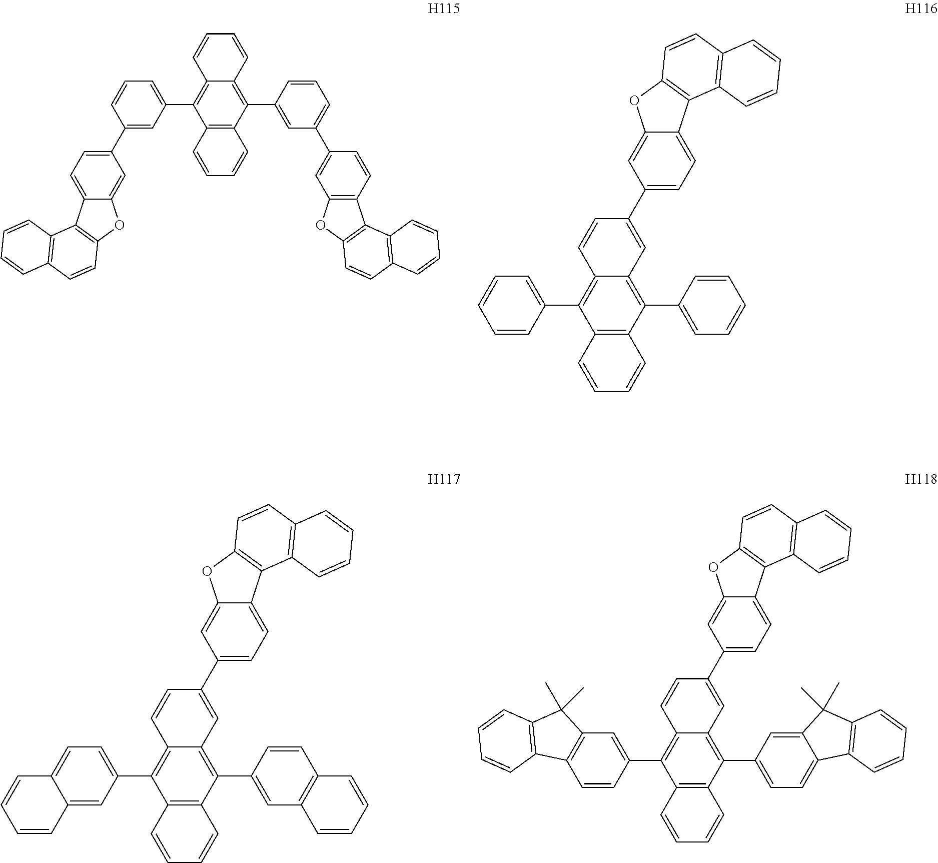 [SMILES: c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

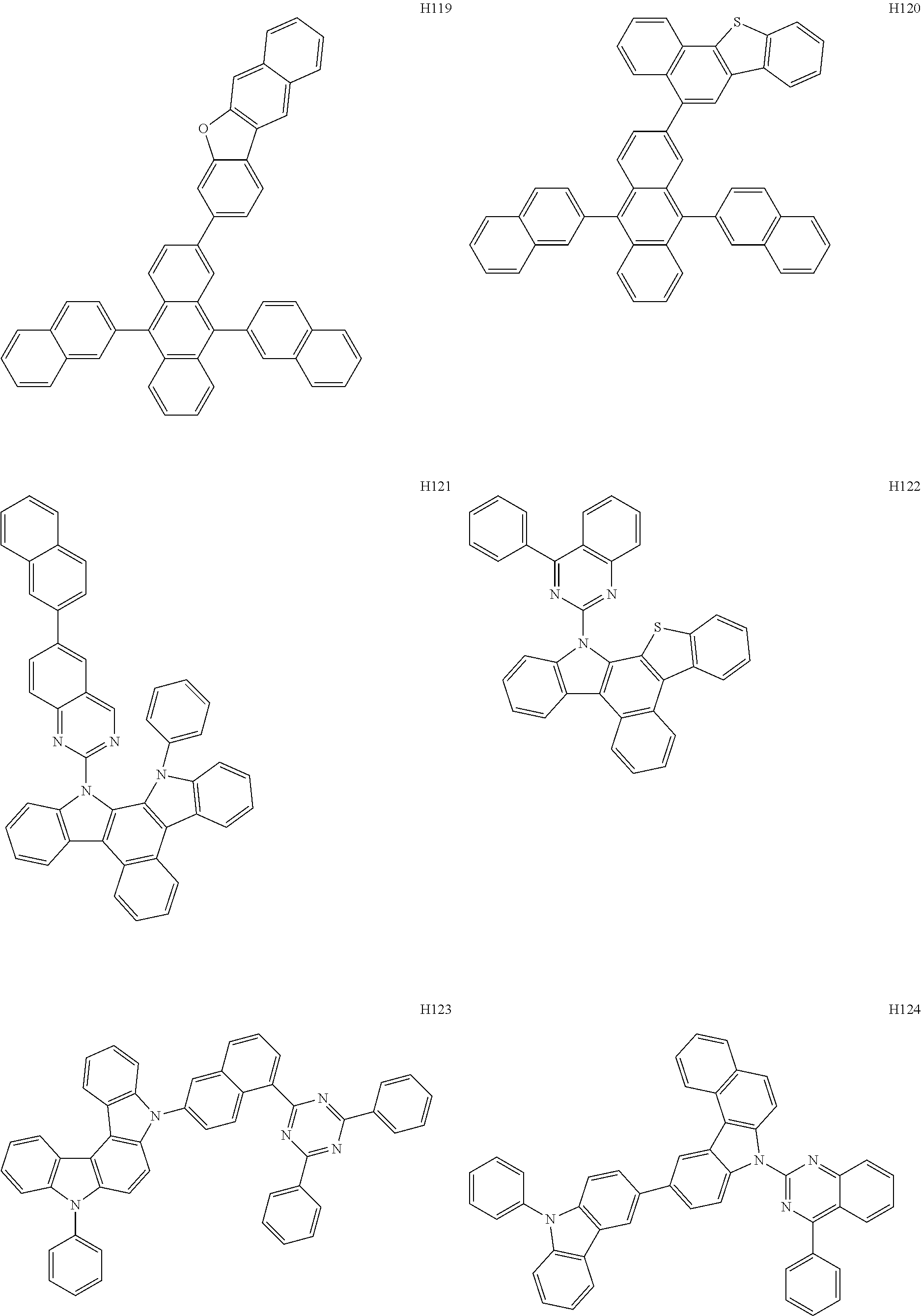

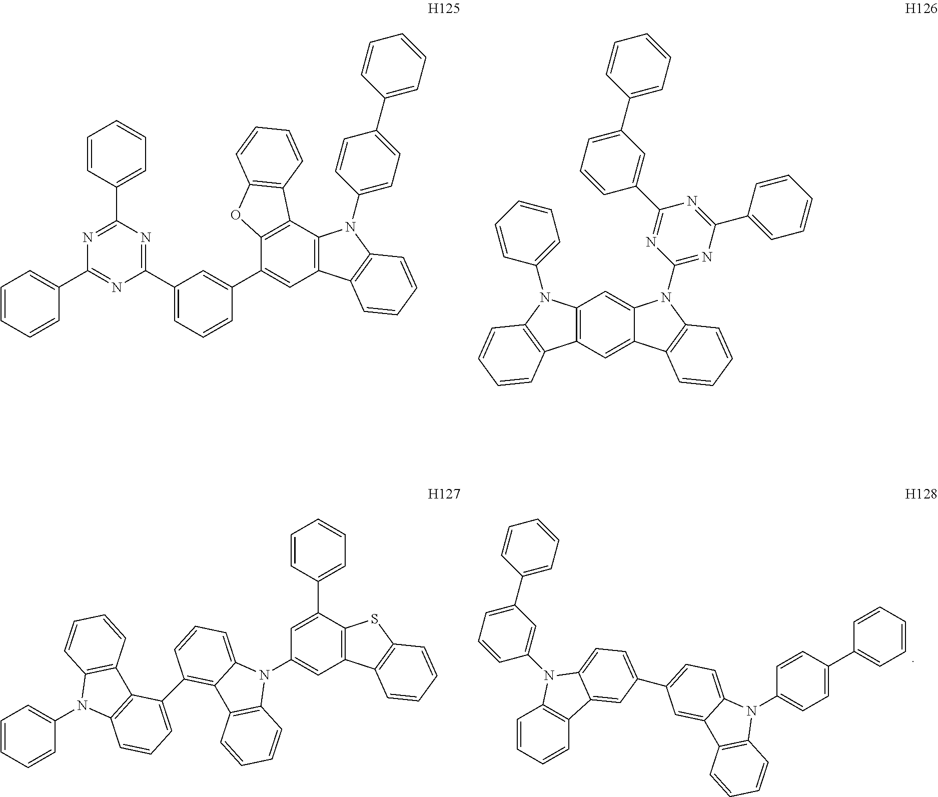 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

### [Phosphorescent Dopant]

The phosphorescent dopant may include at least one transition metal as a core metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

For example, the phosphorescent dopant may include an organometallic compound represented by Formula 401:

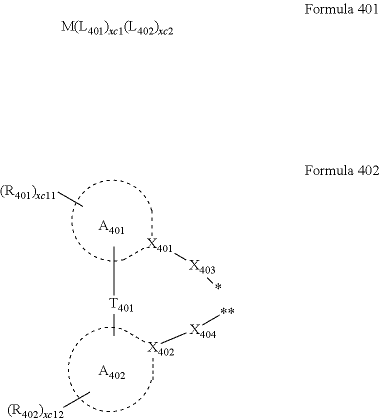

wherein, in Formulae 401 and 402,
M may be a transition metal (for example, iridium (Ir), platinum (Pt), palladium (Pd), osmium (Os), titanium (T₁), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein, when xc1 is 2 or more, two or more of L₄₀₁ may be identical to or different from each other,
L₄₀₂ may be an organic ligand, and xc2 may be 0, 1, 2, 3, or 4, wherein, when xc2 is 2 or more, two or more of L₄₀₂ may be identical to or different from each other,
X₄₀₁ and X₄₀₂ may each independently be N or C,
rings A₄₀₁ and A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)—*′, *—C(Q₄₁₁)(Q₄₁₂)—*′, *—C(Q₄₁₁)═C(Q₄₁₂)—*′, *—C(Q₄₁₁)═*′, or *═C═*′,
X₄₀₃ and X₄₀₄ may each independently be a chemical bond (for example, a covalent bond or a coordination bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ may each be as described herein in connection with Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may each be as described herein in connection with Q₁,
xc11 and xc12 may each independently be an integer from 0 to 10, and
* and *′ in Formula 402 each indicate a binding site to M in Formula 401.

For example, in Formula 402, i) X₄₀₁ may be nitrogen and X₄₀₂ may be carbon, or ii) each of X₄₀₁ and X₄₀₂ may be nitrogen.

In some embodiments, when xc1 in Formula 401 is 2 or more, two ring A₄₀₁(s) among two or more of L₄₀₁ may optionally be linked to each other via T₄₀₂, which is a linking group, and two ring A₄₀₂(s) among two or more of L₄₀₁ may optionally be linked to each other via T₄₀₃, which is a linking group (see Compounds PD1 to PD4 and PD7). T₄₀₂ and T₄₀₃ may each independently be as described herein in connection with T₄₀₁.

In Formula 401, L₄₀₂ may be an organic ligand. For example, L₄₀₂ may include a halogen group, a diketone group (for example, an acetylacetonate group), a carboxylic acid group (for example, a picolinate group), —C(═O), an isonitrile group, a —CN group, a phosphorus group (for example, a phosphine group, a phosphite group, or the like), or any combination thereof.

The phosphorescent dopant may include, for example, one of Compounds PD1 to PD39, or any combination thereof:

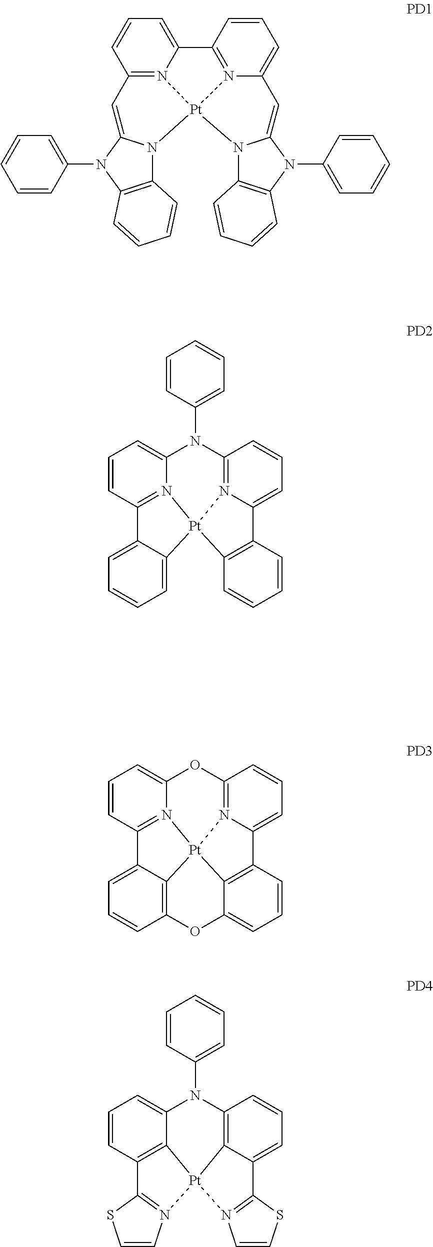

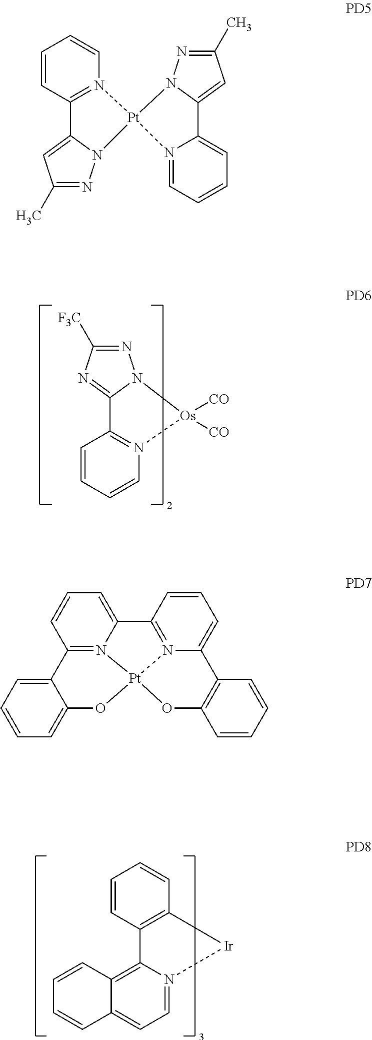

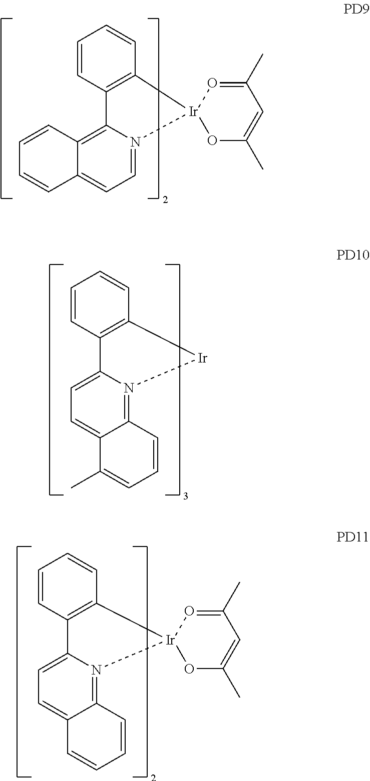

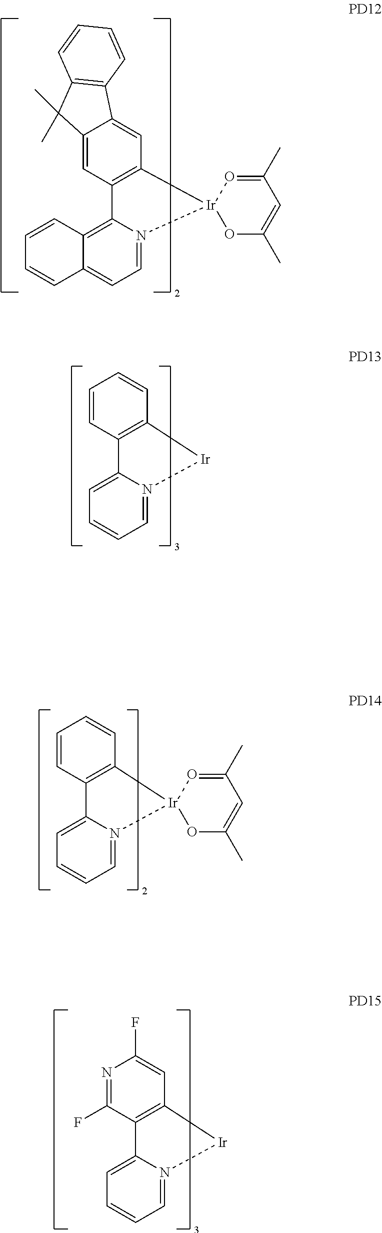

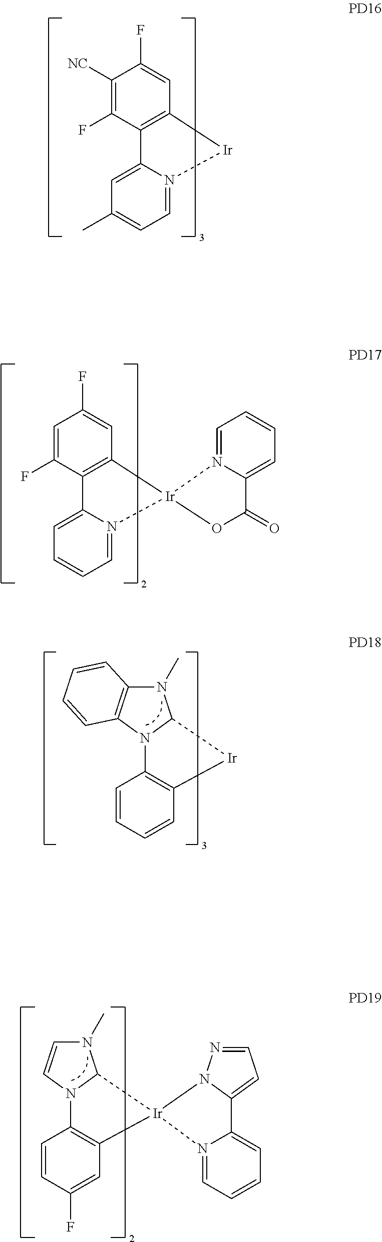

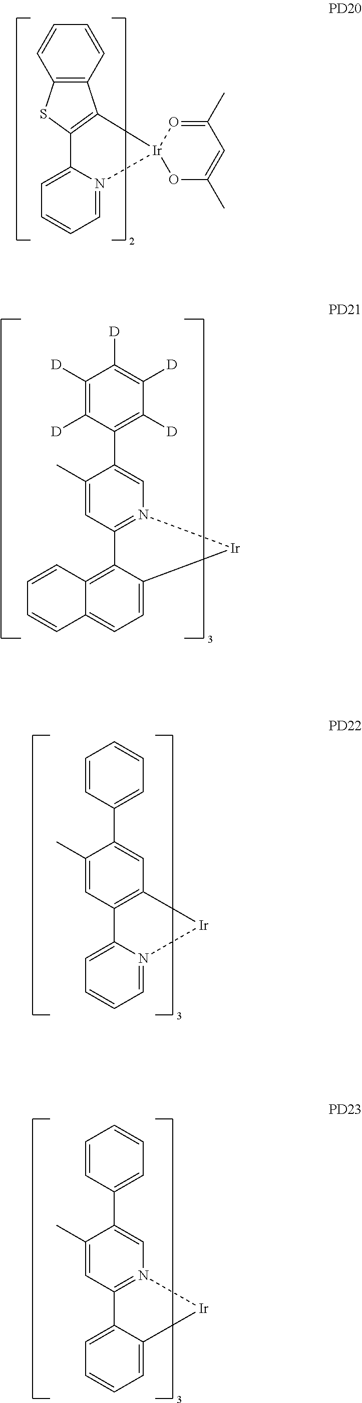

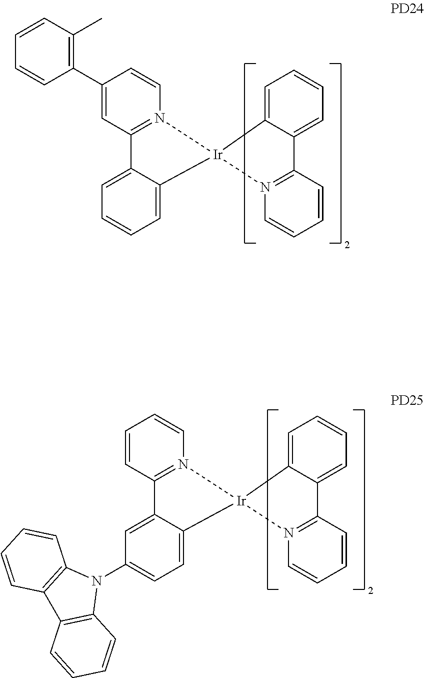

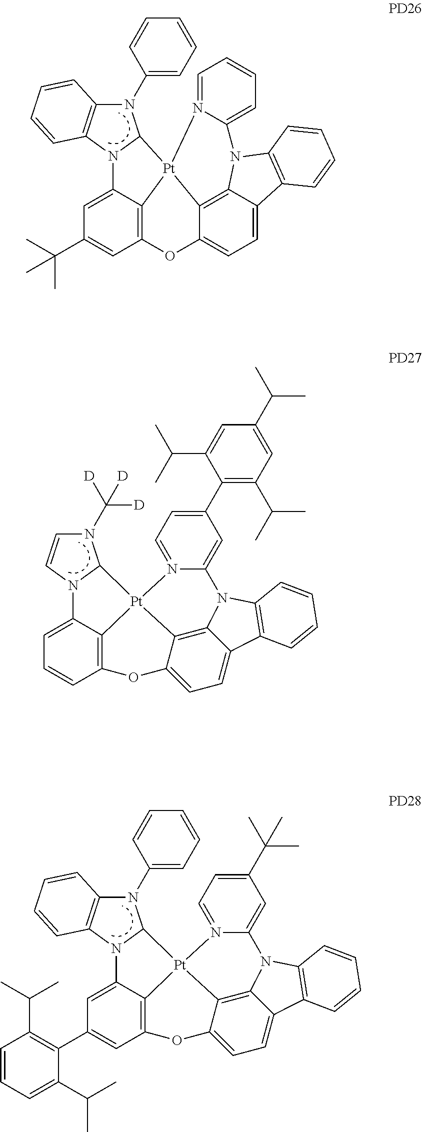

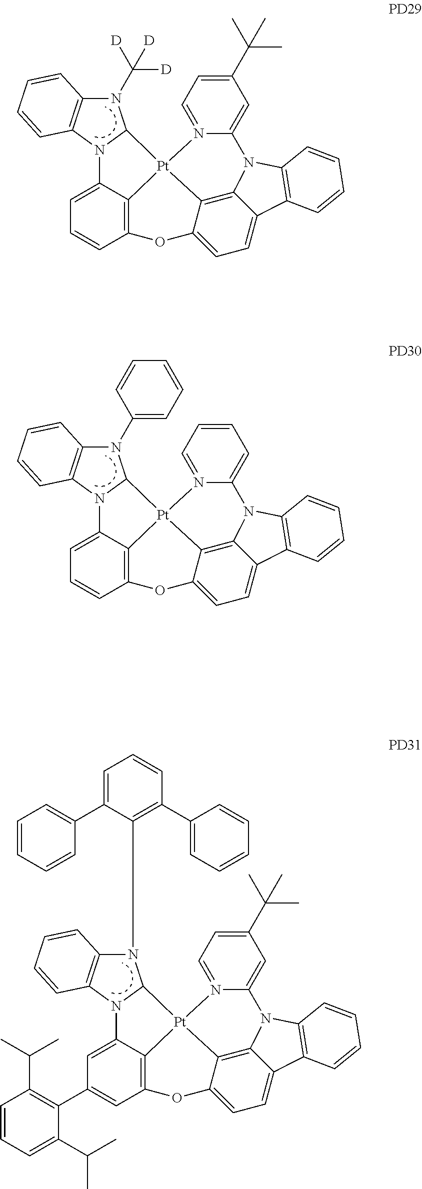

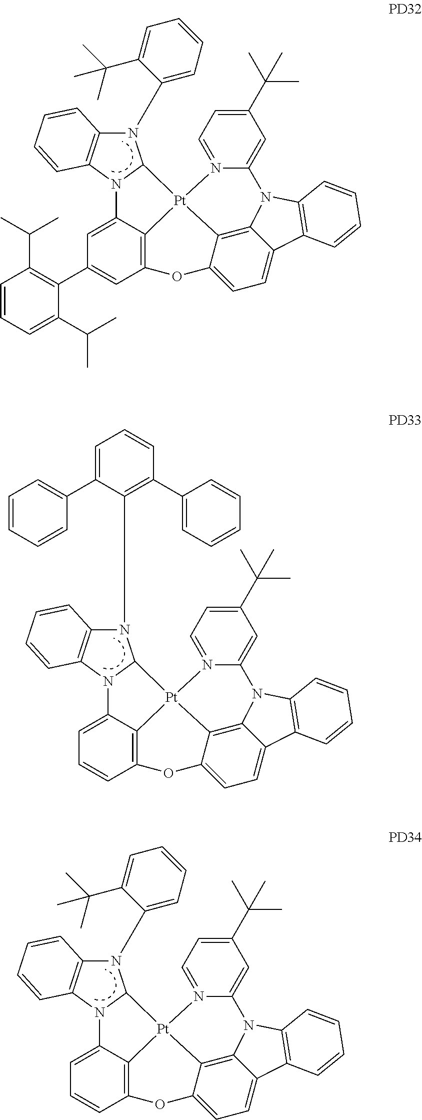

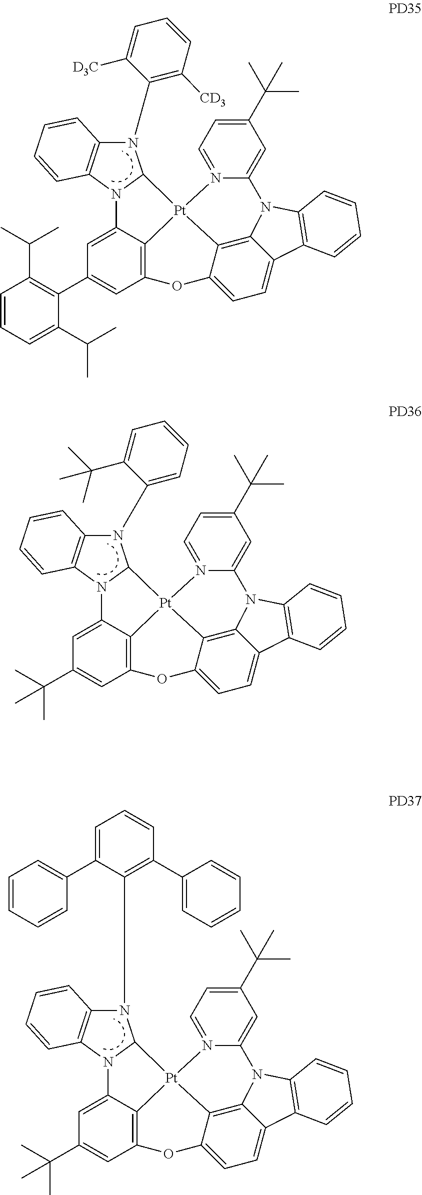

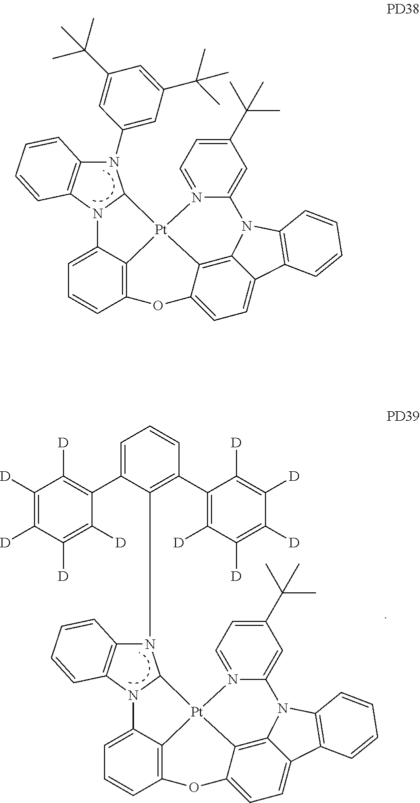

### [Fluorescent Dopant]

The fluorescent dopant may include an amine group-containing compound, a styryl group-containing compound, or any combination thereof.

For example, the fluorescent dopant may include a compound represented by Formula 501:

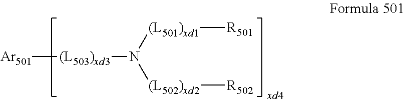

wherein, in Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

For example, Ar₅₀₁ in Formula 501 may be a condensed cyclic group (for example, an anthracene group, a chrysene group, a pyrene group, or the like) in which three or more monocyclic groups are condensed together.

In some embodiments, xd4 in Formula 501 may be 2.

For example, the fluorescent dopant may include: one of Compounds FD1 to FD37; DPVBi; DPAVBi; or any combination thereof:

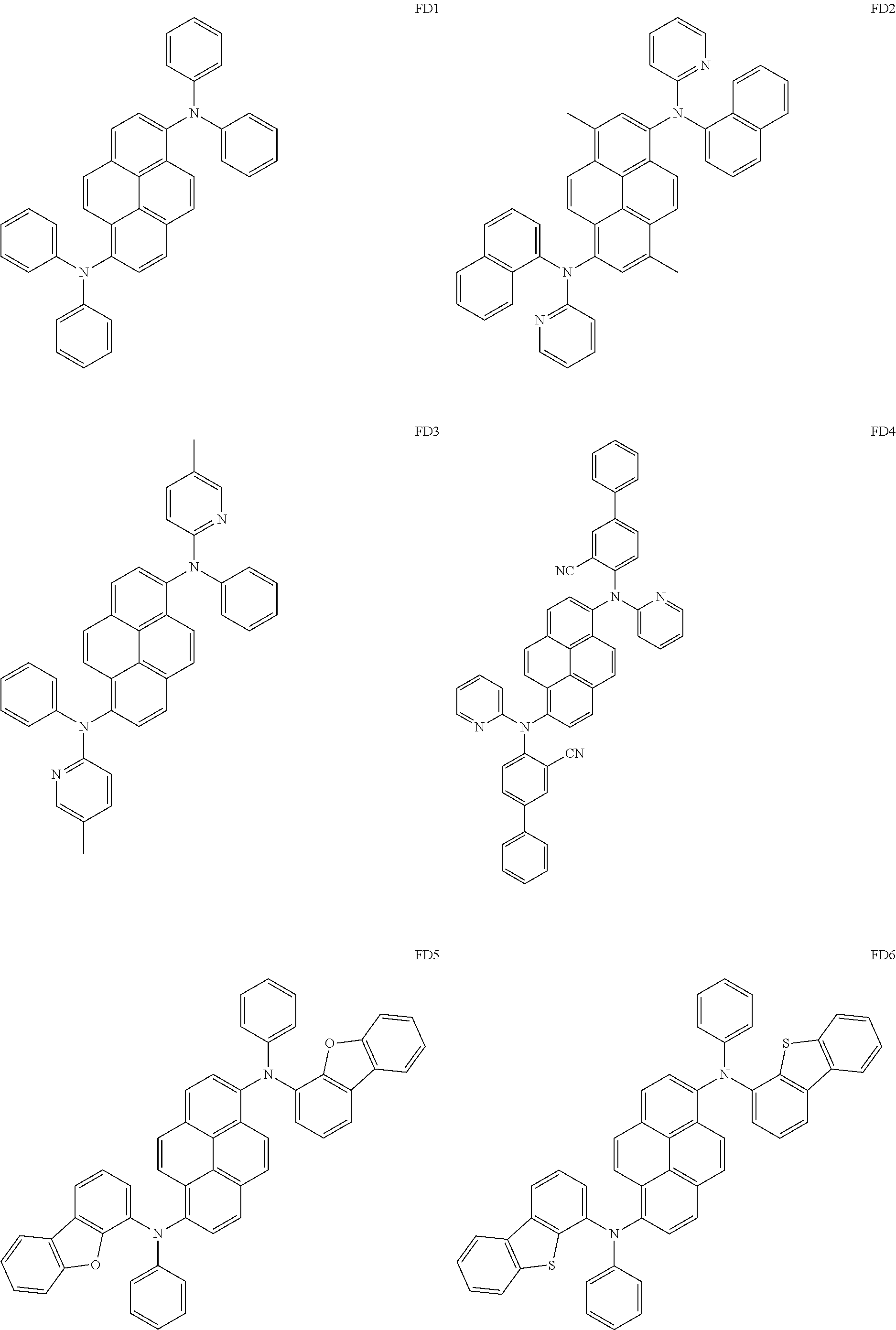

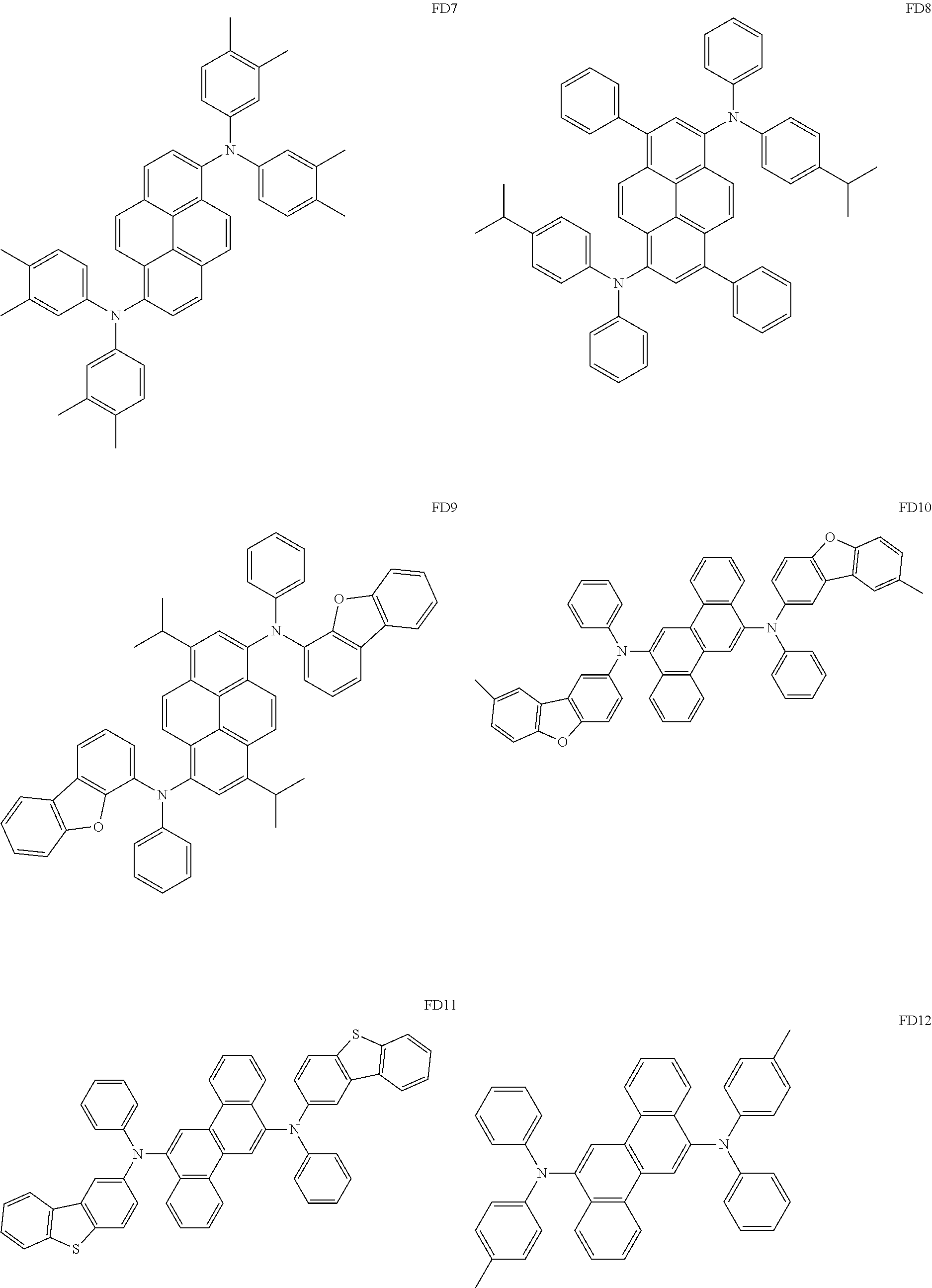

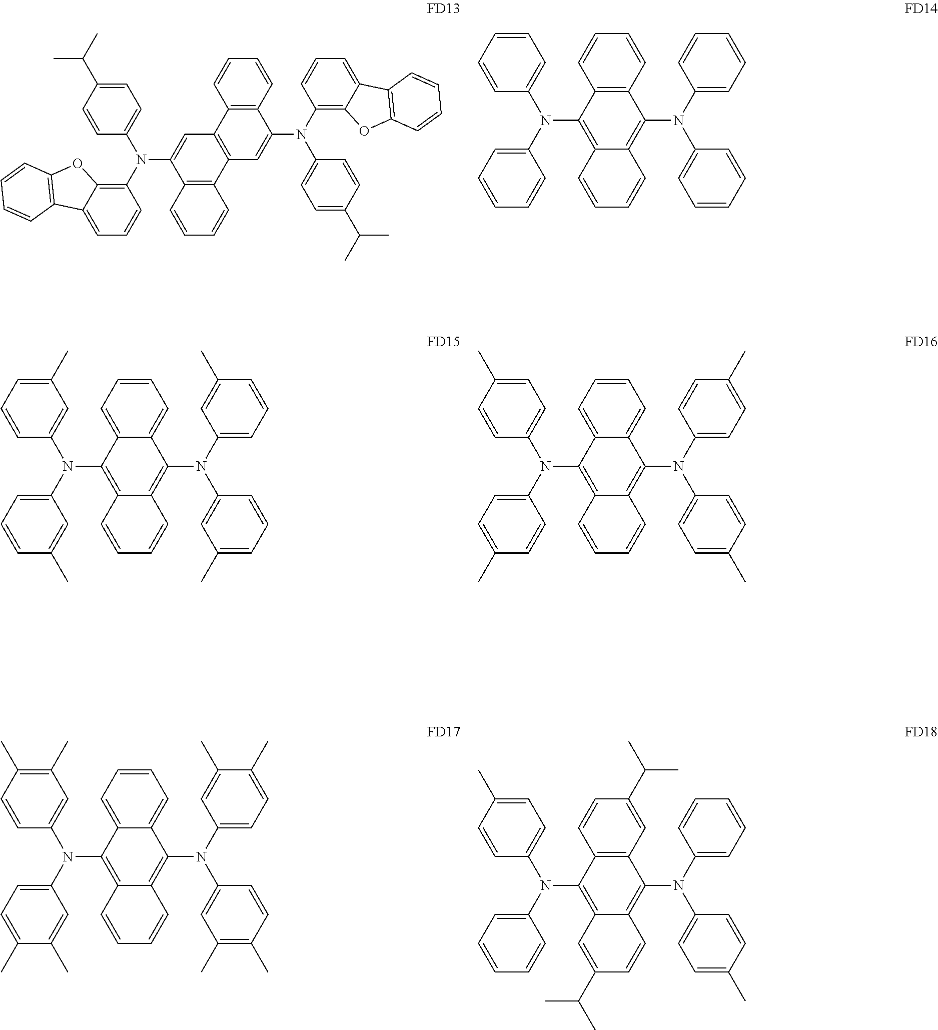

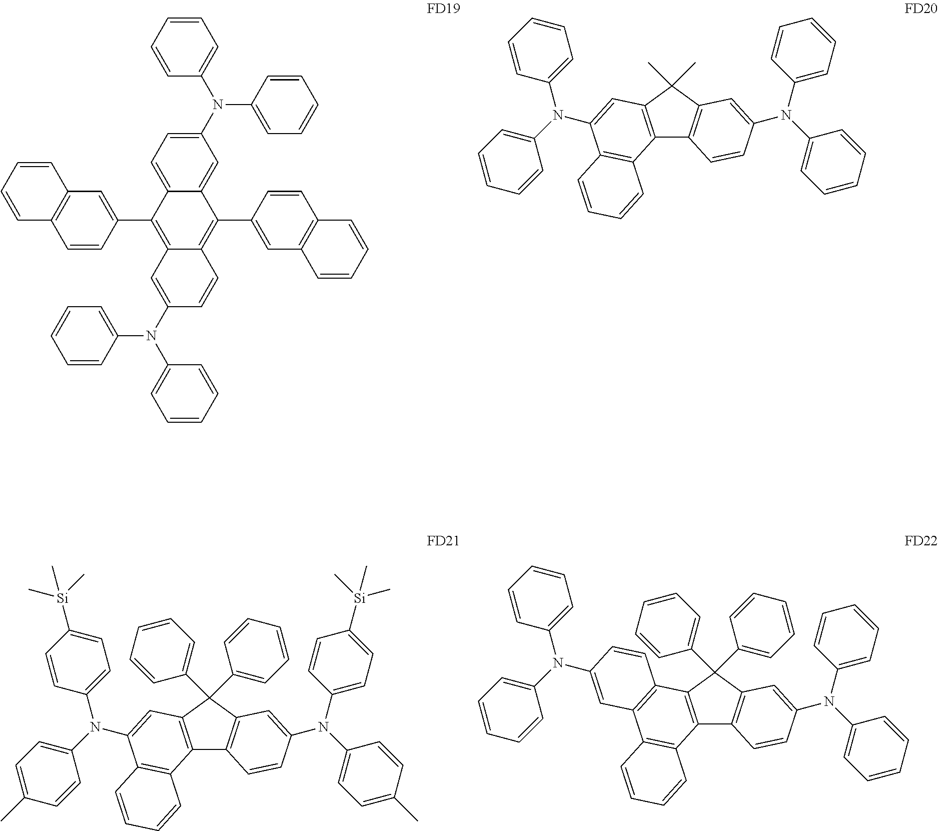

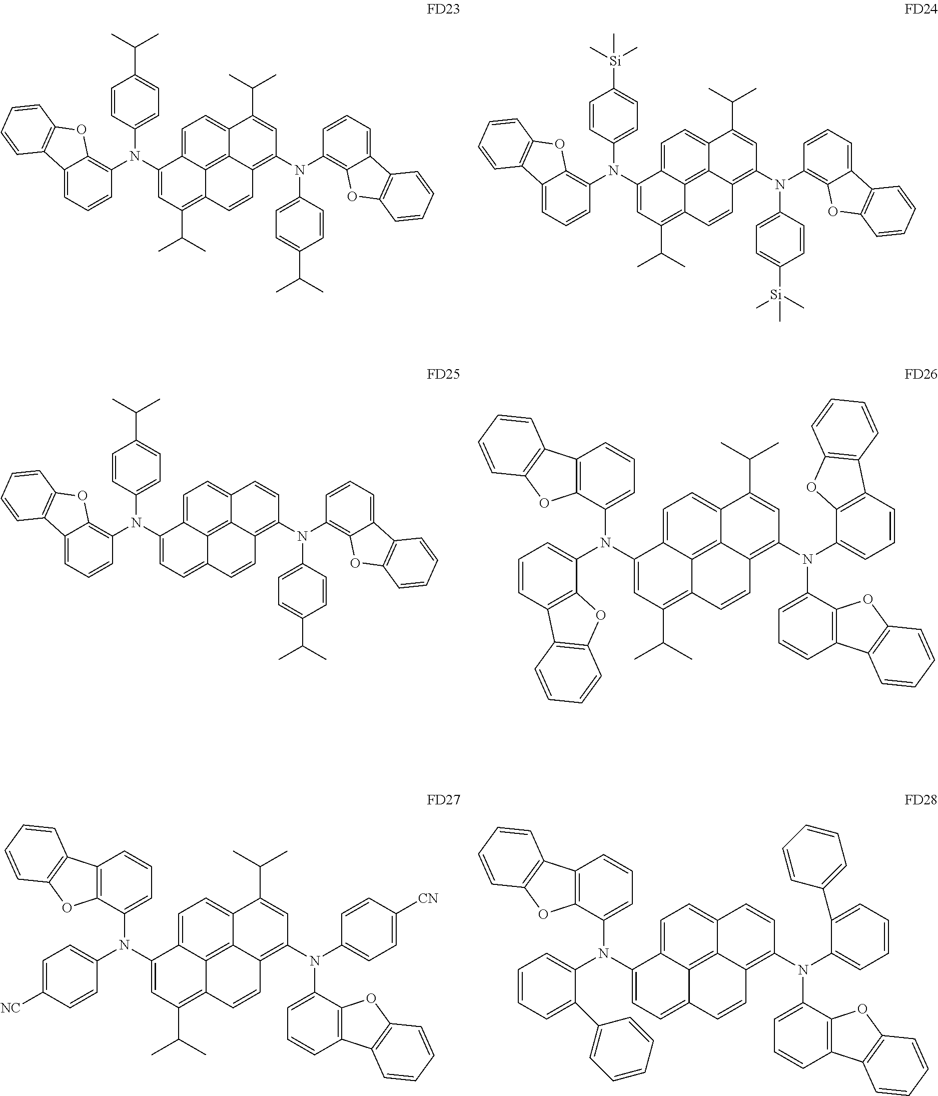

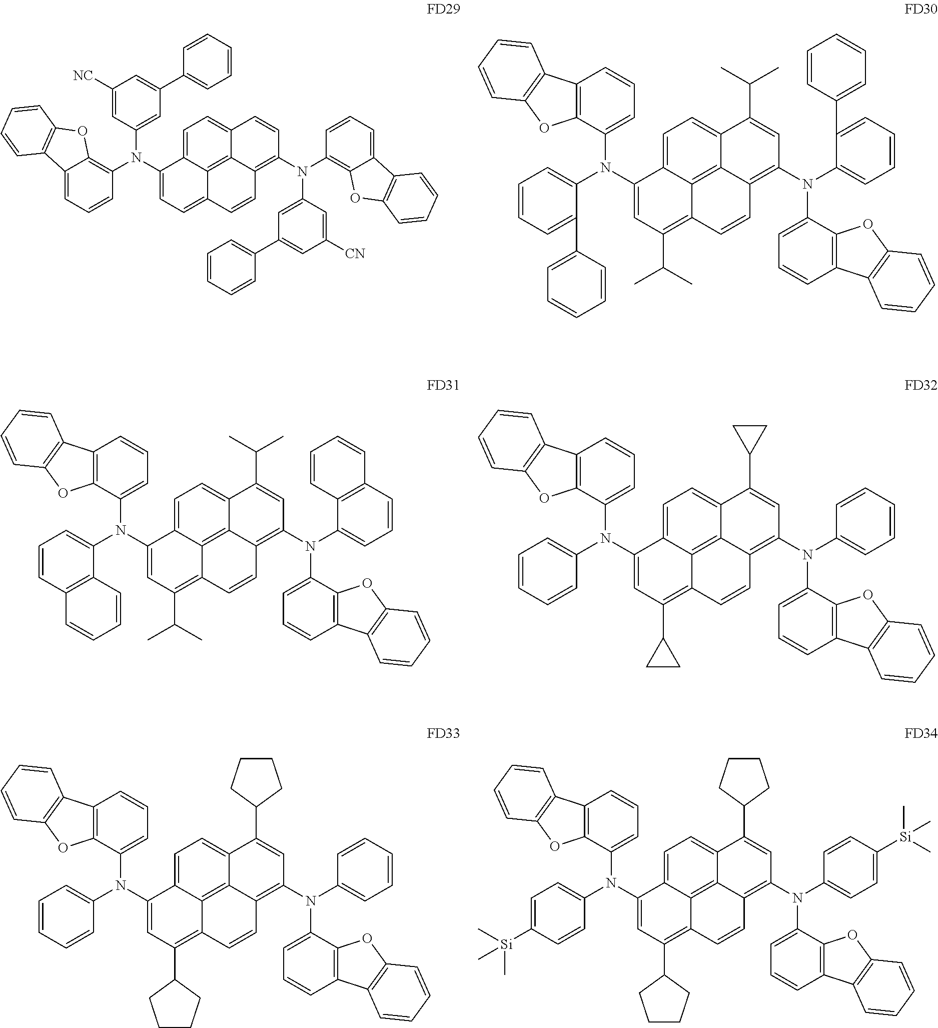

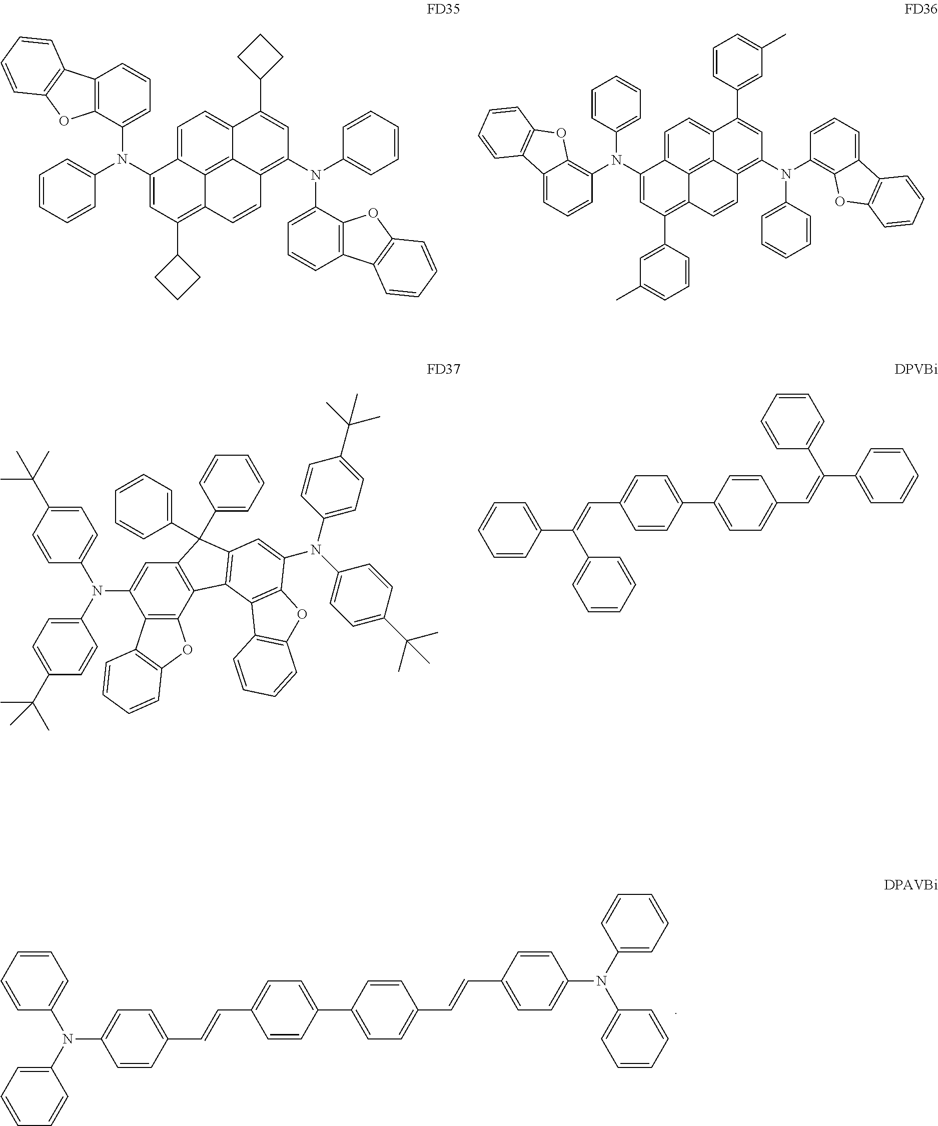

### [Delayed Fluorescence Material]

The emission layer 130 may include a delayed fluorescence material.

In the present specification, the delayed fluorescence material may be selected from compounds capable of emitting delayed fluorescence based on a delayed fluorescence emission mechanism.

The delayed fluorescence material included in the emission layer may act as a host or a dopant depending on the type of other materials included in the emission layer.

In an embodiment, a difference between a triplet energy level (eV) of the delayed fluorescence material and the singlet energy level (eV) of the delayed fluorescence material may be in a range of about 0 eV to about 0.5 eV. When the difference between the triplet energy level (eV) of the delayed fluorescence material and the singlet energy level (eV) of the delayed fluorescence material is within the above range, up-conversion from the triplet state to the singlet state of the delayed fluorescence materials may effectively occur, and thus, the light-emitting device 10 may have improved luminescence efficiency.

For example, the delayed fluorescence material may include: i) a material including at least one electron donor (for example, a π electron-rich C₃-C₆₀ cyclic group and the like, such as a carbazole group) and at least one electron acceptor (for example, a sulfoxide group, a cyano group, a π electron-deficient nitrogen-containing C₁-C₆₀ cyclic group, and the like), ii) a material including a C₈-C₆₀ polycyclic group including at least two cyclic groups condensed to each other while sharing boron (B), and the like.

Examples of the delayed fluorescence material may include at least one of Compounds DF1 to DF14:

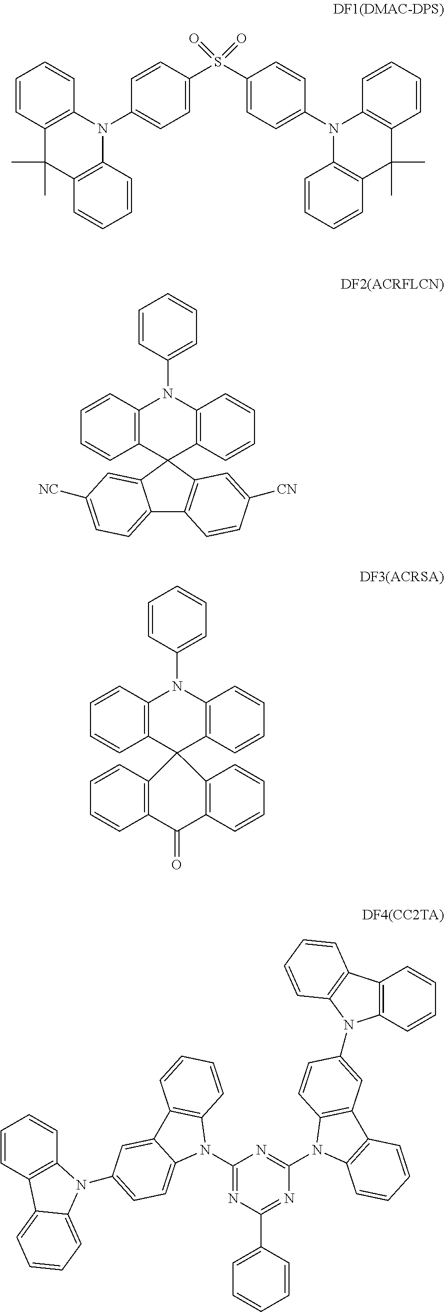

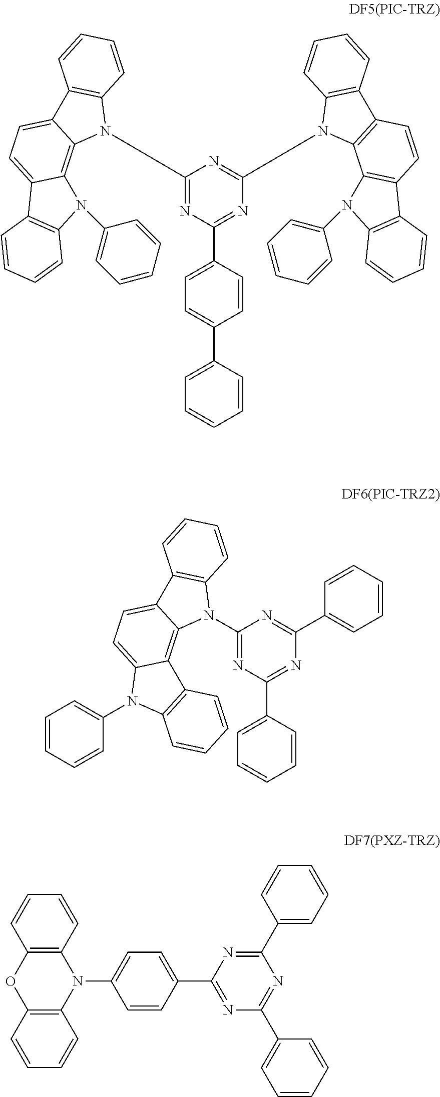 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccc(N4c5ccccc5Oc5ccccc54)cc3)n2)cc1]

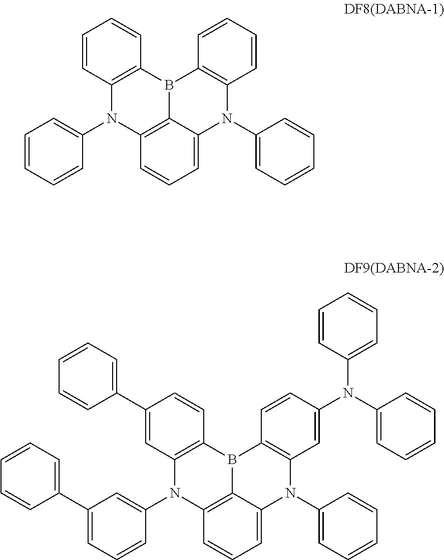 [SMILES: c1ccc(N2c3ccccc3B3c4ccccc4N(c4ccccc4)c4cccc2c43)cc1 ; c1ccc(-c2cccc(N3c4cc(-c5ccccc5)ccc4B4c5ccc(N(c6ccccc6)c6ccccc6)cc5N(c5ccccc5)c5cccc3c54)c2)cc1]

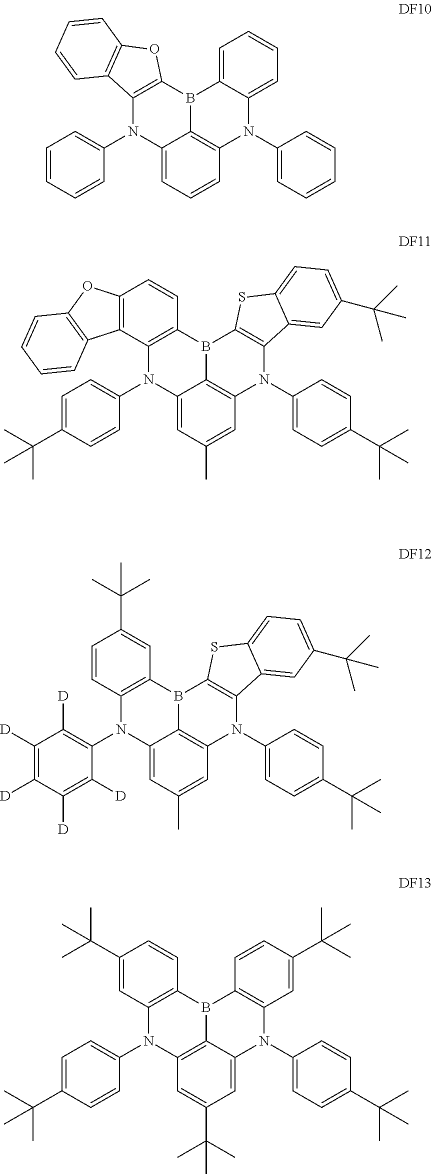

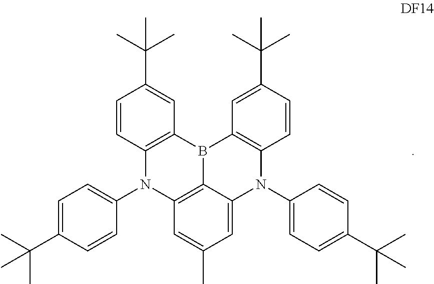 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

### [Quantum Dots]

The emission layer 130 may include quantum dots.

The term “quantum dot” as used herein refers to a crystal of a semiconductor compound, and may include any material capable of emitting light of various emission wavelengths according to the size of the crystal.

A diameter of the quantum dot may be in a range of, for example, about 1 nm to about 10 nm.

The quantum dots may be synthesized by a wet chemical process, a metal organic chemical vapor deposition process, a molecular beam epitaxy process, or any similar process.

The wet chemical process is a method in which an organic solvent and a precursor material are mixed, and then a quantum dot particle crystal is grown. When the crystal grows, the organic solvent naturally serves as a dispersant coordinated on the surface of the quantum dot crystal and may control the growth of the crystal, and thus, the wet chemical method may be easier to perform than the vapor deposition methods, such as metal organic chemical vapor deposition (MOCVD) or molecular beam epitaxy (MBE), and the growth of quantum dot particles may be controlled through a low-cost process.

The quantum dot may include: a Group II-VI semiconductor compound; a Group III-V semiconductor compound; a Group III-VI semiconductor compound; a Group I-III-VI semiconductor compound; a Group IV-VI semiconductor compound; a Group IV element or compound; or any combination thereof.

Examples of the Group II-VI semiconductor compound may include: a binary compound, such as CdS, CdSe, CdTe, ZnS, ZnSe, ZnTe, ZnO, HgS, HgSe, HgTe, MgSe, MgS, or the like; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, or the like; a quaternary compound, such as CdZnSeS, CdZnSeTe, CdZnSTe, CdHgSeS, CdHgSeTe, CdHgSTe, HgZnSeS, HgZnSeTe, HgZnSTe, or the like; or any combination thereof.

Examples of the Group III-V semiconductor compound may include: a binary compound, such as GaN, GaP, GaAs, GaSb, AlN, AlP, AlAs, AlSb, InN, InP, InAs, InSb, or the like; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, InPSb, or the like; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, or the like; or any combination thereof.

In some embodiments, the Group III-V semiconductor compound may further include a Group II element. Examples of the Group III-V semiconductor compound further including the Group II element may include InZnP, InGaZnP, InAlZnP, and the like.

Examples of the Group III-VI semiconductor compound may include: a binary compound, such as GaS, GaSe, Ga₂Se₃, GaTe, InS, InSe, In₂S₃, In₂Se₃, InTe, or the like; a ternary compound, such as InGaS₃, InGaSe₃, or the like; or any combination thereof.

Examples of the Group I-ITT-VI semiconductor compound may include: a ternary compound, such as AgInS, AgInS₂, CuInS, CuInS₂, CuGaO₂, AgGaO₂, AgAlO₂, or the like; a quaternary compound, such as AgInGaS, AgInGaS₂, or the like; or any combination thereof.

Examples of the Group IV-VI semiconductor compound may include: a binary compound, such as SnS, SnSe, SnTe, PbS, PbSe, PbTe, or the like; a ternary compound, such as SnSeS, SnSeTe, SnSTe, PbSeS, PbSeTe, PbSTe, SnPbS, SnPbSe, SnPbTe, or the like; a quaternary compound, such as SnPbSSe, SnPbSeTe, SnPbSTe, or the like; or any combination thereof.

Examples of the Group IV element or compound are: a single element, such as Si, Ge, and the like; a binary compound, such as SiC, SiGe, and the like; or any combination thereof.

Each element included in a multi-element compound, such as the binary compound, the ternary compound, and the quaternary compound, may be present at a uniform concentration or non-uniform concentration in a particle.

In some embodiments, the quantum dot may have a single structure in which the concentration of each element in the quantum dot is uniform, or may have a core-shell dual structure. For example, a material included in the core and a material included in the shell may be different from each other.

The shell of the quantum dot may act as a protective layer that prevents chemical denaturation of the core to maintain semiconductor characteristics, and/or as a charging layer that imparts electrophoretic characteristics to the quantum dot. The shell may be a single layer or a multi-layer. The interface between the core and the shell may have a concentration gradient in which the concentration of an element present in the shell decreases toward the core.

Examples of the shell of the quantum dot may include an oxide of a metal, a metalloid, or a non-metal, a semiconductor compound, or a combination thereof. Examples of the oxide of a metal, a metalloid, or a non-metal may include: a binary compound, such as SiO₂, Al₂O₃, TiO₂, ZnO, MnO, Mn₂O₃, Mn₃O₄, CuO, FeO, Fe₂O₃, Fe₃O₄, CoO, CO₃O₄, NiO, or the like; a ternary compound, such as MgAl₂O₄, CoFe₂O₄, NiFe₂O₄, CoMn₂O₄, or the like; or any combination thereof. Examples of the semiconductor compound may include: as described herein, a Group II-VI semiconductor compound; a Group III-V semiconductor compound; a Group III-VI semiconductor compound; a Group 1-III-VI semiconductor compound; a Group IV-VI semiconductor compound; or any combination thereof. Examples of the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

The quantum dot may have a full width of half maximum (FWHM) of the emission wavelength spectrum of about 45 nm or less, for example, about 40 nm or less, for example, about 30 nm or less. When the FWHM of the quantum dot is within these ranges, color purity or color reproducibility may be improved. In addition, since light emitted through the quantum dot is emitted in all directions, an optical viewing angle may be improved.

In some embodiments, the quantum dot may be, for example, in the form of spherical, pyramidal, multi-arm, or cubic nanoparticles, nanotubes, nanowires, nanofibers, or nanoplate particles.

By adjusting the size of the quantum dot, the energy band gap may be adjusted, thereby obtaining light of various wavelengths in the quantum dot emission layer. Therefore, by using quantum dots of different sizes, a light-emitting device that emits light of various wavelengths may be realized. In some embodiments, the size of the quantum dot may be selected such that the quantum dot may emit red light, green light, and/or blue light. In some embodiments, the size of the quantum dot may be configured such that the quantum dot may emit white light by combination of light of various colors. [Photoactive layer 135]

The optoelectronic device 30 may include the photoactive layer 135 on the hole transport region 120. The photoactive layer 135 may be disposed between the hole transport region 120 and the electron transport region 140. In an embodiment, the photoactive layer 135 may be disposed between the hole transport layer included in the hole transport region 120 and a buffer layer included in the electron transport region 140. In other embodiments, the photoactive layer 135 may be disposed between the emission auxiliary layer included in the hole transport region 120 and a buffer layer included in the electron transport region 140.

The photoactive layer 135 may include the first compound and the second compound. The first compound may be referred to as an electron-donor compound or a p-type compound, and the second compound may be referred to as an electron-acceptor compound or an n-type compound.

The first compound and the second compound may be included in the photoactive layer 135 in a mixed state. For example, the photoactive layer 135 may be a single layer including the first compound and the second compound.

The photoactive layer 135 may produce excitons by absorbing incident light. The excitons are capable of generating holes and electrons. The holes generated by the photoactive layer 135 may move to the first electrode 110 through the hole transport region 120. The electrons generated by the photoactive layer 135 may move to the second electrode 150 through the electron transport region 140.

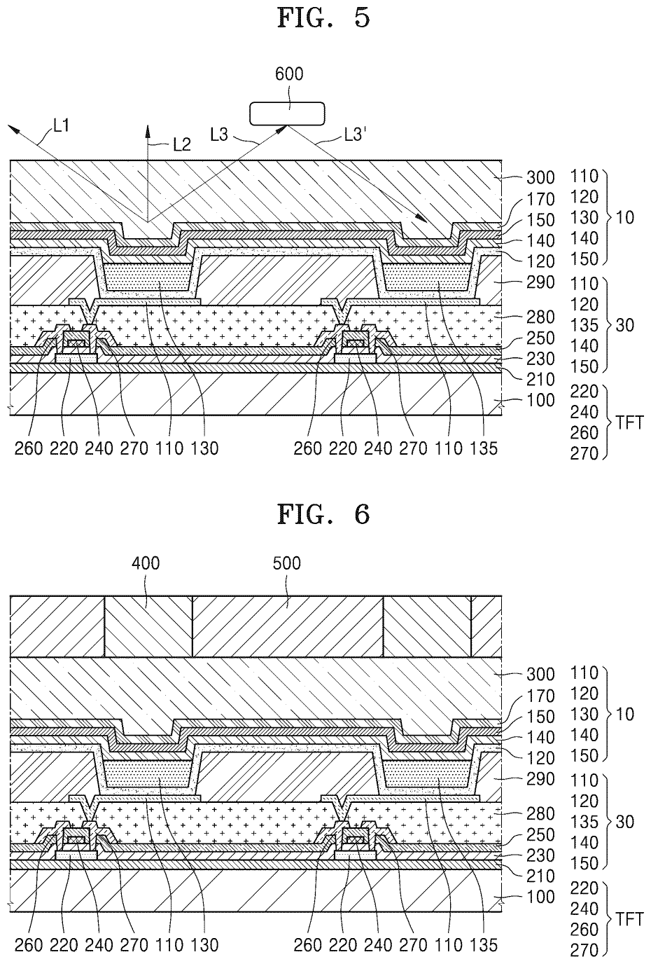

In other embodiments, the photoactive layer 135 may generate electrical signals by absorbing light. In some embodiments, the first compound included in the photoactive layer 135 may serve as a donor for supplying electrons, and the second compound included in the photoactive layer 135 may serve as an acceptor for receiving electrons. Thus, the optoelectronic device 30 including the photoactive layer 135 may serve as an optical sensor. For example, the optoelectronic device 30 may serve as a fingerprint sensor, which will be described below with reference to FIG. 5.

### [Electron Transport Region 140]

The electron transport region may have: i) a single-layered structure of a single layer containing a single material, ii) a single-layered structure of a single layer containing a plurality of different materials, or iii) a multi-layered structure having a plurality of layers including a plurality of different materials.

The electron transport region may include a buffer layer, a hole blocking layer, an electron control layer, an electron transport layer, an electron injection layer, or any combination thereof.

For example, the electron transport region may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, in which constituent layers of each structure are sequentially stacked from the emission layer.

The electron transport region (for example, the buffer layer, the hole blocking layer, the electron control layer, or the electron transport layer in the electron transport region) may include a metal-free compound including at least one π electron-deficient nitrogen-containing C₁-C₆₀ cyclic group.

For example, the electron transport region may include a compound represented by Formula 601:

[Ar₆₀₁]ₓₑ₁₁-[(L₆₀₁)ₓₑ₁-R₆₀₁]ₓₑ₂₁ Formula 601
wherein, in Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each be as described herein in connection with Q₁,
xe21 may be 1, 2, 3, 4, or 5, and
at least one of Ar₆₀₁, L₆₀₁, and R₆₀₁ may each independently be a 71 electron-deficient nitrogen-containing C₁-C₆₀ cyclic group unsubstituted or substituted with at least one R₁₀ₐ.

For example, when xe1 in Formula 601 is 2 or more, two or more of Ar₆₀₁ may be linked to each other via a single bond.

In some embodiments, Ar₆₀₁ in Formula 601 may be an anthracene group unsubstituted or substituted with at least one R₁₀ₐ.

In some embodiments, the electron transport region may include a compound represented by Formula 601-1:

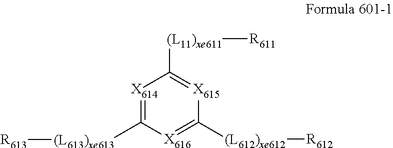

wherein, in Formula 601-1,
X₆₁₄ may be N or C(R₆₁₄), X₆₁₅ may be N or C(R₆₁₅), X₆₁₆ may be N or C(R₆₁₆), and at least one of X₆₁₄ to X₆₁₆ may be N,
L₆₁₁ to L₆₁₃ may each be as described above in connection with L₆₀₁,
xe611 to xe613 may each be as described above in connection with xe1,
R₆₁₁ to R₆₁₃ may each be as described above in connection with R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region may include: one of Compounds ET1 to ET45; 2,9-dimethyl-4,7-diphenyl-1,10-phenanthroline (BCP); 4,7-diphenyl-1,10-phenanthroline (Bphen); Alq₃; BAlq; TAZ; NTAZ; or any combination thereof:

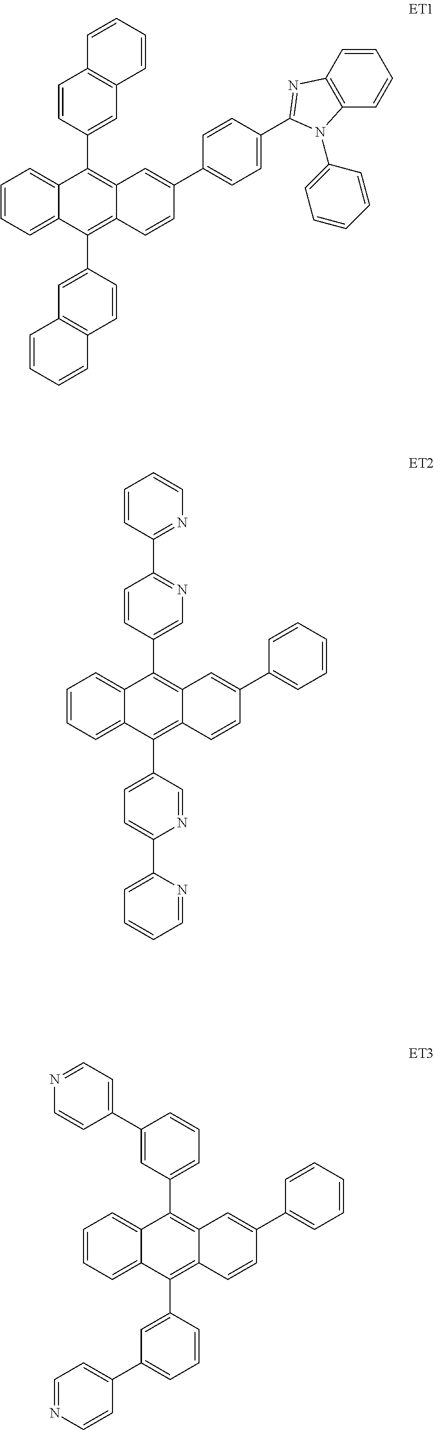 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

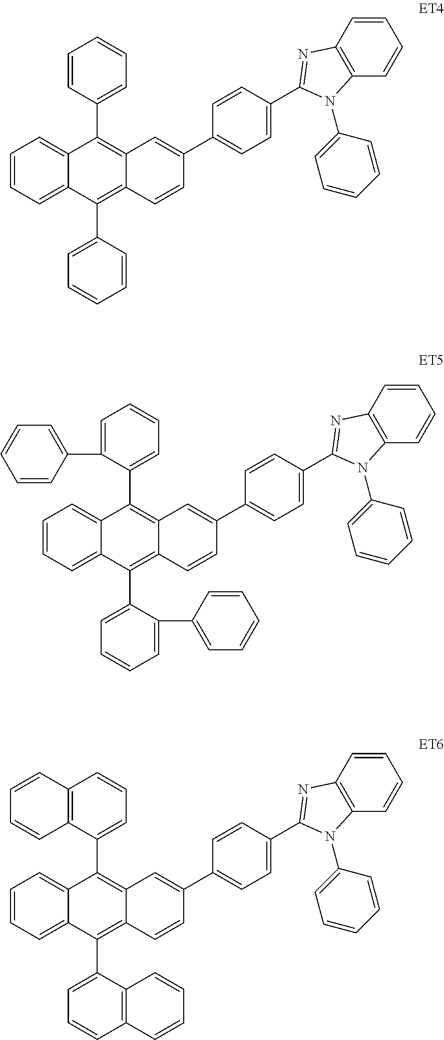 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

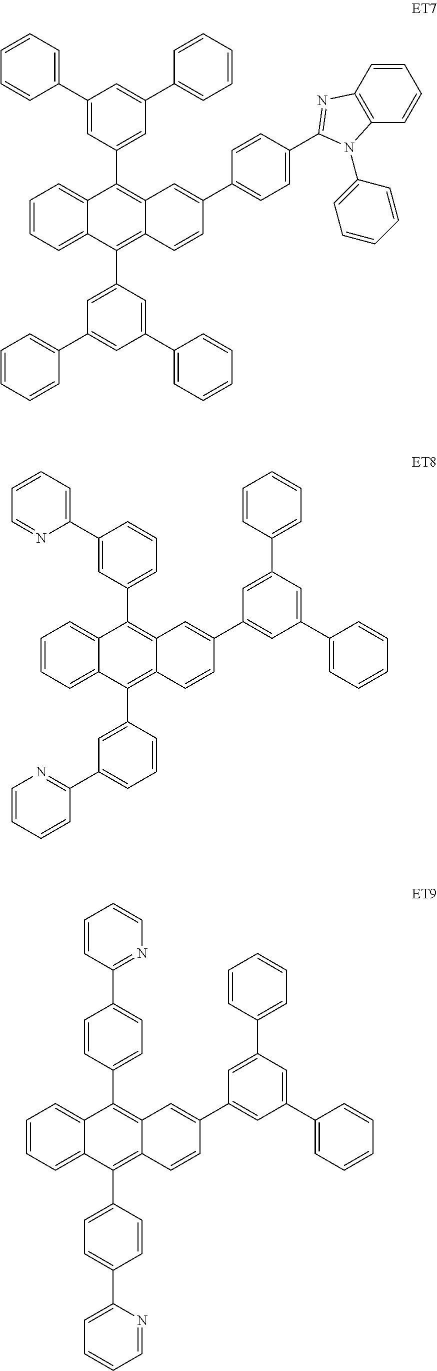 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

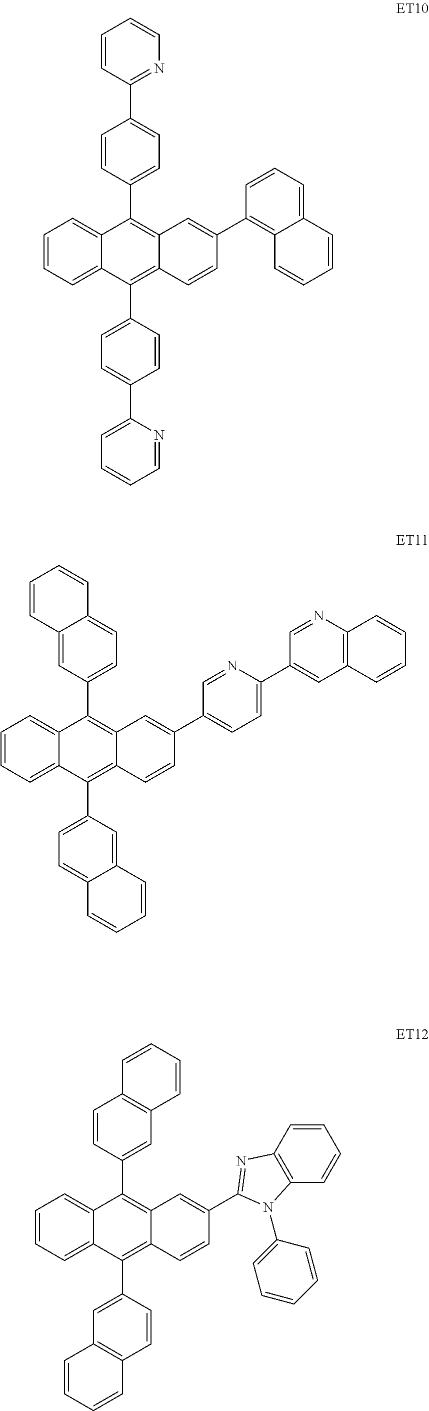 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

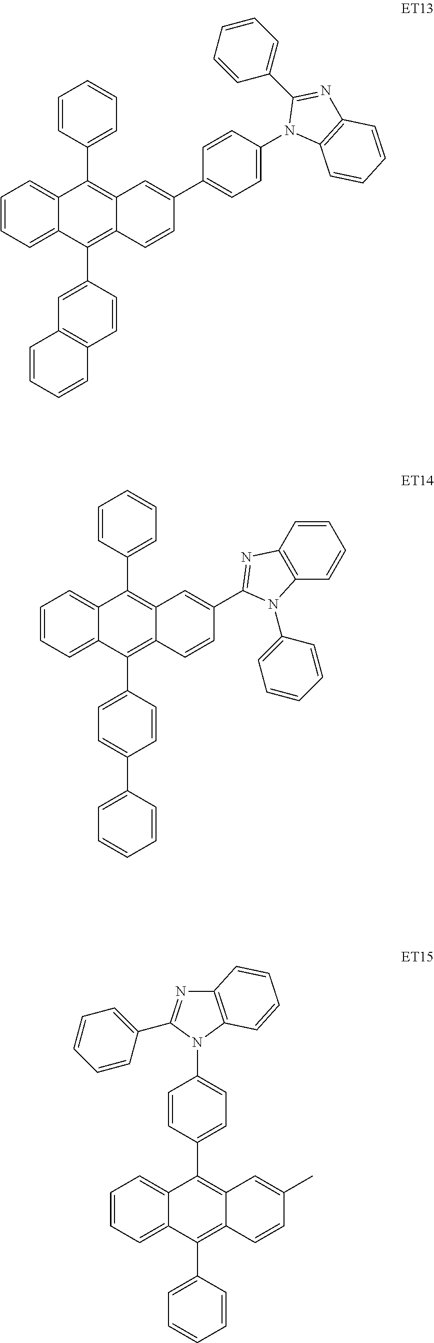 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

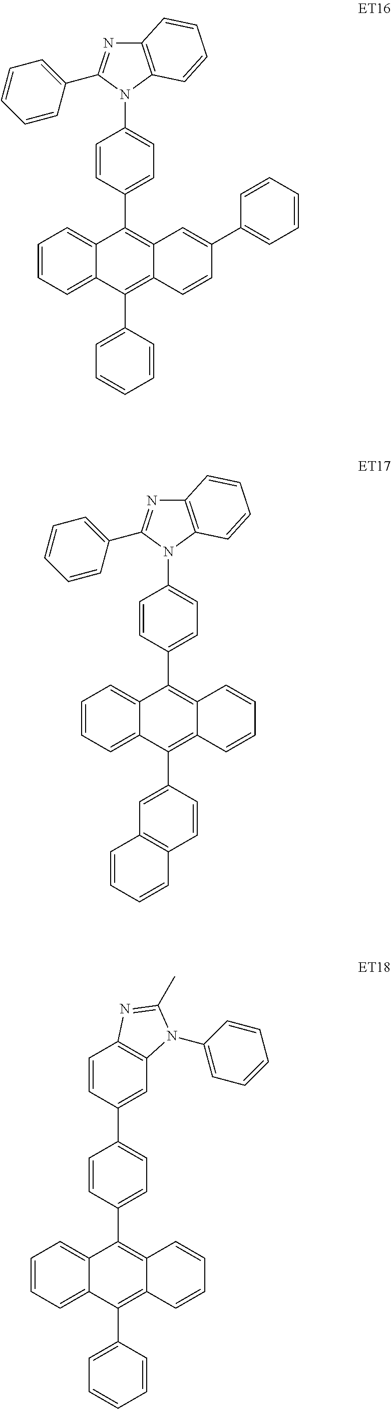 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

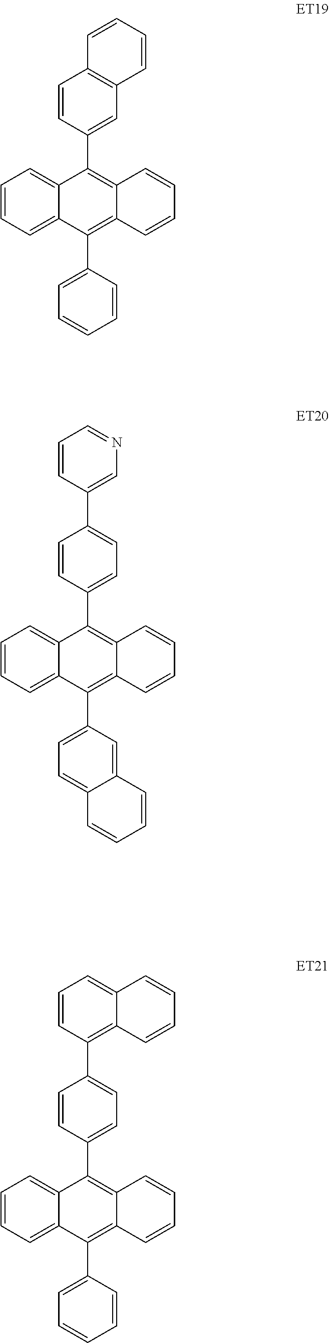

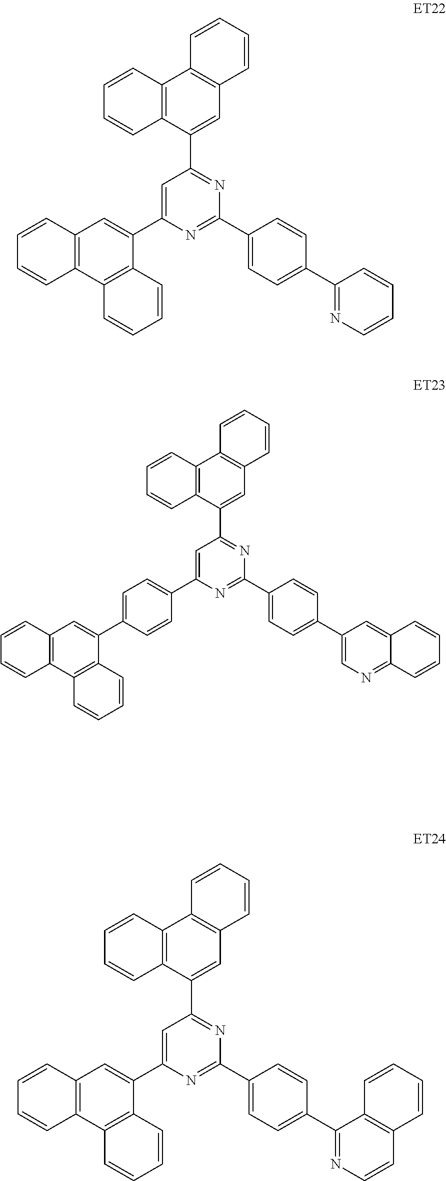 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

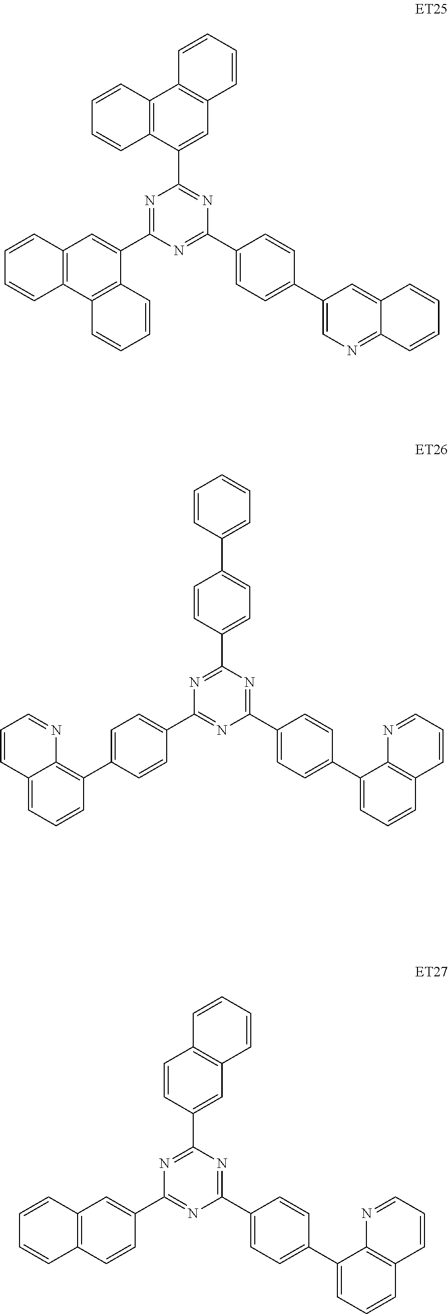 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

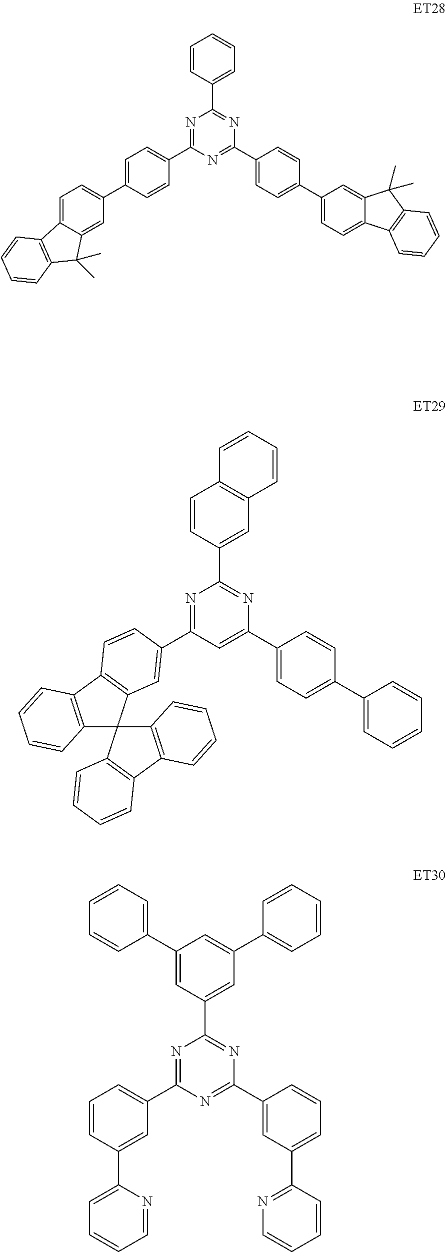 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

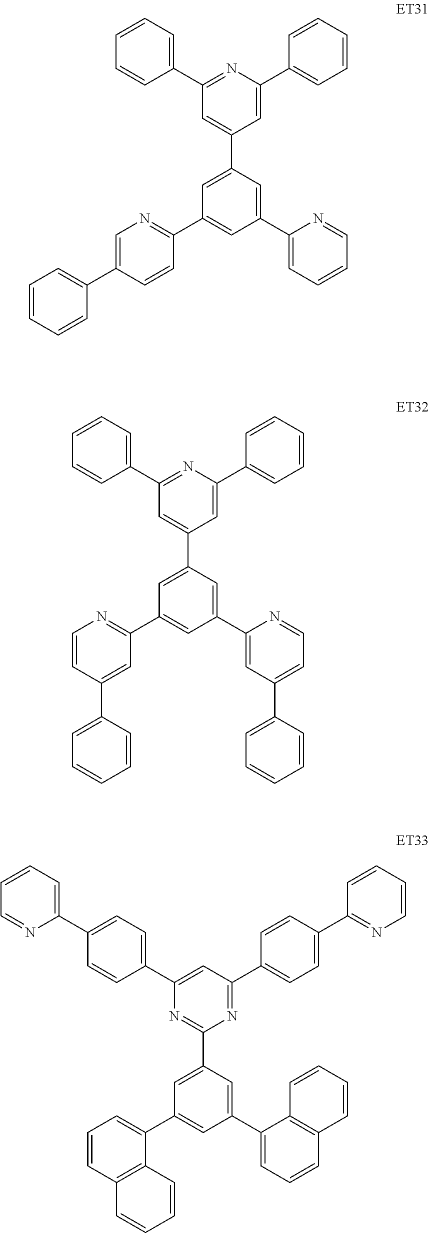 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

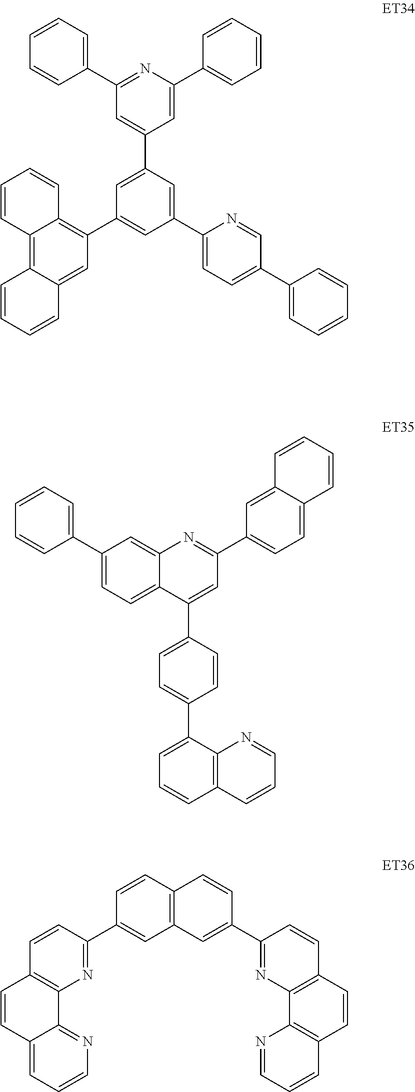 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

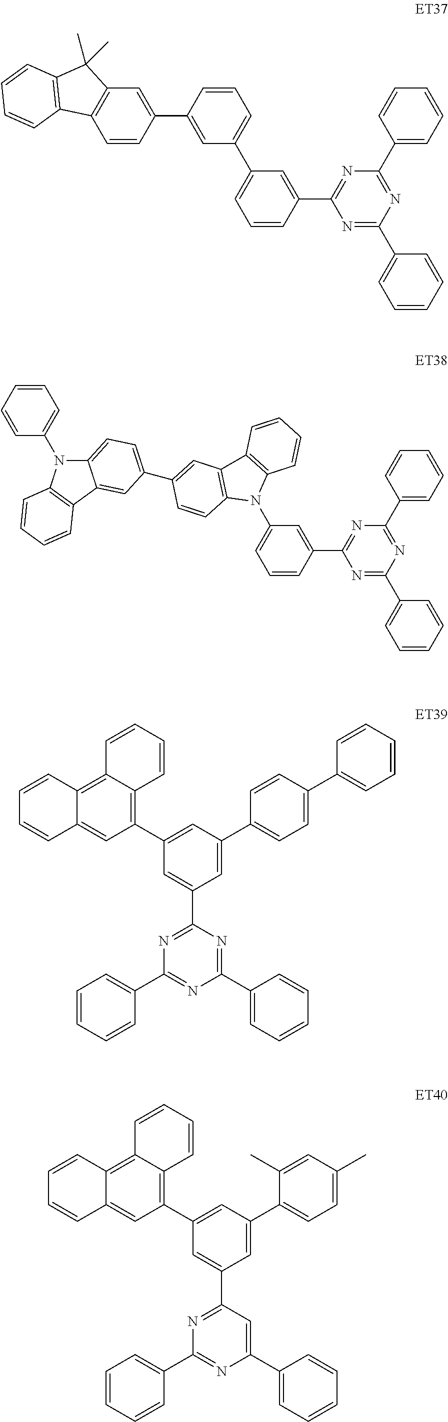 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1]

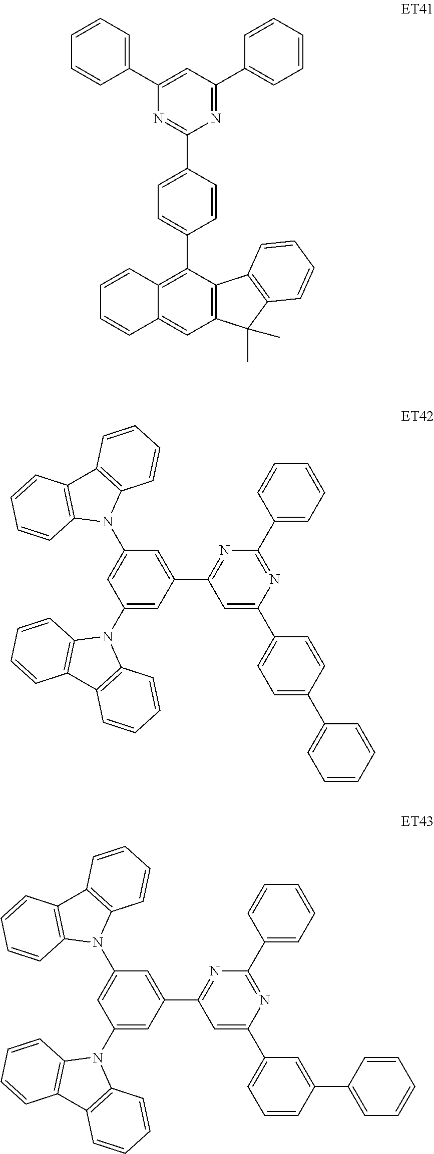 [SMILES: CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

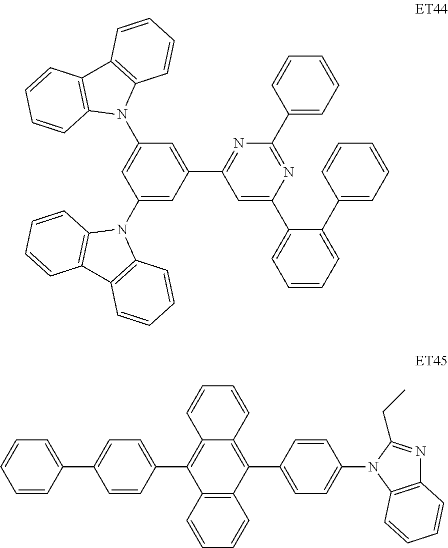 [SMILES: c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

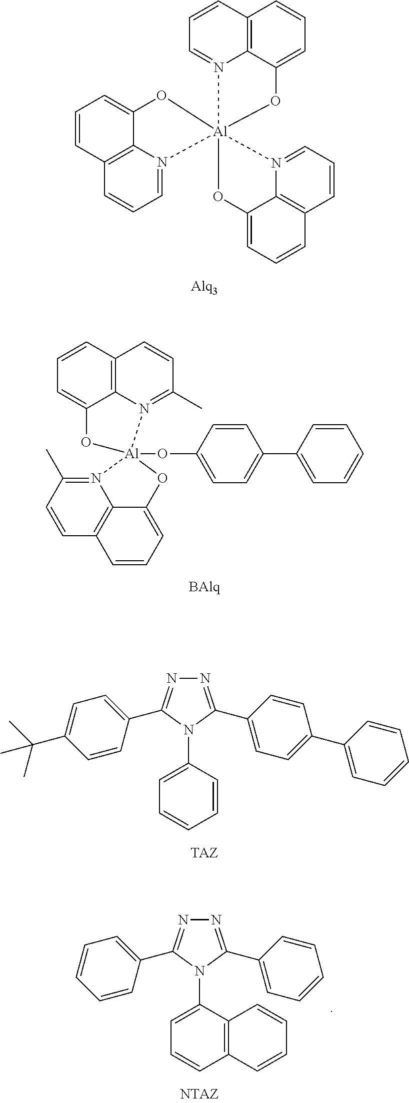

The thickness of the electron transport region may be in a range of about 100 Å to about 5,000 Å, for example, about 160 Å to about 4,000 Å. When the electron transport region includes a buffer layer, a hole blocking layer, an electron control layer, an electron transport layer, or any combination thereof, the thickness of the buffer layer, the hole blocking layer, or the electron control layer may be in a range of about 20 Å to about 1,000 Å, for example, about 30 Å to about 300 Å, and the thickness of the electron transport layer may be in a range of about 100 Å to about 1,000 Å, for example, about 150 Å to about 500 Å. When the thicknesses of the buffer layer, the hole blocking layer, the electron control layer, the electron transport layer, and/or the electron transport region are within these ranges, satisfactory electron transporting characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of each of the alkali metal complex or the alkaline earth-metal complex may include hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, cyclopentadiene, or any combination thereof.

For example, the metal-containing material may include a Li complex. The Li complex may include, for example, Compound ET-D1 (LiQ) or Compound ET-D2:

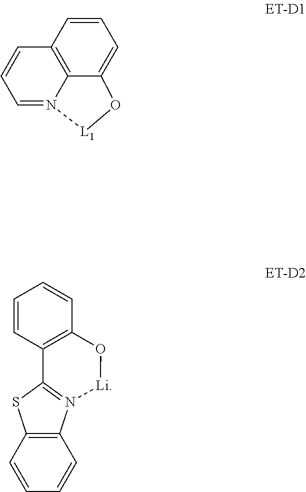

The electron transport region may include an electron injection layer that facilitates the injection of electrons from the second electrode 150. The electron injection layer may directly contact the second electrode 150, but the embodiments of the disclosure are not limited thereto.

The electron injection layer may have: i) a single-layered structure of a single layer containing a single material, ii) a single-layered structure of a single layer that icontains a plurality of different materials, or iii) a multi-layered structure including a plurality of layers including a plurality of different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (for example, fluorides, chlorides, bromides, or iodides), or tellurides, or any combination thereof of each of the alkali metal, the alkaline earth metal, and the rare earth metal.

The alkali metal-containing compound may include: an alkali metal oxide, such as Li₂O, Cs₂O, K₂O, or the like; an alkali metal halide, such as LiF, NaF, CsF, KF, LiI, NaI, CsI, KI, or the like; or any combination thereof. The alkaline earth metal-containing compound may include an alkaline earth metal compound, such as BaO, SrO, CaO, BaₓSr₁₋ₓO (wherein x is a real number satisfying 0<x<1), BaₓCa₁₋ₓO (wherein x is a real number satisfying 0<x<1), or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In some embodiments, the rare earth metal-containing compound may include a lanthanide metal telluride. Examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, and the like.

The alkali metal complex, the alkaline earth-metal complex, and the rare earth metal complex may include: i) one of ions of the alkali metal, the alkaline earth metal, and the rare earth metal; and ii) a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

The electron injection layer may consist of an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In some embodiments, the electron injection layer may further include an organic material (for example, a compound represented by Formula 601).

In an embodiment, the electron injection layer may consist of i) an alkali metal-containing compound (for example, alkali metal halide), ii) a) an alkali metal-containing compound (for example, alkali metal halide) and b) an alkali metal, an alkaline earth metal, a rare earth metal, or any combination thereof. For example, the electron injection layer may be a KI:Yb co-deposited layer, an RbI:Yb co-deposited layer, a LiF:Yb co-deposited layer, or the like.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth-metal complex, the rare earth metal complex, or any combination thereof may be uniformly or non-uniformly dispersed in a matrix including the organic material.

The thickness of the electron injection layer may be in a range of about 1 Å to about 100 Å, for example, about 3 Å to about 90 Å. When the thickness of the electron injection layer is within these ranges, satisfactory electron injection characteristics may be obtained without a substantial increase in driving voltage.

### [Second Electrode 150]

The second electrode 150 may be disposed on the electron transport region 140. The second electrode 150 may be a cathode, which is an electron injection electrode, and as a material for forming the second electrode 150, a metal, an alloy, an electrically conductive compound, or any combination thereof, each having a low-work function, may be used.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure.

### [Capping Layer]

A first capping layer may be arranged outside the first electrode 110. A second capping layer may be arranged outside the second electrode 150 in addition to or instead of the first capping layer. In some embodiments, the light-emitting device 10 may have a structure in which the first capping layer, the first electrode 110, the emission layer 130, and the second electrode 150 are sequentially stacked in the stated order, a structure in which the first electrode 110, the emission layer 130, the second electrode 150, and the second capping layer are sequentially stacked in the stated order, or a structure in which the first capping layer, the first electrode 110, the emission layer 130, the second electrode 150, and the second capping layer are sequentially stacked in the stated order. The capping layer is not shown in FIG. 1 or FIG. 2.

Light generated in the emission layer 130 of the light-emitting device 10 may pass through the first electrode 110, which is a semi-transmissive electrode or a transmissive electrode, and through the first capping layer to the outside. Light generated in the emission layer 130 of the light-emitting device 10 may pass through the second electrode 150, which is a semi-transmissive electrode or a transmissive electrode, and through the second capping layer to the outside.

The first capping layer and the second capping layer may increase external luminescence efficiency on the basis of the principle of constructive interference. Thus, the optical extraction efficiency of the light-emitting device 10 may be increased, thus improving the luminescence efficiency of the light-emitting device 10.

Each of the first capping layer and the second capping layer may include a material having a refractive index of about 1.6 or more (at about 520 nm to about 630 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer and the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may optionally be substituted with a substituent including O, N, S, Se, Si, F, Cl, Br, I, or any combination thereof. In an embodiment, at least one of the first capping layer and the second capping layer may each independently include an amine group-containing compound.

For example, at least one of the first capping layer and the second capping layer may each independently include a compound represented by Formula 201, a compound represented by Formula 202, or any combination thereof.

In some embodiments, at least one of the first capping layer and the second capping layer may each independently include: one of Compounds HT28 to HT33; one of Compounds CP1 to CP6; β-NPB; or any combination thereof:

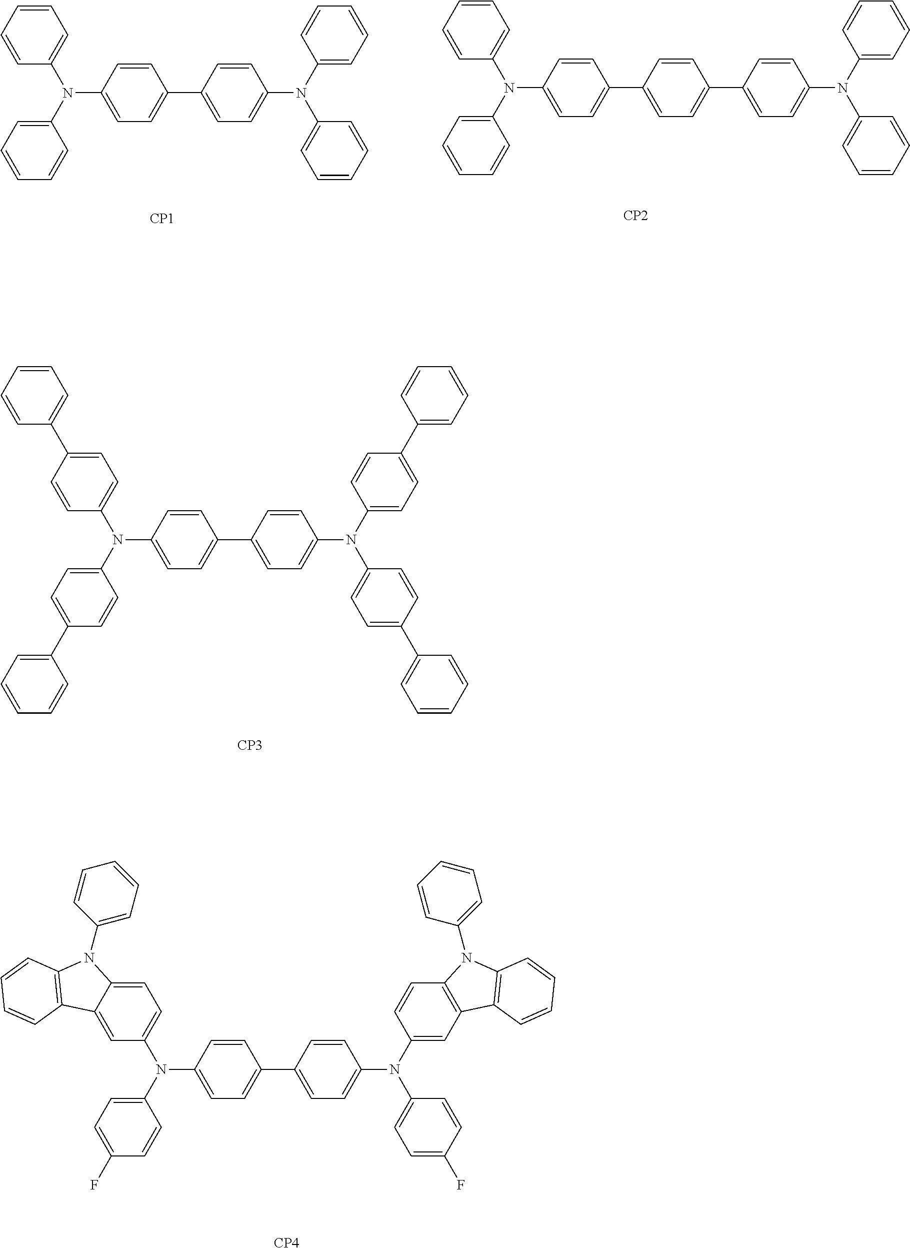

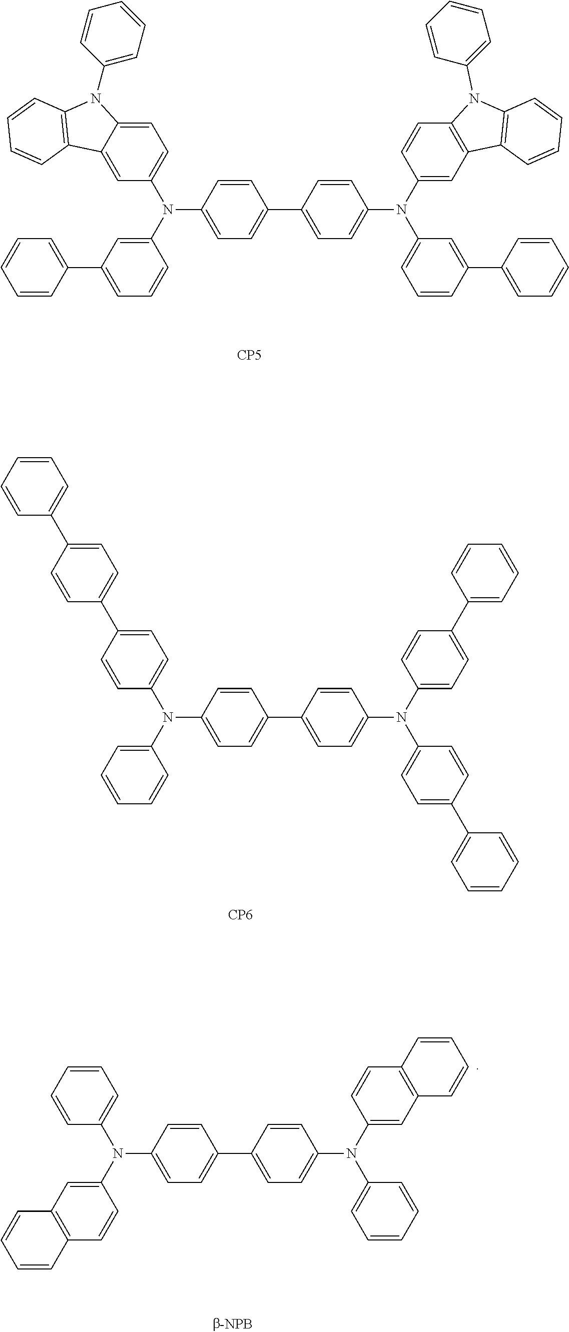 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; "CP5" ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; "CP6" ; c1ccc(N(c2ccc(-c3ccc(N(c4ccccc4)c4ccc5ccccc5c4)cc3)cc2)c2ccc3ccccc3c2)cc1]

### [Film]

According to some embodiments, the electronic apparatus may include a film. The film may be, for example, an optical member (or a light control means) (for example, a color filter, a color conversion member, a capping layer, a light extraction efficiency enhancement layer, a selective light absorbing layer, a polarizing layer, a quantum dot-containing layer, or like), a light-shielding member (for example, a light reflective layer, a light absorbing layer, or the like), a protective member (for example, an insulating layer, a dielectric layer, or the like).

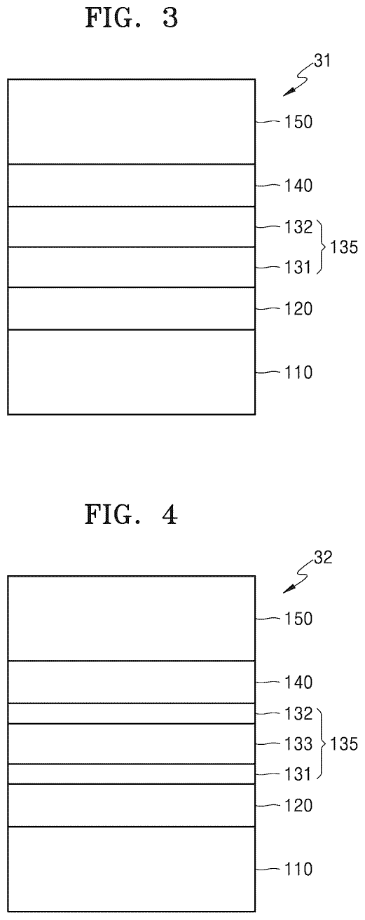

### [Description of FIG. 3]

FIG. 3 is a schematic view of an optoelectronic device 31 according to another embodiment.

The optoelectronic device 31 illustrated in FIG. 3 is the same as and/or similar to the optoelectronic device 30 illustrated in FIG. 1, except for the photoactive layer 135, and thus, descriptions of other components are omitted.

The optoelectronic device 31 may include the photoactive layer 135 between the hole transport region 120 and the electron transport region 140. In an embodiment, the photoactive layer 135 may be disposed between the hole transport layer included in the hole transport region 120 and a buffer layer included in the electron transport region 140. In other embodiments, the photoactive layer 135 may be disposed between the emission auxiliary layer included in the hole transport region 120 and a buffer layer included in the electron transport region 140.

The photoactive layer 135 may include a first layer 131 adjacent to the hole transport region 120 and a second layer 132 adjacent to the electron transport region 140. For example, the first layer 131 may directly contact the second layer 132.

In an embodiment, the first layer 131 may directly contact a hole transport layer included in the hole transport region 120. In other embodiments, the first layer 131 may directly contact an emission auxiliary layer disposed on the hole transport layer.

In an embodiment, the second layer 132 may directly contact a buffer layer included in the electron transport region 140.

The first layer 131 may include the first compound. The first layer 131 may consist of the first compound. For example, the first layer 131 may not include the second compound. The first layer 131 may also be referred to as a p-type photoactive layer or a donor layer.

The second layer 132 may include the second compound. The second layer 132 may consist of the second compound. For example, the second layer 132 may not include the first compound. The second layer 132 may also be referred to as an n-type photoactive layer or an acceptor layer.

In some embodiments, the photoactive layer 135 may have a double-layered structure of the first layer 131 including the first compound and the second layer 132 including the second compound.

The photoactive layer 135 may form excitons by absorbing incident light. The excitons are capable of generating holes and electrons. The holes generated by the photoactive layer 135 may move to the first electrode 110 through the hole transport region 120. The electrons generated by the photoactive layer 135 may move to the second electrode 150 through the electron transport region 140.

In other embodiments, the photoactive layer 135 may generate electrical signals by absorbing light. In some embodiments, the first compound included in the first layer 131 may serve as a donor for supplying electrons, and the second compound included in the second layer 132 may serve as an acceptor for receiving electrons. Thus, the optoelectronic device 31 including the photoactive layer 135 may serve as an optical sensor. For example, the optoelectronic device 31 may serve as a fingerprint recognition sensor, which will be described below with reference to FIG. 5.

### [Description of FIG. 4]

FIG. 4 is a schematic view of an optoelectronic device 32 according to another embodiment.

The optoelectronic device 32 illustrated in FIG. 4 is the same as and/or similar to the optoelectronic device 31 illustrated in FIG. 3, except for the photoactive layer 135. Descriptions of components that are described above in reference to FIG. 3 will be omitted.

The optoelectronic device 32 may include the photoactive layer 135 between the hole transport region 120 and the electron transport region 140. In an embodiment, the photoactive layer 135 may be disposed between the hole transport layer included in the hole transport region 120 and a buffer layer included in the electron transport region 140. In other embodiments, the photoactive layer 135 may be disposed between the emission auxiliary layer included in the hole transport region 120 and a buffer layer included in the electron transport region 140.

The photoactive layer 135 may include the first layer 131 adjacent to the hole transport region 120, the second layer 132 adjacent to the electron transport region 140, and a third layer 133 between the first layer 131 and the second layer 132, as depicted in FIG. 4. For example, the third layer 133 may directly contact the first layer 131 and/or the second layer 132.

In an embodiment, the first layer 131 may directly contact the hole transport layer included in the hole transport region 120. In other embodiments, the first layer 131 may directly contact an emission auxiliary layer disposed on the hole transport layer.

In an embodiment, the second layer 132 may directly contact a buffer layer included in the electron transport region 140.

The first layer 131 may include the first compound. The first layer 131 may consist of the first compound. For example, the first layer 131 may not include the second compound. The first layer 131 may also be referred to as a p-type photoactive layer or a donor layer.

The second layer 132 may include the second compound. The second layer 132 may consist of the second compound. For example, the second layer 132 may not include the first compound.

The second layer 132 may also be referred to as an n-type photoactive layer or an acceptor layer.

The third layer 133 may include the first compound and the second compound. For example, the first compound and the second compound may be included in the third layer 133 in a mixed state. The third layer 133 may also be referred to as a mixing layer.

In some embodiments, the photoactive layer 135 may have a triple-layered structure of a first layer 131 including the first compound, a second layer 132 including the second compound, and a third layer 133 including both the first compound and the second compound.

The photoactive layer 135 may produce excitons by absorbing incident light. The excitons are capable of generating holes and electrons. The holes generated by the photoactive layer 135 may move to the first electrode 110 through the hole transport region 120. The electrons generated by the photoactive layer 135 may move to the second electrode 150 through the electron transport region 140.

In other embodiments, the photoactive layer 135 may generate electrical signals by absorbing light. In some embodiments, the first compound included in each of the first layer 131 and the third layer 133 may serve as a donor for supplying electrons, and the second compound included in each of the second layer 132 and the third layer 133 may serve as an acceptor for receiving electrons. Thus, the optoelectronic device 32 including the photoactive layer 135 may serve as an optical sensor. For example, the optoelectronic device 32 may serve as a fingerprint recognition sensor, which will be described below in reference to FIG. 5.

### [Electronic Apparatus]

The light-emitting device 10 and the optoelectronic devices 30, 31, and 32 may be included in various electronic apparatuses. For example, the electronic apparatus may be a display apparatus, a light-emitting apparatus, an authentication apparatus, or the like.

The electronic apparatus (for example, a light-emitting apparatus) may further include, in addition to the light-emitting device 10 and the optoelectronic devices 30, 31, and 32, i) a color filter, ii) a color conversion layer, or iii) a color filter and a color conversion layer. The color filter and/or the color conversion layer may be arranged in at least one traveling direction of light emitted from the light-emitting device. For example, the light emitted from the light-emitting device may be blue light or white light. Details on the light-emitting device may be referred to the descriptions provided herein. In an embodiment, the color conversion layer may include a quantum dot. The quantum dot may be, for example, the quantum dot as described herein.

The electronic apparatus may include a first substrate. The first substrate may include a plurality of sub-pixel areas, the color filter may include a plurality of color filter areas respectively corresponding to the sub-pixel areas, and the color conversion layer may include a plurality of color conversion areas respectively corresponding to the sub-pixel areas.

A pixel-defining film may be arranged among the sub-pixel areas to define each sub-pixel area.

The color filter may further include a plurality of color filter areas and light-shielding patterns arranged among the color filter areas, and the color conversion layer may further include a plurality of color conversion areas and light-shielding patterns arranged among the color conversion areas.

The plurality of color filter areas (or the plurality of color conversion areas) may include: a first area emitting first color light; a second area emitting second color light; and/or a third area emitting third color light, in which the first color light, the second color light, and/or the third color light may have different maximum emission wavelengths. For example, the first color light may be red light, the second color light may be green light, and the third color light may be blue light.

For example, the plurality of color filter areas (or the plurality of color conversion areas) may include quantum dots. In some embodiments, the first area may include red quantum dots, the second area may include green quantum dots, and the third area may not include a quantum dot.

Details on the quantum dot are provided in other parts of this disclosure. The first area, the second area, and/or the third area may each further include a scatterer.

For example, the light-emitting device may emit first light, the first area may absorb the first light to emit first-1 color light, the second area may absorb the first light to emit second-1 color light, and the third area may absorb the first light to emit third-1 color light. In this regard, the first-1 color light, the second-1 color light, and the third-1 color light may have different maximum emission wavelengths. In some embodiments, the first light may be blue light, the first-1 color light may be red light, the second-1 color light may be green light, and the third-1 color light may be blue light.

The electronic apparatus may further include a thin-film transistor, in addition to the optoelectronic device and the light-emitting device as described above. The thin-film transistor may include a source electrode, a drain electrode, and an active layer, in which any one of the source electrode and the drain electrode may be electrically connected to any one of the first electrode and the second electrode of the light-emitting device.

The thin-film transistor may further include a gate electrode, a gate insulating film, and the like.

The active layer may include crystalline silicon, amorphous silicon, an organic semiconductor, an oxide semiconductor, or the like.

The electronic apparatus may further include an encapsulation unit for encapsulating the optoelectronic device and the light-emitting device. The encapsulation unit may be arranged between the color filter and/or the color conversion layer and the light-emitting device. The encapsulation unit allows light to pass to the outside from the light-emitting device and prevents the air and moisture to permeate into the optoelectronic device and the light-emitting device at the same time. The encapsulation unit may be an encapsulation substrate including a transparent glass substrate or a plastic substrate. The encapsulation unit may be a thin-film encapsulation layer including at least one layer of an organic layer and/or an inorganic layer. When the encapsulation unit is a thin film encapsulation layer, the electronic apparatus may be flexible.

In addition to the color filter and/or the color conversion layer, various functional layers may be further disposed on the encapsulation unit depending on the use of the electronic apparatus. Examples of the functional layers may include a touch screen layer, a polarizing layer, and the like. The touch screen layer may be a resistive touch screen layer, a capacitive touch screen layer, or an infrared beam touch screen layer. The authentication apparatus may be, for example, a biometric authentication apparatus that identifies an individual by using biometric information of a living body (for example, fingertips, pupils, or the like).

The authentication apparatus may further include a biometric information collector, in addition to the optoelectronic device and the light-emitting device as described above.

The electronic apparatus may be applied to various displays, an optical source, lighting, a personal computer (e.g., a mobile personal computer), a mobile phone, a digital camera, an electronic note, an electronic dictionary, an electronic game console, a medical device (e.g., an electronic thermometer, a blood pressure meter, a glucometer, a pulse measurement device, a pulse wave measuring device, an electrocardiogram recorder, an ultrasonic diagnostic device, or an endoscope display), a fish finder, various measurement devices, gauges (e.g., gauges of an automobile, an airplane, or a ship), a projector, a sensor (e.g., an automotive sensor or a home sensor), a solar cell, and the like.

### [Electronic Equipment]

The optoelectronic device may be included in various electronic equipment.

For example, the electronic equipment including the optoelectronic device may be one of a flat panel display, a curved display, a computer monitor, a medical monitor, a television, an advertisement board, an indoor or outdoor lighting and/or signaling light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a 3D display, a virtual or augmented reality display, a vehicle, a video wall including multiple displays tiled together, a theater or stadium screen, a phototherapy device, a signboard, an automotive sensor, a home sensor, and a solar cell.

Since the optoelectronic device has excellent optoelectronic characteristics and the like, the electronic equipment including the optoelectronic device may have the function of an optical sensor such as a fingerprint recognition sensor.

### [Descriptions of FIGS. 5 and 6]

FIG. 5 is a cross-sectional view of an electronic apparatus according to an embodiment.

The electronic apparatus of FIG. 5 includes a substrate 100, a thin-film transistor (TFT), the light-emitting device 10, the optoelectronic device 30, and an encapsulation unit 300. The optoelectronic device 30 of FIG. 5 may be the optoelectronic device 30 described with reference to FIG. 1, but the embodiments of the disclosure is not limited thereto. For example, the optoelectronic device 30 of FIG. 5 may be the optoelectronic device 31 of FIG. 3 or the optoelectronic device 32 of FIG. 4.

The substrate 100 may be a flexible substrate, a glass substrate, or a metal substrate. A buffer layer 210 may be arranged on the substrate 100. The buffer layer 210 may prevent penetration of impurities through the substrate 100 and may provide a flat surface on the substrate 100.

A TFT may be arranged on the buffer layer 210. The TFT may include an active layer 220, a gate electrode 240, a source electrode 260, and a drain electrode 270.

The active layer 220 may include an inorganic semiconductor such as silicon or poly silicon, an organic semiconductor, or an oxide semiconductor, and may include a source region, a drain region, and a channel region.

A gate insulating film 230 for insulating the active layer 220 from the gate electrode 240 may be arranged on the active layer 220, and the gate electrode 240 may be arranged on the gate insulating film 230.

An interlayer insulating film 250 may be arranged on the gate electrode 240. The interlayer insulating film 250 may be arranged between the gate electrode 240 and the source electrode 260 and between the gate electrode 240 and the drain electrode 270, to provide insulation therebetween.

The source electrode 260 and the drain electrode 270 may be arranged on the interlayer insulating film 250. The interlayer insulating film 250 and the gate insulating film 230 may be formed to expose the source region and the drain region of the active layer 220, and the source electrode 260 and the drain electrode 270 may be arranged in contact with the exposed portions of the source region and the drain region of the active layer 220.

The light-emitting device 10 and the optoelectronic device 30 may be disposed on the TFT. The light-emitting device 10 and the optoelectronic device 30 are disposed next to each other and optically coupled. As used herein, two elements being disposed “next to” each other means they are positioned on different parts of the substrate 100, such that they are not covering each other in a cross-sectional view. As used herein, two elements being “optically coupled” means light can travel between the two elements.

The TFT electrically connected to the light-emitting device 10 may transmit an electrical signal for driving the light-emitting device 10. The TFT electrically connected to the optoelectronic device 30 may transmit an electrical signal generated from the optoelectronic device 30. The TFT is covered and protected by a passivation layer 280. The passivation layer 280 may include an inorganic insulating film, an organic insulating film, or any combination thereof. The light-emitting device 10 and the optoelectronic device 30 are provided on the passivation layer 280.

The light-emitting device 10 may include the first electrode 110, the hole transport region 120, the emission layer 130, the electron transport region 140, and the second electrode 150. The optoelectronic device 30 may include the first electrode 110, the hole transport region 120, the photoactive layer 135, the electron transport region 140, and the second electrode 150. The first electrode 110 may be arranged on the passivation layer 280. The passivation layer 280 may be arranged to expose certain portions of the source electrode 260 and the drain electrode 270, not fully covering the source electrode 260 and the drain electrode 270, and the first electrode 110 may be arranged to be connected to the exposed portions of the source electrode 260 and the drain electrode 270.

A pixel defining layer 290 including an insulating material may be arranged on the first electrode 110. The pixel defining layer 290 may have openings extending to a certain region of the first electrode 110. The pixel defining layer 290 may be a polyimide-based organic film or a polyacrylic organic film.

The hole transport region 120 may be disposed on the pixel defining layer 290. The hole transport region 120 included in the light-emitting device 10 may be integrated with the hole transport region 120 included in the optoelectronic device 30. The hole transport region 120 included in the light-emitting device 10 and the hole transport region 120 included in the optoelectronic device 30 may be disposed on the pixel defining layer 290, may be connected to each other and include substantially the same material, and may be formed substantially at the same time.

The emission layer 130 and the photoactive layer 135 may be disposed on the hole transport region 120. Each of the emission layer 130 and the photoactive layer 135 may overlap the certain region of the first electrode 110 exposed by the pixel defining layer 290.

The electron transport region 140 may be disposed on the emission layer 130 and the photoactive layer 135. The electron transport region 140 included in the light-emitting device 10 may be integrated in one body with the electron transport region 140 included in the optoelectronic device 30. The electron transport region 140 included in the light-emitting device 10 and the electron transport region 140 included in the optoelectronic device 30 may be disposed on the pixel defining layer 290, may be connected to each other and include substantially the same material, and may be formed substantially at the same time.

The second electrode 150 may be disposed on the electron transport region 140. The second electrode 150 included in the light-emitting device 10 may be integrated in one body with the second electrode 150 included in the optoelectronic device 30. The second electrode 150 included in the light-emitting device 10 and the second electrode 150 included in the optoelectronic device 30 may be disposed on the pixel defining layer 290, may be connected to each other and include substantially the same material, and may be formed substantially at the same time.

A capping layer 170 may be further disposed on the second electrode 150. The capping layer 170 may be formed to cover the second electrode 150.

The encapsulation unit 300 may be arranged on the capping layer 170. The encapsulation unit 300 may be arranged on the light-emitting device 10 and the optoelectronic device 30 and serve to protect the light-emitting device 10 and the optoelectronic device 30 from moisture or oxygen. The encapsulation unit 300 may include: an inorganic film including silicon nitride (SiNₓ), silicon oxide (SiOₓ), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, or the like), an epoxy-based resin (for example, aliphatic glycidyl ether (AGE), or the like), or any combination thereof; or any combination of the inorganic film and the organic film.

The light-emitting device 10 may emit light beams L1, L2, and L3. For example, light beams L1, L2, and L3 may be red light, green light, blue light, or near-infrared light.

A part L3 of the emitted light beams L1, L2, and L3 may be incident on an object 600 outside the electronic apparatus. For example, the object 600 may be a finger of a user of the electronic apparatus. Light L3′ reflected from the object 600 may be incident on the optoelectronic device 30.

The photoactive layer 135 may form excitons by absorbing the incident light L3′. The excitons are capable of generating holes and electrons. In other embodiments, the photoactive layer 135 may generate electrical signals by absorbing light. In some embodiments, the first compound included in the photoactive layer 135 may serve as a donor for supplying electrons, and the second compound included in the photoactive layer 135 may serve as an acceptor for receiving electrons. In some embodiments, the optoelectronic device 30 may detect energy of the light L3′ and convert the same into an electrical signal. In some embodiments, the optoelectronic device 30 may recognize the object 600 in contact with (or approaching) the electronic apparatus. Thus, the optoelectronic device 30 including the photoactive layer 135 may serve as an optical sensor (e.g., a fingerprint recognition sensor).

FIG. 6 is a cross-sectional view of an electronic apparatus according to another embodiment.

The electronic apparatus of FIG. 6 is the same as the electronic apparatus of FIG. 5, except that a light-shielding pattern 500 and a functional region 400 are further arranged on the encapsulation unit 300. The functional region 400 may be i) a color filter area, ii) a color conversion area, or iii) a combination of the color filter area and the color conversion area. In an embodiment, the light-emitting device included in the electronic apparatus of FIG. 6 may be a tandem light-emitting device.

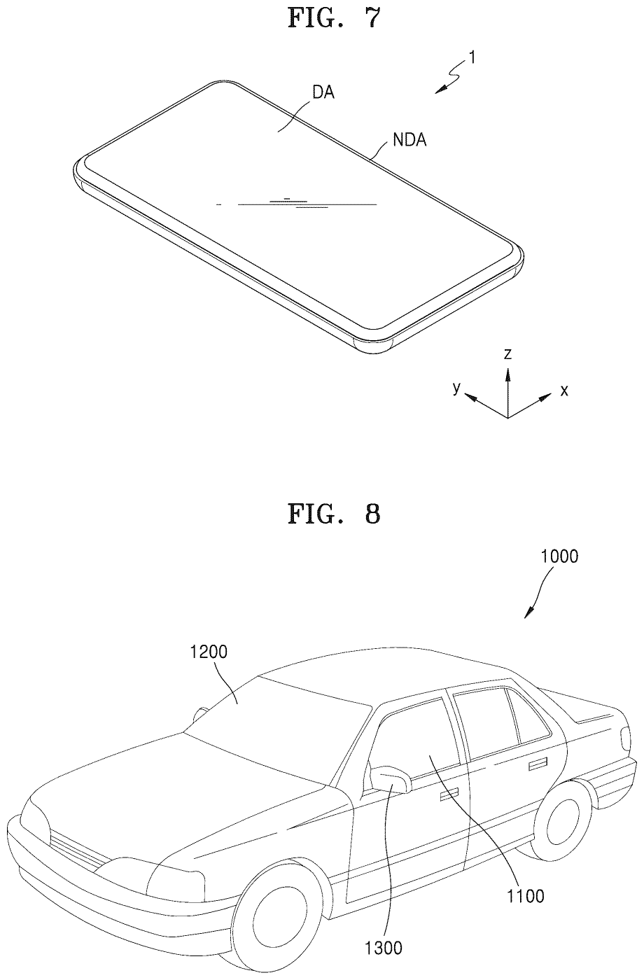

### [Description of FIG. 7]

FIG. 7 is a schematic perspective view of electronic equipment 1 including an optoelectronic device according to an embodiment. The electronic equipment 1 may be, as an apparatus that displays a moving image or still image, portable electronic equipment, such as a mobile phone, a smart phone, a tablet personal computer (PC), a mobile communication terminal, an electronic notebook, an electronic book, a portable multimedia player (PMP), a navigation, or a ultra mobile PC (UMPC), as well as various products, such as a television, a laptop, a monitor, an advertisement board, or an Internet of things (IOT), or a part thereof. In addition, the electronic equipment 1 may be a wearable device, such as a smart watch, a watch phone, a glasses-type display, or a head mounted display (HMD), or a part of the wearable device. However, embodiments of the disclosure are not limited thereto. For example, the electron equipment 1 may include a dashboard of a vehicle, a center fascia of a vehicle, a center information display (CID) arranged on a dashboard of a vehicle, a room mirror display that replaces a side mirror of a vehicle, an entertainment display for the rear seat of a vehicle or a display arranged on the back of the front seat, or a head up display (HUD) installed in the front of a vehicle or projected on a front window glass, or a computer generated hologram augmented reality head up display (CGH AR HUD). FIG. 7 illustrates a case in which the electronic equipment 1 is a smart phone for convenience of explanation.

The electronic equipment 1 may include a display area DA and a non-display area NDA outside the display area DA. A display device may implement an image through an array of a plurality of pixels that are two-dimensionally arranged in the display area DA.

The non-display area NDA is an area that does not display an image, and may entirely surround the display area DA. On the non-display area NDA, a driver for providing electrical signals or power to display devices arranged on the display area DA may be arranged. On the non-display area NDA, a pad, which is an area to which an electronic element, a printing circuit board, or the like may be electrically connected, may be arranged.

In the electronic equipment 1, a length in the x-axis direction and a length in the y-axis direction may be different from each other. For example, as illustrated in FIG. 7, the length in the x-axis direction may be shorter than the length in the y-axis direction. In some embodiments, the length in the x-axis direction may be the same as the length in the y-axis direction. In some embodiments, the length in the x-axis direction may be longer than the length in the y-axis direction.

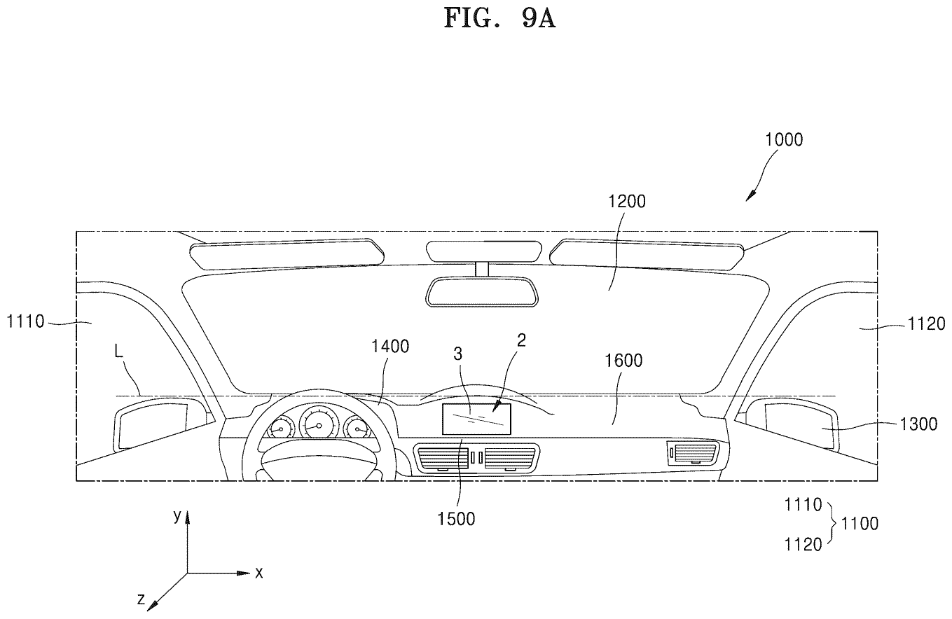

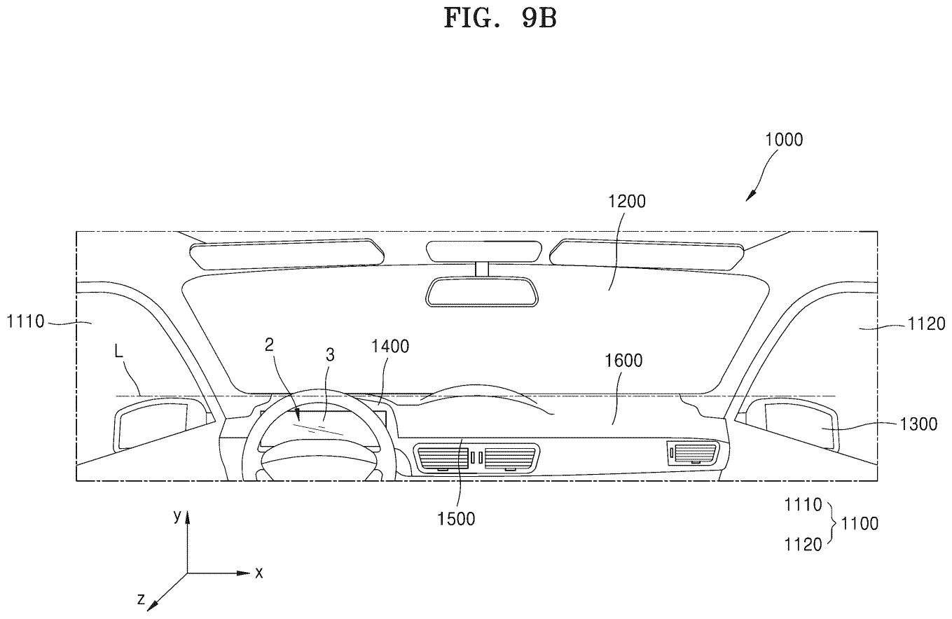

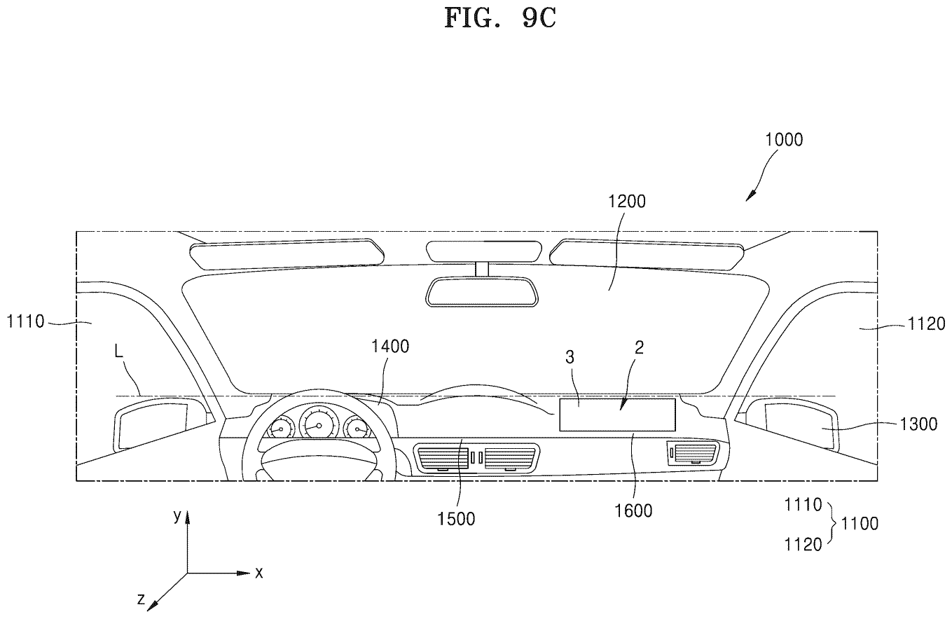

### [Descriptions of FIGS. 8 and 9A to 9C]

FIG. 8 is a schematic view illustrating the exterior of a vehicle 1000 as electronic equipment including a light-emitting device according to an embodiment. FIGS. 9A to 9C are schematic views illustrating the interior of the vehicle 1000 according to various embodiments.

Referring to FIGS. 8, 9A, 9B, and 9C, the vehicle 1000 may refer to various apparatuses for moving a subject to be transported, such as a human, an object, or an animal, from a departure point to a destination point. The vehicle 1000 may include a vehicle traveling on a road or track, a vessel moving over the sea or river, an airplane flying in the sky using the action of air, and the like.

The vehicle 1000 may travel on a road or a track. The vehicle 1000 may move in a predetermined direction according to rotation of at least one wheel. For example, the vehicle 1000 may include a three-wheeled or four-wheeled vehicle, a construction machine, a two-wheeled vehicle, a prime mover device, a bicycle, and a train running on a track.

The vehicle 1000 may include a body having an interior and an exterior, and a chassis in which mechanical apparatuses necessary for driving are installed as other parts except for the body. The exterior of the body may include a front panel, a bonnet, a roof panel, a rear panel, a trunk, a pillar provided at a boundary between doors, and the like. The chassis of the vehicle 1000 may include a power generating device, a power transmitting device, a driving device, a steering device, a braking device, a suspension device, a transmission device, a fuel device, front/rear and left/right wheels, and the like.

The vehicle 1000 may include a side window glass 1100, a front window glass 1200, a side mirror 1300, a cluster 1400, a center fascia 1500, a passenger seat dashboard 1600, and a display device 2.

The side window glass 1100 and the front window glass 1200 may be partitioned by a pillar arranged between the side window glass 1100 and the front window glass 1200.

The side window glass 1100 may be installed on the side of the vehicle 1000. In an embodiment, the side window glass 1100 may be installed on a door of the vehicle 1000. A plurality of side window glasses 1100 may be provided, for example on opposing sides of the vehicle 1000. In an embodiment, the side window glass 1100 may include a first side window glass 1110 and a second side window glass 1120 as depicted in FIG. 9A. In an embodiment, the first side window glass 1110 may be arranged adjacent to the cluster 1400. The second side window glass 1120 may be arranged adjacent to the passenger seat dashboard 1600.

In an embodiment, the side window glasses 1100 may be spaced apart from each other in the x-direction. For example, the first side window glass 1110 and the second side window glass 1120 may be spaced apart from each other in the x direction. In other words, an imaginary straight line L connecting the side window glasses 1100 may extend in the x-direction or the −x-direction. For example, an imaginary straight line L connecting the first side window glass 1110 and the second side window glass 1120 to each other may extend in the x direction or the −x direction.

The front window glass 1200 may be installed in the front of the vehicle 1000. The front window glass 1200 may be arranged between the side window glasses 1100 on opposite sides of the vehicle 1000.

The side mirror 1300 may provide a rear view of the vehicle 1000. The side mirror 1300 may be installed on the exterior of the vehicle body. In one embodiment, a plurality of side mirrors 1300 may be provided. Any one of the plurality of side mirrors 1300 may be arranged outside the first side window glass 1110. The other one of the plurality of side mirrors 1300 may be arranged outside the second side window glass 1120.

The cluster 1400 may be arranged in the front of the steering wheel. The cluster 1400 may include a tachometer, a speedometer, a coolant thermometer, a fuel gauge turn indicator, a high beam indicator, a warning lamp, a seat belt warning lamp, an odometer, an automatic shift selector indicator lamp, a door open warning lamp, an engine oil warning lamp, and/or a low fuel warning light.

The center fascia 1500 may include a control panel on which a plurality of buttons for adjusting an audio device, an air conditioning device, and a heater of a seat are disposed. The center fascia 1500 may be arranged on one side of the cluster 1400.

The passenger seat dashboard 1600 may be spaced apart from the cluster 1400 with the center fascia 1500 arranged therebetween. In an embodiment, the cluster 1400 may be arranged to correspond to a driver seat (not shown), and the passenger seat dashboard 1600 may be disposed to correspond to a passenger seat (not shown). In an embodiment, the cluster 1400 may be adjacent to the first side window glass 1110, and the passenger seat dashboard 1600 may be adjacent to the second side window glass 1120.

In an embodiment, the display device 2 may include a display panel 3, and the display panel 3 may display an image. The display device 2 may be arranged inside the vehicle 1000. In an embodiment, the display device 2 may be arranged between the side window glasses 1100 on opposite sides of the vehicle 1000. The display device 2 may be arranged on at least one of the cluster 1400, the center fascia 1500, and the passenger seat dashboard 1600.

The display device 2 may include an organic light-emitting display device, an inorganic EL display device, a quantum dot display device, and the like. Hereinafter, as the display device 2 according to an embodiment of the disclosure, an organic light-emitting display device including the light-emitting device according to the disclosure will be described as an example, but various types of display devices as described above may be used in embodiments of the disclosure.

Referring to FIG. 9A, the display device 2 may be arranged on the center fascia 1500. In an embodiment, the display device 2 may display navigation information. In an embodiment, the display device 2 may display audio, video, or information regarding vehicle settings.

Referring to FIG. 9B, the display device 2 may be arranged on the cluster 1400. When the display device 2 is arranged on the cluster 1400, the cluster 1400 may display driving information and the like through the display device 2. In some embodiments, the cluster 1400 may be implemented digitally. The digital cluster 1400 may display vehicle information and driving information as images. For example, a needle and a gauge of a tachometer and various warning light icons may be displayed by a digital signal.

Referring to FIG. 9C, the display device 2 may be arranged on the passenger seat dashboard 1600. The display device 2 may be embedded in the passenger seat dashboard 1600 or arranged on the passenger seat dashboard 1600. In an embodiment, the display device 2 arranged on the for the passenger seat dashboard 1600 may display an image related to information displayed on the cluster 1400 and/or information displayed on the center fascia 1500. In some embodiments, the display device 2 arranged on the passenger seat dashboard 1600 may display information different from information displayed on the cluster 1400 and/or information displayed on the center fascia 1500.

### [Manufacturing Method]

Respective layers included in the hole transport region, the emission layer, the photoactive layer, and/or respective layers included in the electron transport region may be formed in a certain region by using various methods, such as vacuum deposition, spin coating, casting, a Langmuir-Blodgett (LB) method, ink-jet printing, laser-printing, laser-induced thermal imaging, and the like.

When respective layers included in the hole transport region, the emission layer, the photoactive layer, and/or respective layers included in the electron transport region are formed by vacuum deposition, the deposition may be performed at a deposition temperature of about 100° C. to about 500° C., a vacuum degree of about 10-8 torr to about 10-3 torr, and a deposition rate of about 0.01 Å/sec to about 100 Å/sec, depending on the material to be included in each layer to be formed and the structure of each layer to be formed.

### Definition of Terms

The term “C₃-C₆₀ carbocyclic group” as used herein refers to a cyclic group having only carbon as a ring-forming atom and having 3 to 60 carbon atoms. The term “C₁-C₆₀ heterocyclic group” as used herein refers to a cyclic group that has 1 to 60 carbon atoms and further has, in addition to carbon, a heteroatom as a ring-forming atom. The C₃-C₆₀ carbocyclic group and the C₁-C₆₀ heterocyclic group may each be a monocyclic group having one ring or a polycyclic group in which two or more rings are condensed with each other. For example, the number of ring-forming atoms of the C₁-C₆₀ heterocyclic group may be from 3 to 61.

The “cyclic group” as used herein may include both the C₃-C₆₀ carbocyclic group and the C₁-C₆₀ heterocyclic group.

The term “π electron-rich C₃-C₆₀ cyclic group” as used herein refers to a cyclic group that has 3 to 60 carbon atoms and does not include *—N═*′ as a ring-forming moiety.

The term “π electron-deficient nitrogen-containing C₁-C₆₀ cyclic group” as used herein refers to a heterocyclic group that has 1 to 60 carbon atoms and includes *—N═*′ as a ring-forming moiety.

### For Example,

the C₃-C₆₀ carbocyclic group may be i) Group T1 or ii) a condensed cyclic group in which two or more of Group T1 are condensed with each other (e.g., a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) Group T2, ii) a condensed cyclic group in which two or more of Group T2 are condensed with each other, or iii) a condensed cyclic group in which at least one Group T2 and at least one Group T1 are condensed with each other (e.g., a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, or the like),
the π electron-rich C₃-C₆₀ cyclic group may be i) Group T1, ii) a condensed cyclic group in which two or more of Group T1 are condensed with each other, iii) Group T3, iv) a condensed cyclic group in which two or more of Group T3 are condensed with each other, or v) a condensed cyclic group in which at least one Group T3 and at least one Group T1 are condensed with each other (e.g., the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, or the like), and
the π electron-deficient nitrogen-containing C₁-C₆₀ cyclic group may be i) Group T4, ii) a condensed cyclic group in which two or more of Group T4 are condensed with each other, iii) a condensed cyclic group in which at least one Group T4 and at least one Group T1 are condensed with each other, iv) a condensed cyclic group in which at least one Group T4 and at least one Group T3 are condensed with each other, or v) a condensed cyclic group in which at least one Group T4, at least one Group T1, and at least one Group T3 are condensed with one another (e.g., a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and the like).

Group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group.

Group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group.

T3 may be a furan group, a thiophene group, a 1H-pyrrole group, a silole group, or a borole group.

Group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group, the C₃-C₆₀ carbocyclic group, the C₁-C₆₀ heterocyclic group, the π electron-rich C₃-C₆₀ cyclic group, or the π electron-deficient nitrogen-containing C₁-C₆₀ cyclic group” as used herein refer to a group condensed to any cyclic group, a monovalent group, or a polyvalent group (e.g., a divalent group, a trivalent group, a tetravalent group, or the like) according to the structure of a formula for which the corresponding term is used.

For example, the “benzene group” may be a benzo group, a phenyl group, a phenylene group, or the like, which may be easily understand by one of ordinary skill in the art according to the structure of a formula including the “benzene group.”

Examples of the monovalent C₃-C₆₀ carbocyclic group and the monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

Examples of the divalent C₃-C₆₀ carbocyclic group and the divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein refers to a linear or branched aliphatic hydrocarbon monovalent group that has 1 to 60 carbon atoms, and examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group.

The term “C₁-C₆₀ alkylene group” as used herein refers to a divalent group having the same structure as the C₁-C₆₀ alkyl group.

The term “C₂-C₆₀ alkenyl group” as used herein refers to a monovalent hydrocarbon group having at least one carbon-carbon double bond in the middle or at the terminus of the C₂-C₆₀ alkyl group, and examples thereof may include an ethenyl group, a propenyl group, a butenyl group, and the like.

The term “C₂-C₆₀ alkenylene group” as used herein refers to a divalent group having the same structure as the C₂-C₆₀ alkenyl group.

The term “C₂-C₆₀ alkynyl group” as used herein refers to a monovalent hydrocarbon group having at least one carbon-carbon triple bond in the middle or at the terminus of the C₂-C₆₀ alkyl group, and examples thereof may include an ethynyl group, a propynyl group, and the like.

The term “C₂-C₆₀ alkynylene group” as used herein refers to a divalent group having the same structure as the C₂-C₆₀ alkynyl group.

The term “C₁-C₆₀ alkoxy group” as used herein refers to a monovalent group represented by —OA₁₀₁ (wherein A₁₀₁ is the C₁-C₆₀ alkyl group), and examples thereof may include a methoxy group, an ethoxy group, an isopropyloxy group, and the like.

The term “C₃-C₁₀ cycloalkyl group” as used herein refers to a monovalent saturated hydrocarbon cyclic group having 3 to 10 carbon atoms, and examples thereof may include a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, a bicyclo[2.2.2]octyl group, and the like.

The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent cyclic group of 1 to 10 carbon atoms, further including, in addition to carbon atoms, at least one heteroatom, as ring-forming atoms, and examples thereof may include a 1,2,3,4-oxatriazolidinyl group, a tetrahydrofuranyl group, a tetrahydrothiophenyl group, and the like.

The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent cyclic group that has 3 to 10 carbon atoms and at least one carbon-carbon double bond in the ring thereof and no aromaticity, and examples thereof may include a cyclopentenyl group, a cyclohexenyl group, a cycloheptenyl group, and the like.

The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent cyclic group of 1 to 10 carbon atoms, further including, in addition to carbon atoms, at least one heteroatom, as ring-forming atoms, and having at least one carbon-carbon double bond in the cyclic structure thereof. Examples of the C₁-C₁₀ heterocycloalkenyl group may include a 4,5-dihydro-1,2,3,4-oxatriazolyl group, a 2,3-dihydrofuranyl group, a 2,3-dihydrothiophenyl group, and the like.

The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system of 6 to 60 carbon atoms.

The term “C₆-C₆₀ arylene group” as used herein refers to a divalent group having a carbocyclic aromatic system of 6 to 60 carbon atoms.

Examples of the C₆-C₆₀ aryl group include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, and the like.

When the C₆-C₆₀ aryl group and the C₆-C₆₀ arylene group each include two or more rings, the rings may be condensed with each other.

The term “C₁-C₆₀ heteroaryl group” as used herein refers to a monovalent group having a heterocyclic aromatic system of 1 to 60 carbon atoms, further including, in addition to carbon atoms, at least one heteroatom, as ring-forming atoms.

The term “C₁-C₆₀ heteroarylene group” as used herein refers to a divalent group having a heterocyclic aromatic system of 1 to 60 carbon atoms, further including, in addition to carbon atoms, at least one heteroatom, as ring-forming atoms.

Examples of the C₁-C₆₀ heteroaryl group include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group.

When the C₁-C₆₀ heteroaryl group and the C₁-C₆₀ heteroarylene group each include two or more rings, the rings may be condensed with each other.

The term “monovalent non-aromatic condensed polycyclic group” as used herein refers to a monovalent group (for example, having 8 to 60 carbon atoms) having two or more rings condensed to each other, only carbon atoms as ring-forming atoms, and no aromaticity in its entire molecular structure. Examples of the monovalent non-aromatic condensed polycyclic group include an indenyl group, a fluorenyl group, a spiro-bifluorenyl group, a benzofluorenyl group, an indenophenanthrenyl group, an indeno anthracenyl group, and the like.

The term “divalent non-aromatic condensed polycyclic group” as used herein refers to a divalent group having the same structure as the monovalent non-aromatic condensed polycyclic group described above.

The term “monovalent non-aromatic condensed heteropolycyclic group” as used herein refers to a monovalent group (for example, having 1 to 60 carbon atoms) having two or more rings condensed to each other, further including, in addition to carbon atoms, at least one heteroatom, as ring-forming atoms, and having non-aromaticity in its entire molecular structure. Examples of the monovalent non-aromatic condensed heteropolycyclic group include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group.

The term “divalent non-aromatic condensed heteropolycyclic group” as used herein refers to a divalent group having the same structure as the monovalent non-aromatic condensed heteropolycyclic group described above.

The term “C₆-C₆₀ aryloxy group” as used herein indicates —OA₁₀₂ (wherein A₁₀₂ is the C₆-C₆₀ aryl group).

The term “C₆-C₆₀ arylthio group” as used herein indicates —SA₁₀₃ (wherein A₁₀₃ is the C₆-C₆₀ aryl group).

The term “C₇-C₆₀ arylalkyl group” as used herein refers to -A₁₀₄A₁₀₅ (wherein A₁₀₄ is a C₁-C₅₄ alkylene group, and A₁₀₅ is a C₆-C₅₉ aryl group).

The term “C₂-C₆₀ heteroarylalkyl group” as used herein refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ” as used herein may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

In the present specification, Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group; a C₇-C₆₀ arylalkyl group; or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

The term “heteroatom” as used herein refers to any atom other than a carbon atom. Examples of the heteroatom include O, S, N, P, Si, B, Ge, Se, and any combination thereof.

In the present specification, the third-row transition metal includes hafnium (Hf), tantalum (Ta), tungsten (W), rhenium (Re), osmium (Os), iridium (Ir), platinum (Pt), gold (Au), and the like.

The term “Ph” as used herein refers to a phenyl group, the term “Me” as used herein refers to a methyl group, the term “Et” as used herein refers to an ethyl group, the term “tert-Bu” or “But” as used herein refers to a tert-butyl group, and the term “OMe” as used herein refers to a methoxy group.

The term “biphenyl group” as used herein refers to “a phenyl group substituted with a phenyl group.” The “biphenyl group” is a substituted phenyl group having a C₆-C₆₀ aryl group as a substituent.

The term “terphenyl group” as used herein refers to “a phenyl group substituted with a biphenyl group.” The “terphenyl group” is a substituted phenyl group having, as a substituent, a C₆-C₆₀ aryl group substituted with a C₆-C₆₀ aryl group.

*, *′ and *″ as used herein, unless defined otherwise, each refer to a binding site to a neighboring atom in a corresponding formula or moiety.

In the present specification, the x-axis, y-axis, and z-axis are not limited to three axes in an orthogonal coordinate system, and may be interpreted in a broad sense including these axes. For example, the x-axis, y-axis, and z-axis may refer to those orthogonal to each other, or may refer to those in different directions that are not orthogonal to each other.

Hereinafter, compounds according to embodiments and light-emitting devices according to embodiments will be described in detail with reference to the following synthesis examples and examples. The wording “B was used instead of A,” as used in synthesis examples, is intended to mean that the amount of B and the amount of A are molar equivalents.

### Synthesis Example 1 (Synthesis of Compound 10)

### Synthesis of Intermediate 10-1

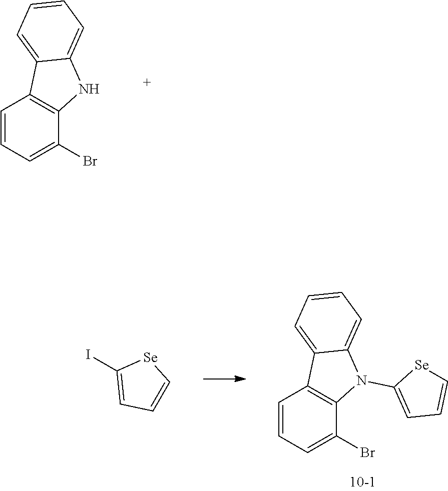 [SMILES: Brc1cccc2c1[nH]c1ccccc12 ; Ic1ccc[se]1 ; Brc1cccc2c3ccccc3n(-c3ccc[se]3)c12]

10 g (38.9 mmol) of 2-iodoselenophene and 7.8 g (32 mmol) of 1-bromo-9H-carbazole were dissolved in 40 ml of dioxane. 0.35 g (1.8 mmol) of copper(I) iodide, 0.70 g (6.09 mmol) of trans-1,2-cyclohexanediamine, and 12.9 g (61.0 mmol) of tripotassium phosphate were added and refluxed by heating for 30 hours. The mixture was purified through silica gel column chromatography to thereby obtain 8.40 g (70%) of Intermediate 10-1.

### Synthesis of Intermediate 10-2

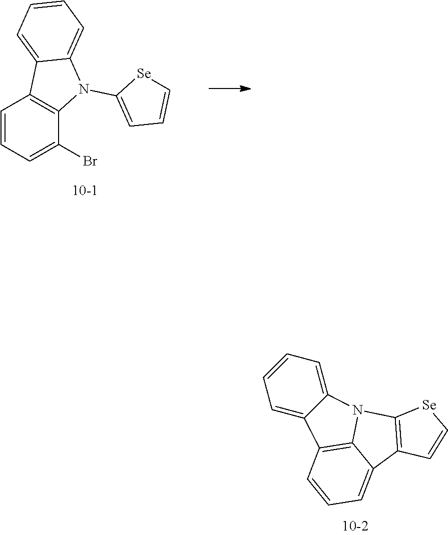 [SMILES: Brc1cccc2c3ccccc3n(-c3ccc[se]3)c12 ; c1ccc2c(c1)c1cccc3c4cc[se]c4n2c13]

8.40 g (22.4 mmol) of Intermediate 10-1, 6.19 g (44.8 mmol) of K₂CO₃, and 70 mg (1.12 mmol) of allyl[1,3-bis(2,6-diisopropylphenyl)imidazol-2-ylidene]chloropalladium(II) were dissolved in DMAc and stirred at 130° C. for 4 hours. The reaction solution was cooled to room temperature, and then an organic layer was obtained through extraction three times with water and DCM. The obtained organic layer was washed with a sodium chloride solution, and then dried with sodium sulfate, followed by separation and purification through column chromatography, to thereby obtain 3.56 g (69%) of Intermediate 10-2.

### Synthesis of Intermediate 10-3

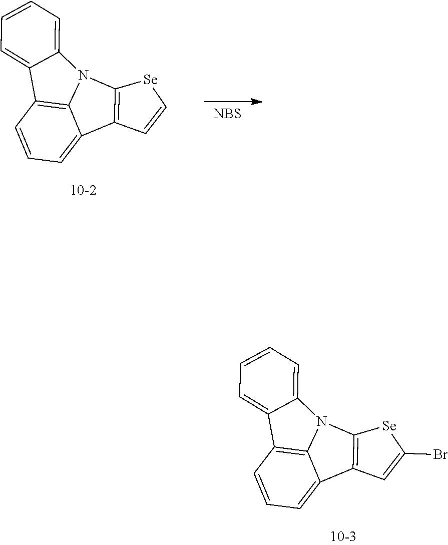

3.56 g (12.1 mmol) of Intermediate 10-2 and 2.58 g (14.5 mmol) of N-bromosuccinimide were dissolved in 120 ml of DMF and stirred at room temperature for 24 hours. 40 ml of water was added to the solution, and extraction was performed three times with 50 ml of ethyl ether to obtain an organic layer. The obtained organic layer was dried with magnesium sulfate, and the residue obtained by evaporating the solvent was separated and purified through silica gel column chromatography, to thereby obtain 3.36 g (74%) of Intermediate 10-3.

### Synthesis of Intermediate 10-4

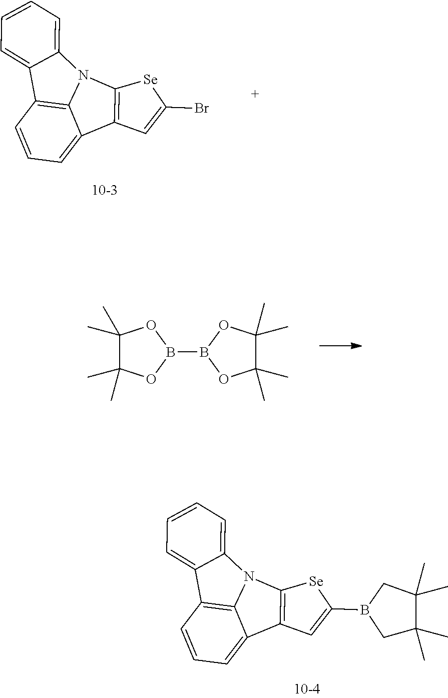

3.36 g (9.0 mmol) of Intermediate 10-3, 2.29 g (9.0 mmol) of bis(pinacolato)diborone, 0.36 g (0.5 mmol) of Pd(dppf)Cl₂, and 2.94 g (30.0 mmol) of KOAc were dissolved in 40 ml of DMSO, and then stirred at 80° C. for 6 hours. The reaction solution was cooled to room temperature, and extraction was performed three times with 50 ml of water and 50 ml of diethyl ether to obtain an organic layer. The obtained organic layer was dried with magnesium sulfate, and the residue obtained by evaporating the solvent was separated and purified through silica gel column chromatography, to thereby obtain 3.03 g (80%) of Intermediate 10-4.

### Synthesis of Intermediate 10-5

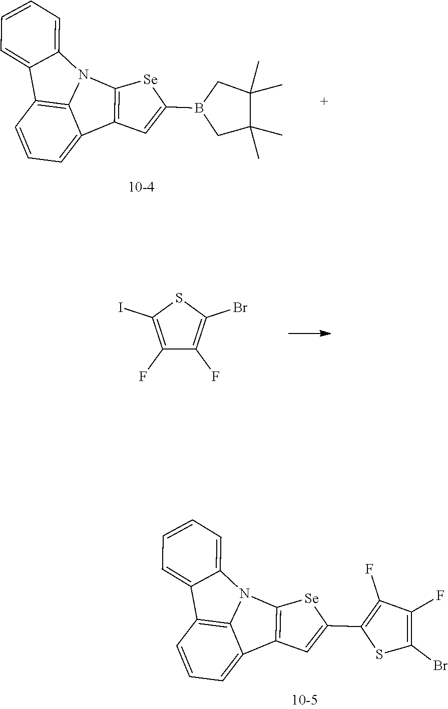 [SMILES: CC1(C)CB(c2cc3c4cccc5c6ccccc6n(c3[se]2)c54)CC1(C)C ; Fc1c(Br)sc(I)c1F ; Fc1c(Br)sc(-c2cc3c4cccc5c6ccccc6n(c3[se]2)c54)c1F]

3.03 g (7.2 mmol) of Intermediate 10-4, 2.34 g (7.2 mmol) of 2-bromo-3,4-difluoro-5-iodothiophene, 116 g (1.0 mmol) of Pd(PPh₃)₄, and 2.49 g (18 mmol) of K₂CO₃ were dissolved in 120 ml of a mixed solution of THF/H₂O (2:1), followed by stirring at 70° C. for 5 hours. The reaction solution was cooled to room temperature, and extraction was performed three times with 60 ml of water and 80 ml of ethyl ether to obtain an organic layer. The obtained organic layer was dried with magnesium sulfate, and the residue obtained by evaporating the solvent was separated and purified through silica gel column chromatography, to thereby obtain 2.62 g (74%) of Intermediate 10-5.

### Synthesis of Intermediate 10-6

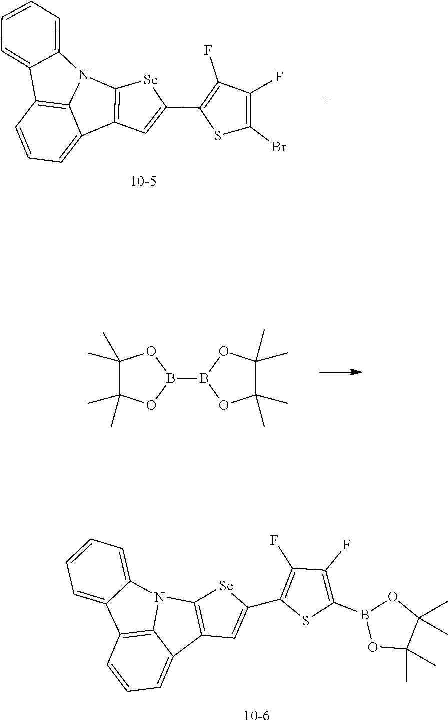

Intermediate 10-6 was synthesized using the same method as that used to synthesize Intermediate 10-4, except that Intermediate 10-5 was used instead of Intermediate 10-3 in the synthesis of Intermediate 10-4 of Synthesis Example 1.

### Synthesis of Intermediate 10-7

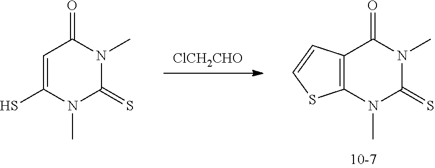

5.2 g (30 mmol) of 6-mercapto-1,3-dimethylthiouracil and 1.2 g (15 mmol) of sodium acetate were dissolved in 50 ml of water, and 10 ml (38 mmol) of a 30% solution of chloroacetaldehyde and 1.2 g (15 mmol) of sodium acetate dissolved in 20 ml of water were added dropwise at room temperature. A precipitate of the reaction solution was filtered to obtain 4.2 g (66%) of Intermediate 10-7.

### Synthesis of Intermediate 10-8

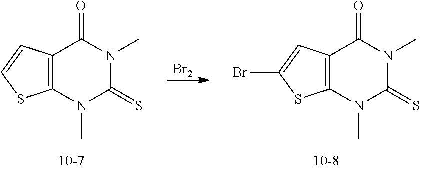

4.2 g (20 mmol) of Intermediate 10-7 was dissolved in 50 ml of acetic acid, and then a solution obtained by dissolving 3.2 g (20 mmol) of bromine in 50 ml of acetic acid was added dropwise. The mixture was stirred at room temperature for 1 hour. 150 ml of water was added, followed by filtration of the precipitate, to thereby obtain 5.36 g (92%) of Intermediate 10-8.

### Synthesis of Compound 10

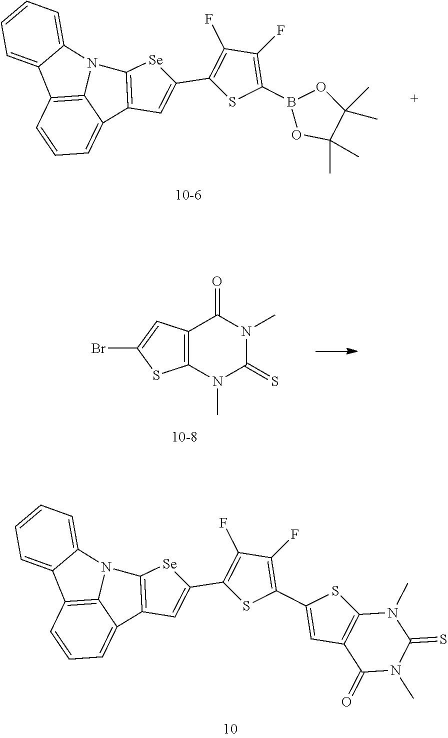 [SMILES: CC1(C)OB(c2sc(-c3cc4c5cccc6c7ccccc7n(c4[se]3)c65)c(F)c2F)OC1(C)C ; Cn1c(=O)c2cc(Br)sc2n(C)c1=S ; Cn1c(=O)c2cc(-c3sc(-c4cc5c6cccc7c8ccccc8n(c5[se]4)c76)c(F)c3F)sc2n(C)c1=S]

2.26 g (4.2 mmol) of Intermediate 10-6, 1.22 g (4.2 mmol) of Intermediate 10-8, 0.69 g (0.6 mmol) of tetrakis(triphenylphosphine)palladium, and 1.45 g (10.5 mmol) of K₂CO₃ were dissolved in 120 ml of a mixed solution of THF/H₂O (2:1), followed by stirring at 70° C. for 5 hours. The reaction solution was cooled to room temperature, and extraction was performed three times with 60 ml of water and 80 ml of ethyl ether to obtain an organic layer. The obtained organic layer was dried with magnesium sulfate, and the residue obtained by evaporating the solvent was separated and purified through silica gel column chromatography, to thereby obtain 1.94 g (74%) of Compound 10.

### Synthesis Example 2 (Synthesis of Compound 12)

### Synthesis of Intermediate 12-1

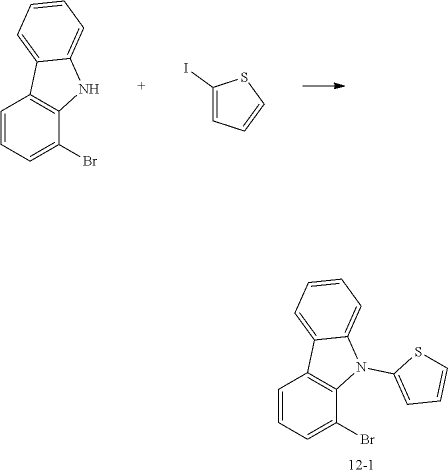 [SMILES: Brc1cccc2c1[nH]c1ccccc12 ; Ic1cccs1 ; Brc1cccc2c3ccccc3n(-c3cccs3)c12]

Intermediate 12-1 was synthesized using the same method as that used to synthesize Intermediate 10-1, except that 2-iodothiophene was used instead of 2-iodoselenophene in the synthesis of Intermediate 10-1 of Synthesis Example 1.

### Synthesis of Intermediate 12-2

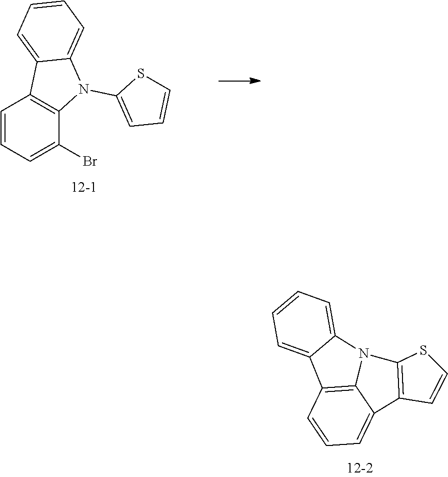 [SMILES: Brc1cccc2c3ccccc3n(-c3cccs3)c12 ; c1ccc2c(c1)c1cccc3c4ccsc4n2c13]

Intermediate 12-2 was synthesized using the same method as that used to synthesize Intermediate 10-2, except that Intermediate 12-1 was used instead of Intermediate 10-1 in the synthesis of Intermediate 10-2 of Synthesis Example 1.

### Synthesis of Intermediate 12-3

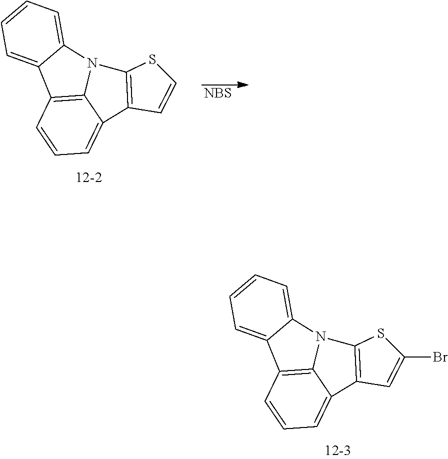

Intermediate 12-3 was synthesized using the same method as that used to synthesize Intermediate 10-3, except that Intermediate 12-2 was used instead of Intermediate 10-2 in the synthesis of Intermediate 10-3 of Synthesis Example 1.

### Synthesis of Intermediate 12-4

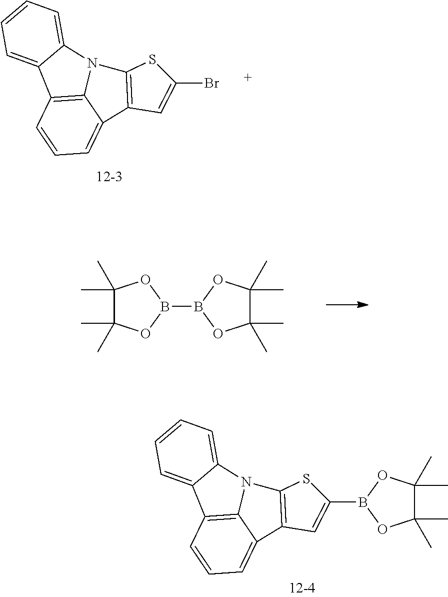

Intermediate 12-4 was synthesized using the same method as that used to synthesize Intermediate 10-4, except that Intermediate 12-3 was used instead of Intermediate 10-3 in the synthesis of Intermediate 10-4 of Synthesis Example 1.

### Synthesis of Intermediate 12-5

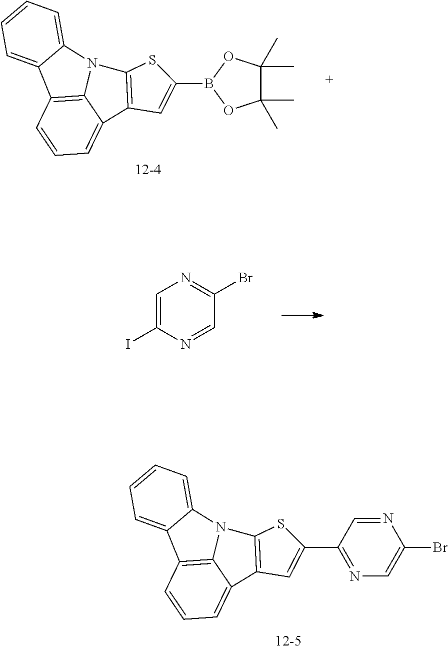 [SMILES: CC1(C)OB(c2cc3c4cccc5c6ccccc6n(c3s2)c54)OC1(C)C ; Brc1cnc(I)cn1 ; Brc1cnc(-c2cc3c4cccc5c6ccccc6n(c3s2)c54)cn1]

Intermediate 12-5 was synthesized using the same method as that used to synthesize Intermediate 10-5, except that Intermediate 12-4 was used instead of Intermediate 10-4, and 2-bromo-5-iodopyrazine was used instead of 2-bromo-3,4-difluoro-5-iodothiophene in the synthesis of Intermediate 10-5 of Synthesis Example 1.

### Synthesis of Intermediate 12-6

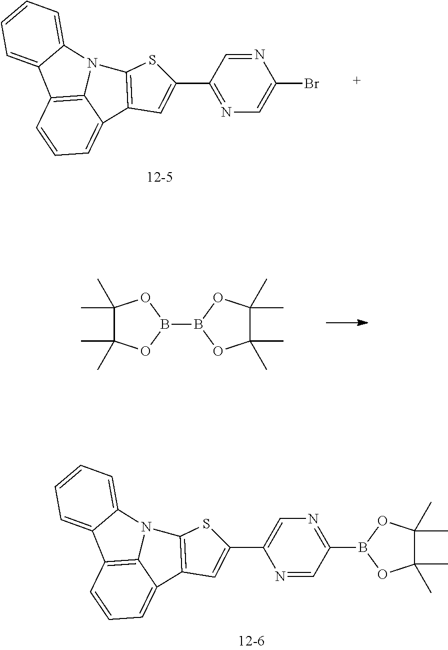

Intermediate 12-6 was synthesized using the same method as that used to synthesize Intermediate 10-4, except that Intermediate 12-5 was used instead of Intermediate 10-3 in the synthesis of Intermediate 10-4 of Synthesis Example 1.

### Synthesis of Compound 12

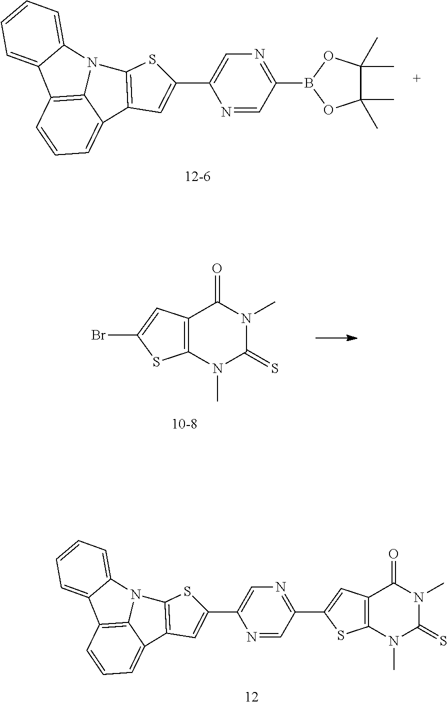 [SMILES: CC1(C)OB(c2cnc(-c3cc4c5cccc6c7ccccc7n(c4s3)c65)cn2)OC1(C)C ; Cn1c(=O)c2cc(Br)sc2n(C)c1=S ; Cn1c(=O)c2cc(-c3cnc(-c4cc5c6cccc7c8ccccc8n(c5s4)c76)cn3)sc2n(C)c1=S]

Compound 12 was synthesized using the same method as that used to synthesize Compound 10, except that Intermediate 12-6 was used instead of Intermediate 10-6 in the synthesis of Compound 10 of Synthesis Example 1.

### Synthesis Example 3 (Synthesis of Compound 59)

### Synthesis of Intermediate 59-1

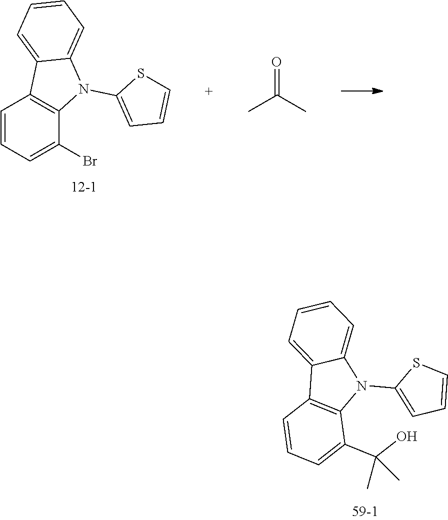

7.35 g (22.4 mmol) of Intermediate 12-1 was dissolved in 250 ml of dehydrated diethyl ether, and 8 ml (32.0 mmol) of a hexane solution containing 2.76 M n-butyl lithium was added dropwise at −50° C., followed by stirring at room temperature for 1 hour. 1.3 g (25 mmol) of dehydrated acetone was added to the mixture at −50° C. and stirred at room temperature for 2 hours. Organic layer extracted from diethyl ether was washed with a sodium chloride solution, and then dried with anhydrous magnesium sulfate. The obtained product was separated and purified through silica gel column chromatography to thereby obtain 4.54 g (66%) of Intermediate 59-1.

### Synthesis of Intermediate 59-2

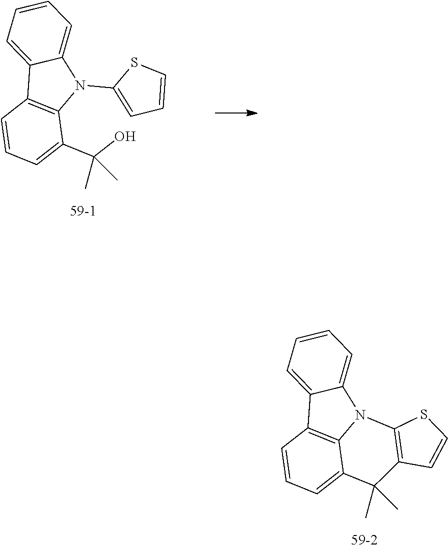 [SMILES: CC(C)(O)c1cccc2c3ccccc3n(-c3cccs3)c12 ; CC1(C)c2ccsc2-n2c3ccccc3c3cccc1c32]

4.54 g (14.8 mmol) of Intermediate 59-1 was dissolved in 180 ml of dichloromethane, and 4.98 g (35.5 mmol) of a boron trifluoride-ethyl ether complex was added dropwise at 0° C., followed by stirring for 2 hours. An organic layer extracted from dichloromethane was washed with a sodium chloride solution, and then dried with anhydrous magnesium sulfate. The obtained product was separated and purified through silica gel column chromatography to thereby obtain 3.51 g (82%) of Intermediate 59-2.

### Synthesis of Intermediate 59-3

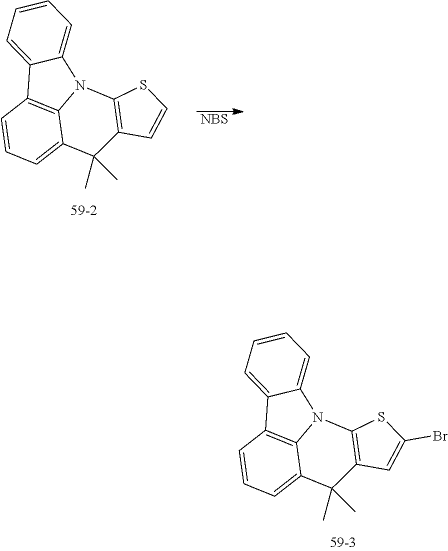

Intermediate 59-3 was synthesized using the same method as that used to synthesize Intermediate 10-3, except that Intermediate 59-2 was used instead of Intermediate 10-2 in the synthesis of Intermediate 10-3 of Synthesis Example 1.

### Synthesis of Intermediate 59-4

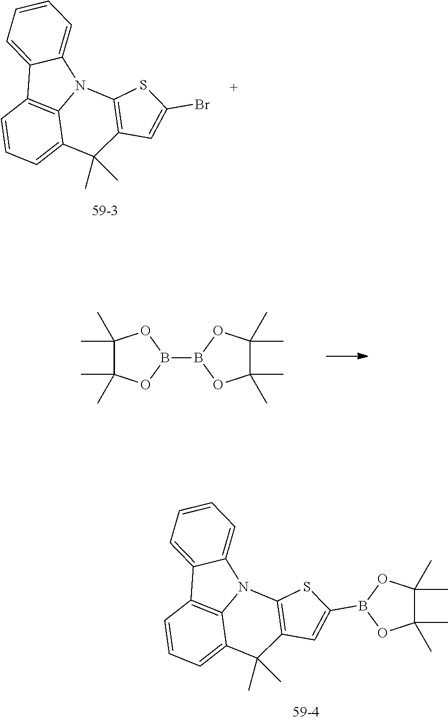

Intermediate 59-4 was synthesized using the same method as that used to synthesize Intermediate 10-4, except that Intermediate 59-3 was used instead of Intermediate 10-3 in the synthesis of Intermediate 10-4 of Synthesis Example 1.

### Synthesis of Intermediate 59-5

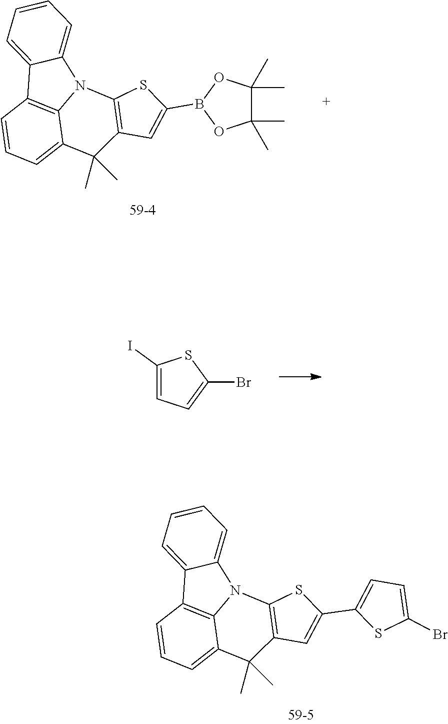 [SMILES: CC1(C)c2cc(B3OC(C)(C)C(C)(C)O3)sc2-n2c3ccccc3c3cccc1c32 ; Brc1ccc(I)s1 ; CC1(C)c2cc(-c3ccc(Br)s3)sc2-n2c3ccccc3c3cccc1c32]

Intermediate 59-5 was synthesized using the same method as that used to synthesize Intermediate 10-5, except that Intermediate 59-4 was used instead of Intermediate 10-4 and 2-bromo-5-iodothiophene was used instead of 2-bromo-3,4-difluoro-5-iodothiophene in the synthesis of Intermediate 10-5 of Synthesis Example 1.

### Synthesis of Intermediate 59-6

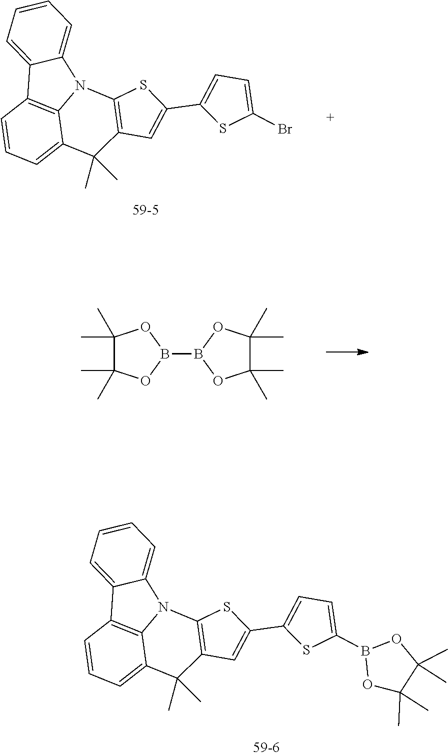

Intermediate 59-6 was synthesized using the same method as that used to synthesize Intermediate 10-4, except that Intermediate 59-5 was used instead of Intermediate 10-3 in the synthesis of Intermediate 10-4 of Synthesis Example 1.

### Synthesis of Intermediate 59-7

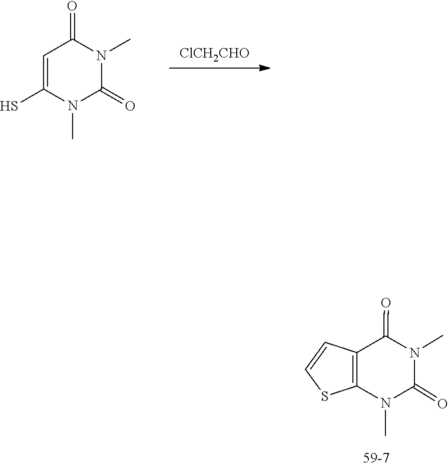

Intermediate 59-7 was synthesized using the same method as that used to synthesize Intermediate 10-7, except that 6-mercapto-1,3-dimethylpyrimidine-2,4(1H,3H)-dione was used instead of 6-mercapto-1,3-dimethyluracil in the synthesis of Intermediate 10-7 of Synthesis Example 1.

### Synthesis of Intermediate 59-8

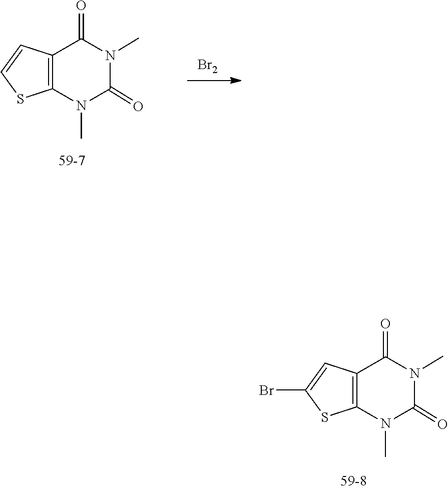

Intermediate 59-8 was synthesized using the same method as that used to synthesize Intermediate 10-8, except that Intermediate 59-7 was used instead of Intermediate 10-7 in the synthesis of Intermediate 10-8 of Synthesis Example 1.

### Synthesis of Compound 59

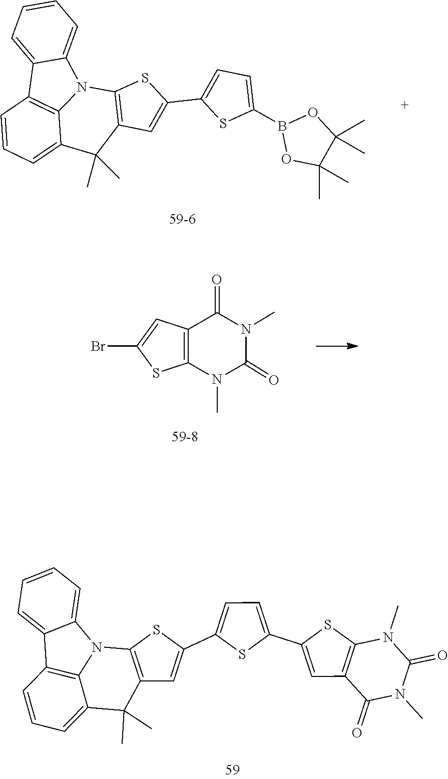 [SMILES: CC1(C)c2cc(-c3ccc(B4OC(C)(C)C(C)(C)O4)s3)sc2-n2c3ccccc3c3cccc1c32 ; Cn1c(=O)c2cc(Br)sc2n(C)c1=O ; Cn1c(=O)c2cc(-c3ccc(-c4cc5c(s4)-n4c6ccccc6c6cccc(c64)C5(C)C)s3)sc2n(C)c1=O]

Compound 59 was synthesized using the same method as that used to synthesize Compound 10, except that Intermediate 59-6 was used instead of Intermediate 10-6, and Intermediate 59-8 was used instead of Intermediate 10-8 in the synthesis of Compound 10 of Synthesis Example 1.

### Synthesis Example 4 (Synthesis of Compound 67)

### Synthesis of Compound 67

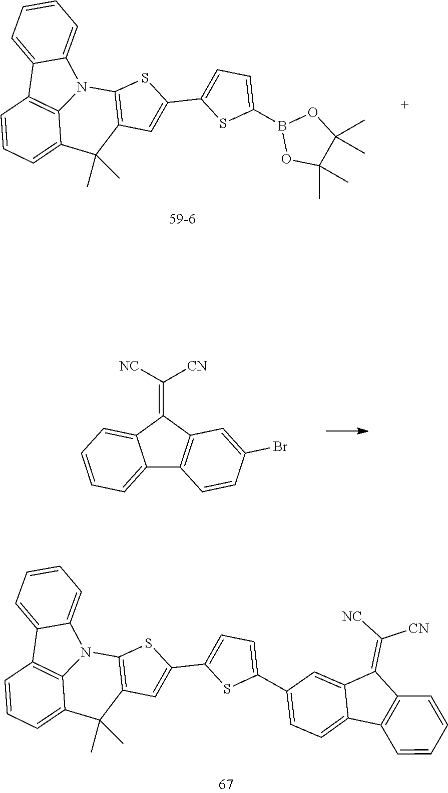 [SMILES: CC1(C)c2cc(-c3ccc(B4OC(C)(C)C(C)(C)O4)s3)sc2-n2c3ccccc3c3cccc1c32 ; N#CC(C#N)=C1c2ccccc2-c2ccc(Br)cc21 ; CC1(C)c2cc(-c3ccc(-c4ccc5c(c4)C(=C(C#N)C#N)c4ccccc4-5)s3)sc2-n2c3ccccc3c3cccc1c32]

Compound 67 was synthesized using the same method as that used to synthesize Compound 10, except that Intermediate 59-6 was used instead of Intermediate 10-6, and 2-(2-bromo-9H-fluoren-9-ylidene)malononitrile was used instead of Intermediate 10-8 in the synthesis of Compound 10 of Synthesis Example 1.

### Synthesis Example 5 (Synthesis of Compound 69)

### Synthesis of Intermediate 69-1

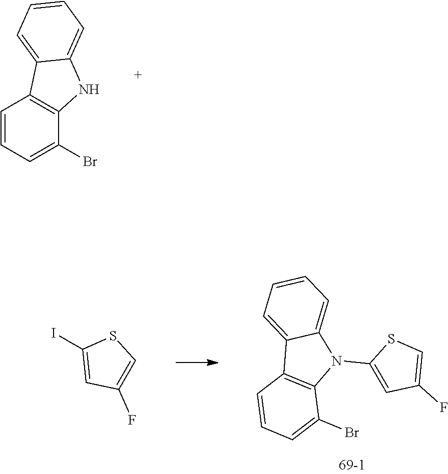 [SMILES: Brc1cccc2c1[nH]c1ccccc12 ; Fc1csc(I)c1 ; Fc1csc(-n2c3ccccc3c3cccc(Br)c32)c1]

Intermediate 69-1 was synthesized using the same method as that used to synthesize Intermediate 10-1, except that 4-fluoro-2-iodothiophene was used instead of 2-iodoselenophene in the synthesis of Intermediate 10-1 of Synthesis Example 1.

### Synthesis of Intermediate 69-2

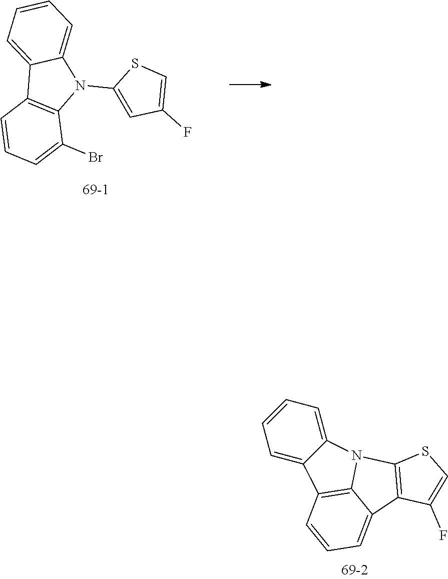 [SMILES: Fc1csc(-n2c3ccccc3c3cccc(Br)c32)c1 ; Fc1csc2c1c1cccc3c4ccccc4n2c31]

Intermediate 69-2 was synthesized using the same method as that used to synthesize Intermediate 10-2, except that Intermediate 69-1 was used instead of Intermediate 10-1 in the synthesis of Intermediate 10-2 of Synthesis Example 1.

### Synthesis of Intermediate 69-3

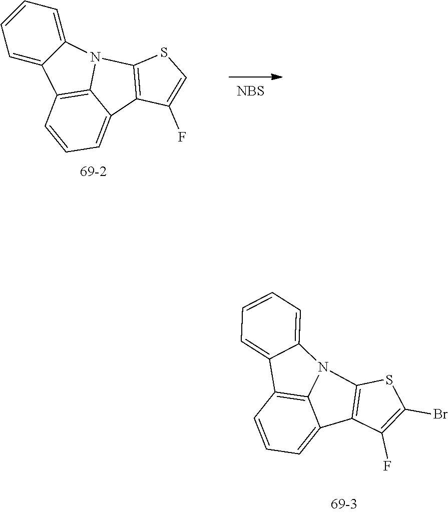

Intermediate 69-3 was synthesized using the same method as that used to synthesize Intermediate 10-3, except that Intermediate 69-2 was used instead of Intermediate 10-2 in the synthesis of Intermediate 10-3 of Synthesis Example 1.

### Synthesis of Intermediate 69-4

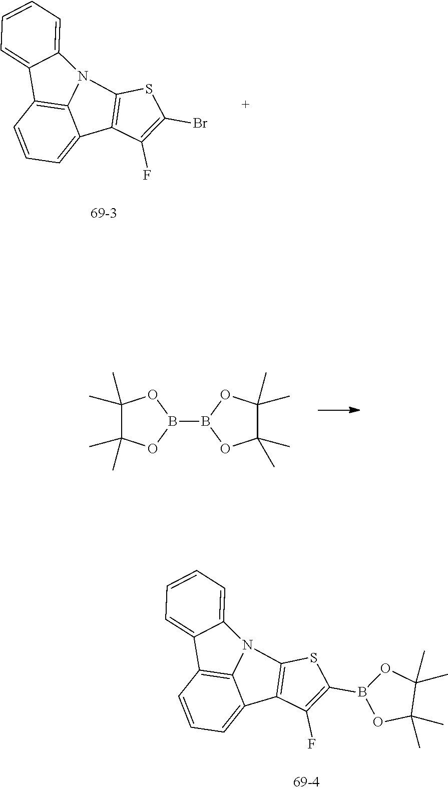

Intermediate 69-4 was synthesized using the same method as that used to synthesize Intermediate 10-4, except that Intermediate 69-3 was used instead of Intermediate 10-3 in the synthesis of Intermediate 10-4 of Synthesis Example 1.

### Synthesis of Intermediate 69-5

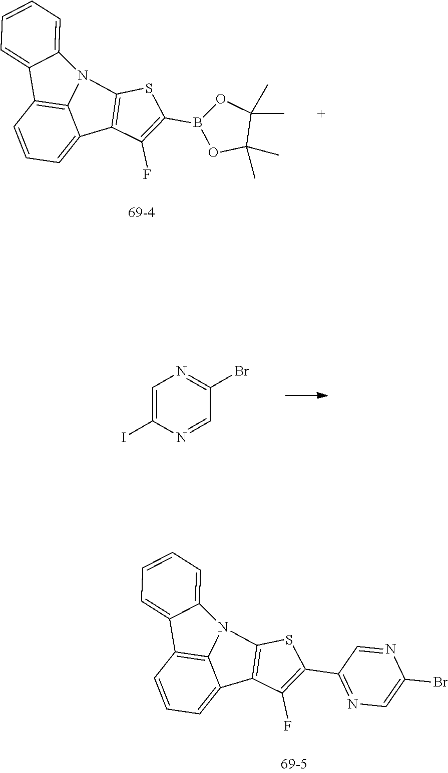 [SMILES: CC1(C)OB(c2sc3c(c2F)c2cccc4c5ccccc5n3c42)OC1(C)C ; Brc1cnc(I)cn1 ; Fc1c(-c2cnc(Br)cn2)sc2c1c1cccc3c4ccccc4n2c31]

Intermediate 69-5 was synthesized using the same method as that used to synthesize Intermediate 10-5, except that Intermediate 69-4 was used instead of Intermediate 10-4, and 2-bromo-5-iodopyrazine was used instead of 2-bromo-3,4-difluoro-5-iodothiophene in the synthesis of Intermediate 10-5 of Synthesis Example 1.

### Synthesis of Intermediate 69-6

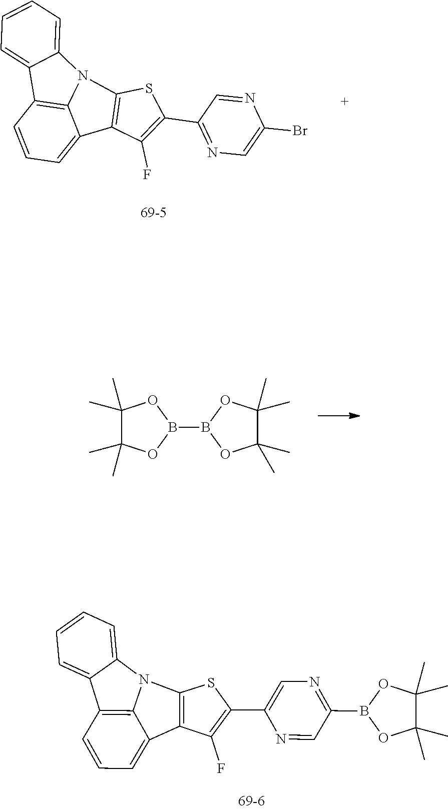

Intermediate 69-6 was synthesized using the same method as that used to synthesize Intermediate 10-4, except that Intermediate 69-5 was used instead of Intermediate 10-3 in the synthesis of Intermediate 10-4 of Synthesis Example 1.

### Synthesis of Intermediate 69-7

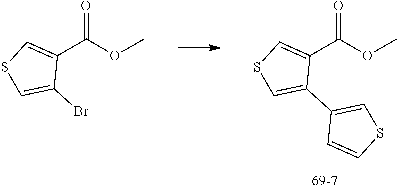

2.37 g (10.73 mmol) of methyl-4-bromothiophene-3-carboxylate, 1.6 g (12.62 mmol) of 3-thienyl boronic acid, and 4.6 g (42.92 mmol) of Na₂CO₃ were dissolved in a THF/H₂O (4:1) solution. 150 mg (0.126 mmol) of Pd(PPh₃)₄ was added in an argon atmosphere, followed by stirring at 70° C. for 16 hours. The mixture was cooled to room temperature, and then an organic layer was obtained through extraction with water and CH₂Cl₂. The obtained organic layer was separated and purified through column chromatography to thereby obtain 2.38 g (99%) of Intermediate 69-7.

### Synthesis of Intermediate 69-8

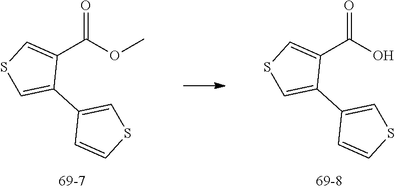

2.37 g (10.58 mmol) of Intermediate 69-7 was dissolved in 20 ml of 4N NaOH and 20 ml of EtOH and stirred at 90° C. for 4 hours. The mixture was cooled at 0° C., and then a 6N HCl solution was added dropwise. The precipitate was filtered, followed by washing with distilled water and drying, to thereby obtain 1.65 g (74%) of Intermediate 69-8.

### Synthesis of Intermediate 69-9

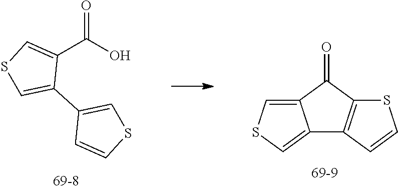 [SMILES: O=C(O)c1cscc1-c1ccsc1 ; O=C1c2cscc2-c2ccsc21]

0.95 g (4.5 mmol) of Intermediate 69-8 was dissolved in 30 ml of CH₂Cl₂. 1 ml of oxalyl chloride was added dropwise to the solution, and then 0.2 ml of DMF was added, followed by stirring at room temperature for 24 hours. The product was diluted in 5 ml of CH₂Cl₂, and added to a solution obtained by dissolving 1.5 g (11 mmol) of AlCl₃ in 30 ml of CH₂Cl₂. The mixture was stirred for 1 hour, and then added to ice water, followed by extraction with CH₂Cl₂. The extract was separated and purified through column chromatography to thereby obtain 0.78 g (90%) of Intermediate 69-9.

### Synthesis of Intermediate 69-10

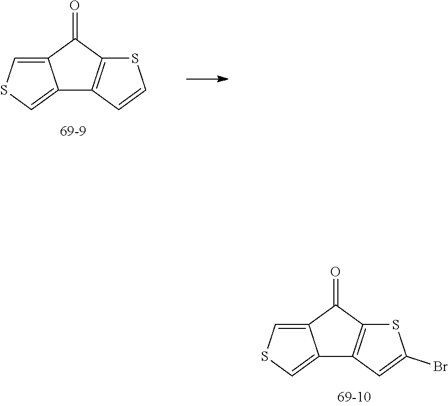 [SMILES: O=C1c2cscc2-c2ccsc21 ; O=C1c2cscc2-c2cc(Br)sc21]

0.58 g (3.0 mmol) of Intermediate 69-9 and 0.756 g (3 mmol) of NaCO₃ were dissolved in 10 ml of CHCl₃. 0.2 ml of Br₂ was added dropwise to the solution and stirred for 1.5 hours. 20 ml of 1N Na₂SO₃ was added, and then an organic layer was obtained through extraction with water. The obtained organic layer was separated and purified through silica gel column chromatography to thereby obtain 0.77 g (95%) of Intermediate 69-10.

### Synthesis of Compound 69

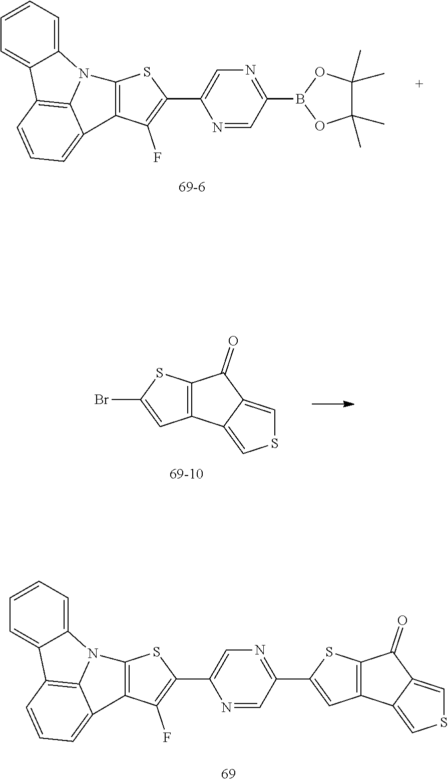 [SMILES: CC1(C)OB(c2cnc(-c3sc4c(c3F)c3cccc5c6ccccc6n4c53)cn2)OC1(C)C ; O=C1c2cscc2-c2cc(Br)sc21 ; O=C1c2cscc2-c2cc(-c3cnc(-c4sc5c(c4F)c4cccc6c7ccccc7n5c64)cn3)sc21]

Compound 69 was synthesized using the same method as that used to synthesize Compound 10, except that Intermediate 69-6 was used instead of Intermediate 10-6, and Intermediate 69-10 was used instead of Intermediate 10-8 in the synthesis of Compound 10 of Synthesis Example 1.

### Example 1

A 15 Ω/cm² (1,200 Å) ITO glass substrate as an anode available from Corning Inc. was cut to a size of 50 mm×50 mm×0.7 mm, sonicated in isopropyl alcohol and pure water for 5 minutes each, and then cleaned by irradiation of ultraviolet rays and exposure to ozone for 30 minutes, and then the glass substrate was mounted on a vacuum deposition apparatus.

2-TNATA was vacuum-deposited on the anode to form a hole injection layer having a thickness of 600 Å. Subsequently, NPB was vacuum-deposited on the hole injection layer to form a hole transport layer having a thickness of 300 Å.

Compound 10 as a p-type semiconductor compound was deposited on the hole transport layer, and then N1 as an n-type semiconductor compound was deposited, to form a photoactive layer having a total thickness of 450 Å.

Alq₃ was vacuum-deposited on the photoactive layer to form an electron transport layer having a thickness of 300 Å. LiF was deposited on the electron transport layer to a thickness of 10 Å to form an electron injection layer. Al was vacuum-deposited on the electron injection layer to a thickness of 3,000 Å to form a LiF/Al cathode, thereby completing the manufacture of an optoelectronic device.

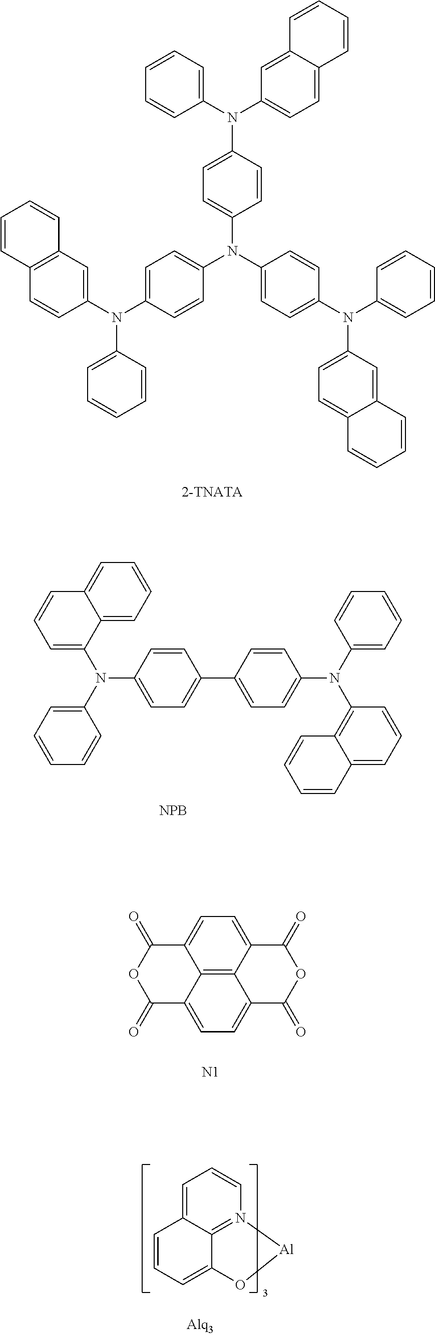

### Examples 2 to 5 and Comparative Examples 1 to 4

Optoelectronic devices were manufactured in the same manner as in Example 1, except that compounds shown in Table 2 were used instead of Compound 10 in the formation of the photoactive layer.

### Evaluation Example 1

To evaluate the properties of the compound used as a p-type semiconductor compound in each of Examples 1 to 5 and Comparative Examples 1 to 4, changes in HOMO energy level, LUMO energy level, maximum absorption wavelength, oscillator strength (OSC), and purity were measured, and the results thereof are shown in Table 1.

For the evaluation of the properties, quantum simulation was performed at the B3LYP/6-311G** level on the basis of time-dependent density functional theory (TD-DFT) methodology using a Gaussian program to evaluate the energy level and oscillator strength in the structural optimization state and excited state.

In addition, a change in the purity of each compound was evaluated by placing each compound in a glass vacuum tube, heating the glass vacuum tube at a deposition process temperature (330° C.), and then maintaining the temperature and measuring the purity of each compound.

TABLE 1

| | HOMO | LUMO | Maximum | | change |
|---|---|---|---|---|---|
| | energy | energy | absorption | | in |
| | level | level | wavelength | Oscillator | purity |
| | [eV] | [eV] | [nm] | strength | [%] |
| Compound 10 | −5.43 | −2.45 | 540 | 1.58 | −0.1 |
| Compound 12 | −5.60 | −2.48 | 520 | 1.64 | −0.1 |
| Compound 59 | −5.86 | −3.26 | 560 | 1.05 | −0.1 |
| Compound 67 | −5.21 | −3.46 | 533 | 1.00 | −0.1 |
| Compound 69 | −5.77 | −2.80 | 547 | 1.00 | −0.1 |
| Comparative | −5.62 | −2.66 | 535 | 0.89 | −7.3 |
| Compound 1C | | | | | |
| Comparative | −5.45 | −2.55 | 555 | 0.64 | −6.9 |
| Compound 2C | | | | | |
| Comparative | −5.40 | −2.11 | 466 | 1.14 | −5.4 |
| Compound 3C | | | | | |
| Comparative | −5.32 | −1.65 | 464 | 0.69 | −0.1 |
| Compound 4C | | | | | |

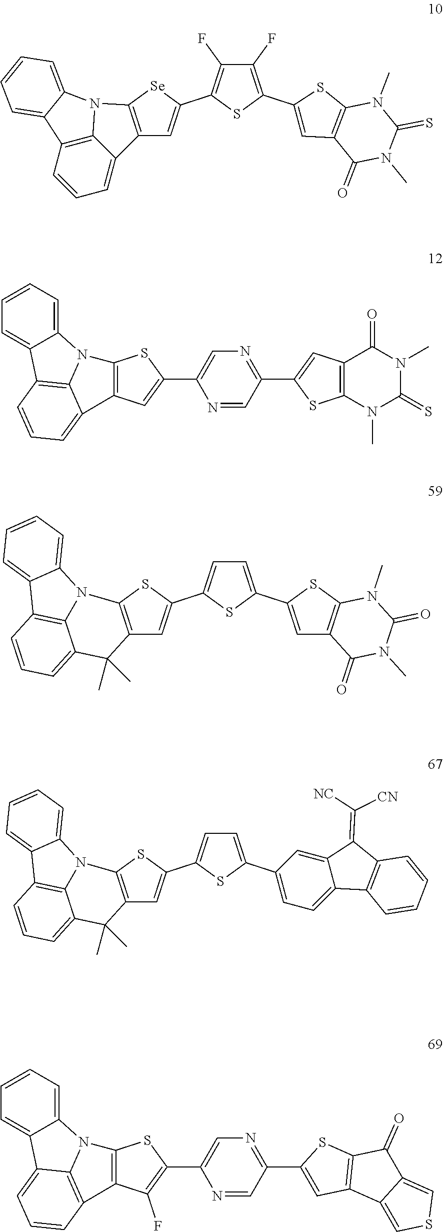 [SMILES: Cn1c(=O)c2cc(-c3sc(-c4cc5c6cccc7c8ccccc8n(c5[se]4)c76)c(F)c3F)sc2n(C)c1=S ; Cn1c(=O)c2cc(-c3cnc(-c4cc5c6cccc7c8ccccc8n(c5s4)c76)cn3)sc2n(C)c1=S ; Cn1c(=O)c2cc(-c3ccc(-c4cc5c(s4)-n4c6ccccc6c6cccc(c64)C5(C)C)s3)sc2n(C)c1=O ; CC1(C)c2cc(-c3ccc(-c4ccc5c(c4)C(=C(C#N)C#N)c4ccccc4-5)s3)sc2-n2c3ccccc3c3cccc1c32 ; O=C1c2cscc2-c2cc(-c3cnc(-c4sc5c(c4F)c4cccc6c7ccccc7n5c64)cn3)sc21]

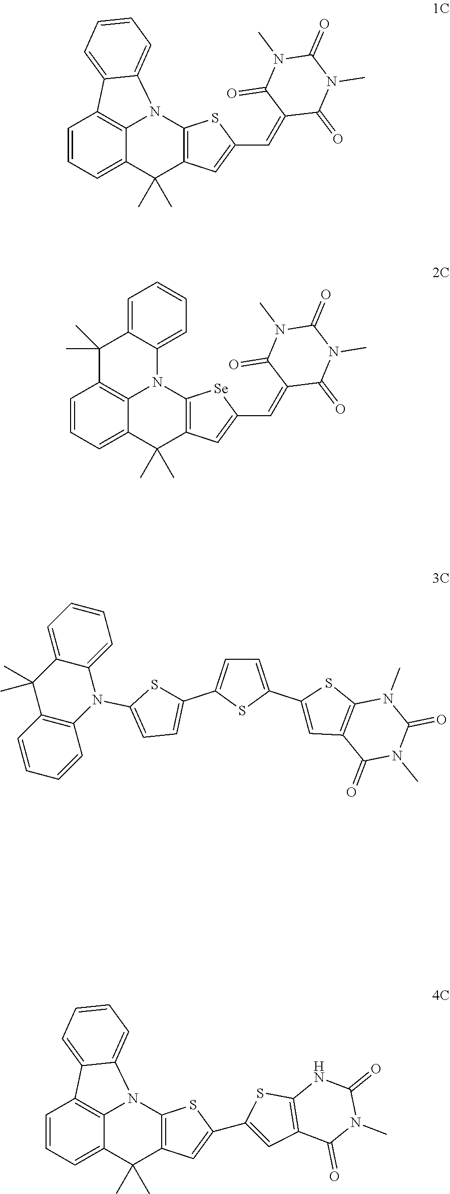

From Table 1, it can be confirmed that the organic compounds used in Examples 1 to 5 have energy levels suitable for use as a p-type semiconductor and have maximum absorption wavelengths in green light. It can also be confirmed that the organic compounds used in Examples 1 to 5 have higher oscillator strengths than Comparative Compounds 1C, 2C and 4C used in Comparative Examples 1, 2 and 4, indicating that the extinction coefficient is high. It can also be confirmed that the organic compounds used in Examples 1 to 5 are hardly denatured at the deposition process temperature.

### Evaluation Example 2

To evaluate the characteristics of the optoelectronic devices manufactured according to Comparative Examples 1 to 4 and Examples 1 to 5, external quantum efficiency (EQE) was measured, and the results thereof are shown in Table 2.

Each optoelectronic device was irradiated with light (530 nm) by using an external quantum efficiency meter (K3100, McScience, Korea). The current generated during light irradiation was measured by using an ammeter (Keithley, Tektronix, USA). The external quantum efficiency (EQE) was calculated using the irradiated light and the measured current.

TABLE 2

| | p-type semiconductor | EQE (@530 nm) |
|---|---|---|
| | compound | [%] |
| Example 1 | Compound 10 | 39.7 |
| Example 2 | Compound 12 | 41.2 |
| Example 3 | Compound 59 | 21.1 |
| Example 4 | Compound 67 | 25.7 |
| Example 5 | Compound 69 | 23.8 |
| Comparative | Comparative | 22.8 |
| Example 1 | Compound 1C | |
| Comparative | Comparative | 13.9 |
| Example 2 | Compound 2C | |
| Comparative | Comparative | 9.5 |
| Example 3 | Compound 3C | |
| Comparative | Comparative | 5.3 |
| Example 4 | Compound 4C | |

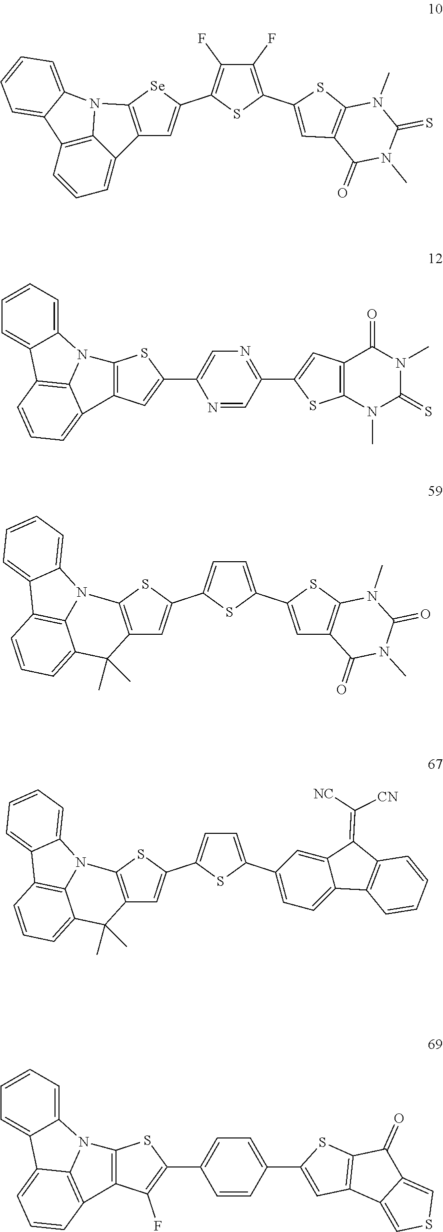 [SMILES: Cn1c(=O)c2cc(-c3sc(-c4cc5c6cccc7c8ccccc8n(c5[se]4)c76)c(F)c3F)sc2n(C)c1=S ; Cn1c(=O)c2cc(-c3cnc(-c4cc5c6cccc7c8ccccc8n(c5s4)c76)cn3)sc2n(C)c1=S ; Cn1c(=O)c2cc(-c3ccc(-c4cc5c(s4)-n4c6ccccc6c6cccc(c64)C5(C)C)s3)sc2n(C)c1=O ; CC1(C)c2cc(-c3ccc(-c4ccc5c(c4)C(=C(C#N)C#N)c4ccccc4-5)s3)sc2-n2c3ccccc3c3cccc1c32 ; O=C1c2cscc2-c2cc(-c3ccc(-c4sc5c(c4F)c4cccc6c7ccccc7n5c64)cc3)sc21]

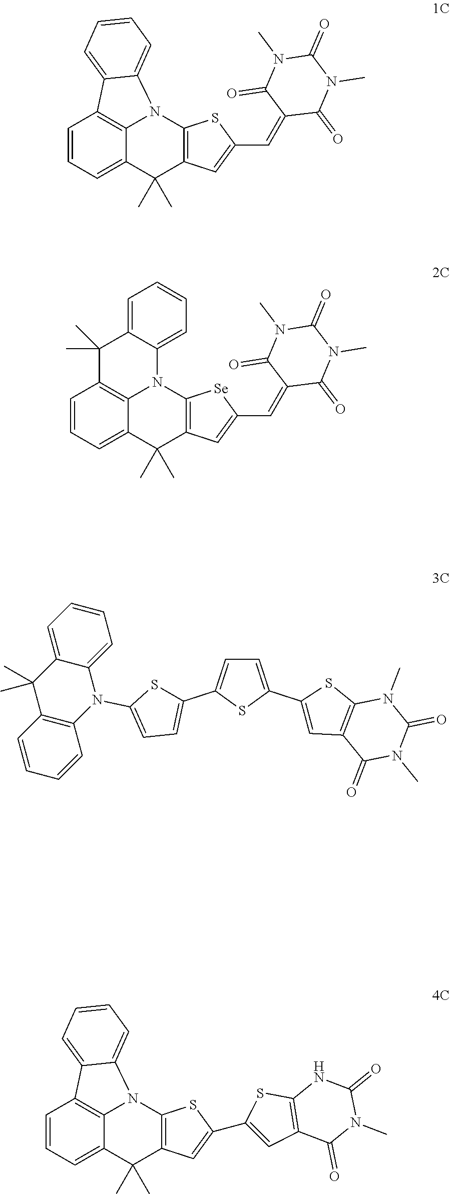

From Table 2, it can be confirmed that the optoelectronic devices of Examples 1 to 5 exhibit higher external quantum efficiency (EQE) in a green wavelength region during the deposition process, than the optoelectronic devices of Comparative Examples 1 to 4.

Thus, from Tables 1 and 2, it can be confirmed that the optoelectronic devices of Examples 1 to 5 have excellent light absorption characteristics, charge transfer characteristics, and thermal characteristics, compared to the optoelectronic devices of Comparative Examples 1 to 4. From the above results, it can be confirmed that the optoelectronic devices of Examples 1 to 5 have excellent optoelectronic characteristics.

According to some embodiments, provided are a compound capable of absorbing light in a green wavelength region and having excellent absorption strength and external quantum efficiency and high heat resistance, an optoelectronic device which absorbs green light and maintains high external quantum efficiency under high-temperature process conditions, and an electronic apparatus and electronic equipment which include the optoelectronic device.

It should be understood that embodiments described herein should be considered in a descriptive sense only and not for purposes of limitation. Descriptions of features or aspects within each embodiment should typically be considered as available for other similar features or aspects in other embodiments. While some embodiments have been described with reference to the figures, it will be understood by those of ordinary skill in the art that various changes in form and details may be made therein without departing from the spirit and scope as defined by the following claims.

What is claimed is:

1. An optoelectronic device comprising:
a first electrode;
a second electrode on the first electrode;
a photoactive layer between the first electrode and the second electrode; and
a first compound represented by Formula 1: 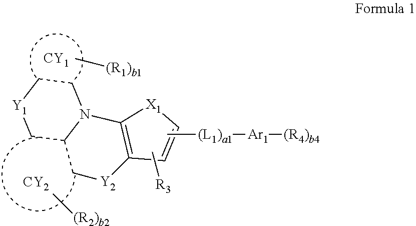
wherein, in Formula 1,
X₁ is selected from oxygen (O), sulfur (S), selenium (Se), and tellurium (Te),
Y₁ and Y₂ are each independently selected from a single bond, *—O—*′, *—S—*′, *—Se—*′, *—C(R₅)(R₆)—*′, *—Si(R₅)(R₆)—*′, *—Ge(R₅)(R₆)—*′, *—N(R₅)—*′, *—P(R₅)—*′, *—C(═O)—*′, *—C(═S)—*′, *—S(═O)—*′, and *—S(═O)₂—*′,
* and *′ each indicate a binding site to a neighboring atom,
L₁ is a C₃-C₆₀ carbocyclic group unsubstituted or substituted with at least one R₁₀ₐ or a C₁-C₆₀ heterocyclic group unsubstituted or substituted with at least one R₁₀ₐ,
a1 is an integer from 1 to 5,
Ar₁ is a C₄-C₆₀ polycyclic group in which two or more cyclic groups are condensed with each other, and the two or more cyclic groups are each independently a C₃-C₃₀ carbocyclic group or a C₁-C₃₀ heterocyclic group,
rings CY₁ and CY₂ are each independently a C₅-C₆₀ carbocyclic group or a C₂-C₆₀ heterocyclic group,
b1, b2, and b4 are each independently an integer from 0 to 10,
R₁ to R₆ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
two or more of R₁ to R₆ are optionally linked to each other to form a C₅-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, cyano group, a C₁-C₆₀ alkyl group, a C₁-C₆₀ alkoxy group, a phenyl group, a biphenyl group, or any combination thereof.

2. The optoelectronic device of claim 1, further comprising a second compound represented by one of Formulae 2-1 to 2-6: 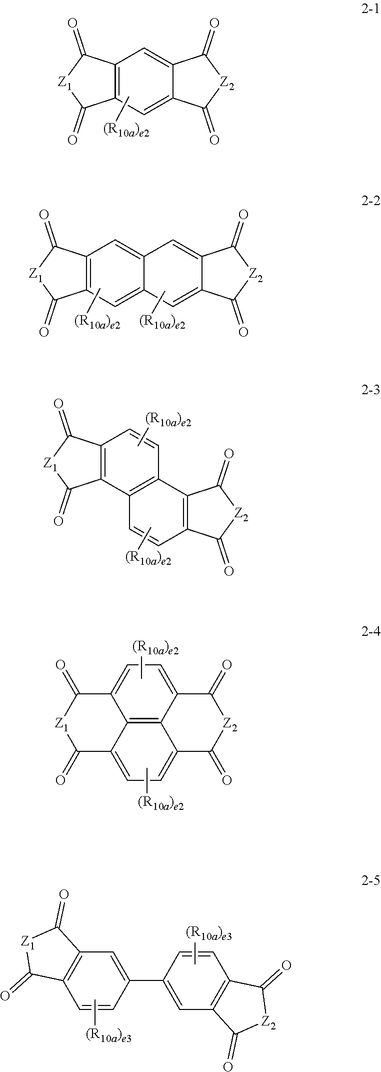 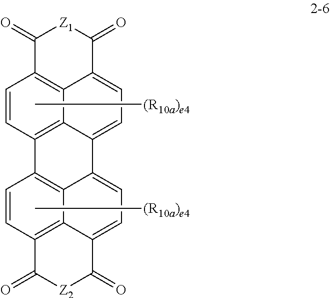
wherein, in Formulae 2-1 to 2-6,
Z₁ and Z₂ are each independently selected from O, S, N(R₂₄), C(R₂₄)(R₂₅), C(═O), C(═S), and C═C(R₂₄)(R₂₅),
R₂₄ and R₂₅ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
Q₁ to Q₃ and Rima are as described in claim 1,
e2 is an integer from 0 to 2,
e3 is an integer from 0 to 3, and
e4 is an integer from 0 to 4.

3. The optoelectronic device of claim 2, wherein R₂₄ and R₂₅ are each independently a C₁-C₆₀ alkyl group, a C₃-C₁₀ cycloalkyl group, a C₆-C₆₀ aryl group, or a C₁-C₆₀ heteroaryl group, each unsubstituted or substituted with deuterium, —F, —Cl, —Br, —I, a cyano group, a C₁-C₆₀ alkyl group, a C₁-C₆₀ alkoxy group, or any combination thereof.

4. The optoelectronic device of claim 2, wherein the second compound is one of Compounds N1 to N43:

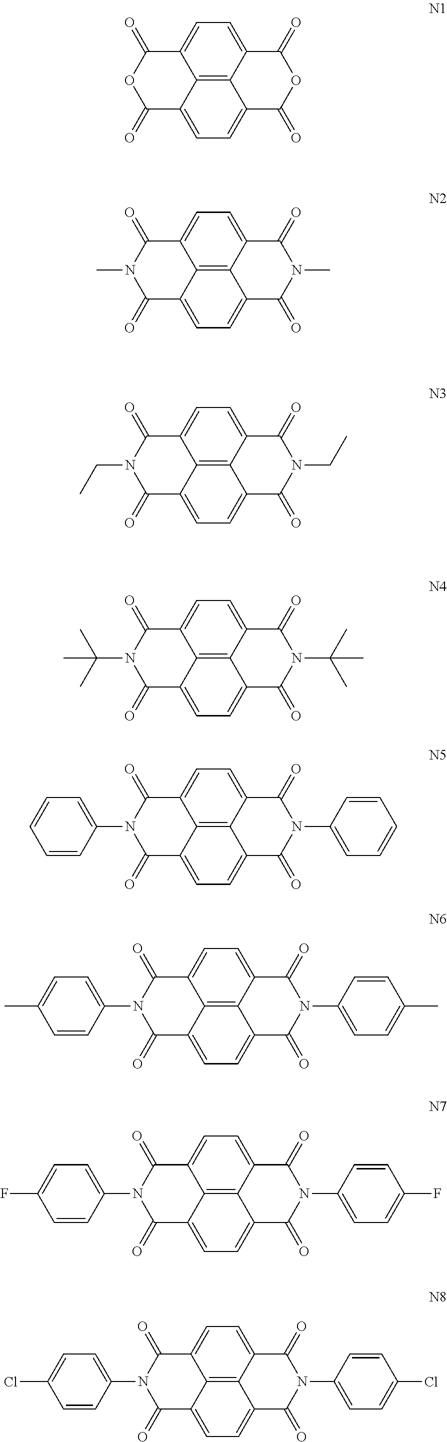

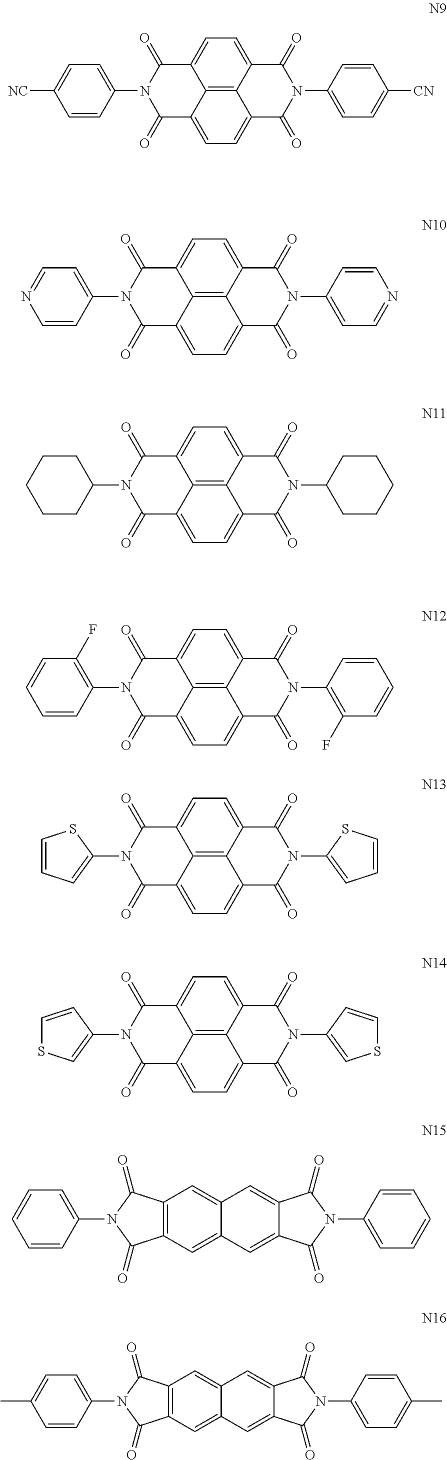

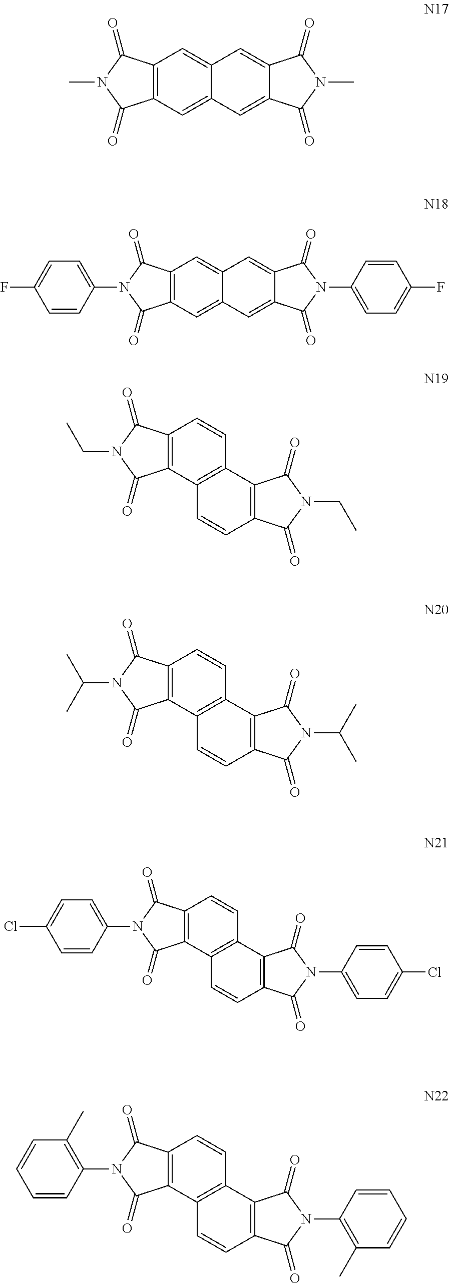

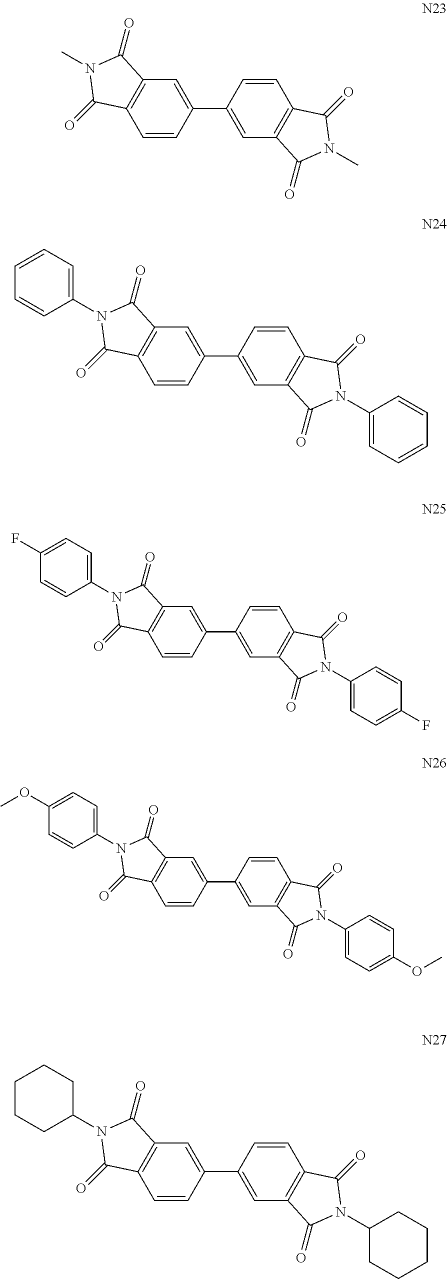

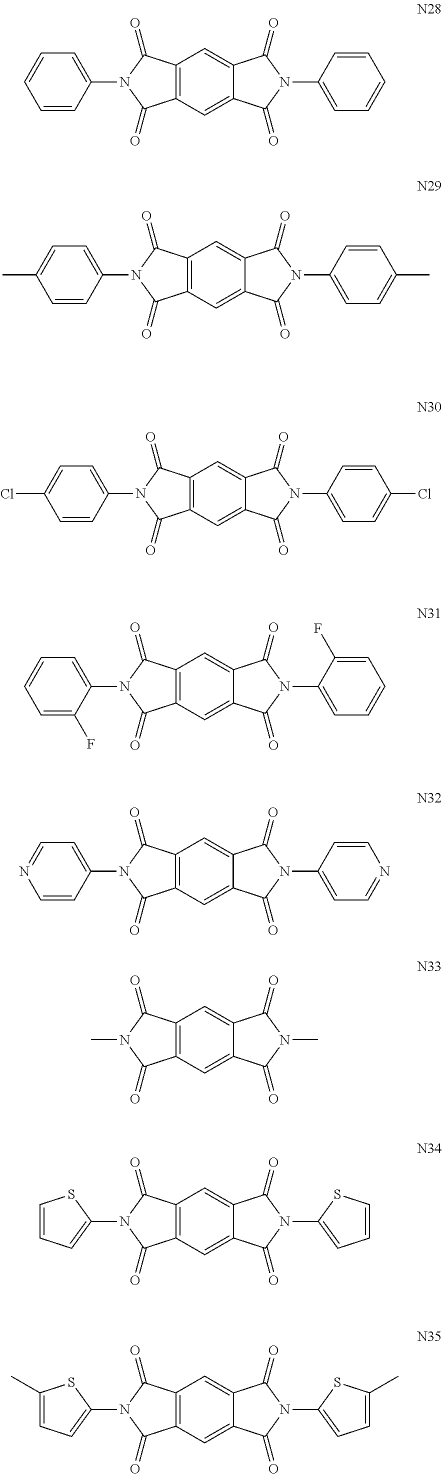

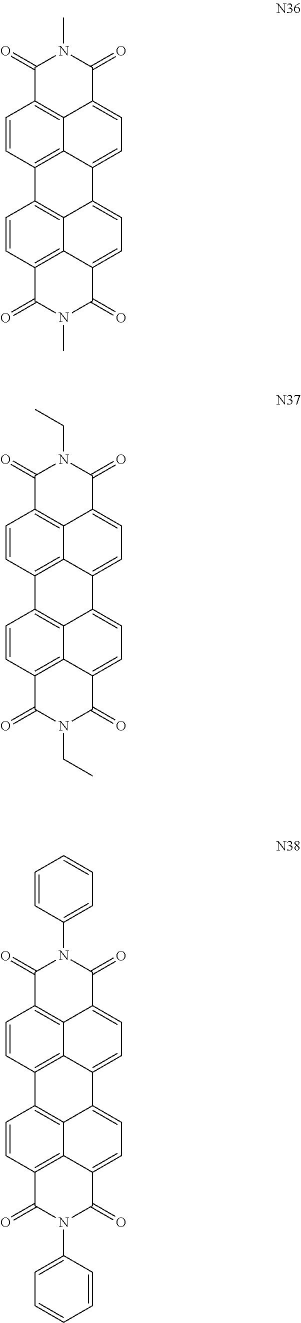 [SMILES: CN1C(=O)c2ccc3c4ccc5c6c(ccc(c7ccc(c2c37)C1=O)c64)C(=O)N(C)C5=O ; CCN1C(=O)c2ccc3c4ccc5c6c(ccc(c7ccc(c2c37)C1=O)c64)C(=O)N(CC)C5=O ; O=C1c2ccc3c4ccc5c6c(ccc(c7ccc(c2c37)C(=O)N1c1ccccc1)c64)C(=O)N(c1ccccc1)C5=O]

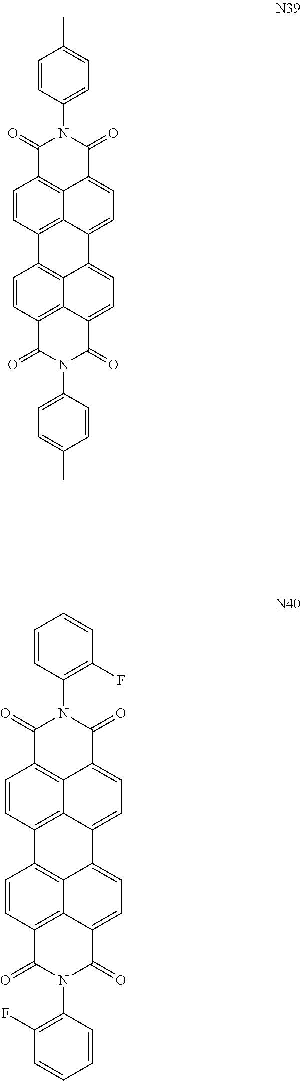 [SMILES: Cc1ccc(N2C(=O)c3ccc4c5ccc6c7c(ccc(c8ccc(c3c48)C2=O)c75)C(=O)N(c2ccc(C)cc2)C6=O)cc1 ; O=C1c2ccc3c4ccc5c6c(ccc(c7ccc(c2c37)C(=O)N1c1ccccc1F)c64)C(=O)N(c1ccccc1F)C5=O]

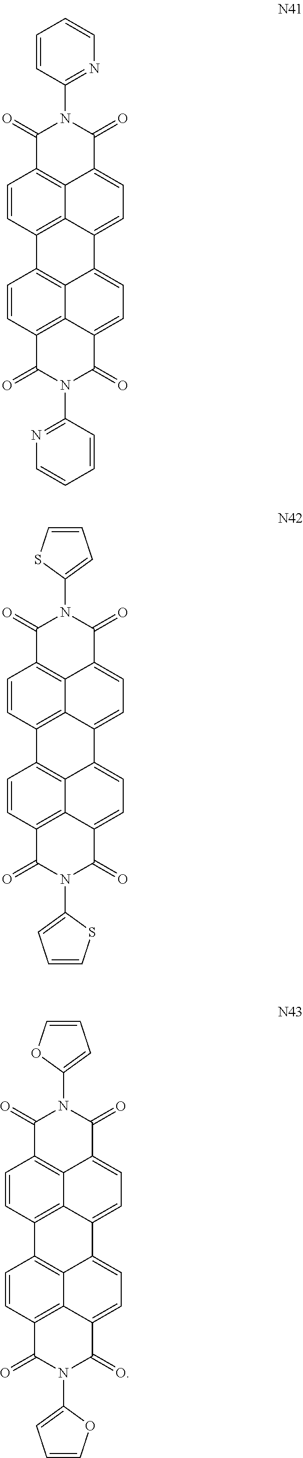

5. The optoelectronic device of claim 1, wherein the photoactive layer comprises the first compound.

6. The optoelectronic device of claim 1, wherein the photoactive layer comprises a first layer adjacent to the first electrode and a second layer adjacent to the second electrode.

7. The optoelectronic device of claim 6, wherein the first layer comprises the first compound.

8. An electronic apparatus comprising:
the optoelectronic device of claim 1;
a light-emitting device next to the optoelectronic device; and
at least one of a color filter, a color conversion layer, a touch screen layer, and a polarizing layer.

9. Electronic equipment comprising the optoelectronic device of claim 1, wherein the electronic equipment is one of a flat panel display, a curved display, a computer monitor, a medical monitor, a television, an advertisement board, an indoor or outdoor lighting and/or signaling light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, a signboard, an automotive sensor, a home sensor, and a solar cell.

10. An organic compound represented by Formula 1: 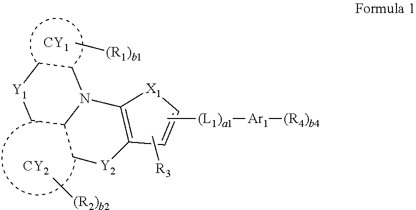
wherein, in Formula 1,
X₁ is selected from oxygen (O), sulfur (S), selenium (Se), and tellurium (Te),
Y₁ and Y₂ are each independently selected from a single bond, *—O—*′, *—S—*′, *—Se—*′, *—C(R₅)(R₆)—*′, *—Si(R₅)(R₆)—*′, *—Ge(R₅)(R₆)—*′, *—N(R₅)—*′, *—P(R₅)—*′, *—C(═O)—*′, *—C(═S)—*′, *—S(═O)—*′, and *—S(═O)₂—*′,
* and *′ each indicate a binding site to a neighboring atom,
L₁ is a C₃-C₆₀ carbocyclic group unsubstituted or substituted with at least one R₁₀ₐ or a C₁-C₆₀ heterocyclic group unsubstituted or substituted with at least one R₁₀ₐ,
a1 is an integer from 1 to 5,
An is a C₄-C₆₀ polycyclic group in which two or more cyclic groups are condensed with each other, and the two or more cyclic groups are each independently a C₃-C₃₀ carbocyclic group or a C₁-C₃₀ heterocyclic group,
rings CY₁ and CY₂ are each independently a C₅-C₆₀ carbocyclic group or a C₂-C₆₀ heterocyclic group,
b1, b2, and b4 are each independently an integer from 0 to 10,
R₁ to R₆ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
two or more of R₁ to R₆ are optionally linked to each other to form a C₈-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, cyano group, a C₁-C₆₀ alkyl group, a C₁-C₆₀ alkoxy group, a phenyl group, a biphenyl group, or any combination thereof.

11. The organic compound of claim 10, wherein the organic compound has a maximum absorption wavelength of about 510 nm to about 565 nm.

12. The organic compound of claim 10, wherein Y₁ and Y₂ are each independently selected from a single bond, *—O—*′, *—S—*′, *—Se—*′, *—C(R₅)(R₆)—*′, *—Si(R₅)(R₆)—*′, and *—N(R₅)—*′.

13. The organic compound of claim 10, wherein L₁ is a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a selenophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluoren-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group, each unsubstituted or substituted with at least one R₁₀ₐ.

14. The organic compound of claim 10, wherein L₁ is a group represented by one of 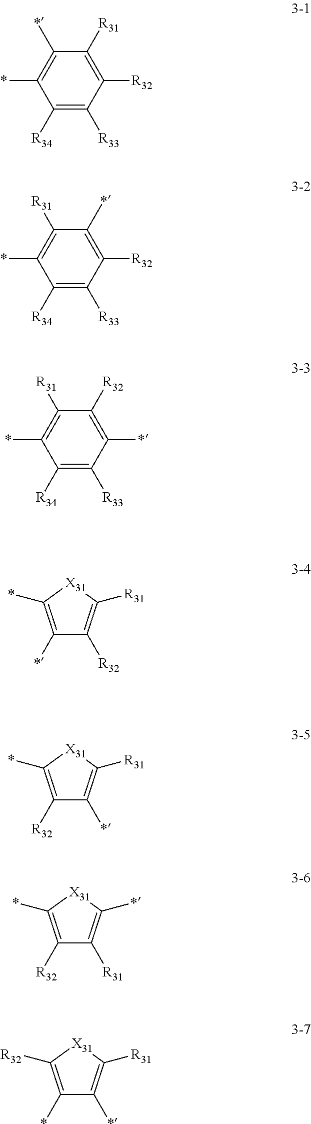 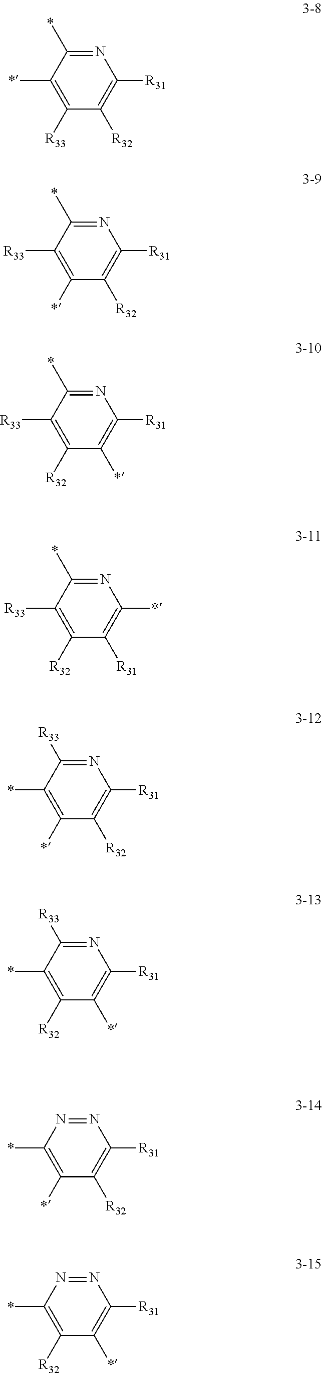 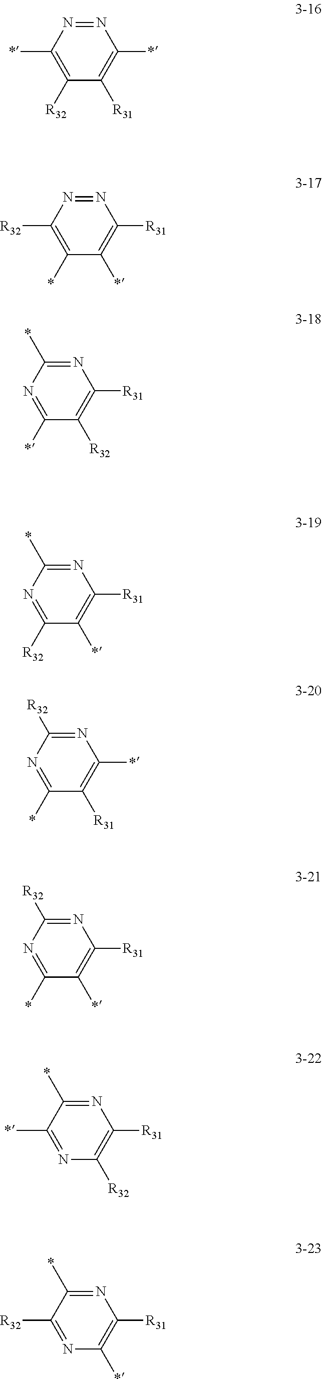 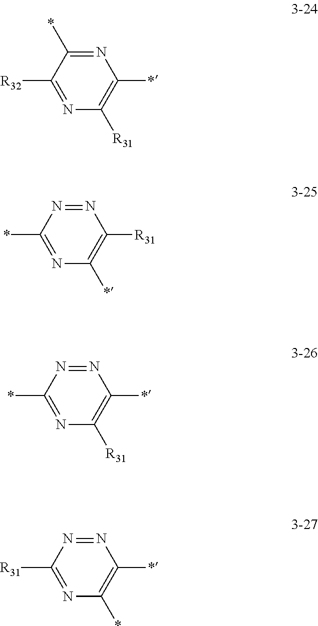
wherein, in Formulae 3-1 to 3-27,
X₃₁ is selected from O, S, Se, and Te,
R₃₁ to R₃₄ are each independently as described in claim 10 in connection with R₁₀ₐ, and
* and *′ each indicate a binding site to a neighboring atom.

15. The organic compound of claim 10, wherein a1 is 1.

16. The organic compound of claim 10, wherein the group represented by *—Ar₁—(R₄)_{b4} is a group represented by one of Formulae 4-1 to 4-6: 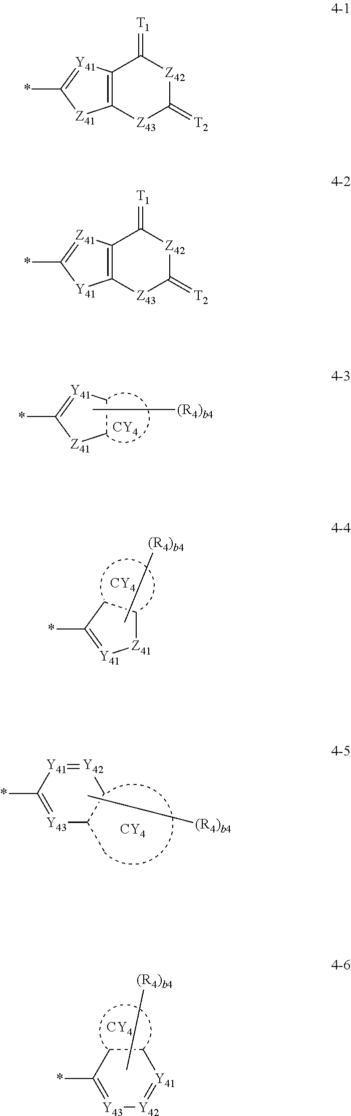
wherein, in Formulae 4-1 to 4-6,
T₁ and T₂ are each independently O, S, Se, and Te,
Z₄₁ to Z₄₃ are each independently selected from O, S, Se, N(R₄₁), C(R₄₁)(R₄₂), C(═O), C(═S), and C═C(R₄₁)(R₄₂),
Y₄₁ to Y₄₃ are each independently N or C(R₄₃),
ring CY₄ is a C₃-C₃₀ carbocyclic group or a C₁-C₃₀ heterocyclic group,
R₄₁ to R₄₃ are each independently as described in claim 10 in connection with R₄,
R₄ and b4 are as described in claim 10, and
* indicates a binding site to a neighboring atom.

17. The organic compound of claim 10, wherein the group represented by *—Ar₁—(R₄)_{b4} is a group represented by one of Formulae 5-1 to 5-15: 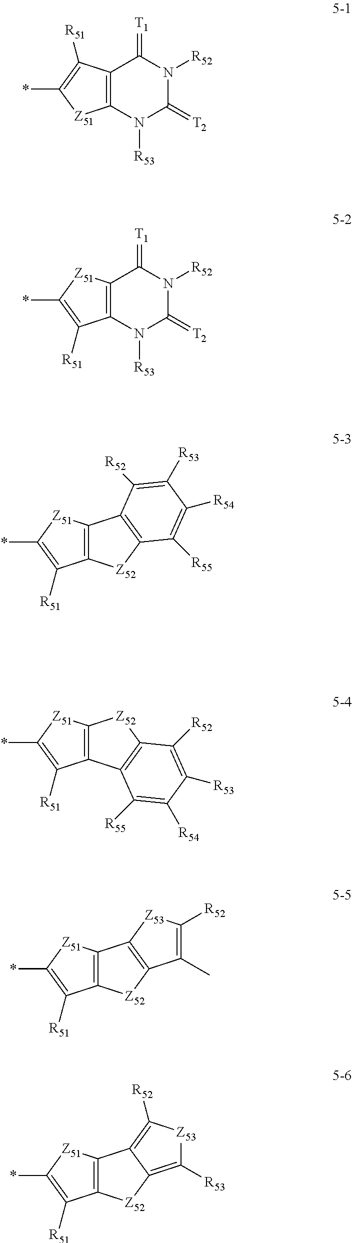 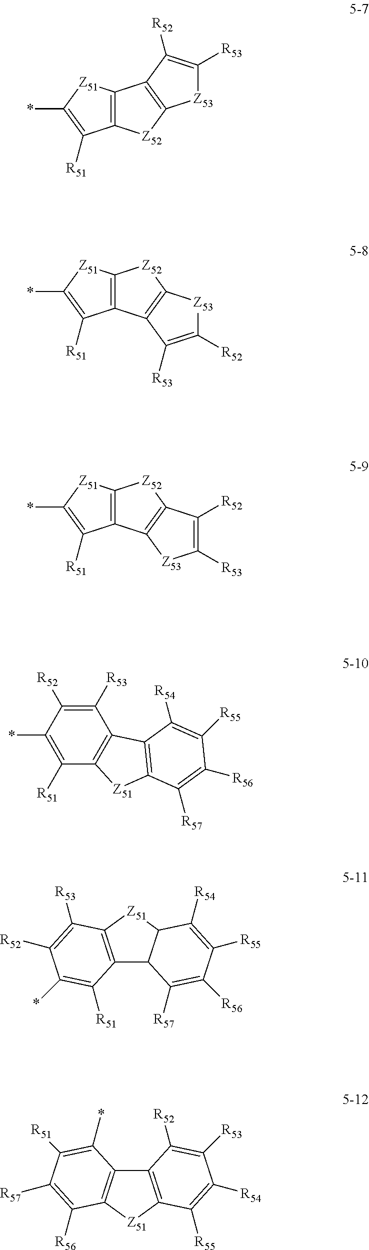 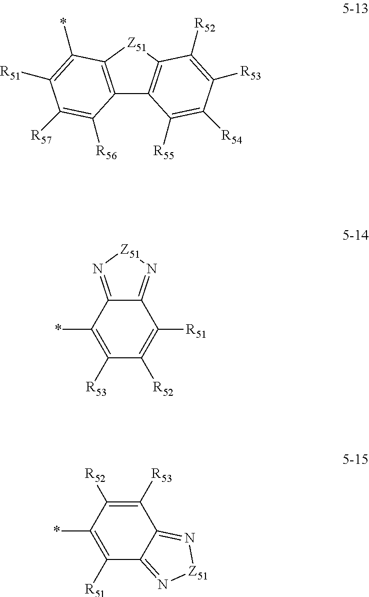
wherein, in Formulae 5-1 to 5-15,
T₁ and T₂ are each independently O, S, Se, and Te,
Z₅₁ to Z₅₃ are each independently selected from O, S, Se, N(R₄₁), C(R₄₁)(R₄₂), C(═O), C(═S), and C═C(R₄₁)(R₄₂),
R₄₁, R₄₂, and R₅₁ to R₅₇ are each independently as described in claim 10 in connection with R₄,
two or more of R₅₁ to R₅₇ are optionally linked to each other to form a C₅-C₃₀ carbocyclic group unsubstituted or substituted with at least one R₁₀ₐ or a C₁-C₃₀ heterocyclic group unsubstituted or substituted with at least one R₁₀ₐ, and
* indicates a binding site to a neighboring atom.

18. The organic compound of claim 10, wherein rings CY₁ and CY₂ are each independently a benzene group or a naphthalene group.

19. The organic compound of claim 10, wherein the organic compound is represented by one of Formulae 1-1 and 1-2: 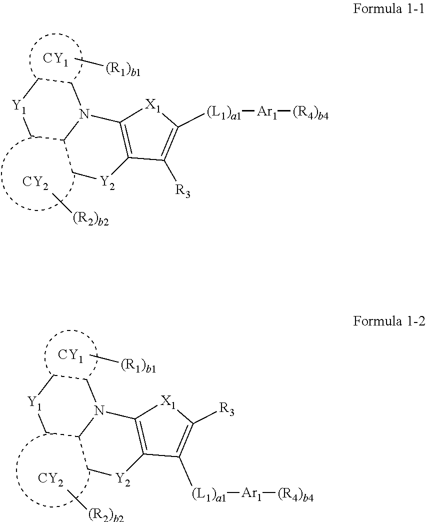
wherein, in Formulae 1-1 and 1-2,
X₁, Y₁, Y₂, L₁, a1, Ar₁, rings CY₁ and CY₂, b1, b2, b4, and R₁ to R₄ are as described in claim 10.

20. The organic compound of claim 10, wherein the organic compound is one of Compounds 1 to 72: 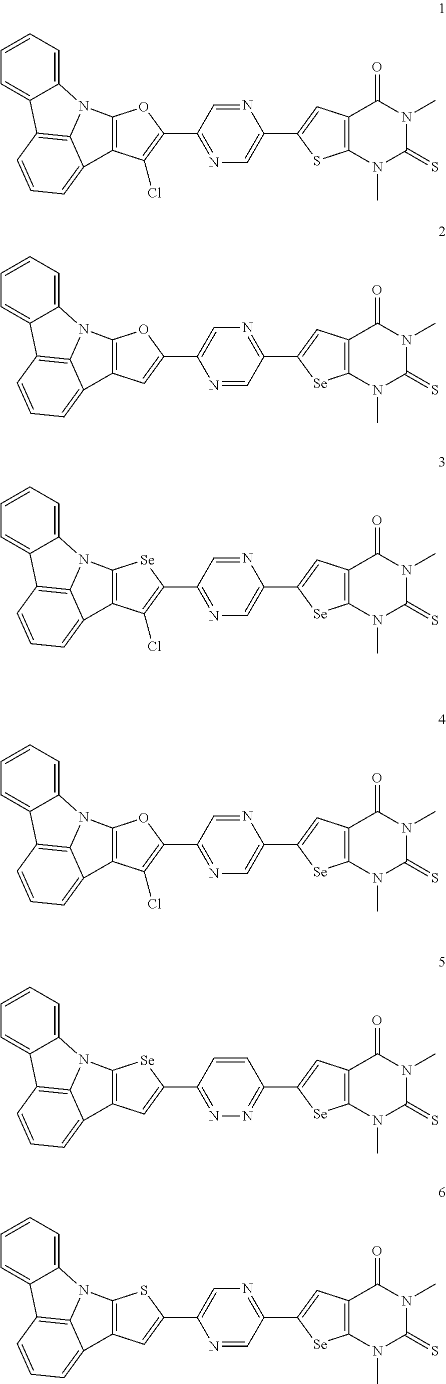 [SMILES: Cn1c(=O)c2cc(-c3cnc(-c4oc5c(c4Cl)c4cccc6c7ccccc7n5c64)cn3)sc2n(C)c1=S ; Cn1c(=O)c2cc(-c3cnc(-c4cc5c6cccc7c8ccccc8n(c5o4)c76)cn3)[se]c2n(C)c1=S ; Cn1c(=O)c2cc(-c3cnc(-c4[se]c5c(c4Cl)c4cccc6c7ccccc7n5c64)cn3)[se]c2n(C)c1=S ; Cn1c(=O)c2cc(-c3cnc(-c4oc5c(c4Cl)c4cccc6c7ccccc7n5c64)cn3)[se]c2n(C)c1=S ; Cn1c(=O)c2cc(-c3ccc(-c4cc5c6cccc7c8ccccc8n(c5[se]4)c76)nn3)[se]c2n(C)c1=S ; Cn1c(=O)c2cc(-c3cnc(-c4cc5c6cccc7c8ccccc8n(c5s4)c76)cn3)[se]c2n(C)c1=S] 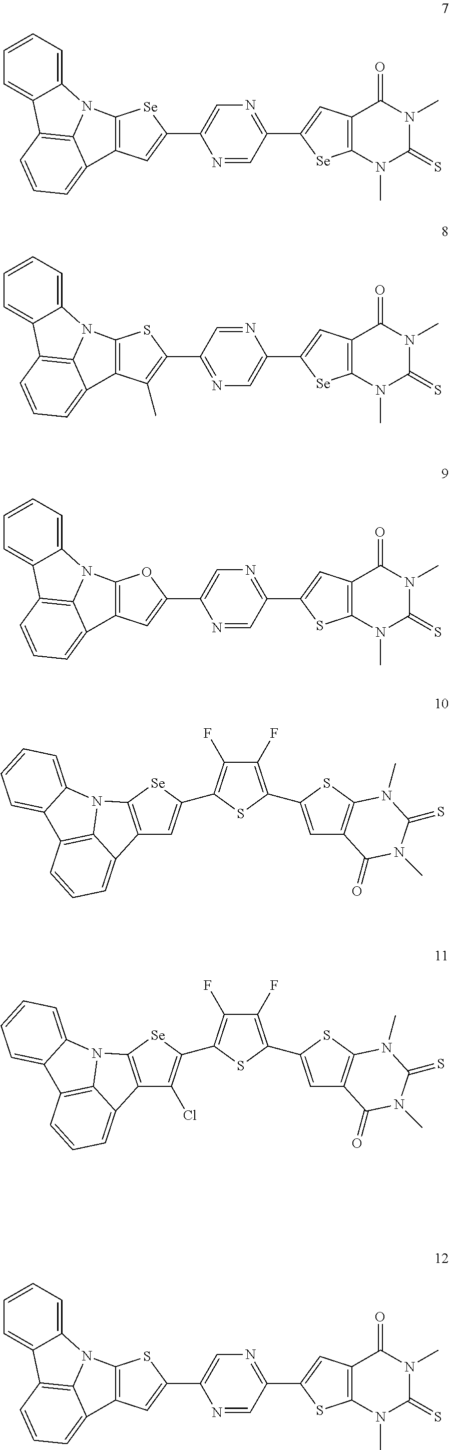 [SMILES: Cn1c(=O)c2cc(-c3cnc(-c4cc5c6cccc7c8ccccc8n(c5[se]4)c76)cn3)[se]c2n(C)c1=S ; Cc1c(-c2cnc(-c3cc4c(=O)n(C)c(=S)n(C)c4[se]3)cn2)sc2c1c1cccc3c4ccccc4n2c31 ; Cn1c(=O)c2cc(-c3cnc(-c4cc5c6cccc7c8ccccc8n(c5o4)c76)cn3)sc2n(C)c1=S ; Cn1c(=O)c2cc(-c3sc(-c4cc5c6cccc7c8ccccc8n(c5[se]4)c76)c(F)c3F)sc2n(C)c1=S ; Cn1c(=O)c2cc(-c3sc(-c4[se]c5c(c4Cl)c4cccc6c7ccccc7n5c64)c(F)c3F)sc2n(C)c1=S ; Cn1c(=O)c2cc(-c3cnc(-c4cc5c6cccc7c8ccccc8n(c5s4)c76)cn3)sc2n(C)c1=S] 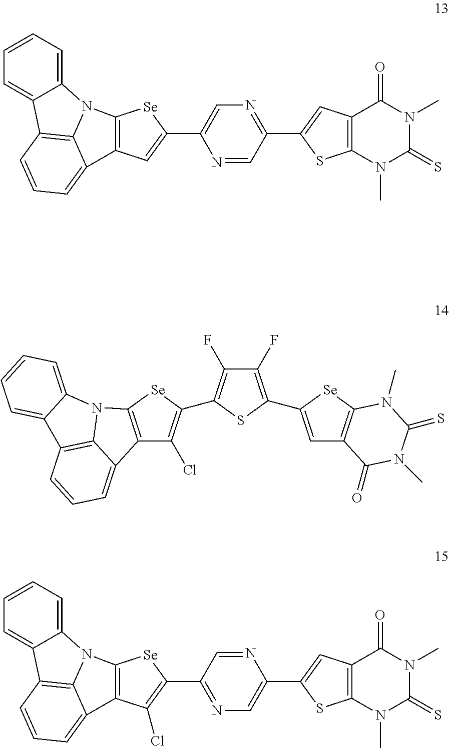 [SMILES: Cn1c(=O)c2cc(-c3cnc(-c4cc5c6cccc7c8ccccc8n(c5[se]4)c76)cn3)sc2n(C)c1=S ; Cn1c(=O)c2cc(-c3sc(-c4[se]c5c(c4Cl)c4cccc6c7ccccc7n5c64)c(F)c3F)[se]c2n(C)c1=S ; Cn1c(=O)c2cc(-c3cnc(-c4[se]c5c(c4Cl)c4cccc6c7ccccc7n5c64)cn3)sc2n(C)c1=S] 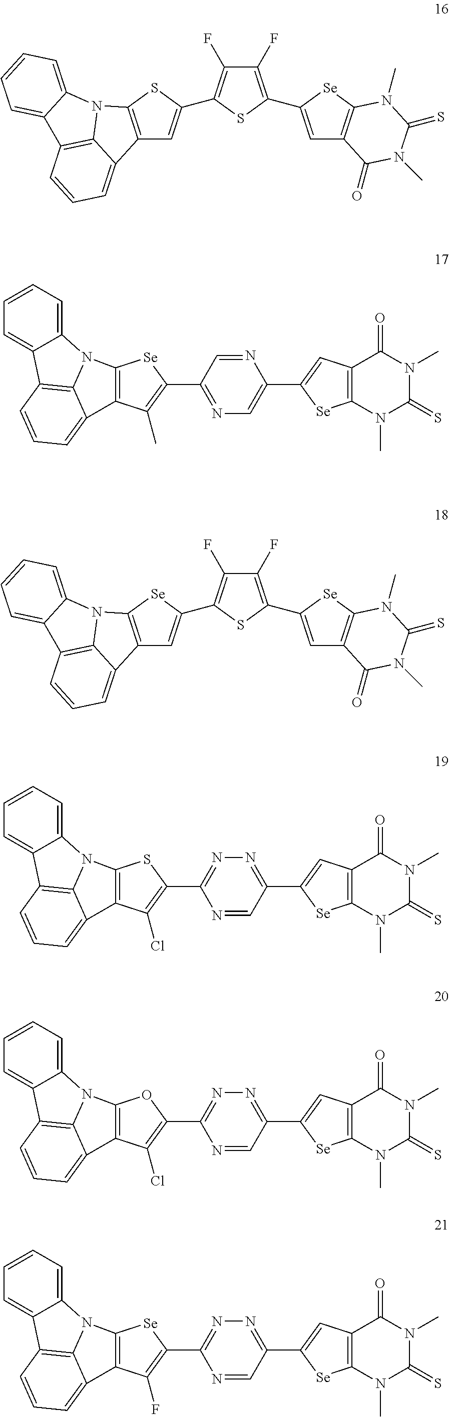 [SMILES: Cn1c(=O)c2cc(-c3sc(-c4cc5c6cccc7c8ccccc8n(c5s4)c76)c(F)c3F)[se]c2n(C)c1=S ; Cc1c(-c2cnc(-c3cc4c(=O)n(C)c(=S)n(C)c4[se]3)cn2)[se]c2c1c1cccc3c4ccccc4n2c31 ; Cn1c(=O)c2cc(-c3sc(-c4cc5c6cccc7c8ccccc8n(c5[se]4)c76)c(F)c3F)[se]c2n(C)c1=S ; Cn1c(=O)c2cc(-c3cnc(-c4sc5c(c4Cl)c4cccc6c7ccccc7n5c64)nn3)[se]c2n(C)c1=S ; Cn1c(=O)c2cc(-c3cnc(-c4oc5c(c4Cl)c4cccc6c7ccccc7n5c64)nn3)[se]c2n(C)c1=S ; Cn1c(=O)c2cc(-c3cnc(-c4[se]c5c(c4F)c4cccc6c7ccccc7n5c64)nn3)[se]c2n(C)c1=S] 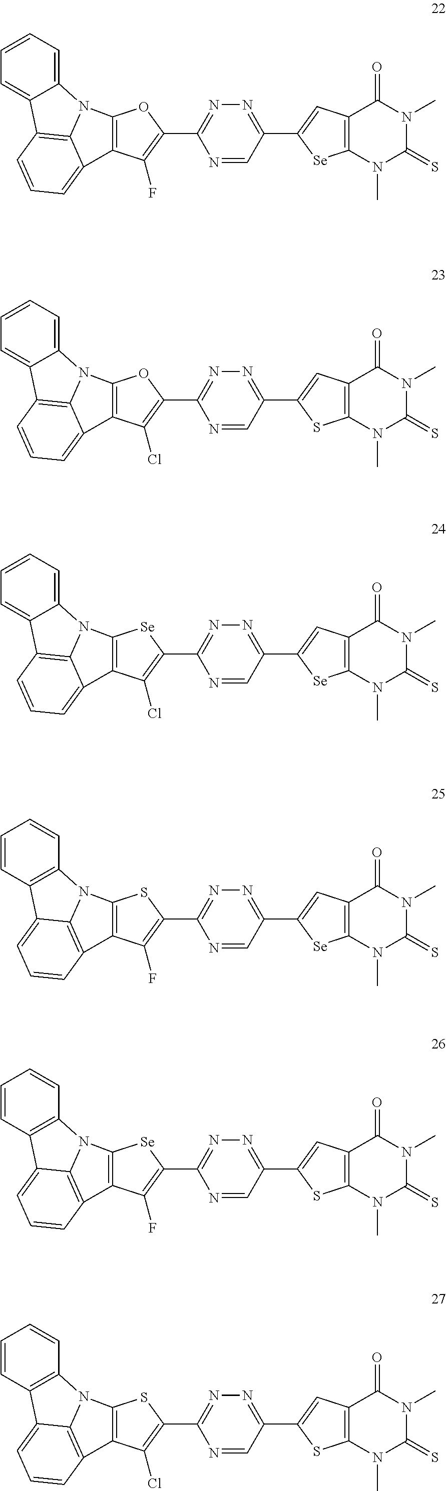 [SMILES: Cn1c(=O)c2cc(-c3cnc(-c4oc5c(c4F)c4cccc6c7ccccc7n5c64)nn3)[se]c2n(C)c1=S ; Cn1c(=O)c2cc(-c3cnc(-c4oc5c(c4Cl)c4cccc6c7ccccc7n5c64)nn3)sc2n(C)c1=S ; Cn1c(=O)c2cc(-c3cnc(-c4[se]c5c(c4Cl)c4cccc6c7ccccc7n5c64)nn3)[se]c2n(C)c1=S ; Cn1c(=O)c2cc(-c3cnc(-c4sc5c(c4F)c4cccc6c7ccccc7n5c64)nn3)[se]c2n(C)c1=S ; Cn1c(=O)c2cc(-c3cnc(-c4[se]c5c(c4F)c4cccc6c7ccccc7n5c64)nn3)sc2n(C)c1=S ; Cn1c(=O)c2cc(-c3cnc(-c4sc5c(c4Cl)c4cccc6c7ccccc7n5c64)nn3)sc2n(C)c1=S] 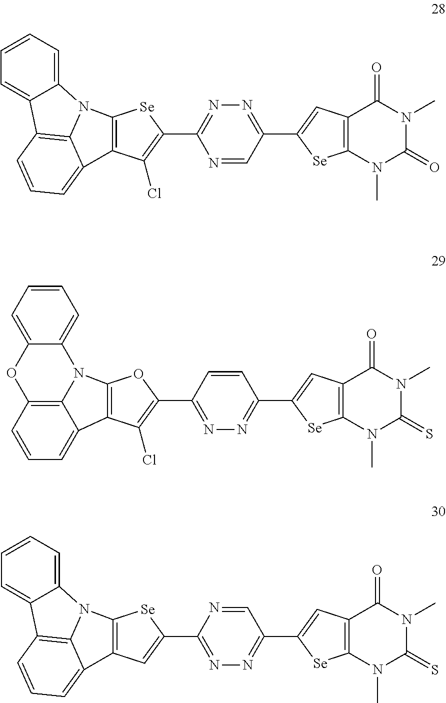 [SMILES: Cn1c(=O)c2cc(-c3cnc(-c4[se]c5c(c4Cl)c4cccc6c7ccccc7n5c64)nn3)[se]c2n(C)c1=O ; Cn1c(=O)c2cc(-c3ccc(-c4oc5c(c4Cl)c4cccc6c4n5-c4ccccc4O6)nn3)[se]c2n(C)c1=S ; Cn1c(=O)c2cc(-c3cnc(-c4cc5c6cccc7c8ccccc8n(c5[se]4)c76)nn3)[se]c2n(C)c1=S] 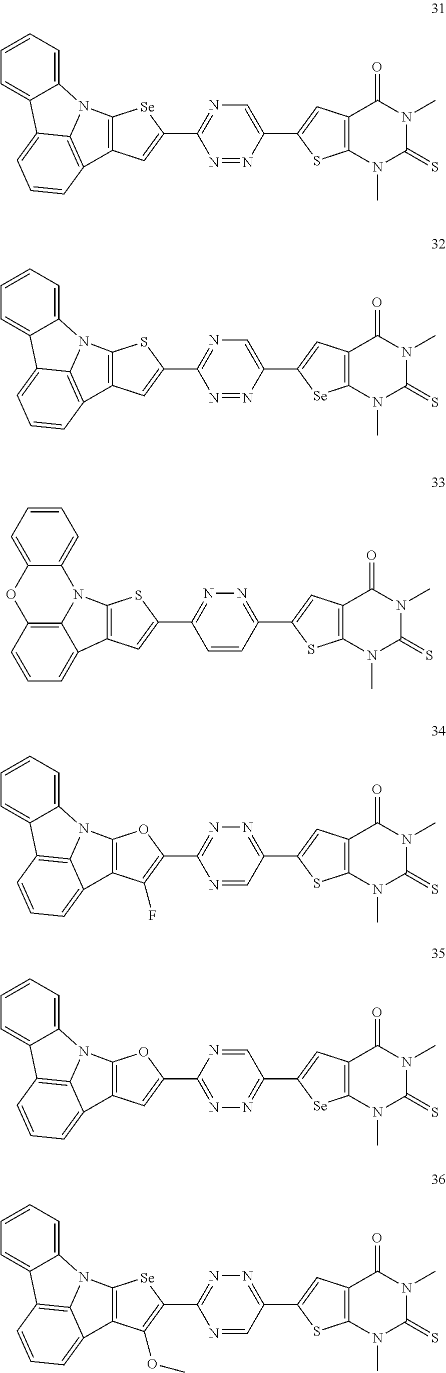 [SMILES: Cn1c(=O)c2cc(-c3cnc(-c4cc5c6cccc7c8ccccc8n(c5[se]4)c76)nn3)sc2n(C)c1=S ; Cn1c(=O)c2cc(-c3cnc(-c4cc5c6cccc7c8ccccc8n(c5s4)c76)nn3)[se]c2n(C)c1=S ; Cn1c(=O)c2cc(-c3ccc(-c4cc5c6cccc7c6n(c5s4)-c4ccccc4O7)nn3)sc2n(C)c1=S ; Cn1c(=O)c2cc(-c3cnc(-c4oc5c(c4F)c4cccc6c7ccccc7n5c64)nn3)sc2n(C)c1=S ; Cn1c(=O)c2cc(-c3cnc(-c4cc5c6cccc7c8ccccc8n(c5o4)c76)nn3)[se]c2n(C)c1=S ; COc1c(-c2ncc(-c3cc4c(=O)n(C)c(=S)n(C)c4s3)nn2)[se]c2c1c1cccc3c4ccccc4n2c31] 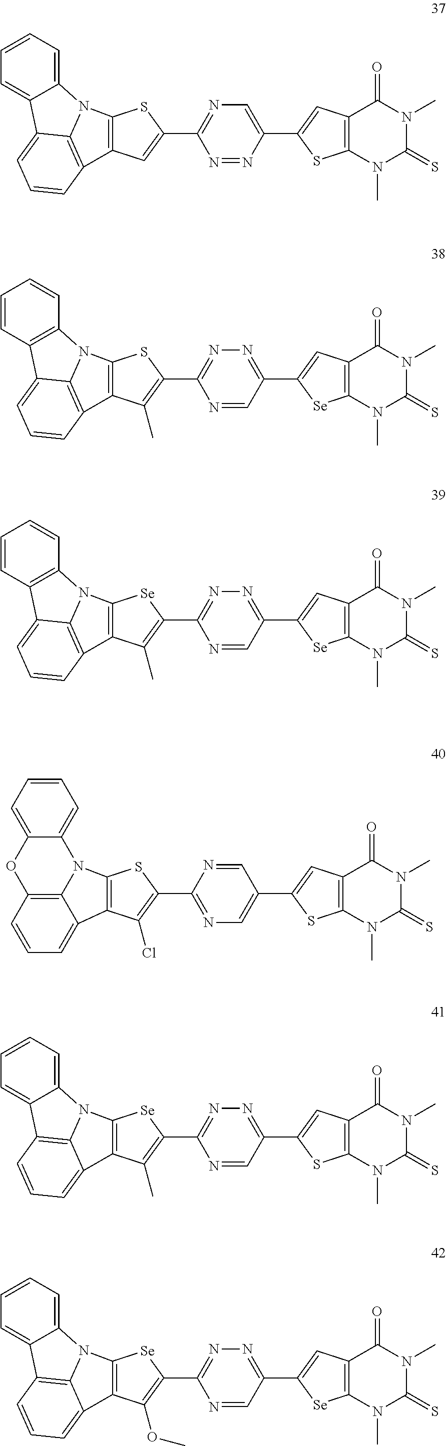 [SMILES: Cn1c(=O)c2cc(-c3cnc(-c4cc5c6cccc7c8ccccc8n(c5s4)c76)nn3)sc2n(C)c1=S ; Cc1c(-c2ncc(-c3cc4c(=O)n(C)c(=S)n(C)c4[se]3)nn2)sc2c1c1cccc3c4ccccc4n2c31 ; Cc1c(-c2ncc(-c3cc4c(=O)n(C)c(=S)n(C)c4[se]3)nn2)[se]c2c1c1cccc3c4ccccc4n2c31 ; Cn1c(=O)c2cc(-c3cnc(-c4sc5c(c4Cl)c4cccc6c4n5-c4ccccc4O6)nc3)sc2n(C)c1=S ; Cc1c(-c2ncc(-c3cc4c(=O)n(C)c(=S)n(C)c4s3)nn2)[se]c2c1c1cccc3c4ccccc4n2c31 ; COc1c(-c2ncc(-c3cc4c(=O)n(C)c(=S)n(C)c4[se]3)nn2)[se]c2c1c1cccc3c4ccccc4n2c31] 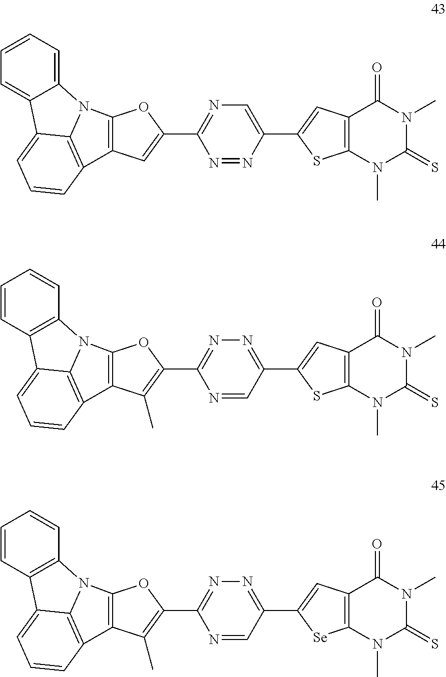 [SMILES: Cn1c(=O)c2cc(-c3cnc(-c4cc5c6cccc7c8ccccc8n(c5o4)c76)nn3)sc2n(C)c1=S ; Cc1c(-c2ncc(-c3cc4c(=O)n(C)c(=S)n(C)c4s3)nn2)oc2c1c1cccc3c4ccccc4n2c31 ; Cc1c(-c2ncc(-c3cc4c(=O)n(C)c(=S)n(C)c4[se]3)nn2)oc2c1c1cccc3c4ccccc4n2c31] 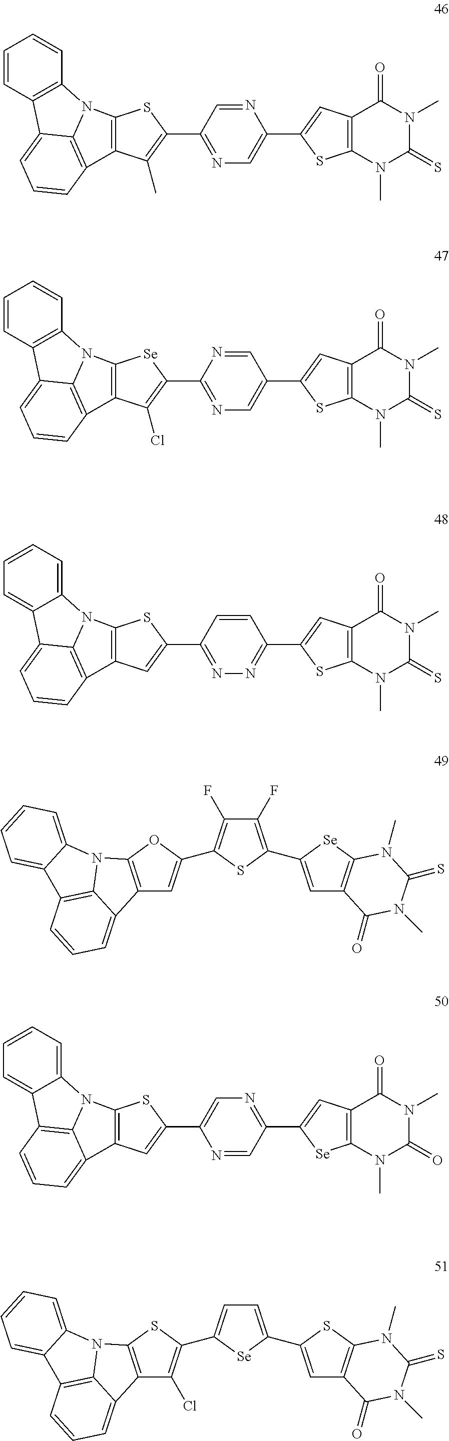 [SMILES: Cc1c(-c2cnc(-c3cc4c(=O)n(C)c(=S)n(C)c4s3)cn2)sc2c1c1cccc3c4ccccc4n2c31 ; Cn1c(=O)c2cc(-c3cnc(-c4[se]c5c(c4Cl)c4cccc6c7ccccc7n5c64)nc3)sc2n(C)c1=S ; Cn1c(=O)c2cc(-c3ccc(-c4cc5c6cccc7c8ccccc8n(c5s4)c76)nn3)sc2n(C)c1=S ; Cn1c(=O)c2cc(-c3sc(-c4cc5c6cccc7c8ccccc8n(c5o4)c76)c(F)c3F)[se]c2n(C)c1=S ; Cn1c(=O)c2cc(-c3cnc(-c4cc5c6cccc7c8ccccc8n(c5s4)c76)cn3)[se]c2n(C)c1=O ; Cn1c(=O)c2cc(-c3ccc(-c4sc5c(c4Cl)c4cccc6c7ccccc7n5c64)[se]3)sc2n(C)c1=S] 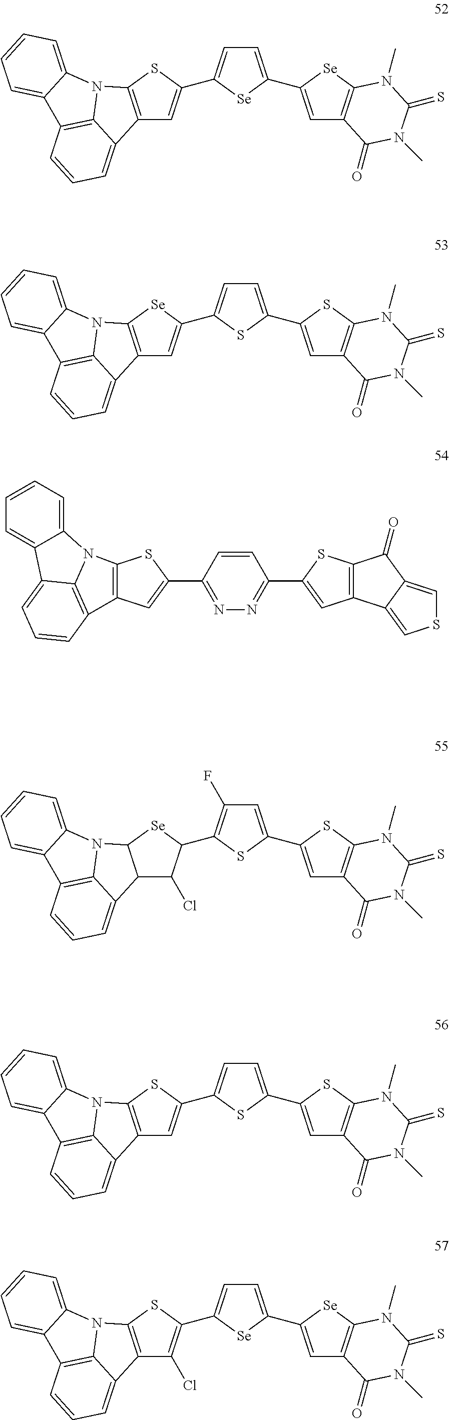 [SMILES: Cn1c(=O)c2cc(-c3ccc(-c4cc5c6cccc7c8ccccc8n(c5s4)c76)[se]3)[se]c2n(C)c1=S ; Cn1c(=O)c2cc(-c3ccc(-c4cc5c6cccc7c8ccccc8n(c5[se]4)c76)s3)sc2n(C)c1=S ; O=C1c2cscc2-c2cc(-c3ccc(-c4cc5c6cccc7c8ccccc8n(c5s4)c76)nn3)sc21 ; Cn1c(=O)c2cc(-c3cc(F)c(C4[Se]C5C(c6cccc7c8ccccc8n5c67)C4Cl)s3)sc2n(C)c1=S ; Cn1c(=O)c2cc(-c3ccc(-c4cc5c6cccc7c8ccccc8n(c5s4)c76)s3)sc2n(C)c1=S ; Cn1c(=O)c2cc(-c3ccc(-c4sc5c(c4Cl)c4cccc6c7ccccc7n5c64)[se]3)[se]c2n(C)c1=S] 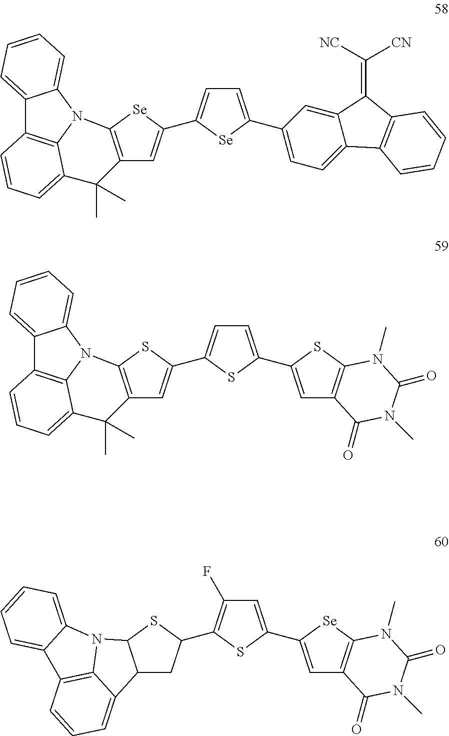 [SMILES: CC1(C)c2cc(-c3ccc(-c4ccc5c(c4)C(=C(C#N)C#N)c4ccccc4-5)[se]3)[se]c2-n2c3ccccc3c3cccc1c32 ; Cn1c(=O)c2cc(-c3ccc(-c4cc5c(s4)-n4c6ccccc6c6cccc(c64)C5(C)C)s3)sc2n(C)c1=O ; Cn1c(=O)c2cc(-c3cc(F)c(C4CC5c6cccc7c8ccccc8n(c67)C5S4)s3)[se]c2n(C)c1=O] 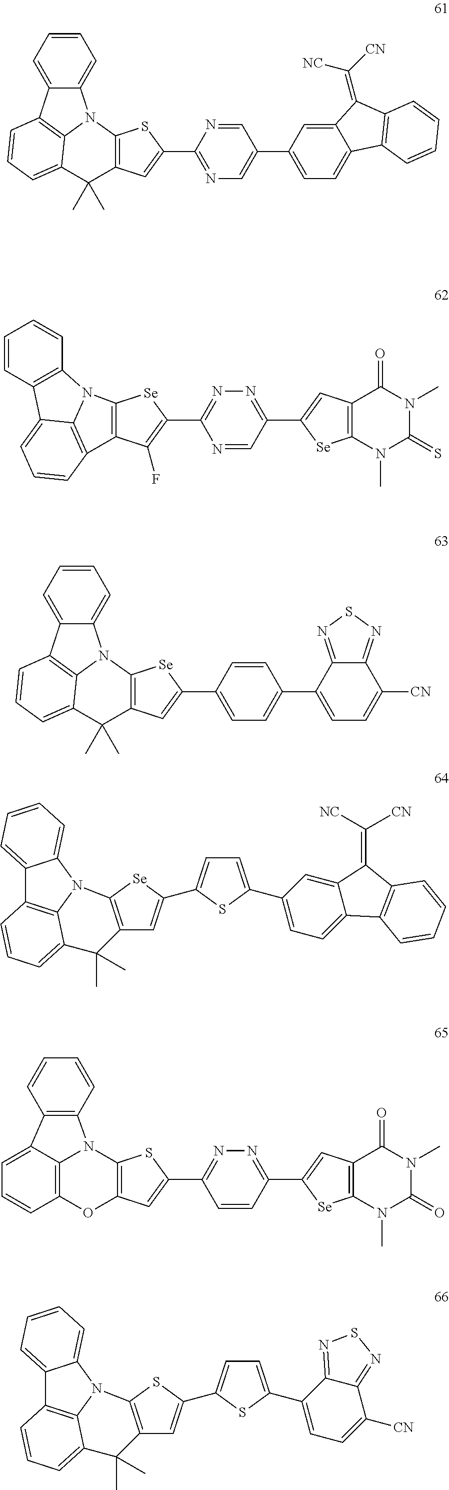 [SMILES: CC1(C)c2cc(-c3ncc(-c4ccc5c(c4)C(=C(C#N)C#N)c4ccccc4-5)cn3)sc2-n2c3ccccc3c3cccc1c32 ; Cn1c(=O)c2cc(-c3cnc(-c4[se]c5c(c4F)c4cccc6c7ccccc7n5c64)nn3)[se]c2n(C)c1=S ; CC1(C)c2cc(-c3ccc(-c4ccc(C#N)c5nsnc45)cc3)[se]c2-n2c3ccccc3c3cccc1c32 ; CC1(C)c2cc(-c3ccc(-c4ccc5c(c4)C(=C(C#N)C#N)c4ccccc4-5)s3)[se]c2-n2c3ccccc3c3cccc1c32 ; Cn1c(=O)c2cc(-c3ccc(-c4cc5c(s4)-n4c6ccccc6c6cccc(c64)O5)nn3)[se]c2n(C)c1=O ; CC1(C)c2cc(-c3ccc(-c4ccc(C#N)c5nsnc45)s3)sc2-n2c3ccccc3c3cccc1c32] 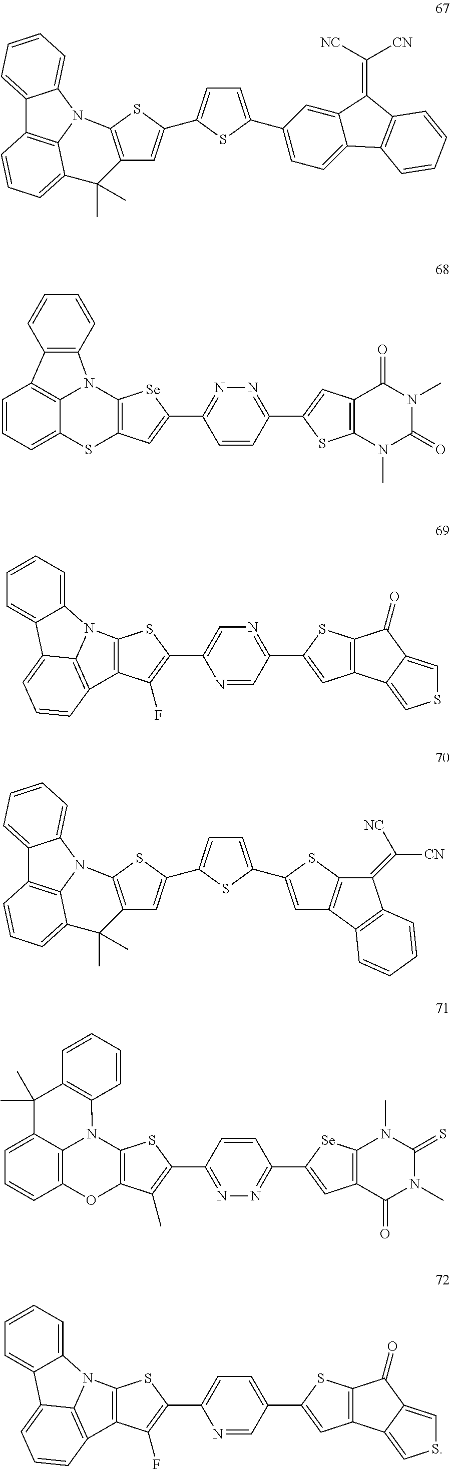 [SMILES: CC1(C)c2cc(-c3ccc(-c4ccc5c(c4)C(=C(C#N)C#N)c4ccccc4-5)s3)sc2-n2c3ccccc3c3cccc1c32 ; Cn1c(=O)c2cc(-c3ccc(-c4cc5c([se]4)-n4c6ccccc6c6cccc(c64)S5)nn3)sc2n(C)c1=O ; O=C1c2cscc2-c2cc(-c3cnc(-c4sc5c(c4F)c4cccc6c7ccccc7n5c64)cn3)sc21 ; CC1(C)c2cc(-c3ccc(-c4cc5c(s4)C(=C(C#N)C#N)c4ccccc4-5)s3)sc2-n2c3ccccc3c3cccc1c32 ; Cc1c(-c2ccc(-c3cc4c(=O)n(C)c(=S)n(C)c4[se]3)nn2)sc2c1Oc1cccc3c1N2c1ccccc1C3(C)C ; O=C1c2cscc2-c2cc(-c3ccc(-c4sc5c(c4F)c4cccc6c7ccccc7n5c64)nc3)sc21]
